SEQUENCE LISTING (103 sequences)

SEQ ID NO: 1 - protein, 612 aa, Trichoderma reesei
MAKLSTLRLASLLSLVSVQVSASVHLLESLEKLPHGWKAAETPSPSSQIVLQVALTQQNIDQLESRLAAVSTPTSSTYGKYLDVDEINSIFAPSDASSSAVESWLQSHGVTSYTKQGSSIWFQTNISTANAMLSTNFHTYSDLTGAKKVRTLKYSIPESLIGHVDLISPTTYFGTTKAMRKLKSSGVSPAADALAARQEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 2 - protein, 590 aa, Trichoderma reesei
SVHLLESLEKLPHGWKAAETPSPSSQIVLQVALTQQNIDQLESRLAAVSTPTSSTYGKYLDVDEINSIFAPSDASSSAVESWLQSHGVTSYTKQGSSIWFQTNISTANAMLSTNFHTYSDLTGAKKVRTLKYSIPESLIGHVDLISPTTYFGTTKAMRKLKSSGVSPAADALAARQEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 3 - protein, 578 aa, Aspergillus oryzae
EAFEKLSAVPKGWHYSSTPKGNTEVCLKIALAQKDAAGFEKTVLEMSDPDHPSYGQHFTTHDEMKRMLLPRDDTVDAVRQWLENGGVTDFTQDADWINFCTTVDTANKLLNAQFKWYVSDVKHIRRLRTLQYDVPESVTPHINTIQPTTRFGKISPKKAVTHSKPSQLDVTALAAAVVAKNISHCDSIITPTCLKELYNIGDYQADANSGSKIAFASYLEEYARYADLENFENYLAPWAKGQNFSVTTFNGGLNDQNSSSDSGEANLDLQYILGVSAPLPVTEFSTGGRGPLVPDLTQPDPNSNSNEPYLEFFQNVLKLDQKDLPQVISTSYGENEQEIPEKYARTVCNLIAQLGSRGVSVLFSSGDSGVGEGCMTNDGTNRTHFPPQFPAACPWVTSVGATFKTTPERGTYFSSGGFSDYWPRPEWQDEAVSSYLETIGDTFKGLYNSSGRAFPDVAAQGMNFAVYDKGTLGEFDGTSASAPAFSAVIALLNDARLRAGKPTLGFLNPWLYKTGRQGLQDITLGASIGCTGRARFGGAPDGGPVVPYASWNATQGWDPVTGLGTPDFAELKKLALGN

SEQ ID NO: 4 - protein, 574 aa, Phaeosphaeria nodorum
EPFEKLFSTPEGWKMQGLATNEQIVKLQIALQQGDVAGFEQHVIDISTPSHPSYGAHYGSHEEMKRMIQPSSETVASVSAWLKAAGINDAEIDSDWVTFKTTVGVANKMLDTKFAWYVSEEAKPRKVLRTLEYSVPDDVAEHINLIQPTTRFAAIRQNHEVAHEIVGLQFAALANNTVNCDATITPQCLKTLYKIDYKADPKSGSKVAFASYLEQYARYNDLALFEKAFLPEAVGQNFSVVQFSGGLNDQNTTQDSGEANLDLQYIVGVSAPLPVTEFSTGGRGPWVADLDQPDEADSANEPYLEFLQGVLKLPQSELPQVISTSYGENEQSVPKSYALSVCNLFAQLGSRGVSVIFSSGDSGPGSACQSNDGKNTTKFQPQYPAACPFVTSVGSTRYLNETATGFSSGGFSDYWKRPSYQDDAVKAYFHHLGEKFKPYFNRHGRGFPDVATQGYGFRVYDQGKLKGLQGTSASAPAFAGVIGLLNDARLKAKKPTLGFLNPLLYSNSDALNDIVLGGSKGCDGHARFNGPPNGSPVIPYAGWNATAGWDPVTGLGTPNFPKLLKAAVPSRYRA

SEQ ID NO: 5 - protein, 590 aa, Trichoderma atroviride
NAAVLLDSLDKVPVGWQAASAPAPSSKITLQVALTQQNIDQLESKLAAVSTPNSSNYGKYLDVDEINQIFAPSSASTAAVESWLKSYGVDYKVQGSSIWFQTDVSTANKMLSTNFHTYTDSVGAKKVRTLQYSVPETLADHIDLISPTTYFGTSKAMRALKIQNAASAVSPLAARQEPSSCKGTIEFENRTFNVFQPDCLRTEYSVNGYKPSAKSGSRIGFGSFLNQSASSSDLALFEKHFGFASQGFSVELINGGSNPQPPTDANDGEANLDAQNIVSFVQPLPITEFIAGGTAPYFPDPVEPAGTPDENEPYLEYYEYLLSKSNKELPQVITNSYGDEEQTVPQAYAVRVCNLIGLMGLRGISILESSGDEGVGASCLATNSTTTPQFNPIFPATCPYVTSVGGTVSFNPEVAWDGSSGGFSYYFSRPWYQEAAVGTYLNKYVSEETKEYYKSYVDFSGRGFPDVAAHSVSPDYPVFQGGELTPSGGTSAASPIVASVIALLNDARLRAGKPALGFLNPLIYGYAYKGFTDITSGQAVGCNGNNTQTGGPLPGAGVIPGAFWNATKGWDPTTGFGVPNFKKLLELVRY

SEQ ID NO: 6 - protein, 580 aa, Arthroderma benhamiae
KPTPGASHKVIEHLDFVPEGWQMVGAADPAAIIDFWLAIERENPEKLYDTIYDVSTPGRAQYGKHLKREELDDLLRPRAETSESIINWLTNGGVNPQHIRDEGDWVRFSTNVKTAETLMNTRFNVFKDNLNSVSKIRTLEYSVPVAISAHVQMIQPTTLFGRQKPQNSLILNPLTKDLESMSVEEFAASQCRSLVTTACLRELYGLGDRVTQARDDNRIGVSGFLEEYAQYRDLELFLSRFEPSAKGFNFSEGLIAGGKNTQGGPGSSTEANLDMQYVVGLSHKAKVTYYSTAGRGPLIPDLSQPSQASNNNEPYLEQLRYLVKLPKNQLPSVLTTSYGDTEQSLPASYTKATCDLFAQLGTMGVSVIFSSGDTGPGSSCQTNDGKNATRFNPIYPASCPFVTSIGGTVGTGPERAVSFSSGGFSDRFPRPQYQDNAVKDYLKILGNQWSGLFDPNGRAFPDIAAQGSNYAVYDKGRMTGVSGTSASAPAMAAIIAQLNDFRLAKGSPVLGFLNPWIYSKGFSGFTDIVDGGSRGCTGYDIYSGLKAKKVPYASWNATKGWDPVTGFGTPNFQALTKVLP

SEQ ID NO: 7 - protein, 580 aa, Fusarium graminearum
KSYSHHAEAPKGWKVDDTARVASTGKQQVFSIALTMQNVDQLESKLLDLSSPDSKNYGQWMSQKDVTTAFYPSKEAVSSVTKWLKSKGVKHYNVNGGFIDFALDVKGANALLDSDYQYYTKEGQTKLRTLSYSIPDDVAEHVQFVDPSTNFGGTLAFAPVTHPSRTLTERKNKPTKSTVDASCQTSITPSCLKQMYNIGDYTPKVESGSTIGFSSFLGESAIYSDVFLFEEKFGIPTQNFTTVLINNGTDDQNTAHKNFGEADLDAENIVGIAHPLPFTQYITGGSPPFLPNIDQPTAADNQNEPYVPFFRYLLSQKEVPAVVSTSYGDEEDSVPREYATMTCNLIGLLGLRGISVIFSSGDIGVGAGCLGPDHKTVEFNAIFPATCPYLTSVGGTVDVTPEIAWEGSSGGFSKYFPRPSYQDKAVKTYMKTVSKQTKKYYGPYTNWEGRGFPDVAGHSVSPNYEVIYAGKQSASGGTSAAAPVWAAIVGLLNDARFRAGKPSLGWLNPLVYKYGPKVLTDITGGYAIGCDGNNTQSGKPEPAGSGIVPGARWNATAGWDPVTGYGTPDFGKLKDLVLSF

SEQ ID NO: 8 - protein, 603 aa, Acremonium alcalophilum
AVVIRAAVLPDAVKLMGKAMPDDIISLQFSLKQQNIDQLETRLRAVSDPSSPEYGQYMSESEVNEFFKPRDDSFAEVIDWVAASGFQDIHLTPQAAAINLAATVETADQLLGANFSWFDVDGTRKLRTLEYTIPDRLADHVDLISPTTYFGRARLDGPRETPTRLDKRQRDPVADKAYFHLKWDRGTSNCDLVITPPCLEAAYNYKNYMPDPNSGSRVSFTSFLEQAAQQSDLTKFLSLTGLDRLRPPSSKPASFDTVLINGGETHQGTPPNKTSEANLDVQWLAAVIKARLPITQWITGGRPPFVPNLRLRHEKDNTNEPYLEFFEYLVRLPARDLPQVISNSYAEDEQTVPEAYARRVCNLIGIMGLRGVTVLTASGDSGVGAPCRANDGSDRLEFSPQFPTSCPYITAVGGTEGWDPEVAWEASSGGFSHYFLRPWYQANAVEKYLDEELDPATRAYYDGNGFVQFAGRAYPDLSAHSSSPRYAYIDKLAPGLTGGTSASCPVVAGIVGLLNDARLRRGLPTMGFINPWLYTRGFEALQDVTGGRASGCQGIDLQRGTRVPGAGIIPWASWNATPGWDPATGLGLPDFWAMRGLALGRGT

SEQ ID NO: 9 - protein, 614 aa, Sodiomyces alkalinus
AVVIRAAPLPESVKLVRKAAAEDGINLQLSLKRQNMDQLEKFLRAVSDPFSPKYGQYMSDAEVHEIFRPTEDSFDQVIDWLTKSGFGNLHITPQAAAINVATTVETADQLFGANFSWFDVDGTPKLRTGEYTIPDRLVEHVDLVSPTTYFGRMRPPPRGDGVNDWITENSPEQPAPLNKRDTKTESDQARDHPSWDSRTPDCATIITPPCLETAYNYKGYIPDPKSGSRVSFTSFLEQAAQQADLTKFLSLTRLEGFRTPASKKKTFKTVLINGGESHEGVHKKSKTSEANLDVQWLAAVTQTKLPITQWITGGRPPFVPNLRIPTPEANTNEPYLEFLEYLFRLPDKDLPQVISNSYAEDEQSVPEAYARRVCGLLGIMGLRGVTVLTASGDSGVGAPCRANDGSGREEFSPQFPSSCPYITTVGGTQAWDPEVAWKGSSGGFSNYFPRPWYQVAAVEKYLEEQLDPAAREYYEENGFVRFAGRAFPDLSAHSSSPKYAYVDKRVPGLTGGTSASCPVVAGIVGLLNDARLRRGLPTMGFINPWLYAKGYQALEDVTGGAAVGCQGIDIQTGKRVPGAGIIPGASWNATPDWDPATGLGLPNFWAMRELALED

SEQ ID NO: 10 - protein, 575 aa, Aspergillus kawachii
VVHEKLAAVPSGWHHLEDAGSDHQISLSIALARKNLDQLESKLKDLSTPGESQYGQWLDQEEVDTLFPVASDKAVISWLRSANITHIARQGSLVNFATTVDKVNKLLNTTFAYYQRGSSQRLRTTEYSIPDDLVDSIDLISPTTFFGKEKTSAGLTQRSQKVDNHVAKRSNSSSCADTITLSCLKEMYNFGNYTPSASSGSKLGFASFLNESASYSDLAKFERLFNLPSQNFSVELINGGVNDQNQSTASLTEADLDVELLVGVGHPLPVTEFITSGEPPFIPDPDEPSAADNENEPYLQYYEYLLSKPNSALPQVISNSYGDDEQTVPEYYAKRVCNLIGLVGLRGISVLESSGDEGIGSGCRTTDGTNSTQFNPIFPATCPYVTAVGGTMSYAPEIAWEASSGGFSNYFERAWFQKEAVQNYLANHITNETKQYYSQFANFSGRGFPDVSAHSFEPSYEVIFYGARYGSGGTSAACPLFSALVGMLNDARLRAGKSTLGFLNPLLYSKGYKALTDVTAGQSIGCNGIDPQSDEAVAGAGIIPWAHWNATVGWDPVTGLGLPDFEKLRQLVLSL

SEQ ID NO: 11 - protein, 582 aa, Talaromyces stipitatus
AAALVGHESLAALPVGWDKVSTPAAGTNIQLSVALALQNIEQLEDHLKSVSTPGSASYGQYLDSDGIAAQYGPSDASVEAVTNWLKEAGVTDIYNNGQSIHFATSVSKANSLLGADFNYYSDGSATKLRTLAYSVPSDLKEAIDLVSPTTYFGKTTASRSIQAYKNKRASTTSKSGSSSVQVSASCQTSITPACLKQMYNVGNYTPSVAHGSRVGFGSFLNQSAIFDDLFTYEKVNDIPSQNFTKVIIANASNSQDASDGNYGEANLDVQNIVGISHPLPVTEFLTGGSPPFVASLDTPTNQNEPYIPYYEYLLSQKNEDLPQVISNSYGDDEQSVPYKYAIRACNLIGLTGLRGISVLESSGDLGVGAGCRSNDGKNKTQFDPIFPATCPYVTSVGGTQSVTPEIAWVASSGGFSNYFPRTWYQEPAIQTYLGLLDDETKTYYSQYTNFEGRGFPDVSAHSLTPDYQVVGGGYLQPSGGTSAASPVFAGIIALLNDARLAAGKPTLGFLNPFFYLYGYKGLNDITGGQSVGCNGINGQTGAPVPGGGIVPGAAWNSTTGWDPATGLGTPDFQKLKELVLSF

SEQ ID NO: 12 - protein, 579 aa, Fusarium oxysporum
KSFSHHAEAPQGWQVQKTAKVASNTQHVFSLALTMQNVDQLESKLLDLSSPDSANYGNWLSHDELTSTFSPSKEAVASVTKWLKSKGIKHYKVNGAFIDFAADVEKANTLLGGDYQYYTKDGQTKLRTLSYSIPDDVAGHVQFVDPSTNFGGTVAFNPVPHPSRTLQERKVSPSKSTVDASCQTSITPSCLKQMYNIGDYTPDAKSGSEIGFSSFLGQAAIYSDVFKFEELFGIPKQNYTTILINNGTDDQNTAHGNFGEANLDAENIVGIAHPLPFKQYITGGSPPFVPNIDQPTEKDNQNEPYVPFFRYLLGQKDLPAVISTSYGDEEDSVPREYATLTCNMIGLLGLRGISVIFSSGDIGVGSGCLAPDYKTVEFNAIFPATCPYLTSVGGTVDVTPEIAWEGSSGGFSKYFPRPSYQDKAIKKYMKTVSKETKKYYGPYTNWEGRGFPDVAGHSVAPDYEVIYNGKQARSGGTSAAAPVWAAIVGLLNDARFKAGKKSLGWLNPLIYKHGPKVLTDITGGYAIGCDGNNTQSGKPEPAGSGLVPGARWNATAGWDPTTGYGTPNFQKLKDLVLSL

SEQ ID NO: 13 - protein, 590 aa, Trichoderma virens
SVLVESLEKLPHGWKAASAPSPSSQITLQVALTQQNIDQLESRLAAVSTPNSKTYGNYLDLDEINEIFAPSDASSAAVESWLHSHGVTKYTKQGSSIWFQTEVSTANAMLSTNFHTYSDAAGVKKLRTLQYSIPESLVGHVDLISPTTYFGTSNAMRALRSKSVASVAQSVAARQEPSSCKGTLVFEGRTFNVFQPDCLRTEYNVNGYTPSAKSGSRIGFGSFLNQSASFSDLALFEKHFGFSSQNFSVVLINGGTDLPQPPSDDNDGEANLDVQNILTIAHPLPITEFITAGSPPYFPDPVEPAGTPDENEPYLQYFEYLLSKPNRDLPQVITNSYGDEEQTVPQAYAVRVCNLIGLMGLRGISILESSGDEGVGASCVATNSTTPQFNPIFPATCPYVTSVGGTVNFNPEVAWDGSSGGFSYYFSRPWYQEEAVGNYLEKHVSAETKKYYGPYVDFSGRGFPDVAAHSVSPDYPVFQGGQLTPSGGTSAASPVVASIIALLNDARLREGKPTLGFLNPLIYQYAYKGFTDITSGQSDGCNGNNTQTDAPLPGAGVVLGAHWNATKGWDPTTGFGVPNFKKLLELIRYI

SEQ ID NO: 14 - protein, 569 aa, Trichoderma atroviride
AVLVESLKQVPNGWNAVSTPDPSTSIVLQIALAQQNIDELEWRLAAVSTPNSGNYGKYLDIGEIEGIFAPSNASYKAVASWLQSHGVKNFVKQAGSIWFYTTVSTANKMLSTDFKHYSDPVGIEKLRTLQYSIPEELVGHVDLISPTTYFGNNHPATARTPNMKAINVTYQIFHPDCLKTKYGVDGYAPSPRCGSRIGFGSFLNETASYSDLAQFEKYFDLPNQNLSTLLINGAIDVQPPSNKNDSEANMDVQTILTFVQPLPITEFVVAGIPPYIPDAALPIGDPVQNEPWLEYFEFLMSRTNAELPQVIANSYGDEEQTVPQAYAVRVCNQIGLLGLRGISVIASSGDTGVGMSCMASNSTTPQFNPMFPASCPYITTVGGTQHLDNEIAWELSSGGFSNYFTRPWYQEDAAKTYLERHVSTETKAYYERYANFLGRGFPDVAALSLNPDYPVIIGGELGPNGGTSAAAPVVASIIALLNDARLCLGKPALGFLNPLIYQYADKGGFTDITSGQSWGCAGNTTQTGPPPPGAGVIPGAHWNATKGWDPVTGFGTPNFKKLLSLALSV

SEQ ID NO: 15 - protein, 565 aa, Agaricus bisporus
SPLARRWDDFAEKHAWVEVPRGWEMVSEAPSDHTFDLRIGVKSSGMEQLIENLMQTSDPTHSRYGQHLSKEELHDFVQPHPDSTGAVEAWLEDFGISDDFIDRTGSGNWVTVRVSVAQAERMLGTKYNVYRHSESGESVVRTMSYSLPSELHSHIDVVAPTTYFGTMKSMRVTSFLQPEIEPVDPSAKPSAAPASCLSTTVITPDCLRDLYNTADYVPSATSRNAIGIAGYLDRSNRADLQTFFRRFRPDAVGFNYTTVQLNGGGDDQNDPGVEANLDIQYAAGIAFPTPATYWSTGGSPPFIPDTQTPTNTNEPYLDWINFVLGQDEIPQVISTSYGDDEQTVPEDYATSVCNLFAQLGSRGVTVFFSSGDFGVGGGDCLTNDGSNQVLFQPAFPASCPFVTAVGGTVRLDPEIAVSFSGGGFSRYFSRPSYQNQTVAQFVSNLGNTFNGLYNKNGRAYPDLAAQGNGFQVVIDGIVRSVGGTSASSPTVAGIFALLNDFKLSRGQSTLGFINPLIYSSATSGFNDIRAGTNPGCGTRGFTAGTGWDPVTGLGTPDFLRLQGLI

SEQ ID NO: 16 - protein, 583 aa, Magnaporthe oryzae
RVFDSLPHPPRGWSYSHAAESTEPLTLRIALRQQNAAALEQVVLQVSNPRHANYGQHLTRDELRSYTAPTPRAVRSVTSWLVDNGVDDYTVEHDWVTLRTTVGAADRLLGADFAWYAGPGETLQLRTLSYGVDDSVAPHVDLVQPTTRFGGPVGQASHIFKQDDFDEQQLKTLSVGFQVMADLPANGPGSIKAACNESGVTPLCLRTLYRVNYKPATTGNLVAFASFLEQYARYSDQQAFTQRVLGPGVPLQNFSVETVNGGANDQQSKLDSGEANLDLQYVMAMSHPIPILEYSTGGRGPLVPTLDQPNANNSSNEPYLEFLTYLLAQPDSAIPQTLSVSYGEEEQSVPRDYAIKVCNMFMQLGARGVSVMFSSGDSGPGNDCVRASDNATFFGSTFPAGCPYVTSVGSTVGFEPERAVSFSSGGFSIYHARPDYQNEVVPKYIESIKASGYEKFFDGNGRGIPDVAAQGARFVVIDKGRVSLISGTSASSPAFAGMVALVNAARKSKDMPALGFLNPMLYQNAAAMTDIVNGAGIGCRKQRTEFPNGARFNATAGWDPVTGLGTPLFDKLLAVGAPGVPNA

SEQ ID NO: 17 - protein, 594 aa, Togninia minima
SDVVLESLREVPQGWKRLRDADPEQSIKLRIALEQPNLDLFEQTLYDISSPDHPKYGQHLKSHELRDIMAPREESTAAVIAWLQDAGLSGSQIEDDSDWINIQTTVAQANDMLNTTFGLFAQEGTEVNRIRALAYSVPEEIVPHVKMIAPIIRFGQLRPQMSHIFSHEKVEETPSIGTIKAAAIPSVDLNVTACNASITPECLRALYNVGDYEADPSKKSLFGVCGYLEQYAKHDQLAKFEQTYAPYAIGADFSVVTINGGGDNQTSTIDDGEANLDMQYAVSMAYKTPITYYSTGGRGPLVPDLDQPDPNDVSNEPYLDFVSYLLKLPDSKLPQTITTSYGEDEQSVPRSYVEKVCTMFGALGARGVSVIFSSGDTGVGSACQTNDGKNTTRFLPIFPAACPYVTSVGGTRYVDPEVAVSFSSGGFSDIFPTPLYQKGAVSGYLKILGDRWKGLYNPHGRGFPDVSGQSVRYHVFDYGKDVMYSGTSASAPMFAALVSLLNNARLAKKLPPMGFLNPWLYTVGFNGLTDIVHGGSTGCTGTDVYSGLPTPFVPYASWNATVGWDPVTGLGTPLFDKLLNLSTPNFHLPHIGGH

SEQ ID NO: 18 - protein, 595 aa, Bipolaris maydis
STTSHVEGEVVERLHGVPEGWSQVGAPNPDQKLRFRIAVRSADSELFERTLMEVSSPSHPRYGQHLKRHELKDLIKPRAKSTSNILNWLQESGIEARDIQNDGEWISFYAPVKRAEQMMSTTFKTYQNEARANIKKIRSLDYSVPKHIRDDIDIIQPTTRFGQIQPERSQVFSQEEVPFSALVVNATCNKKITPDCLANLYNFKDYDASDANVTIGVSGFLEQYARFDDLKQFISTFQPKAAGSTFQVTSVNAGPFDQNSTASSVEANLDIQYTTGLVAPDIETRYFTVPGRGILIPDLDQPTESDNANEPYLDYFTYLNNLEDEELPDVLTTSYGESEQSVPAEYAKKVCNLIGQLGARGVSVIFSSGDTGPGSACQTNDGKNTTRFLPIFPASCPYVTSVGGTVGVEPEKAVSFSSGGFSDLWPRPAYQEKAVSEYLEKLGDRWNGLYNPQGRGFPDVAAQGQGFQVFDKGRLISVGGTSASAPVFASVVALLNNARKAAGMSSLGFLNPWIYEQGYKGLTDIVAGGSTGCTGRSIYSGLPAPLVPYASWNATEGWDPVTGYGTPDFKQLLTLATAPKSGERRVRRGGLGGQA

SEQ ID NO: 19 - protein, 613 aa, Aspergillus kawachii
MLSSFLSQGAAVSLALLSLLPSPVAAEIFEKLSGVPNGWRYANNPHGNEVIRLQIALQQHDVAGFEQAVMDMSTPGHADYGKHFRTHDEMKRMLLPSDTAVDSVRDWLESAGVHNIQVDADWVKFHTTVNKANALLDADFKWYVSEAKHIRRLRTLQYSIPDALVSHINMIQPTTRFGQIQPNRATMRSKPKHADETFLTAATLAQNTSHCDSIITPHCLKQLYNIGDYQADPKSGSKVGFASYLEEYARYADLERFEQHLAPNAIGQNFSVVQFNGGLNDQLSLSDSGEANLDLQYILGVSAPVPVTEYSTGGRGELVPDLSSPDPNDNSNEPYLDFLQGILKLDNSDLPQVISTSYGEDEQTIPVPYARTVCNLYAQLGSRGVSVIFSSGDSGVGAACLTNDGTNRTHFPPQFPASCPWVTSVGATSKTSPEQAVSFSSGGFSDLWPRPSYQQAAVQTYLTQHLGNKFSGLFNASGRAFPDVAAQGVNYAVYDKGMLGQFDGTSCSAPTFSGVIALLNDARLRAGLPVMGFLNPFLYGVGSESGALNDIVNGGSLGCDGRNRFGGTPNGSPVVPFASWNATTGWDPVSGLGTPDFAKLRGVALGEAKAYGN

SEQ ID NO: 20 - protein, 658 aa, Aspergillus nidulans
MAATGRFTAFWNVASVPALIGILPLAGSHLRAVLCPVCIWRHSKAVCAPDTLQAMRAFTRVTAISLAGFSCFAAAAAAAFESLRAVPDGWIYESTPDPNQPLRLRIALKQHNVAGFEQALLDMSTPGHSSYGQHFGSYHEMKQLLLPTEEASSSVRDWLSAAGVEFEQDADWINFRTTVDQANALLDADFLWYTTTGSTGNPTRILRTLSYSVPSELAGYVNMIQPTTRFGGTHANRATVRAKPIFLETNRQLINAISSGSLEHCEKAITPSCLADLYNTEGYKASNRSGSKVAFASFLEEYARYDDLAEFEETYAPYAIGQNFSVISINGGLNDQDSTADSGEANLDLQYIIGVSSPLPVTEFTTGGRGKLIPDLSSPDPNDNTNEPFLDFLEAVLKLDQKDLPQVISTSYGEDEQTIPEPYARSVCNLYAQLGSRGVSVLFSSGDSGVGAACQTNDGKNTTHFPPQFPASCPWVTAVGGTNGTAPESGVYFSSGGFSDYWARPAYQNAAVESYLRKLGSTQAQYFNRSGRAFPDVAAQAQNFAVVDKGRVGLFDGTSCSSPVFAGIVALLNDVRLKAGLPVLGFLNPWLYQDGLNGLNDIVDGGSTGCDGNNRFNGSPNGSPVIPYAGWNATEGWDPVTGLGTPDFAKLKALVLDA

SEQ ID NO: 21 - protein, 604 aa, Aspergillus ruber
MLSFVRRGALSLALVSLLTSSVAAEVFEKLHVVPEGWRYASTPNPKQPIRLQIALQQHDVTGFEQSLLEMSTPDHPNYGKHFRTHDEMKRMLLPNENAVHAVREWLQDAGISDIEEDADWVRFHTTVDQANDLLDANFLWYAHKSHRNTARLRTLEYSIPDSIAPQVNVIQPTTRFGQIRANRATHSSKPKGGLDELAISQAATADDDSICDQITTPHCLRKLYNVNGYKADPASGSKIGFASFLEEYARYSDLVLFEENLAPFAEGENFTVVMYNGGKNDQNSKSDSGEANLDLQYIVGMSAGAPVTEFSTAGRAPVIPDLDQPDPSAGTNEPYLEFLQNVLHMDQEHLPQVISTSYGENEQTIPEKYARTVCNMYAQLGSRGVSVIFSSGDSGVGSACMTNDGTNRTHFPPQFPASCPWVTSVGATEKMAPEQATYFSSGGFSDLFPRPKYQDAAVSSYLQTLGSRYQGLYNGSNRAFPDVSAQGTNFAVYDKGRLGQFDGTSCSAPAFSGIIALLNDVRLQNNKPVLGFLNPWLYGAGSKGLNDVVHGGSTGCDGQERFAGKANGSPVVPYASWNATQGWDPVTGLGTPDFGKLKDLALSA

SEQ ID NO: 22 - protein, 600 aa, Aspergillus terreus
MLPSLVNNGALSLAVLSLLTSSVAGEVFEKLSAVPKGWHFSHAAQADAPINLKIALKQHDVEGFEQALLDMSTPGHENYGKHFHEHDEMKRMLLPSDSAVDAVQTWLTSAGITDYDLDADWINLRTTVEHANALLDTQFGWYENEVRHITRLRTLQYSIPETVAAHINMVQPTTRFGQIRPDRATFHAHHTSDARILSALAAASNSTSCDSVITPKCLKDLYKVGDYEADPDSGSQVAFASYLEEYARYADMVKFQNSLAPYAKGQNFSVVLYNGGVNDQSSSADSGEANLDLQTIMGLSAPLPITEYITGGRGKLIPDLSQPNPNDNSNEPYLEFLQNILKLDQDELPQVISTSYGEDEQTIPRGYAESVCNMLAQLGSRGVSVVFSSGDSGVGAACQTNDGRNQTHFNPQFPASCPWVTSVGATTKTNPEQAVYFSSGGFSDFWKRPKYQDEAVAAYLDTLGDKFAGLFNKGGRAFPDVAAQGMNYAIYDKGTLGRLDGTSCSAPAFSAIISLLNDARLREGKPTMGFLNPWLYGEGREALNDVVVGGSKGCDGRDRFGGKPNGSPVVPFASWNATQGWDPVTGLGTPNFAKMLELAP

SEQ ID NO: 23 - protein, 601 aa, Penicillium digitatum
MIASLFNRRALTLALLSLFASSATADVFESLSAVPQGWRYSRTPSANQPLKLQIALAQGDVAGFEAAVIDMSTPDHPSYGNHFNTHEEMKRMLQPSAESVDSIRNWLESAGISKIEQDADWMTFYTTVKTANELLAANFQFYINGVKKIERLRTLKYSVPDALVSHINMIQPTTRFGQLRAQRAILHTEVKDNDEAFRSNAMSANPDCNSIITPQCLKDLYSIGDYEADPTNGNKVAFASYLEEYARYSDLALFEKNIAPFAKGQNFSVVQYNGGGNDQQSSSGSSEANLDLQYIVGVSSPVPVTEFSTGGRGELVPDLDQPNPNDNNNEPYLEFLQNVLKLHKKDLPQVISTSYGEDEQSVPEKYARAVCNLYSQLGSRGVSVIFSSGDSGVGAACQTNDGRNATHFPPQFPAACPWVTSVGATTHTAPERAVYFSSGGFSDLWDRPTWQEDAVSEYLENLGDRWSGLFNPKGRAFPDVAAQGENYAIYDKGSLISVDGTSCSAPAFAGVIALLNDARIKANRPPMGFLNPWLYSEGRSGLNDIVNGGSTGCDGHGRFSGPTNGGTSIPGASWNATKGWDPVSGLGSPNFAAMRKLANAE

SEQ ID NO: 24 - protein, 601 aa, Penicillium oxalicum
MHVPLLNQGALSLAVVSLLASTVSAEVFDKLVAVPEGWRFSRTPSGDQPIRLQVALTQGDVEGFEKAVLDMSTPDHPNYGKHFKSHEEVKRMLQPAGESVEAIHQWLEKAGITHIQQDADWMTFYTTVEKANNLLDANFQYYLNENKQVERLRTLEYSVPDELVSHINLVTPTTRFGQLHAEGVTLHGKSKDVDEQFRQAATSPSSDCNSAITPQCLKDLYKVGDYKASASNGNKVAFTSYLEQYARYSDLALFEQNIAPYAQGQNFTVIQYNGGLNDQSSPADSSEANLDLQYIIGTSSPVPVTEFSTGGRGPLVPDLDQPDINDNNNEPYLDFLQNVIKMSDKDLPQVISTSYGEDEQSVPASYARSVCNLIAQLGGRGVSVIFSSGDSGVGSACQTNDGKNTTRFPAQFPAACPWVTSVGATTGISPERGVFFSSGGFSDLWSRPSWQSHAVKAYLHKLGKRQDGLFNREGRAFPDVSAQGENYAIYAKGRLGKVDGTSCSAPAFAGLVSLLNDARIKAGKSSLGFLNPWLYSHPDALNDITVGGSTGCDGNARFGGRPNGSPVVPYASWNATEGWDPVTGLGTPNFQKLLKSAVKQK

SEQ ID NO: 25 - protein, 601 aa, Penicillium roqueforti
MIASLFSRGALSLAVLSLLASSAAADVFESLSAVPQGWRYSRRPRADQPLKLQIALTQGDTAGFEEAVMEMSTPDHPSYGHHFTTHEEMKRMLQPSAESAESIRDWLEGAGITRIEQDADWMTFYTTVETANELLAANFQFYVSNVRHIERLRTLKYSVPKALVPHINMIQPTTRFGQLRAHRGILHGQVKESDEAFRSNAVSAQPDCNSIITPQCLKDIYNIGDYQANDTNGNKVGFASYLEEYARYSDLALFEKNIAPSAKGQNFSVTRYNGGLNDQSSSGSSSEANLDLQYIVGVSSPVPVTEFSVGGRGELVPDLDQPDPNDNNNEPYLEFLQNVLKLDKKDLPQVISTSYGEDEQSIPEKYARSVCNLYSQLGSRGVSVIFSSGDSGVGSACLTNDGRNATRFPPQFPAACPWVTSVGATTHTAPEQAVYFSSGGFSDLWARPKWQEEAVSEYLEILGNRWSGLFNPKGRAFPDVTAQGRNYAIYDKGSLTSVDGTSCSAPAFAGVVALLNDARLKVNKPPMGFLNPWLYSTGRAGLKDIVDGGSTGCDGKSRFGGANNGGPSIPGASWNATKGWDPVSGLGSPNFATMRKLANAE

SEQ ID NO: 26 - protein, 601 aa, Penicillium rubens Wisconsin
MIASLFNRGALSLAVLSLLASSASADVFESLSAVPQGWRYSRRPRADQPLKLQIALAQGDTAGFEEAVMDMSTPDHPSYGNHFHTHEEMKRMLQPSAESADSIRDWLESAGINRIEQDADWMTFYTTVETANELLAANFQFYANSAKHIERLRTLQYSVPEALMPHINMIQPTTRFGQLRVQGAILHTQVKETDEAFRSNAVSTSPDCNSIITPQCLKNMYNVGDYQADDDNGNKVGFASYLEEYARYSDLELFEKNVAPFAKGQNFSVIQYNGGLNDQHSSASSSEANLDLQYIVGVSSPVPVTEFSVGGRGELVPDLDQPDPNDNNNEPYLEFLQNVLKMEQQDLPQVISTSYGENEQSVPEKYARTVCNLFSQLGSRGVSVIFASGDSGVGAACQTNDGRNATRFPAQFPAACPWVTSVGATTHTAPEKAVYFSSGGFSDLWDRPKWQEDAVSDYLDTLGDRWSGLFNPKGRAFPDVSAQGQNYAIYDKGSLTSVDGTSCSAPAFAGVIALLNDARLKANKPPMGFLNPWLYSTGRDGLNDIVHGGSTGCDGNARFGGPGNGSPRVPGASWNATKGWDPVSGLGSPNFATMRKLANGE

SEQ ID NO: 27 - protein, 594 aa, Neosartorya fischeri
MLSSTLYAGLLCSLAAPALGVVHEKLSAVPSGWTLVEDASESDTTTLSIALARQNLDQLESKLTTLATPGNAEYGKWLDQSDIESLFPTASDDAVIQWLKDAGVTQVSRQGSLVNFATTVGTANKLFDTKFSYYRNGASQKLRTTQYSIPDSLTESIDLIAPTVFFGKEQDSALPPHAVKLPALPRRAATNSSCANLITPDCLVEMYNLGDYKPDASSGSRVGFGSFLNQSANYADLAAYEQLFNIPPQNFSVELINGGANDQNWATASLGEANLDVELIVAVSHALPVVEFITGGSPPFVPNVDEPTAADNQNEPYLQYYEYLLSKPNSHLPQVISNSYGDDEQTVPEYYARRVCNLIGLMGLRGITVLESSGDTGIGSACMSNDGTNTPQFTPTFPGTCPFITAVGGTQSYAPEVAWDASSGGFSNYFSRPWYQYFAVENYLNNHITKDTKKYYSQYTNFKGRGFPDVSAHSLTPDYEVVLTGKHYKSGGTSAACPVFAGIVGLLNDARLRAGKSTLGFLNPLLYSILAEGFTDITAGSSIGCNGINPQTGKPVPGGGIIPYAHWNATAGWDPVTGLGVPDFMKLKELVLSL

SEQ ID NO: 28 - protein, 568 aa, Aspergillus fumigatus
MLSSTLYAGWLLSLAAPALCVVQEKLSAVPSGWTLIEDASESDTITLSIALARQNLDQLESKLTTLATPGNPEYGKWLDQSDIESLFPTASDDAVLQWLKAAGITQVSRQGSLVNFATTVGTANKLFDTKFSYYRNGASQKLRTTQYSIPDHLTESIDLIAPTVFFGKEQNSALSSHAVKLPALPRRAATNSSCANLITPDCLVEMYNLGDYKPDASSGSRVGFGSFLNESANYADLAAYEQLFNIPPQNFSVELINRGVNDQNWATASLGEANLDVELIVAVSHPLPVVEFITGALPPVLRVLALQTQLPSSSGDFQLTVPEYYARRVCNLIGLMGLRGITVLESSGDTGIGSACMSNDGTNKPQFTPTFPGTCPFITAVGGTQSYAPEVAWDGSSGGFSNYFSRPWYQSFAVDNYLNNHITKDTKKYYSQYTNFKGRGFPDVSAHSLTPYYEVVLTGKHYKSGGTSAASPVFAGIVGLLNDARLRAGKSTLGFLNPLLYSILAEGFTDITAGSSIGCNGINPQTGKPVPGGGIIPYAHWNATAGWDPVTGLGVPDFMKLKELVLSL

SEQ ID NO: 29 - protein, 415 aa, Trichoderma reesei
QEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 30 - protein, 391 aa, Aspergillus oryzae
CDSIITPTCLKELYNIGDYQADANSGSKIAFASYLEEYARYADLENFENYLAPWAKGQNFSVTTFNGGLNDQNSSSDSGEANLDLQYILGVSAPLPVTEFSTGGRGPLVPDLTQPDPNSNSNEPYLEFFQNVLKLDQKDLPQVISTSYGENEQEIPEKYARTVCNLIAQLGSRGVSVLFSSGDSGVGEGCMTNDGTNRTHFPPQFPAACPWVTSVGATFKTTPERGTYFSSGGFSDYWPRPEWQDEAVSSYLETIGDTFKGLYNSSGRAFPDVAAQGMNFAVYDKGTLGEFDGTSASAPAFSAVIALLNDARLRAGKPTLGFLNPWLYKTGRQGLQDITLGASIGCTGRARFGGAPDGGPVVPYASWNATQGWDPVTGLGTPDFAELKKLA

SEQ ID NO: 31 - protein, 388 aa, Phaeosphaeria nodorum
CDATITPQCLKTLYKIDYKADPKSGSKVAFASYLEQYARYNDLALFEKAFLPEAVGQNFSVVQFSGGLNDQNTTQDSGEANLDLQYIVGVSAPLPVTEFSTGGRGPWVADLDQPDEADSANEPYLEFLQGVLKLPQSELPQVISTSYGENEQSVPKSYALSVCNLFAQLGSRGVSVIFSSGDSGPGSACQSNDGKNTTKFQPQYPAACPFVTSVGSTRYLNETATGFSSGGFSDYWKRPSYQDDAVKAYFHHLGEKFKPYFNRHGRGFPDVATQGYGFRVYDQGKLKGLQGTSASAPAFAGVIGLLNDARLKAKKPTLGFLNPLLYSNSDALNDIVLGGSKGCDGHARFNGPPNGSPVIPYAGWNATAGWDPVTGLGTPNFPKLLKAA

SEQ ID NO: 32 - protein, 395 aa, Trichoderma atroviride
VFQPDCLRTEYSVNGYKPSAKSGSRIGFGSFLNQSASSSDLALFEKHFGFASQGFSVELINGGSNPQPPTDANDGEANLDAQNIVSFVQPLPITEFIAGGTAPYFPDPVEPAGTPDENEPYLEYYEYLLSKSNKELPQVITNSYGDEEQTVPQAYAVRVCNLIGLMGLRGISILESSGDEGVGASCLATNSTTTPQFNPIFPATCPYVTSVGGTVSFNPEVAWDGSSGGFSYYFSRPWYQEAAVGTYLNKYVSEETKEYYKSYVDFSGRGFPDVAAHSVSPDYPVFQGGELTPSGGTSAASPIVASVIALLNDARLRAGKPALGFLNPLIYGYAYKGFTDITSGQAVGCNGNNTQTGGPLPGAGVIPGAFWNATKGWDPTTGFGVPNFKKLLELV

SEQ ID NO: 33 - protein, 389 aa, Arthroderma benhamiae
CRSLVTTACLRELYGLGDRVTQARDDNRIGVSGFLEEYAQYRDLELFLSRFEPSAKGFNFSEGLIAGGKNTQGGPGSSTEANLDMQYVVGLSHKAKVTYYSTAGRGPLIPDLSQPSQASNNNEPYLEQLRYLVKLPKNQLPSVLTTSYGDTEQSLPASYTKATCDLFAQLGTMGVSVIFSSGDTGPGSSCQTNDGKNATRFNPIYPASCPFVTSIGGTVGTGPERAVSFSSGGFSDRFPRPQYQDNAVKDYLKILGNQWSGLFDPNGRAFPDIAAQGSNYAVYDKGRMTGVSGTSASAPAMAAIIAQLNDFRLAKGSPVLGFLNPWIYSKGFSGFTDIVDGGSRGCTGYDIYSGLKAKKVPYASWNATKGWDPVTGFGTPNFQALTKVL

SEQ ID NO: 34 - protein, 397 aa, Fusarium graminearum
CQTSITPSCLKQMYNIGDYTPKVESGSTIGFSSFLGESAIYSDVFLFEEKFGIPTQNFTTVLINNGTDDQNTAHKNFGEADLDAENIVGIAHPLPFTQYITGGSPPFLPNIDQPTAADNQNEPYVPFFRYLLSQKEVPAVVSTSYGDEEDSVPREYATMTCNLIGLLGLRGISVIFSSGDIGVGAGCLGPDHKTVEFNAIFPATCPYLTSVGGTVDVTPEIAWEGSSGGFSKYFPRPSYQDKAVKTYMKTVSKQTKKYYGPYTNWEGRGFPDVAGHSVSPNYEVIYAGKQSASGGTSAAAPVWAAIVGLLNDARFRAGKPSLGWLNPLVYKYGPKVLTDITGGYAIGCDGNNTQSGKPEPAGSGIVPGARWNATAGWDPVTGYGTPDFGKLKDLVLS

SEQ ID NO: 35 - protein, 408 aa, Acremonium alcalophilum
CDLVITPPCLEAAYNYKNYMPDPNSGSRVSFTSFLEQAAQQSDLTKFLSLTGLDRLRPPSSKPASFDTVLINGGETHQGTPPNKTSEANLDVQWLAAVIKARLPITQWITGGRPPFVPNLRLRHEKDNTNEPYLEFFEYLVRLPARDLPQVISNSYAEDEQTVPEAYARRVCNLIGIMGLRGVTVLTASGDSGVGAPCRANDGSDRLEFSPQFPTSCPYITAVGGTEGWDPEVAWEASSGGFSHYFLRPWYQANAVEKYLDEELDPATRAYYDGNGFVQFAGRAYPDLSAHSSSPRYAYIDKLAPGLTGGTSASCPVVAGIVGLLNDARLRRGLPTMGFINPWLYTRGFEALQDVTGGRASGCQGIDLQRGTRVPGAGIIPWASWNATPGWDPATGLGLPDFWAMRGL

SEQ ID NO: 36 - protein, 410 aa, Sodiomyces alkalinus
CATIITPPCLETAYNYKGYIPDPKSGSRVSFTSFLEQAAQQADLTKFLSLTRLEGFRTPASKKKTFKTVLINGGESHEGVHKKSKTSEANLDVQWLAAVTQTKLPITQWITGGRPPFVPNLRIPTPEANTNEPYLEFLEYLFRLPDKDLPQVISNSYAEDEQSVPEAYARRVCGLLGIMGLRGVTVLTASGDSGVGAPCRANDGSGREEFSPQFPSSCPYITTVGGTQAWDPEVAWKGSSGGFSNYFPRPWYQVAAVEKYLEEQLDPAAREYYEENGFVRFAGRAFPDLSAHSSSPKYAYVDKRVPGLTGGTSASCPVVAGIVGLLNDARLRRGLPTMGFINPWLYAKGYQALEDVTGGAAVGCQGIDIQTGKRVPGAGIIPGASWNATPDWDPATGLGLPNFWAMRELA

SEQ ID NO: 37 - protein, 400 aa, Aspergillus kawachii
CADTITLSCLKEMYNFGNYTPSASSGSKLGFASFLNESASYSDLAKFERLFNLPSQNFSVELINGGVNDQNQSTASLTEADLDVELLVGVGHPLPVTEFITSGEPPFIPDPDEPSAADNENEPYLQYYEYLLSKPNSALPQVISNSYGDDEQTVPEYYAKRVCNLIGLVGLRGISVLESSGDEGIGSGCRTTDGTNSTQFNPIFPATCPYVTAVGGTMSYAPEIAWEASSGGFSNYFERAWFQKEAVQNYLANHITNETKQYYSQFANFSGRGFPDVSAHSFEPSYEVIFYGARYGSGGTSAACPLFSALVGMLNDARLRAGKSTLGFLNPLLYSKGYKALTDVTAGQSIGCNGIDPQSDEAVAGAGIIPWAHWNATVGWDPVTGLGLPDFEKLRQLVLS

SEQ ID NO: 38 - protein, 396 aa, Talaromyces stipitatus
CQTSITPACLKQMYNVGNYTPSVAHGSRVGFGSFLNQSAIFDDLFTYEKVNDIPSQNFTKVIIANASNSQDASDGNYGEANLDVQNIVGISHPLPVTEFLTGGSPPFVASLDTPTNQNEPYIPYYEYLLSQKNEDLPQVISNSYGDDEQSVPYKYAIRACNLIGLTGLRGISVLESSGDLGVGAGCRSNDGKNKTQFDPIFPATCPYVTSVGGTQSVTPEIAWVASSGGFSNYFPRTWYQEPAIQTYLGLLDDETKTYYSQYTNFEGRGFPDVSAHSLTPDYQVVGGGYLQPSGGTSAASPVFAGIIALLNDARLAAGKPTLGFLNPFFYLYGYKGLNDITGGQSVGCNGINGQTGAPVPGGGIVPGAAWNSTTGWDPATGLGTPDFQKLKELVLS

SEQ ID NO: 39 - protein, 397 aa, Fusarium oxysporum
CQTSITPSCLKQMYNIGDYTPDAKSGSEIGFSSFLGQAAIYSDVFKFEELFGIPKQNYTTILINNGTDDQNTAHGNFGEANLDAENIVGIAHPLPFKQYITGGSPPFVPNIDQPTEKDNQNEPYVPFFRYLLGQKDLPAVISTSYGDEEDSVPREYATLTCNMIGLLGLRGISVIFSSGDIGVGSGCLAPDYKTVEFNAIFPATCPYLTSVGGTVDVTPEIAWEGSSGGFSKYFPRPSYQDKAIKKYMKTVSKETKKYYGPYTNWEGRGFPDVAGHSVAPDYEVIYNGKQARSGGTSAAAPVWAAIVGLLNDARFKAGKKSLGWLNPLIYKHGPKVLTDITGGYAIGCDGNNTQSGKPEPAGSGLVPGARWNATAGWDPTTGYGTPNFQKLKDLVLS

SEQ ID NO: 40 - protein, 395 aa, Trichoderma virens
VFQPDCLRTEYNVNGYTPSAKSGSRIGFGSFLNQSASFSDLALFEKHFGFSSQNFSVVLINGGTDLPQPPSDDNDGEANLDVQNILTIAHPLPITEFITAGSPPYFPDPVEPAGTPDENEPYLQYFEYLLSKPNRDLPQVITNSYGDEEQTVPQAYAVRVCNLIGLMGLRGISILESSGDEGVGASCVATNSTTPQFNPIFPATCPYVTSVGGTVNFNPEVAWDGSSGGFSYYFSRPWYQEEAVGNYLEKHVSAETKKYYGPYVDFSGRGFPDVAAHSVSPDYPVFQGGQLTPSGGTSAASPVVASIIALLNDARLREGKPTLGFLNPLIYQYAYKGFTDITSGQSDGCNGNNTQTDAPLPGAGVVLGAHWNATKGWDPTTGFGVPNFKKLLELI

SEQ ID NO: 41 - protein, 398 aa, Trichoderma atroviride
QIFHPDCLKTKYGVDGYAPSPRCGSRIGFGSFLNETASYSDLAQFEKYFDLPNQNLSTLLINGAIDVQPPSNKNDSEANMDVQTILTFVQPLPITEFVVAGIPPYIPDAALPIGDPVQNEPWLEYFEFLMSRTNAELPQVIANSYGDEEQTVPQAYAVRVCNQIGLLGLRGISVIASSGDTGVGMSCMASNSTTPQFNPMFPASCPYITTVGGTQHLDNEIAWELSSGGFSNYFTRPWYQEDAAKTYLERHVSTETKAYYERYANFLGRGFPDVAALSLNPDYPVIIGGELGPNGGTSAAAPVVASIIALLNDARLCLGKPALGFLNPLIYQYADKGGFTDITSGQSWGCAGNTTQTGPPPPGAGVIPGAHWNATKGWDPVTGFGTPNFKKLLSLALS

SEQ ID NO: 42 - protein, 363 aa, Agaricus bisporus
TVITPDCLRDLYNTADYVPSATSRNAIGIAGYLDRSNRADLQTFFRRFRPDAVGFNYTTVQLNGGGDDQNDPGVEANLDIQYAAGIAFPTPATYWSTGGSPPFIPDTQTPTNTNEPYLDWINFVLGQDEIPQVISTSYGDDEQTVPEDYATSVCNLFAQLGSRGVTVFFSSGDFGVGGGDCLTNDGSNQVLFQPAFPASCPFVTAVGGTVRLDPEIAVSFSGGGFSRYFSRPSYQNQTVAQFVSNLGNTFNGLYNKNGRAYPDLAAQGNGFQVVIDGIVRSVGGTSASSPTVAGIFALLNDFKLSRGQSTLGFINPLIYSSATSGFNDIRAGTNPGCGTRGFTAGTGWDPVTGLGTPDFLRLQ

SEQ ID NO: 43 - protein, 376 aa, Magnaporthe oryzae
GVTPLCLRTLYRVNYKPATTGNLVAFASFLEQYARYSDQQAFTQRVLGPGVPLQNFSVETVNGGANDQQSKLDSGEANLDLQYVMAMSHPIPILEYSTGGRGPLVPTLDQPNANNSSNEPYLEFLTYLLAQPDSAIPQTLSVSYGEEEQSVPRDYAIKVCNMFMQLGARGVSVMFSSGDSGPGNDCVRASDNATFFGSTFPAGCPYVTSVGSTVGFEPERAVSFSSGGFSIYHARPDYQNEVVPKYIESIKASGYEKFFDGNGRGIPDVAAQGARFVVIDKGRVSLISGTSASSPAFAGMVALVNAARKSKDMPALGFLNPMLYQNAAAMTDIVNGAGIGCRKQRTEFPNGARFNATAGWDPVTGLGTPLFDKLLA

SEQ ID NO: 44 - protein, 388 aa, Togninia minima
CNASITPECLRALYNVGDYEADPSKKSLFGVCGYLEQYAKHDQLAKFEQTYAPYAIGADFSVVTINGGGDNQTSTIDDGEANLDMQYAVSMAYKTPITYYSTGGRGPLVPDLDQPDPNDVSNEPYLDFVSYLLKLPDSKLPQTITTSYGEDEQSVPRSYVEKVCTMFGALGARGVSVIFSSGDTGVGSACQTNDGKNTTRFLPIFPAACPYVTSVGGTRYVDPEVAVSFSSGGFSDIFPTPLYQKGAVSGYLKILGDRWKGLYNPHGRGFPDVSGQSVRYHVFDYGKDVMYSGTSASAPMFAALVSLLNNARLAKKLPPMGFLNPWLYTVGFNGLTDIVHGGSTGCTGTDVYSGLPTPFVPYASWNATVGWDPVTGLGTPLFDKLLNL

SEQ ID NO: 45 - protein, 390 aa, Bipolaris maydis
CNKKITPDCLANLYNFKDYDASDANVTIGVSGFLEQYARFDDLKQFISTFQPKAAGSTFQVTSVNAGPFDQNSTASSVEANLDIQYTTGLVAPDIETRYFTVPGRGILIPDLDQPTESDNANEPYLDYFTYLNNLEDEELPDVLTTSYGESEQSVPAEYAKKVCNLIGQLGARGVSVIFSSGDTGPGSACQTNDGKNTTRFLPIFPASCPYVTSVGGTVGVEPEKAVSFSSGGFSDLWPRPAYQEKAVSEYLEKLGDRWNGLYNPQGRGFPDVAAQGQGFQVFDKGRLISVGGTSASAPVFASVVALLNNARKAAGMSSLGFLNPWIYEQGYKGLTDIVAGGSTGCTGRSIYSGLPAPLVPYASWNATEGWDPVTGYGTPDFKQLLTLAT

SEQ ID NO: 46 - protein, 393 aa, Aspergillus kawachii
CDSIITPHCLKQLYNIGDYQADPKSGSKVGFASYLEEYARYADLERFEQHLAPNAIGQNFSVVQFNGGLNDQLSLSDSGEANLDLQYILGVSAPVPVTEYSTGGRGELVPDLSSPDPNDNSNEPYLDFLQGILKLDNSDLPQVISTSYGEDEQTIPVPYARTVCNLYAQLGSRGVSVIFSSGDSGVGAACLTNDGTNRTHFPPQFPASCPWVTSVGATSKTSPEQAVSFSSGGFSDLWPRPSYQQAAVQTYLTQHLGNKFSGLFNASGRAFPDVAAQGVNYAVYDKGMLGQFDGTSCSAPTFSGVIALLNDARLRAGLPVMGFLNPFLYGVGSESGALNDIVNGGSLGCDGRNRFGGTPNGSPVVPFASWNATTGWDPVSGLGTPDFAKLRGV

SEQ ID NO: 47 - protein, 392 aa, Aspergillus nidulans
CEKAITPSCLADLYNTEGYKASNRSGSKVAFASFLEEYARYDDLAEFEETYAPYAIGQNFSVISINGGLNDQDSTADSGEANLDLQYIIGVSSPLPVTEFTTGGRGKLIPDLSSPDPNDNTNEPFLDFLEAVLKLDQKDLPQVISTSYGEDEQTIPEPYARSVCNLYAQLGSRGVSVLFSSGDSGVGAACQTNDGKNTTHFPPQFPASCPWVTAVGGTNGTAPESGVYFSSGGFSDYWARPAYQNAAVESYLRKLGSTQAQYFNRSGRAFPDVAAQAQNFAVVDKGRVGLFDGTSCSSPVFAGIVALLNDVRLKAGLPVLGFLNPWLYQDGLNGLNDIVDGGSTGCDGNNRFNGSPNGSPVIPYAGWNATEGWDPVTGLGTPDFAKLKALVL

SEQ ID NO: 48 - protein, 392 aa, Aspergillus ruber
CDQITTPHCLRKLYNVNGYKADPASGSKIGFASFLEEYARYSDLVLFEENLAPFAEGENFTVVMYNGGKNDQNSKSDSGEANLDLQYIVGMSAGAPVTEFSTAGRAPVIPDLDQPDPSAGTNEPYLEFLQNVLHMDQEHLPQVISTSYGENEQTIPEKYARTVCNMYAQLGSRGVSVIFSSGDSGVGSACMTNDGTNRTHFPPQFPASCPWVTSVGATEKMAPEQATYFSSGGFSDLFPRPKYQDAAVSSYLQTLGSRYQGLYNGSNRAFPDVSAQGTNFAVYDKGRLGQFDGTSCSAPAFSGIIALLNDVRLQNNKPVLGFLNPWLYGAGSKGLNDVVHGGSTGCDGQERFAGKANGSPVVPYASWNATQGWDPVTGLGTPDFGKLKDLAL

SEQ ID NO: 49 - protein, 391 aa, Aspergillus terreus
CDSVITPKCLKDLYKVGDYEADPDSGSQVAFASYLEEYARYADMVKFQNSLAPYAKGQNFSVVLYNGGVNDQSSSADSGEANLDLQTIMGLSAPLPITEYITGGRGKLIPDLSQPNPNDNSNEPYLEFLQNILKLDQDELPQVISTSYGEDEQTIPRGYAESVCNMLAQLGSRGVSVVFSSGDSGVGAACQTNDGRNQTHFNPQFPASCPWVTSVGATTKTNPEQAVYFSSGGFSDFWKRPKYQDEAVAAYLDTLGDKFAGLFNKGGRAFPDVAAQGMNYAIYDKGTLGRLDGTSCSAPAFSAIISLLNDARLREGKPTMGFLNPWLYGEGREALNDVVVGGSKGCDGRDRFGGKPNGSPVVPFASWNATQGWDPVTGLGTPNFAKMLELA

SEQ ID NO: 50 - protein, 391 aa, Penicillium digitatum
CNSIITPQCLKDLYSIGDYEADPTNGNKVAFASYLEEYARYSDLALFEKNIAPFAKGQNFSVVQYNGGGNDQQSSSGSSEANLDLQYIVGVSSPVPVTEFSTGGRGELVPDLDQPNPNDNNNEPYLEFLQNVLKLHKKDLPQVISTSYGEDEQSVPEKYARAVCNLYSQLGSRGVSVIFSSGDSGVGAACQTNDGRNATHFPPQFPAACPWVTSVGATTHTAPERAVYFSSGGFSDLWDRPTWQEDAVSEYLENLGDRWSGLFNPKGRAFPDVAAQGENYAIYDKGSLISVDGTSCSAPAFAGVIALLNDARIKANRPPMGFLNPWLYSEGRSGLNDIVNGGSTGCDGHGRFSGPTNGGTSIPGASWNATKGWDPVSGLGSPNFAAMRKLA

SEQ ID NO: 51 - protein, 391 aa, Penicillium oxalicum
CNSAITPQCLKDLYKVGDYKASASNGNKVAFTSYLEQYARYSDLALFEQNIAPYAQGQNFTVIQYNGGLNDQSSPADSSEANLDLQYIIGTSSPVPVTEFSTGGRGPLVPDLDQPDINDNNNEPYLDFLQNVIKMSDKDLPQVISTSYGEDEQSVPASYARSVCNLIAQLGGRGVSVIFSSGDSGVGSACQTNDGKNTTRFPAQFPAACPWVTSVGATTGISPERGVFFSSGGFSDLWSRPSWQSHAVKAYLHKLGKRQDGLFNREGRAFPDVSAQGENYAIYAKGRLGKVDGTSCSAPAFAGLVSLLNDARIKAGKSSLGFLNPWLYSHPDALNDITVGGSTGCDGNARFGGRPNGSPVVPYASWNATEGWDPVTGLGTPNFQKLLKSAV

SEQ ID NO: 52 - protein, 391 aa, Penicillium roqueforti
CNSIITPQCLKDIYNIGDYQANDTNGNKVGFASYLEEYARYSDLALFEKNIAPSAKGQNFSVTRYNGGLNDQSSSGSSSEANLDLQYIVGVSSPVPVTEFSVGGRGELVPDLDQPDPNDNNNEPYLEFLQNVLKLDKKDLPQVISTSYGEDEQSIPEKYARSVCNLYSQLGSRGVSVIFSSGDSGVGSACLTNDGRNATRFPPQFPAACPWVTSVGATTHTAPEQAVYFSSGGFSDLWARPKWQEEAVSEYLEILGNRWSGLFNPKGRAFPDVTAQGRNYAIYDKGSLTSVDGTSCSAPAFAGVVALLNDARLKVNKPPMGFLNPWLYSTGRAGLKDIVDGGSTGCDGKSRFGGANNGGPSIPGASWNATKGWDPVSGLGSPNFATMRKLA

SEQ ID NO: 53 - protein, 391 aa, Penicillium rubens Wisconsin
CNSIITPQCLKNMYNVGDYQADDDNGNKVGFASYLEEYARYSDLELFEKNVAPFAKGQNFSVIQYNGGLNDQHSSASSSEANLDLQYIVGVSSPVPVTEFSVGGRGELVPDLDQPDPNDNNNEPYLEFLQNVLKMEQQDLPQVISTSYGENEQSVPEKYARTVCNLFSQLGSRGVSVIFASGDSGVGAACQTNDGRNATRFPAQFPAACPWVTSVGATTHTAPEKAVYFSSGGFSDLWDRPKWQEDAVSDYLDTLGDRWSGLFNPKGRAFPDVSAQGQNYAIYDKGSLTSVDGTSCSAPAFAGVIALLNDARLKANKPPMGFLNPWLYSTGRDGLNDIVHGGSTGCDGNARFGGPGNGSPRVPGASWNATKGWDPVSGLGSPNFATMRKLA

SEQ ID NO: 54 - protein, 400 aa, Neosartorya fischeri
CANLITPDCLVEMYNLGDYKPDASSGSRVGFGSFLNQSANYADLAAYEQLFNIPPQNFSVELINGGANDQNWATASLGEANLDVELIVAVSHALPVVEFITGGSPPFVPNVDEPTAADNQNEPYLQYYEYLLSKPNSHLPQVISNSYGDDEQTVPEYYARRVCNLIGLMGLRGITVLESSGDTGIGSACMSNDGTNTPQFTPTFPGTCPFITAVGGTQSYAPEVAWDASSGGFSNYFSRPWYQYFAVENYLNNHITKDTKKYYSQYTNFKGRGFPDVSAHSLTPDYEVVLTGKHYKSGGTSAACPVFAGIVGLLNDARLRAGKSTLGFLNPLLYSILAEGFTDITAGSSIGCNGINPQTGKPVPGGGIIPYAHWNATAGWDPVTGLGVPDFMKLKELVLS

SEQ ID NO: 55 - protein, 374 aa, Aspergillus fumigatus
CANLITPDCLVEMYNLGDYKPDASSGSRVGFGSFLNESANYADLAAYEQLFNIPPQNFSVELINRGVNDQNWATASLGEANLDVELIVAVSHPLPVVEFITGALPPVLRVLALQTQLPSSSGDFQLTVPEYYARRVCNLIGLMGLRGITVLESSGDTGIGSACMSNDGTNKPQFTPTFPGTCPFITAVGGTQSYAPEVAWDGSSGGFSNYFSRPWYQSFAVDNYLNNHITKDTKKYYSQYTNFKGRGFPDVSAHSLTPYYEVVLTGKHYKSGGTSAASPVFAGIVGLLNDARLRAGKSTLGFLNPLLYSILAEGFTDITAGSSIGCNGINPQTGKPVPGGGIIPYAHWNATAGWDPVTGLGVPDFMKLKELVLS

SEQ ID NO: 56 - DNA, 2027 bp, Trichoderma reesei
atggcaaagttgagcactctccggcttgcgagccttctttcccttgtcagtgtgcaggtatctgcctctgtccatctattggagagtctggagaagctgcctcatggatggaaagcagctgaaaccccgagcccttcgtctcaaatcgtcttgcaggttgctctgacgcagcagaacattgaccagcttgaatcgaggctcgcagctgtatccacacccacttctagcacctacggcaaatacttggatgtagacgagatcaacagcatcttcgctccaagtgatgctagcagttctgccgtcgagtcttggcttcagtcccacggagtgacgagttacaccaagcaaggcagcagcatttggtttcaaacaaacatctccactgcaaatgcgatgctcagcaccaatttccacacgtacagcgatctcaccggcgcgaagaaggtgcgcactctcaagtactcgatcccggagagcctcatcggccatgtcgatctcatctctcccacgacctattttggcacgacaaaggccatgaggaagttgaaatccagtggcgtgagcccagccgctgatgctctagccgctcgccaagaaccttccagctgcaaaggaactctagtctttgagggagaaacgttcaatgtctttcagccagactgtctcaggaccgagtatagtgttgatggatacaccccgtctgtcaagtctggcagcagaattgggtttggttcctttctcaatgagagcgcaagcttcgcagatcaagcactctttgagaagcacttcaacatccccagtcaaaacttctccgttgtcctgatcaacggtggaacggatctccctcagccgccttctgacgccaacgatggcgaagccaacctggacgctcaaaccattttgaccatcgcacatcctctccccatcaccgaattcatcaccgccggcagtccgccatacttccccgatccagttgaacctgcgggaacacccaacgagaacgagccttatttacagtattacgaatttctgttgtccaagtccaacgctgaaattccgcaagtcattaccaactcctacggcgacgaggagcaaactgtgccgcggtcatatgccgttcgagtttgcaatctgattggtctgctaggactacgcggtatctctgtccttcattcctcgggcgacgagggtgtgggcgcctcttgcgttgctaccaacagcaccacgcctcagtttaaccccatctttcctgtaggtcttctacgtcaacacttccagacaaccattttctcctactaaccactctaccctactctctgttcacataggctacatgtccttatgttacaagtgttggcggaaccgtgagcttcaatcccgaggttgcctgggctggttcatctggaggtttcagctactacttctctagaccctggtaccagcaggaagctgtgggtacttaccttgagaaatatgtcagtgctgagacaaagaaatactatggaccttatgtcgatttctccggacgaggtttccccgatgttgcagcccacagcgtcagccccgagtgagttctattcctacctatgcaaatcatagaatgtatgctaactcgccatgaagctatcctgtgtttcagggcggtgaactcaccccaagcggaggcacttcagcagcctctcctgtcgtagcagccatcgtggcgctgttgaacgatgcccgtctccgcgaaggaaaacccacgcttggatttctcaatccgctgatttacctacacgcctccaaagggttcaccgacatcacctcgggccaatctgaagggtgcaacggcaataacacccagacgggcagtcctctcccaggagtatgcagaacatcaagaagccttctatcagacgccaatgctaacttgtggataggccggcttcattgcaggcgcacactggaacgcgaccaagggatgggacccgacgactggatttggtgttccaaacctcaaaaagctcctcgcacttgtccggttctaa

SEQ ID NO: 57 - DNA, 1803 bp, Aspergillus oryzae
atgttcttcagtcgtggagcgctttcgctcgcagtgctttcactgctcagctcctccgccgcaggggaggcttttgagaagctgtctgccgttccaaagggatggcactattctagtacccctaaaggcaacactgaggtttgtctgaagatcgccctcgcgcagaaggatgctgctgggttcgaaaagaccgtcttggagatgtcggatcccgaccaccccagctacggccagcacttcaccacccacgacgagatgaagcgcatgcttcttcccagagatgacaccgttgatgccgttcgacaatggctcgaaaacggcggcgtgaccgactttacccaggatgccgactggatcaacttctgtactaccgtcgataccgcgaacaaactcttgaatgcccagttcaaatggtacgtcagcgatgtgaagcacatccgccgtctcagaacactgcagtacgacgtccccgagtcggtcacccctcacatcaacaccatccaaccgaccacccgttttggcaagattagccccaagaaggccgttacccacagcaagccctcccagttggacgtgaccgcccttgctgccgctgtcgttgcaaagaacatctcgcactgtgattctatcattacccccacctgtctgaaggagctttacaacattggtgattaccaggccgatgcaaactcgggcagcaagatcgccttcgccagctatctggaggagtacgcgcgctacgctgacctggagaactttgagaactaccttgctccctgggctaagggccagaacttctccgttaccaccttcaacggcggtctcaatgatcagaactcctcgtccgatagcggtgaggccaacctggacctgcagtacattcttggtgtcagcgctccactgcccgttactgaattcagcaccggaggccgtggtcccctcgttcctgatctgacccagccggatcccaactctaacagcaatgagccgtaccttgagttcttccagaatgtgttgaagctcgaccagaaggacctcccccaggtcatctcgacctcctatggagagaacgaacaggaaatccccgaaaagtacgctcgcaccgtctgcaacctgatcgctcagcttggcagccgcggtgtctccgttctcttctcctccggtgactctggtgttggcgagggctgcatgaccaacgacggcaccaaccggactcacttcccaccccagttccccgccgcttgcccgtgggtcacctccgtcggcgccaccttcaagaccactcccgagcgcggcacctacttctcctcgggcggtttctccgactactggccccgtcccgaatggcaggatgaggccgtgagcagctacctcgagacgatcggcgacactttcaagggcctctacaactcctccggccgtgctttccccgacgtcgcagcccagggcatgaacttcgccgtctacgacaagggcaccttgggcgagttcgacggcacctccgcctccgccccggccttcagcgccgtcatcgctctcctgaacgatgcccgtctccgcgccggcaagcccactctcggcttcctgaacccctggttgtacaagaccggccgccagggtctgcaagatatcaccctcggtgctagcattggctgcaccggtcgcgctcgcttcggcggcgcccctgacggtggtcccgtcgtgccttacgctagctggaacgctacccagggctgggatcccgtcactggtctcggaactcccgatttcgccgagctcaagaagcttgcccttggcaactaa

SEQ ID NO: 58 - DNA, 1782 bp, Phaeosphaeria nodorum
atggcgcccatcctctcgttccttgttggctctctcctggcggttcgcgctcttgctgagccatttgagaagctgttcagcaccccggaaggatggaagatgcaaggtcttgctaccaatgagcagatcgtcaagctccagattgctcttcagcaaggcgatgttgcaggtttcgagcaacatgtgattgacatctcaacgcctagccacccgagctatggtgctcactatggctcgcatgaggagatgaagaggatgatccagccaagcagcgagacagtcgcttctgtgtctgcatggctgaaggccgccggtatcaacgacgctgagattgacagcgactgggtcaccttcaagacgaccgttggcgttgccaacaagatgctcgacaccaagttcgcttggtacgtgagcgaggaggccaagccccgcaaggtccttcgcacactcgagtactctgtaccagatgatgttgcagaacacatcaacttgatccagcccactactcggtttgctgcgatccgccaaaaccacgaggttgcgcacgagattgttggtcttcagttcgctgctcttgccaacaacaccgttaactgcgatgccaccatcactccccagtgcttgaagactctttacaagattgactacaaggccgatcccaagagtggttccaaggtcgcttttgcttcgtatttggagcagtacgcgcgttacaatgacctcgccctcttcgagaaggccttcctccccgaagcagttggccagaacttctctgtcgtccagttcagcggcggtctcaacgaccagaacaccacgcaagacagtggcgaggccaacttggacttgcagtacattgtcggtgtcagcgctcctcttcccgtcaccgagttcagcaccggtggtcgcggcccatgggtcgctgacctagaccaacctgacgaggcggacagcgccaacgagccctaccttgaattccttcagggtgtgctcaaacttccccagtctgagctacctcaggtcatctccacatcctatggcgagaatgagcagagtgtacctaagtcatacgctctctccgtctgcaacttgttcgcccaactcggttcccgtggcgtctccgtcatcttctcttctggtgacagcggccctggatccgcatgccagagcaacgacggcaagaacacgaccaagttccagcctcagtaccccgctgcctgcccctttgtcacctcggttggatcgactcgctacctcaacgagaccgcaaccggcttctcatctggtggtttctccgactactggaagcgcccatcgtaccaggacgatgctgttaaggcgtatttccaccacctcggtgagaaattcaagccatacttcaaccgccacggccgtggattccccgacgttgcaacccagggatatggcttccgcgtctacgaccagggcaagctcaagggtctccaaggtacttctgcctccgcgcctgcattcgccggtgtgattggtctcctcaacgacgcgcgattgaaggcgaagaagcctaccttgggattcctaaacccactgctttactctaactcagacgcgctaaatgacattgttctcggtggaagcaagggatgcgatggtcatgctcgctttaacgggccgccaaatggcagcccagtaatcccatatgcgggatggaacgcgactgctgggtgggatccagtgactggtcttggaacgccgaacttccccaagcttcttaaggctgcggtgcctagccggtacagggcgtga

SEQ ID NO: 59 - DNA, 1830 bp, Trichoderma atroviride
atggcgaaactgacagctcttgccggtctcctgacccttgccagcgtgcaggcaaatgccgccgtgctcttggacagcctcgacaaggtgcctgttggatggcaggctgcttcggccccggccccgtcatccaagatcaccctccaagttgccctcacgcagcagaacattgatcagttggaatcaaagctcgctgccgtctccacgcccaactccagcaactatggaaagtacctggatgtcgatgagattaaccaaatcttcgctcccagcagcgccagcaccgctgctgttgagtcctggctcaagtcgtacggcgtggactacaaggtgcagggcagcagcatctggttccagacggatgtctccacggccaacaagatgctcagcacaaacttccacacttacaccgactcggttggtgccaagaaagtgcgaactctccagtactcggtccccgagaccctggccgaccacatcgatctgatttcgcccacaacctactttggcacgtccaaggccatgcgggcgttgaagatccagaacgcggcctctgccgtctcgcccctggctgctcgtcaggagccctccagctgcaagggcacaattgagtttgagaaccgcacattcaacgtcttccagcccgactgtctcaggaccgagtacagcgtcaacggatacaagccctcagccaagtccggtagcaggattggcttcggctctttcctgaaccagagcgccagctcctcagatctcgctctgttcgagaagcactttggctttgccagccagggcttctccgtcgagctcatcaatggcggatcaaacccccagccgcccacagacgccaatgacggcgaggccaacctggacgcccagaacattgtgtcgtttgtgcagcctctgcccatcaccgagtttattgctggaggaactgcgccgtacttcccagaccccgttgagccggctggaactcccgatgagaacgagccttacctcgagtactacgagtacctgctctccaagtcaaacaaggagcttccccaagtcatcaccaactcctacggtgatgaggagcagactgttccccaggcatatgccgtccgcgtgtgcaacctcattggattgatgggccttcgtggtatctctatcctcgagtcatccggtgatgagggtgttggtgcctcttgtctcgctaccaacagcaccaccactccccagttcaaccccatcttcccggctacatgcccctatgtcaccagtgttggtggaaccgtcagcttcaaccccgaggttgcctgggacggctcatccggaggcttcagctactacttctcaagaccttggtaccaggaggccgcagtcggcacataccttaacaagtatgtcagcgaggagaccaaggaatactacaagtcgtatgtcgacttttccggacgtggcttccccgatgttgcagctcacagcgtgagccccgattaccccgtgttccaaggcggcgagcttacccccagcggcggtacttctgcggcctctcccatcgtggccagtgttattgccctcctgaacgatgctcgtctccgtgcaggcaagcctgctctcggattcttgaaccctctgatctacggatatgcctacaagggctttaccgatatcacgagtggccaagctgtcggctgcaacggcaacaacactcaaactggaggccctcttcctggtgcgggtgttattccaggtgctttctggaacgcgaccaagggctgggatcctacaactggattcggtgtccccaacttcaagaagctgcttgagcttgtccgatacatttag

SEQ ID NO: 60 - DNA, 1791 bp, Arthroderma benhamiae
atgcgtcttctcaaatttgtgtgcctgttggcatcagttgccgccgcaaagcctactccaggggcgtcacacaaggtcattgaacatcttgactttgttccagaaggatggcagatggttggtgccgcggaccctgctgctatcattgatttctggcttgccatcgagcgcgaaaacccagaaaagctctacgacaccatctatgacgtctccacccctggacgcgcacaatatggcaaacatttgaagcgtgaggaattggatgacttactacgcccaagggcagagacgagtgagagcatcatcaactggctcaccaatggtggagtcaacccacaacatattcgggatgaaggggactgggtcagattctctaccaatgtcaagactgccgaaacgttgatgaatacccgcttcaacgtcttcaaggacaacctaaattccgtttcaaaaattcgaactttggagtattccgtccctgtagctatatcagctcatgtccaaatgatccagccaactaccttatttggacgacagaagccacagaacagtttgatcctaaaccccttgaccaaggatctagaatccatgtccgttgaagaatttgctgcttctcagtgcaggtccttagtgactactgcctgccttcgagaattgtacggacttggtgaccgtgtcactcaggctagggatgacaaccgtattggagtatccggctttttggaggagtacgcccaataccgcgatcttgagctcttcctctctcgctttgagccatccgccaaaggatttaatttcagtgaaggccttattgccggaggaaagaacactcagggtggtcctggaagctctactgaggccaaccttgatatgcaatatgtcgtcggtctgtcccacaaggcaaaggtcacctattactccaccgctggccgtggcccattaattcccgatctatctcagccaagccaagcttcaaacaacaacgaaccataccttgaacagctgcggtacctcgtaaagctccccaagaaccagcttccatctgtattgacaacttcctatggagacacagaacagagcttgcccgccagctataccaaagccacttgcgacctctttgctcagctaggaactatgggtgtgtctgttatcttcagcagtggtgataccgggcccggaagctcatgccagaccaacgatggcaagaatgcgactcgcttcaaccctatctacccagcttcttgcccgtttgtgacctccatcggtggaaccgttggtaccggtcctgagcgtgcagtttcattctcctctggtggcttctcagacaggttcccccgcccacaatatcaggataacgctgttaaagactacctgaaaattttgggcaaccagtggagcggattgtttgaccccaacggccgtgctttcccagatatcgcagctcagggatcaaattatgctgtctatgacaagggaaggatgactggagtctccggcaccagtgcatccgcccctgccatggctgccatcattgcccagcttaacgatttccgactggcaaagggctctcctgtgctgggattcttgaacccatggatatattccaagggtttctctggctttacagatattgttgatggcggttccaggggttgcactggttacgatatatacagcggcttgaaagcgaagaaggttccctacgcaagctggaatgcaactaagggatgggacccagtaacgggatttggtactcccaacttccaagctctcactaaagtgctgccctaa

SEQ ID NO: 61 - DNA, 1803 bp, Fusarium graminearum
atgtatatcacctcatcccgcctcgtgctggccttagcggcacttccgacagcatttggtaaatcatactcccaccatgccgaagcaccaaagggatggaaggtcgacgacaccgctcgtgttgcctccaccggtaaacaacaggtcttcagcatcgcactgaccatgcaaaatgttgatcagctcgagtccaagctccttgacctctccagccccgacagcaagaactatggccagtggatgtctcaaaaggacgtaacaactgctttctatccttcgaaagaagctgtttccagtgtgacaaagtggctcaagtccaagggtgtcaagcactacaacgtcaacggtggtttcattgactttgctctcgatgtcaagggtgccaatgcgctacttgatagtgactatcaatactacaccaaagagggccagaccaagttgcgaactctgtcttactctatccctgatgatgtagccgaacacgttcagttcgtcgacccaagcaccaactttggcggcacactggctttcgcccctgtcactcacccatcgcgtactctaaccgagcgcaagaacaagcccaccaagagcacagtcgatgcttcatgccaaaccagcatcacaccctcatgcttgaagcagatgtacaacattggtgactacactcccaaggtcgagtctggaagcactattggtttcagcagcttccttggcgagtccgccatctactccgatgttttcctgtttgaggagaagtttggaattcccacgcagaactttaccactgttctcatcaacaacggcactgatgaccagaacactgctcacaagaactttggcgaggctgacttggatgccgagaacattgttggaattgcccaccctcttcccttcacccagtacatcactggcggttcaccaccttttcttcccaacatcgatcagccaactgctgccgataaccagaacgagccttatgtgcctttcttccgctaccttctatcgcagaaggaagtccctgcagttgtctctacctcgtatggtgacgaagaagatagcgtccctcgcgaatatgctaccatgacctgcaacctgattggtcttctcggacttcgaggaatcagtgtcatcttctcctctggcgatatcggcgttggtgctggatgtctcggccctgaccacaagactgtcgagttcaacgccatcttccctgccacctgcccttacctcacctccgtcggcggtaccgttgatgtcacccccgaaatcgcctgggaaggttcttctggtggtttcagcaagtacttcccccgacccagctaccaggacaaggctgtcaagacgtacatgaagactgtctccaagcagacaaagaagtactacggcccttacaccaactgggaaggccgaggcttccctgatgttgctggccacagtgtctctcccaactatgaggttatctatgctggtaagcagagtgcaagcggaggtaccagtgctgctgctcctgtttgggctgccattgtcggtctgctcaacgatgcccgtttcagagctgggaagccaagcttgggatggttgaaccctcttgtttacaagtatggaccaaaggtgttgactgacatcactggtggttacgccattggatgtgatggcaacaacacccagtccggaaagcctgagcctgcaggatccggtattgtgcccggtgccagatggaatgccactgccggatgggatcctgtcactggttatggtacacccgactttggaaagttgaaggatttggttcttagcttctaa

SEQ ID NO: 62 - DNA, 1788 bp, Aspergillus kawachii
atgcgttcctccggtctttacgcagcactgctgtgctctctggccgcatcgaccaacgcagttgttcatgagaagctcgccgcggtcccctcgggctggcaccatctcgaagatgctggctccgatcaccagattagcctgtcgatcgcattggcacgcaagaacctcgatcagcttgaatccaagctgaaagacttgtccacaccaggtgaatcgcagtatggccagtggctggatcaagaggaagtcgacacactgttcccagtggccagcgacaaggccgtgatcagctggttgcgcagcgccaacatcacccatattgcccggcagggcagcttggtgaactttgcgaccaccgtcgacaaggtgaacaagcttctcaacaccacttttgcttactaccaaagaggttcttcccagagactgcgcacgacagagtactccattcccgatgatctggtcgactcgatcgacctcatctccccgacaacctttttcggcaaggaaaagaccagtgctggcctgacccagcggtcgcagaaagtcgacaaccatgtggccaaacgctccaacagctcgtcctgcgccgataccatcacgttatcctgcctgaaggagatgtacaactttggcaactacactcccagcgcctcgtcaggaagcaagctgggattcgccagcttcctgaacgagtccgcctcgtattccgatcttgccaagttcgagagactgttcaacttgccgtctcagaacttctccgtggagctgatcaacggcggcgtcaatgaccagaaccaatcgacggcttctctgaccgaggctgacctcgatgtggaattgctcgttggcgtaggtcatcctcttccggtgaccgagtttatcacttctggcgaacctcctttcattcccgaccccgatgagccgagtgccgccgataatgagaatgagccttaccttcagtactacgagtacctcctctccaagcccaactcggccctgccccaagtgatttccaactcctacggtgacgacgaacagaccgttccagaatactacgccaagcgagtctgcaacctgatcggactggtcggcctgcgcggcatcagcgtcctggaatcatccggtgacgaaggaattggatctggctgccgcaccaccgacggcactaacagcacccaattcaatcccatcttccccgccacctgtccctacgtgaccgccgtaggaggcaccatgtcctacgcgcccgaaattgcctgggaagccagttccggtggtttcagcaactacttcgagcgagcctggttccagaaggaagccgtgcagaactacctggcgaaccacatcaccaacgagacgaagcagtattactcacaattcgctaactttagcggtcgcggatttcccgatgtttcggcccatagctttgagccttcgtacgaagttatcttctacggcgcccgttacggctccggcggtacttccgccgcatgtcctctgttctctgcgctagtgggcatgttgaacgatgctcgtctgcgggcgggcaagtccacgcttggtttcttgaaccccctgctgtacagtaaggggtacaaggcgctgacagatgtcacggcgggacaatcgatcgggtgcaatggcattgatccgcagagtgatgaggctgttgcgggcgcgggcattatcccgtgggcgcattggaatgccacagtcggatgggatccggtgacgggattgggacttcctgattttgagaagttgaggcagttggtgctgtcgttgtag

SEQ ID NO: 63 - DNA, 1818 bp, Talaromyces stipitatus
atgagtcgaaatctcctcgttggtgctggcctgttggccctcgcccaattgagcggtcaagctctcgctgccgctgccctcgtcggccatgaatccctagctgcgctgccagttggctgggataaggtcagcacgccagctgcagggacgaacattcaattgtccgtcgccctcgctctgcaaaacatcgagcagctggaagaccacttgaagtctgtgtcaacccccggttctgccagctacggtcagtacctggattccgacggtattgccgctcaatacggtcccagcgacgcatccgttgaggctgtcaccaactggctgaaggaggccggtgtcactgacatctacaacaacggccagtcgattcacttcgcaaccagtgtcagcaaggccaacagcttgctcggggccgatttcaactactattctgatggtagtgcgaccaagttgcgtaccttagcttattccgttcccagtgacctcaaagaggccatcgaccttgtctcgcccaccacctatttcggcaagaccactgcttctcgtagcatccaggcttacaagaacaagcgcgcctctactacttccaagtctggatcgagctctgtgcaagtatctgcttcctgccagaccagcatcactcctgcctgcttgaaacagatgtacaatgttggcaactacacacccagcgtcgctcacggcagtcgtgtcggattcggtagcttcttgaatcaatctgccatctttgacgacttgttcacctacgaaaaggtcaatgatattccatcacagaatttcactaaggtgattattgcaaatgcatccaacagccaagatgccagcgatggcaactacggcgaagccaaccttgacgtgcaaaacattgtcggcatctctcatcctctccccgtgactgaattcctcactggtggctcacctcccttcgttgctagcctcgacacccctaccaaccagaacgagccatatattccttactacgaatatcttttgtctcagaagaacgaggatctcccccaggtcatttccaactcttacggagacgacgagcagtctgtgccgtacaagtatgccatccgtgcatgcaacctgatcggcctgacaggtttacgaggtatctcggtcttggaatccagcggtgatctcggcgttggagccggctgtcgcagcaacgatggcaagaacaagactcaatttgaccccatcttccctgccacttgcccctacgttacctctgttggtggtacccaatccgttacccctgaaattgcctgggtcgccagctccggtggtttcagcaactacttccctcgtacctggtaccaggaacccgcaattcagacctatctcggactccttgacgatgagaccaagacatactattctcaatacaccaactttgaaggccgtggtttccccgatgtttccgcccacagcttgacccctgattaccaggtcgtcggtggtggctatctccagccaagcggtggtacttccgctgcttctcctgtctttgccggcatcattgcgcttttgaacgacgctcgtctcgctgctggcaagcccactcttggcttcttgaacccgttcttctacctttatggatacaagggtttaaacgatatcactggaggacagtcagtgggttgcaacggtatcaacggccaaactggggctcctgttcccggtggtggcattgttcctggagcggcctggaactctactactggctgggacccagccactggtctcggaacacccgacttccagaagttgaaagaactcgtacttagcttttaa

SEQ ID NO: 64 - DNA, 1800 bp, Fusarium oxysporum
atgtatatctcctcccaaaatctggtactcgccttatcggcgctgccttcagcatttggcaaatccttctctcaccatgctgaagctcctcaaggctggcaagtccaaaagactgccaaagtcgcttccaacacgcagcatgtcttcagtcttgcactaaccatgcaaaacgtggatcagctcgaatccaagcttcttgacctctccagccccgacagcgccaactacggtaactggctctcccacgatgagctcacaagcactttctctccttccaaggaggcggtggctagtgtgacaaagtggctcaagtcaaagggcatcaagcactacaaggtcaacggtgctttcattgactttgctgctgatgttgagaaggccaatacgcttctcggaggtgattaccagtactacactaaggatggtcagacgaagctgagaacgctgtcttactccattcctgatgatgtcgccggtcacgttcaatttgttgatcctagcacaaacttcggtggcaccgttgcgttcaaccctgtgcctcacccctcgcgcaccctccaagagcgcaaggtctctccctccaagagcaccgttgatgcttcatgccagacaagcatcaccccttcttgcctcaagcagatgtacaacattggagactacactcccgatgccaagtctggaagtgagattggtttcagcagctttctcggccaggctgctatttactctgatgtcttcaagtttgaggagctgtttggtattcctaagcagaactacaccactattctgatcaacaatggcaccgatgatcagaatactgcgcatggaaactttggagaggctaaccttgatgctgagaacattgttggaatcgctcatcctcttcctttcaagcagtacattactggaggttcaccacctttcgttcccaacatcgatcagcccaccgagaaggataaccagaacgagccctacgtgcctttcttccgttacctcttgggccagaaggatctcccagccgtcatctccacttcctacggcgatgaagaagacagcgttcctcgtgagtatgctacactcacctgcaacatgatcggtcttctcggtctccgtggcatcagtgtcatcttctcttccggtgacatcggtgtcggttccggctgccttgctcccgactacaagaccgtcgagttcaatgccatcttccccgccacatgcccctacctcacctccgtcggcggtaccgtcgacgtcacccccgagatcgcctgggagggatcctccggcggattcagcaagtacttcccccgacccagctaccaggacaaggccatcaagaagtacatgaagacagtctccaaggagaccaagaagtactacggcccttacaccaactgggagggccgaggtttccctgatgtcgctggacacagtgttgcgcctgactacgaggttatctacaatggtaagcaggctcgaagtggaggtaccagcgctgctgcccctgtttgggctgctatcgttggtctgttgaacgatgcccgcttcaaggctggtaagaagagcttgggatggttgaaccctcttatctacaagcatggacccaaggtcttgactgacatcaccggtggctatgctattggatgtgacggtaacaacactcagtctggaaagcccgagcccgctggatctggtcttgttcccggtgctcgatggaacgccacagctggatgggatcctaccactggctatggaactcccaacttccagaagttgaaggacttggttctcagcttgtaa

SEQ ID NO: 65 - DNA, 1839 bp, Trichoderma virens
atgcctaagtccacagcgcttcggcttgttagcctcctttccctggccagtgtgccgatatctgcctccgtccttgtggaaagtctcgaaaagctgcctcacggatggaaagctgcttcggctcctagcccttcctcccagataaccctacaagtcgctcttacgcagcagaacatcgatcagctggaatcgaggctcgcggctgtatccacaccaaattccaagacatacggaaattatctggatcttgatgagatcaatgagatcttcgcgccaagcgatgccagcagcgcagccgtggagtcttggctccattctcacggtgtgacaaaatacacgaagcaaggcagcagtatctggttccaaaccgaagtttctacagcaaatgcaatgttgagcacaaacttccacacttacagtgatgctgctggcgttaagaagttgcgaactcttcagtattcaattccggagagtcttgtgggccatgtcgatctcatctcacccacgacctactttggcacctctaacgctatgagagctttgagatctaaaagcgtggcttcagttgctcaaagtgtggcagcccgccaagaaccttctagctgcaagggaactctggttttcgaaggaagaacgttcaatgtcttccaaccagattgtcttaggacagagtacaatgtcaatggatacactccatcagccaagtctggtagtagaataggatttggttccttcttaaaccaaagtgcaagcttttcagacctcgcactctttgaaaaacactttgggttttccagccaaaatttctccgtcgttctgatcaatggtggaacggacctgccccaaccaccctctgacgacaacgatggcgaggccaatttggatgtccaaaacattttgacaatcgcacaccctctgcccatcactgaattcatcactgccggaagcccgccgtacttcccagatcccgttgaacctgcaggaactcccgatgagaacgagccttacttgcagtactttgagtatctgttgtcgaagcccaacagagatcttcctcaggtcattaccaactcttacggtgatgaggagcaaacagtacctcaggcttatgctgtccgagtgtgcaacctaattggattgatgggactgcgtggtatcagtatcctcgagtcctccggcgatgagggagtgggtgcttcctgcgttgctaccaacagcaccactcctcaatttaaccccattttcccggcaacatgcccctatgtcactagcgtaggtggaactgtgaacttcaacccagaagttgcctgggacggttcatctggaggtttcagctactatttctccaggccatggtaccaagaggaagcagttggaaactacctagagaagcatgtcagcgccgaaacaaagaagtactacgggccttatgtcgatttctctggacgtggcttccctgatgttgcagctcacagcgtgagccccgattatcctgtgttccaaggcggccagctcactcctagcggaggcacttctgcggcttctcccgtcgtagccagtatcattgcccttctgaacgatgcacgcctccgtgaaggcaagcccacacttgggttcctgaacccgctgatttaccaatatgcttacaagggtttcacggatatcacatccggccagtctgatggctgcaatggcaacaacacccaaacggatgcccctcttcctggagctggcgttgtcctaggagcacactggaatgcgaccaaaggatgggatcctacgacaggatttggtgtccctaactttaagaagctactcgagctgatccgatatatatag

SEQ ID NO: 66 - DNA, 1776 bp, Trichoderma atroviride
atggctaaactgacggcacttcggctcgtcagccttctttgccttgcggctgcgcaggcctctgctgctgtgctcgtggaaagcctcaaacaagtgcccaacgggtggaatgcagtctcgaccccagacccttcgacatcgattgtcttgcaaatcgccctcgcgcaacagaatatcgatgaattggaatggcgtctcgcggctgtatccacgcccaactctggcaattatggcaaatacctggatattggagagattgaaggaattttcgccccaagcaatgcctcttacaaagccgtggcatcgtggctccagtctcatggggtgaagaacttcgtcaaacaagccggcagtatttggttctacactactgtctctaccgcaaacaagatgcttagcacagatttcaaacactatagcgatcctgttggcattgagaagctgcgtactcttcagtactcgatcccagaagaactagtcggccatgttgatctcatctcgcctacaacatattttggaaacaaccaccccgcgacagcgagaacacccaacatgaaggccattaacgtaacctaccaaatctttcacccagactgccttaaaacgaaatacggcgttgatggctatgccccatctccaagatgtggcagcaggattggttttggctcattcctcaacgaaactgccagttattcggatcttgcgcagtttgagaagtactttgaccttcccaaccaaaacctttccaccttattgatcaatggcgcaatcgacgttcagccaccttccaacaaaaacgacagcgaggccaacatggacgttcagaccatcttgacctttgtccaacctcttcctattactgagtttgttgttgccggaatcccgccgtatattcctgatgcggctttgccgatcggcgaccctgtccaaaacgagccgtggctggaatactttgagtttttgatgtccaggaccaacgcagagcttccccaggtcattgccaactcatacggtgacgaggaacaaacggtaccacaggcgtatgccgtccgagtatgcaaccagattgggctgttgggccttcgcggtatatccgttatcgcatcatctggcgatacgggtgttggaatgtcttgtatggcttcgaacagcactactcctcagtttaaccccatgttcccggcttcgtgtccttatatcaccactgtcggtggaactcagcaccttgataatgagattgcttgggagctttcatcgggaggcttcagtaactatttcacaaggccatggtatcaagaagacgcagccaaaacatatcttgaacgtcatgtcagcaccgagacaaaggcatattacgaacgttacgccaatttcttgggacgcggctttcccgacgttgcagcacttagtctcaaccccgattatccagtgattattggcggagaacttggtcccaatggaggcacttctgcggccgcacccgtcgtcgctagtattattgcactcttgaacgatgcacgcctttgcctaggcaaacctgcccttgggttcttgaaccccctgatctatcaatatgctgataagggtggcttcacggatatcacgtccggccagtcttggggctgtgccggaaataccactcagacggggcctcctccccctggagctggtgtcattccgggggcacactggaatgcgaccaagggatgggatcctgtaacaggatttggaaccccgaacttcaagaaattactctcactggccctgtccgtctaa

SEQ ID NO: 67 - DNA, 1749 bp, Agaricus bisporus
atgttttggcgtccagcttttgtccttcttctcgctcagcttgtcactgctagtcctttagctcgacgctgggatgatttcgcagaaaaacatgcctgggttgaagttcctcgcgggtgggaaatggtctccgaggctcccagtgaccatacctttgatcttcgcattggagtaaagtcaagtggcatggagcagctcattgaaaacttgatgcaaaccagcgatcctactcattccagatatggtcaacatcttagtaaagaagagctccatgatttcgttcagcctcatcctgattctaccggagcggtcgaagcatggcttgaagatttcggtatctccgatgatttcattgatcgtactggaagtggcaactgggttactgttcgagtttcagtagcccaggctgaacgtatgcttggtaccaagtataacgtctaccgccattctgaatcaggggaatcggttgtacgaacaatgtcttattcgcttcccagcgaacttcactcccacatagatgttgtcgcacccaccacttatttcggcacgatgaaaagcatgcgggtgaccagcttcttacagccggaaatagagcctgttgacccaagcgctaaaccatcggctgctccagcttcctgtttgagtaccactgtcataacccccgattgcctccgtgacctttataatacggctgactacgttccttccgccacttcacggaatgccattggtattgctgggtacttggatcgttcaaatcgtgcagatcttcagactttcttccgacgcttccggcccgatgccgttggcttcaattacacgactgtccaactaaatggcggaggagacgaccagaatgatcccggtgtagaggccaacctcgatattcaatacgccgctggtattgctttccccacaccagctacatactggagtactggcggctctccacctttcattccagatactcaaaccccgacaaacaccaatgagccctacctggattggatcaattttgtcctaggccaggacgagattccacaggtgatttcaacgtcctatggtgacgacgagcaaacagttcctgaagattacgctactagcgtgtgtaatctcttcgcgcaactcggcagccgtggcgttacagtattcttctccagcggtgactttggtgttggtggtggagattgcctcacgaatgatggctcaaaccaagtccttttccagccggctttccccgcttcctgcccattcgtaacagctgttggcggaactgtcaggcttgatcctgagattgctgtcagtttctctggaggaggcttttcccgttacttctccaggccatcgtaccagaatcaaactgtggctcaatttgtttctaatcttgggaatacattcaacggactctacaataaaaatggaagggcctacccagatcttgcagcacagggcaatggcttccaagttgttatagacggcatcgtccgttcggttggagggaccagcgccagctctccgacggttgccggtatctttgcgcttttgaatgacttcaagctctcaagaggccagtcgacactcggatttatcaacccacttatatactcctccgctacatccggcttcaatgacatcagggcgggtacaaaccctggttgtggtactcgcggatttaccgctggtactggttgggatccggtcactggtctgggcactcccgattttttgaggcttcagggacttatttaa

SEQ ID NO: 68 - DNA, 1806 bp, Magnaporthe oryzae
atgctcgaccgtatccttctccccctcggcctcctggcctcccttgccaccgctcgtgtctttgacagcctaccccaccctccccgaggctggtcatactcgcacgcggcggaatcgacggagccgctgaccctgcgcatcgccctccgccagcaaaatgccgccgccctggagcaggtggtgctgcaggtctcgaaccccaggcacgccaattacggccagcacctgacgcgcgacgagctgcgcagctacacggcgcccacgccgcgggccgtccgcagcgtgacgtcgtggctggtcgacaacggcgtcgacgactacacggtcgagcacgactgggtgacgctgcgcacgacggtcggggccgcggacaggctgctcggcgcagactttgcctggtatgccggcccgggcgagacgctgcagctgcggacgctctcgtacggcgtcgacgactcggtggcgccgcacgtcgacctcgtgcagcccacgacgcggtttggcggtcccgtcgggcaggcgtcgcacatcttcaagcaggacgactttgacgagcagcagctcaagaccttgtcggtggggttccaggtcatggctgacctgccggccaacgggcctgggtcgatcaaggcggcatgtaacgagtctggcgtgacgcccctgtgcctgcgaactctgtacagggtcaactacaagccggcaaccacggggaacctggtcgctttcgcgtcgttcctggagcagtacgccaggtacagtgatcagcaggcattcactcagcgggtccttggccctggtgttccgttgcagaacttttcggtcgaaacggtcaacggtggagccaatgaccagcagagcaaacttgacagcggcgaggcgaacctcgatctgcagtacgtcatggcaatgagccaccctattccaattttggagtacagcactggaggcagaggacccctcgtcccaactctggaccagcccaacgccaacaacagcagcaatgagccttacctggagttcctgacgtacctcctggcccaacccgactcagccatccctcagaccctgtcggtgtcgtatggcgaggaggaacagtcggtgccgcgcgactacgccatcaaggtttgcaacatgttcatgcagctcggcgcccgcggcgtgtcggttatgttttcgtcgggcgactcgggcccgggtaatgactgtgttcgagcctcggacaacgcaaccttttttggctcaacattccccgcaggctgcccctacgtcacgtcggtgggctccaccgtcggcttcgagccggagcgcgccgtctccttttcctcgggcggcttcagcatttaccacgctcgccccgactaccaaaacgaagtggtccccaagtacattgaatcgatcaaggcttcgggctacgaaaagttctttgacggcaacggccgcggaattcccgacgtggctgcccagggcgcccgcttcgtcgtcatcgacaagggccgcgtttctctaatctcggggaccagcgccagctcacctgcgtttgctggcatggtggcgctcgtcaacgccgcccgcaagtcaaaggacatgccggccttgggcttcctcaaccccatgctgtaccagaacgccgcggccatgacggacattgtcaacggcgctggcatcggctgcaggaagcaacgtacagaattcccgaatggcgccaggttcaacgccacggccggctgggatcccgtcacagggctggggacgccgttgtttgacaagctgctggctgttggcgcacctggagttcccaacgcgtga

SEQ ID NO: 69 - DNA, 1845 bp, Togninia minima
atgcgtagccagttgctcttctgcacagcatttgctgctctccagtcgcttgtggagggcagcgatgtggtgttggagtcattgcgagaggtccctcagggctggaagaggcttcgagatgcggaccccgagcagtccatcaagctgcgcattgcgcttgagcagcctaacctggacctgttcgagcagaccctctacgacatctcgtcaccggatcacccaaaatatggccagcatctcaagagccacgagttacgggatattatggcacctcgcgaggagtcaactgctgctgtcatcgcttggctgcaagacgctgggctttctggctcgcagattgaggacgacagcgactggatcaacatccagacgacagtcgcccaagccaacgacatgctgaacacgactttcggtctcttcgcccaggaaggcaccgaggtcaatcgaattcgagctctggcatattccgtgcctgaggagatcgtccctcacgtcaagatgattgctcccatcatccgcttcggtcagttgagacctcagatgagccacatcttctcgcatgagaaagtcgaggagaccccgtctattggcaccatcaaggccgccgctatcccatctgtggatcttaacgtcaccgcttgcaatgccagcatcacccccgagtgcctccgagcgctttacaacgttggtgattacgaggcggacccatcgaagaagtctcttttcggagtctgtggctacttggagcaatatgccaagcacgatcagctggccaagtttgagcagacctacgctccgtatgctatcggtgccgacttcagcgtcgtgaccatcaatggcggaggcgacaaccagaccagtacgatcgatgatggagaagccaacctggatatgcagtatgctgtcagcatggcatacaagacgccaatcacatactattcaactgggggtcgaggacctcttgttccagatctcgaccaacctgatcccaacgacgtctcaaacgagccgtaccttgattttgtgagctaccttctcaagctgcccgactccaaattgccgcagaccatcacaacttcgtacggagaggatgagcaatccgttccacgctcctacgtggagaaggtctgcaccatgttcggcgcgctcggtgcccgaggcgtgtctgtgatcttctcctctggtgataccggtgtcggctcagcgtgccagaccaacgacggcaagaacaccacccgcttcctgcctatattccctgctgcgtgcccttatgtgacctcggttggaggcactcgctatgtcgacccggaagtcgctgtgtccttctcgtctggaggcttctcggacatcttccctacgccactctaccagaagggcgctgtctctggctacctgaagatcctcggcgatcgctggaagggcctctataaccctcacggccgcggtttccctgacgtctccggacagagtgtcagataccacgtcttcgactacggcaaggacgtcatgtactctggcacaagtgcctctgcaccgatgttcgccgcgcttgtctcgctgctgaacaacgcccgtctcgcaaagaagttgccgcccatgggattcctgaatccctggctgtataccgttggttttaacgggctgacggatattgtgcacggtggatctactgggtgcactggcacagacgtgtacagcggcctgcccacacctttcgttccgtatgcgtcttggaacgcaaccgtgggatgggaccccgttactggacttggcacgcctctctttgataagctgctcaatttgagcacgccaaacttccacttgccgcacattggcggtcactag

SEQ ID NO: 70 - DNA, 1914 bp, Bipolaris maydis
atgaagtacaacacactcctcaccggcctgctggctgttgcccatggcagtgccgtttccgcttcaactacttcacatgtcgagggtgaagttgtcgagcgacttcatggcgttcctgagggttggagtcaagtgggcgcccccaatccagaccagaagctgcgctttcgcatcgcagtacgctcggtgagtaattgcttttgtgaacccatgtttgaatcttgcggtgctttttactgaacataacaggcggatagcgagctgtttgagaggacgcttatggaggtttcttctcccagccatcctcgctacggacagcacctaaagcgacacgaactcaaggacctcatcaaaccgcgcgccaagtcaacttcaaacatcctgaactggctgcaagagtctggaattgaggccagagatatccagaacgatggcgagtggatcagcttctatgctccggttaaacgtgccgagcaaatgatgagcactacattcaagacctatcagaacgaggcccgagcgaatatcaagaagatccgctctctagactactcggtgccgaagcacattcgagatgacatcgacatcatccagcctacgactcgcttcggccagatccaaccggagcgtagccaagtctttagtcaagaagaggtcccattctcagcgcttgttgtcaatgcgacgtgtaacaagaaaatcactcccgactgcctcgccaacctctacaacttcaaagactatgatgccagcgatgccaatgtcactatcggagtcagcggcttcctggagcaatatgctcgctttgacgacttgaagcaattcatcagcactttccaaccaaaagcagctggttccacattccaagttacatctgtcaatgcagggccttttgaccagaactcgacagccagcagtgttgaagccaatcttgacattcagtacacaacaggtcttgttgcgcccgacattgaaacccgctacttcactgttcccggtcgcggtatcctgatccctgatctggaccagcctacggagagcgacaacgctaatgagccgtatctggattactttacatatcttaataacctcgaagacgaagaactccccgacgtgctgaccacatcttacggcgagagcgagcagagtgtacccgccgaatatgcaaaaaaggtgtgcaatttgatcggccagttgggtgctcgtggtgtgtccgtcatcttctccagcggtgatactggccctggctctgcatgccaaaccaatgatggaaaaaacacgacacgtttcttgcccatcttccctgcttcttgcccctacgtcacttcggttggcggcactgttggtgttgagcccgaaaaggctgtcagcttctcttcgggcggcttttctgacctatggcctcgacccgcttatcaagagaaggccgtatcagaatatcttgaaaagctcggagaccgctggaacgggctttacaaccctcaaggacgcggatttcctgatgtagctgctcagggccaaggcttccaggtgtttgacaagggcaggctgatttcggtcggaggaacgagcgcttcagctcctgttttcgcatccgtagtcgcactcctgaacaatgctcgcaaggctgccggcatgtcttcactcggcttcttgaacccatggatctacgagcaaggctacaagggcttgaccgatatcgttgctggaggctcgacaggatgcacaggaagatccatctattcaggcctcccagcaccactcgtgccgtatgcttcttggaatgccaccgaaggatgggatccggtgacgggctatggtacacctgatttcaagcaattgctcaccctcgcgacggcacccaagtctggcgagcgtcgcgttcgtcgtggcggtctcggtggccaggcttag

SEQ ID NO: 71 - DNA, 1842 bp, Aspergillus kawachii
atgttatcttccttccttagccagggagcagccgtatccctcgcgttattgtcgctgctcccttcgcctgtagccgcggagatcttcgagaagctgtccggcgtccccaatggctggagatacgccaacaatcctcacggcaacgaggtcattcgccttcaaatcgcccttcagcagcacgatgttgccggtttcgaacaagccgtgatggacatgtccacccccggtcacgccgactatggaaagcatttccgcacacatgatgagatgaagcgcatgctgctccccagcgacactgccgtcgactcagttcgcgactggctggaatccgccggagtccacaatatccaggtcgacgccgactgggtcaagttccataccaccgtcaacaaggccaatgccctgctggatgccgacttcaagtggtatgtcagcgaggccaagcacattcgtcgtctacgcaccctgcaatactccatccccgacgccctggtctcgcacatcaacatgatccagcccaccactcgctttggccagatccagccgaaccgtgccaccatgcgcagcaagcccaagcacgccgacgagacattcctgaccgcagccaccttggcccagaacacctcccactgcgactccatcatcacgccgcactgtctgaagcagctctacaacatcggtgactaccaggccgaccccaagtccggtagcaaggtcggcttcgccagctacctcgaagaatacgcccggtatgccgatctcgaaaggttcgagcagcacctggctcccaacgccatcggccagaacttcagcgtcgttcaattcaacggcggcctcaacgaccagctttcattgagcgacagcggcgaagccaacctcgacctgcagtacatcctgggcgtcagcgctcccgtcccggtcactgaatacagcactggcggacgcggcgaactggtccccgacctgagctccccggaccccaacgacaacagcaacgagccctacctcgacttcctccagggtattctcaaactcgacaattccgacctcccccaagtcatctctacctcctacggcgaagacgaacagaccatccccgtcccctacgcccgcacagtctgcaatctctacgcccaactcggcagccgcggtgtctccgtgatcttctcgagcggcgactccggcgtcggcgccgcctgcctcaccaacgacggcaccaaccgcacccacttccctcctcaattcccggcctcctgcccctgggtaacctccgtcggtgccaccagcaaaacctccccggagcaagccgtctccttctcctcaggaggcttctccgacctctggccccgcccctcctaccaacaggctgccgtccaaacctacctcacccagcacctgggcaacaagttctcaggcctcttcaacgcctccggccgcgccttccccgacgtcgccgcgcagggcgtcaactacgccgtctacgacaagggcatgcttggccagttcgatggaaccagttgctccgcgccgacgttcagtggtgtcattgccttgttgaatgacgccagactgagggcgggtttgcccgttatgggattcctgaacccgttcctctatggagttggtagtgagagtggcgcgttgaatgatattgtcaacggcgggagcctgggttgtgatggtaggaatcgatttggaggcacgcccaatggaagtcccgttgtgccgtttgctagttggaatgcgaccaccgggtgggatccggtttctgggctgggaacgccggattttgcgaagttgaggggtgtggcgttgggtgaagctaaggcgtatggtaattaa

SEQ ID NO: 72 - DNA, 1977 bp, Aspergillus nidulans
auggcagcgacuggacgauucacugccuucuggaaugucgcgagcgugcccgccuugauuggcauucucccccuugcuggaucucauuuaagagcuguccuuugcccugucuguaucuggcgucacucgaaggccguuugugcaccagacacuuugcaagccaugcgcgccuucacccguguaacggccaucucccuggccgguuucuccugcuucgcugcugcggcggcugcggcuuuugagagccugcgagcugucccugacggcuggaucuacgagagcacccccgacccuaaccaaccgcugcgucuacgcaucgcgcugaaacagcacaaugucgccggcuucgagcaggcacugcuggauauguccacacccggucacuccagcuacgggcagcauuucggcuccuaccacgagaugaagcagcugcuucucccuaccgaggaggcguccuccucggugcgagacuggcucucggcggcgggcguugaguucgaacaggacgccgacuggaucaacuuccgcacgaccgucgaccaggcuaacgcccuccucgacgccgauuuccucugguacacaacgaccggcucgacgggcaacccgacgcggauccuccgaacccucuccuacagcguucccagcgagcucgcuggauacgucaacaugauccagccgacuacgcguuucggcggcacgcaugccaaccgggccaccguucgcgcgaagccgaucuuccucgagaccaaccggcagcucaucaacgccaucuccucuggcucgcucgagcacugcgagaaggccaucaccccaucgugccuggcggaucuguacaacacugaaggguacaaggcguccaaccgcagcgggagcaagguggccuuugccuccuuccucgaagaguacgcgcgcuacgacgaucucgccgaguucgaggagaccuacgcucccuaugcgaucgggcagaacuucucgguuaucuccaucaacggcggccucaacgaccaggacuccacggccgacagcggcgaggcgaaccucgaccugcaguacaucaucggcgucucgucgccgcuaccugugaccgaguucacaaccgguggccgcggcaagcucauuccugaccucuccucccccgacccgaaugacaacaccaacgagccuuuccuugacuuccuugaggccguccucaagcucgaucagaaagaccugccccaggucaucucgaccuccuacggcgaggacgagcagacaaucccugagccguacgcccgcuccgucugcaaccuguacgcucagcucgguucccgcggcgugucugugcucuucuccucgggugacucuggcgucggcgccgccugccagaccaacgauggcaaaaacacgacgcacuucccgccgcaguucccggccucuugccccugggugaccgccgucggcggcacgaacggcacagcgcccgaauccgguguauacuucuccagcggcggguucuccgacuacugggcgcgcccggcguaccagaacgccgcgguugagucauaccugcgcaaacucgguagcacacaggcgcaguacuucaaccgcagcggacgcgccuucccggacgucgcagcgcaggcgcagaacuucgcugucgucgacaagggccgugucggucucuucgacggaacgagcugcaguucgccuguauuugcgggcaucguggcguugcucaacgacgugcgucugaaggcaggccugcccgugcugggauuccucaacccuuggcucuaccaggauggccugaacgggcucaacgauaucguggauggagggagcaccggcugcgacgggaacaaccgguuuaacggaucgccaaaugggagccccguaaucccguaugcggguuggaacgcgacggaggggugggauccugugacggggcugggaacgccggauuucgcgaagcugaaagcgcucgugcuugaugcuuag

SEQ ID NO: 73 - DNA, 1815 bp, Aspergillus ruber
atgttgtcatttgttcgtcggggagctctctccctcgctctcgtttcgctgttgacctcgtctgtcgccgccgaggtcttcgagaagctgcatgttgtgcccgaaggttggagatatgcctccactcctaaccccaaacaacccattcgtcttcagatcgctctgcagcagcacgatgtcaccggtttcgaacagtccctcttggagatgtcgactcccgaccatcccaactacggaaaacacttccgcacccacgatgagatgaagcgcatgcttctccccaatgaaaatgccgttcacgccgtccgcgaatggctgcaagacgccggaatcagcgacatcgaagaagacgccgattgggtccgtttccacaccaccgtggaccaggccaacgacctcctcgacgccaacttcctctggtacgcgcacaagagccatcgtaacacggcgcgtctccgcactctcgagtactcgatcccagactctattgcgccgcaggtcaacgtgatccagccaaccacgcgattcggacagatccgtgccaaccgggctacgcatagcagcaagcccaagggtgggcttgacgagttggctatctcgcaggcagctacggcggatgatgatagcatttgtgaccagatcaccaccccacactgtctgcggaagctgtacaatgtcaatggctacaaggccgatcccgctagtggtagcaagatcggttttgctagtttcctggaggaatacgcgcggtactctgatctggtactgttcgaggagaacctggcaccgtttgcggagggtgagaactttactgtcgtcatgtacaacggcggcaagaatgaccagaactccaagagcgacagcggcgaggccaacctcgatctgcagtacatcgtgggaatgagcgcgggcgcgcccgtgaccgagttcagcaccgccggtcgcgcacccgtcatcccggacctggaccagcccgaccccagcgccggtaccaacgagccgtacctcgagttcctgcagaacgtgctacacatggaccaggagcacctgccgcaggtgatctctacttcctacggtgagaacgaacagaccatccccgaaaagtacgcccgcaccgtttgcaacatgtacgcgcagctgggcagccgcggtgtgtcggtgattttctcgtcgggcgactccggcgtcggctctgcctgtatgaccaacgacggtacaaaccgcacccacttccccccgcagttcccggcgtcctgcccctgggtgacatcggtcggggccactgagaagatggcccccgagcaagcgacatatttctcctcgggcggcttctctgacctcttcccgcgcccaaagtaccaggacgctgctgtcagcagctaccttcagaccctcggatcccggtaccagggcttgtacaacggttccaaccgtgcattccctgacgtctcggcgcagggtaccaactttgctgtgtacgacaagggccgtctaggccagttcgatggtacttcttgctctgctcccgcgtttagcggtatcatcgccttgctcaacgacgtccgtctccagaacaacaagcccgtcctgggcttcttgaacccctggttgtatggcgctgggagcaagggcctgaacgacgtcgtgcacggtggcagtacaggatgcgatggacaggagcggtttgcaggaaaggccaatggaagccccgtcgtgccgtacgctagctggaatgctacgcaaggctgggatccagtcactggccttggaacgccggatttcggcaagttgaaggatttggctctgtcggcttaa

SEQ ID NO: 74 - DNA, 1803 bp, Aspergillus terreus
auguugcccucucuuguaaacaacggggcgcugucccuggcugugcuuucgcugcucaccucguccgucgccggcgagguguuugagaagcugucggccgugccgaaaggauggcacuucucccacgcugcccaggccgacgcccccaucaaccugaagaucgcccugaagcagcaugaugucgagggcuucgagcaggcccugcuggacauguccaccccgggccacgagaacuacggcaagcacuuccacgagcacgacgagaugaaacgcaugcugcuccccagcgacuccgccgucgacgccguccagaccuggcugaccuccgccggcaucaccgacuacgaccucgacgccgacuggaucaaccugcgcaccaccgucgagcacgccaacgcccugcuggacacgcaguucggcugguacgagaacgaagugcgccacaucacgcgccugcgcacccugcaauacuccauccccgagaccgucgccgcgcacaucaacauggugcagccgaccacgcgcuuuggccagauccggcccgaccgcgcgaccuuccacgcgcaccacaccuccgacgcgcgcauccuguccgcccuggccgccgccagcaacagcaccagcugcgacucagucaucacccccaagugccucaaggaccucuacaaggucggcgacuacgaggccgacccggacucgggcagccaggucgccuucgccagcuaccucgaggaauacgcccgcuacgccgacauggucaaguuccagaacucgcucgcccccuacgccaagggccagaacuucucggucguccuguacaacggcggcgucaacgaccagucguccagcgccgacuccggcgaggccaaccucgaccugcagaccaucaugggccucagcgcgccgcuccccaucaccgaguacaucaccggcggccgcggcaagcucauccccgaucucagccagcccaaccccaacgacaacagcaacgagcccuaccucgaguuccuccagaacauccucaagcuggaccaggacgagcugccgcaggugaucucgaccuccuacggcgaggacgagcagacaaucccccguggcuacgccgaauccgucugcaacaugcuggcccagcucggcagccgcggcgugucgguggucuucucgucaggcgauucgggcgucggcgccgccugccagaccaacgacggccgcaaccaaacccacuucaacccgcaguucccggccagcugcccgugggugacgucggucggggccacgaccaagaccaacccggagcaggcgguguacuucucgucgggcggguucucggacuucuggaagcgcccgaaguaccaggacgaggcgguggccgcguaccuggacacgcugggcgacaaguucgcggggcuguucaacaagggcgggcgcgcguucccggacgucgcggcgcagggcaugaacuacgccaucuacgacaagggcacgcugggccggcuggacggcaccucgugcucggcgccggccuucucggccaucaucucgcugcugaacgaugcgcgccugcgcgaggguaagccgaccaugggcuucuugaacccguggcuguauggugagggccgcgaggcgcugaaugauguugucgugggugggagcaagggcugugaugggcgcgaccgguuuggcggcaagcccaaugggagcccugucgugccuuuugcuagcuggaaugcuacgcagggcugggacccgguuacugggcuggggacgccgaacuuugcgaagauguuggagcuggcgccauag

SEQ ID NO: 75 - DNA, 1806 bp, Penicillium digitatum
atgattgcatcattattcaaccgtagggcattgacgctcgctttattgtcactttttgcatcctctgccacagccgatgtttttgagagtttgtctgctgttcctcagggatggagatattctcgcacaccgagtgctaatcagcccttgaagctacagattgctctggctcagggagatgttgctgggttcgaggcagctgtgatcgatatgtcaacccccgaccaccccagttacgggaaccacttcaacacccacgaggaaatgaagcggatgctgcagcctagcgcggagtccgtagactcgatccgtaactggctcgaaagtgccggtatttccaagatcgaacaggacgctgactggatgaccttctataccaccgtgaagacagcgaatgagctgctggcagccaacttccagttctacatcaatggagtcaagaaaatagagcgtctccgcacactcaagtactctgtcccggacgctttggtgtcccacattaacatgatccagccaaccacccgtttcggccagctgcgcgcccagcgcgccattttacacaccgaggtcaaggataacgacgaggctttccgctcaaatgccatgtccgctaatccggactgcaacagcatcatcactccccagtgtctcaaggatttgtacagtatcggtgactatgaggccgaccccaccaatgggaacaaggtcgcgtttgccagctacctagaggagtatgcccgatactccgatctcgcattatttgagaaaaacatcgccccctttgccaagggacagaatttctccgttgtccagtataacggcggtggtaatgatcaacaatcgagcagtggcagtagtgaggcgaatcttgacttgcagtacatcgttggagtcagctctcctgttcccgttacagagtttagcactggaggtcgcggtgaacttgttccggatctcgaccagccgaatcccaatgacaacaacaacgagccataccttgaattcctccagaacgtgctcaagttgcacaagaaggacctcccccaggtgatttccacctcttatggcgaggacgagcagagcgttccagagaagtacgcccgcgccgtttgcaacctgtactcccaactcggtagccgtggtgtgtccgtaatcttttcatccggcgactctggcgttggcgccgcgtgtcagacgaacgacggccggaacgcgacccacttcccaccccagttcccggccgcctgcccctgggtgacatcagtcggtgcgacaacccacactgcgcccgaacgagccgtttacttctcatctggcggtttctccgatctctgggatcgccctacgtggcaagaagatgctgtgagtgagtacctcgagaacctgggcgaccgctggtctggcctcttcaaccctaagggccgtgccttccccgacgtcgcagcccagggtgaaaactacgccatctacgataagggttctttgatcagcgtcgatggcacctcttgctcggcacctgcgtttgccggagtcatcgccctcctcaacgacgcccgcatcaaggccaatagaccacccatgggcttcctcaacccttggctgtactctgaaggccgcagcggcctaaacgacattgtcaacggcggtagcactggctgcgacggtcatggccgcttctccggccccactaacggtggtacgtcgattccaggtgccagctggaacgctactaagggctgggaccctgtctccggtcttggatcgcccaactttgctgccatgcgcaaactcgccaacgctgagtag

SEQ ID NO: 76 - DNA, 1806 bp, Penicillium oxalicum
atgcatgttcctctgttgaaccaaggcgcgctgtcgctggccgtcgtctcgctgttggcctccacggtctcggccgaagtattcgacaagcttgtcgctgtccctgaaggatggcgattctcccgcactcccagtggagaccagcccatccgactgcaggttgccctcacacagggtgacgttgagggcttcgagaaggccgttctggacatgtcaactcccgaccaccccaactatggcaagcacttcaagtcacacgaggaagttaagcgcatgctgcagcctgcaggcgagtccgtcgaagccatccaccagtggctcgagaaggccggcatcacccacattcaacaggatgccgactggatgaccttctacaccaccgttgagaaggccaacaacctgctggatgccaacttccagtactacctcaacgagaacaagcaggtcgagcgtctgcgcaccttggagtactcggttcctgacgagctcgtctcgcacattaaccttgtcaccccgaccactcgcttcggccagctgcacgccgagggtgtgacgctgcacggcaagtctaaggacgtcgacgagcaattccgccaggctgctacttcccctagcagcgactgcaacagtgctatcaccccgcagtgcctcaaggacctgtacaaggtcggcgactacaaggccagtgcctccaatggcaacaaggtcgccttcaccagctacctggagcagtacgcccggtactcggacctggctctgtttgagcagaacattgccccctatgctcagggccagaacttcaccgttatccagtacaacggtggtctgaacgaccagagctcgcctgcggacagcagcgaggccaacctggatctccagtacattatcggaacgagctctcccgtccccgtgactgagttcagcaccggtggtcgtggtcccttggtccccgacttggaccagcctgacatcaacgacaacaacaacgagccttacctcgacttcttgcagaatgtcatcaagatgagcgacaaggatcttccccaggttatctccacctcgtacggtgaggacgagcagagcgtccccgcaagctacgctcgtagcgtctgcaacctcatcgctcagctcggcggccgtggtgtctccgtgatcttctcatctggtgattccggtgtgggctctgcctgtcagaccaacgacggcaagaacaccactcgcttccccgctcagttccccgccgcctgcccctgggtgacctctgttggtgctactaccggtatctcccccgagcgcggtgtcttcttctcctccggtggcttctccgacctctggagccgcccctcgtggcaaagccacgccgtcaaggcctaccttcacaagcttggcaagcgtcaagacggtctcttcaaccgcgaaggccgtgcgttccccgacgtgtcagcccagggtgagaactacgctatctacgcgaagggtcgtctcggcaaggttgacggcacttcctgctcggctcccgctttcgccggtctggtttctctgctgaacgacgctcgcatcaaggcgggcaagtccagcctcggcttcctgaacccctggttgtactcgcaccccgatgccttgaacgacatcaccgtcggtggaagcaccggctgcgacggcaacgctcgcttcggtggtcgtcccaacggcagtcccgtcgtcccttacgctagctggaacgctactgagggctgggaccccgtcaccggtctgggtactcccaacttccagaagctgctcaagtctgccgttaagcagaagtaa

SEQ ID NO: 77 - DNA, 1806 bp, Penicillium roqueforti
atgattgcatccctatttagtcgtggagcattgtcgctcgcggtcttgtcgcttctcgcgtcctctgctgcagccgatgtatttgagagtttgtctgctgttcctcaaggatggagatattctcgcaggccgcgtgctgatcagcccttgaagttacagatcgctctgacacagggggatactgccggcttcgaagaggctgtgatggagatgtcaacccccgatcaccctagctacgggcaccacttcaccacccacgaagaaatgaagcggatgctacagcccagtgcggagtccgcggagtcaatccgtgactggctcgaaggcgcgggtattaccaggatcgaacaggatgcagattggatgaccttctacaccaccgtggagacggcaaatgagctgctggcagccaatttccagttctacgtcagtaatgtcaggcacattgagcgtcttcgcacactcaagtactcagtcccgaaggctctggtgccacacatcaacatgatccagccaaccacccgtttcggccagctgcgcgcccatcggggcatattacacggccaggtcaaggaatccgacgaggctttccgctcaaacgccgtgtccgctcagccggattgcaacagtatcatcactcctcagtgtctcaaggatatatataatatcggtgattaccaggccaatgataccaatgggaacaaggtcgggtttgccagctacctagaggagtatgcacgatactccgatctggcactatttgagaaaaatatcgcgccctctgccaagggccagaacttctccgtcaccaggtacaacggcggtcttaatgatcaaagttccagcggtagcagcagcgaggcgaacctggacttgcagtacattgttggagtcagctctcctgttcccgtcaccgaatttagcgttggcggccgtggtgaacttgttcccgatctcgaccagcctgatcccaatgataacaacaacgagccataccttgaattcctccagaacgtgctcaagctggacaaaaaggaccttccccaggtgatttctacctcctatggtgaggacgagcagagcattcccgagaagtacgcccgcagtgtttgcaacttgtactcgcagctcggtagccgtggtgtatccgtcattttctcatctggcgactccggcgttgggtccgcgtgcctgacgaacgacggcaggaacgcgacccgcttcccaccccagttccccgccgcctgcccgtgggtgacatcagtcggcgcgacaacccataccgcgcccgaacaggccgtgtacttctcgtccggcggcttttccgatctctgggctcgcccgaaatggcaagaggaggccgtgagtgagtacctcgagatcctgggtaaccgctggtctggcctcttcaaccctaagggtcgtgccttccccgatgtcacagcccaaggtcgcaattacgctatatacgataagggctcgttgaccagcgtcgacggcacctcctgctcggcacctgccttcgccggagtcgtcgccctcctcaacgacgctcgcctcaaagtcaacaaaccaccaatgggcttccttaatccttggctgtactcgacagggcgcgccggcctaaaggacattgtcgatggcggcagcacgggttgcgatggcaagagccgcttcggtggtgccaataacggtggtccgtcgatcccaggtgctagctggaacgctactaagggttgggaccctgtttctggtctcgggtcgcccaactttgctaccatgcgcaagcttgcgaacgctgagtag

SEQ ID NO: 78 - DNA, 1806 bp, Penicillium rubens Wisconsin
augauugcaucucuauuuaaccguggagcauugucgcucgcgguauugucgcuucucgcgucuucggcuuccgcugauguauuugagaguuugucugcuguuccucaaggauggagauauucucgcagaccgcgugcugaucagccccugaagcuacagauugcucuggcacaaggggauacugccggauucgaagaggcugugauggacaugucaaccccugaucaccccagcuacgggaaccacuuccacacccacgaggaaaugaagcggaugcugcagcccagcgcggaguccgcagacucgauccgugacuggcuugaaagugcggguaucaauagaauugaacaggaugccgacuggaugacauucuacaccaccgucgagacggcaaaugagcugcuggcagccaauuuccaguucuaugccaacagugccaagcacauugagcgucuucgcacacuccaguacuccgucccggaggcucugaugccacacaucaacaugauccagccaaccacucguuucggccagcugcgcguccagggggccauauugcacacccaggucaaggaaaccgacgaggcuuuccgcucaaacgccguguccacuucaccggacugcaacaguaucaucacuccucagugucucaagaauauguacaaugugggugacuaccaggccgacgacgacaaugggaacaaggucggauuugccagcuaccuagaggaguaugcacgguacuccgauuuggaacuauuugagaaaaaugucgcacccuucgccaagggccagaacuucuccgucauccaguauaacggcggucuuaacgaucaacacucgagugcuagcagcagcgaggcgaaccuugacuuacaguacauuguuggaguuagcucuccuguuccaguuacagaguuuagcguuggcggucguggugaacuuguucccgaucuugaccagccugaucccaaugauaacaacaacgagccauaccuugaauuccuccagaacgugcucaagauggaacaacaggaccucccccaggugauuuccaccucuuauggcgagaacgagcagaguguucccgagaaauacgcccgcaccguaugcaacuuguucucgcagcuuggcagccgugguguguccgucaucuucgcaucuggcgacuccggcguuggcgccgcgugccagacgaaugacggcaggaacgcgacccgcuucccggcccaguucccugcugccugcccaugggugacaucggucggcgcgacaacccacaccgcgcccgagaaggccguguacuucucguccgguggcuucuccgaucuuugggaucgcccgaaauggcaagaagacgccgugagugacuaccucgacacccugggcgaccgcugguccggccucuucaauccuaagggccgugccuuccccgacgucucagcccaaggucaaaacuacgccauauacgauaagggcucguugaccagcgucgacggcaccucgugcucggcacccgccuucgccggugucaucgcccuccucaacgacgcccgccucaaggccaacaaaccacccaugggcuuccucaaucccuggcuguacucgacaggccgugacggccugaacgacauuguucauggcggcagcacuggcugugauggcaacgcccgcuucggcggccccgguaacggcaguccgaggguuccaggugccagcuggaacgcuacuaagggcugggacccuguuucuggucuuggaucacccaacuuugcuaccaugcgcaagcucgcgaacggugaguag

SEQ ID NO: 79 - DNA, 1785 bp, Neosartorya fischeri
augcuguccucgacucucuacgcaggguugcucugcucccucgcagccccagcccuugguguggugcacgagaagcucucagcuguuccuaguggcuggacacucgucgaggaugcaucggagagcgacacgaccacucucucaauugcccuugcucggcagaaccucgaccagcucgaguccaaguugaccacacuggcgaccccagggaacgcggaguacggcaaguggcuggaccaguccgacauugagucccuauuuccuacugcaagcgaugacgcuguuauccaauggcucaaggaugccggggucacccaagugucucgucagggcagcuuggugaacuuugccaccacugugggaacggcgaacaagcucuuugacaccaaguucuccuacuaccgcaauggugcuucccagaaacugcguaccacgcaguacuccauucccgauagccugacagagucgaucgaucugauugcccccacugucuucuuuggcaaggagcaagacagcgcacugccaccucacgcagugaagcuuccagcccuucccaggagggcagccaccaacaguucuugcgccaaccugaucacucccgacugccuaguggagauguacaaccucggcgacuacaagccugaugcaucuucgggcagucgagucggcuuugguagcuucuugaaucagucagccaacuaugcagaucuggcugcuuaugagcaacuguucaacaucccaccccagaauuucucagucgaauugauuaacggaggcgccaaugaucagaauugggccacugcuucccucggcgaggccaaucuggacguggaguugauuguagccgucagccacgcccugccaguaguggaguuuaucacuggcgguucaccuccguuuguucccaaugucgacgagccaaccgcugcggacaaccagaaugagcccuaccuccaguacuacgaguacuugcucuccaaacccaacucccaucuuccucaggugauuuccaacucguauggugacgaugaacagacuguucccgaguacuacgccaggagaguuugcaacuugaucggcuugaugggucuucgugguaucacugugcucgaguccucuggugauaccggaaucggcucggcgugcauguccaaugacggcaccaacacgccucaguucacuccuacauucccuggcaccugccccuucaucaccgcaguuggugguacacaguccuaugcuccugaaguugccugggacgccagcucggguggauucagcaacuacuucagccgucccugguaccaguauuucgcgguggagaacuaccucaauaaucacauuaccaaggacaccaagaaguacuauucgcaguacaccaacuucaagggccguggauucccugauguuucugcccauagcuugaccccugacuacgaggucguccuaacuggcaaacauuacaaguccgguggcacaucggccgccugccccgucuuugcugguaucgucggccuguugaaugacgcccgucugcgcgccggcaaguccacccuuggcuuccugaacccauugcuguauagcauacucgcggaaggauucaccgauaucacugccggaaguucuaucgguuguaaugguaucaacccacagaccggaaagccaguccccgguggugguaucauccccuacgcucacuggaacgcuacugccggcugggauccuguuacaggucuugggguuccugauuucaugaaguugaaggaguugguuuugucguuguaa

SEQ ID NO: 80 - DNA, 1707 bp, Aspergillus fumigatus
augcuguccucgacucucuacgcaggguggcuccucucccucgcagccccagcccuuuguguggugcaggagaagcucucagcuguuccuaguggcuggacacucaucgaggaugcaucggagagcgacacgaucacucucucaauugcccuugcucggcagaaccucgaccagcuugaguccaagcugaccacgcuggcgaccccagggaacccggaguacggcaaguggcuggaccaguccgacauugagucccuauuuccuacugcaagcgaugaugcuguucuccaauggcucaaggcggccgggauuacccaagugucucgucagggcagcuuggugaacuucgccaccacugugggaacagcgaacaagcucuuugacaccaaguucucuuacuaccgcaauggugcuucccagaaacugcguaccacgcaguacuccauccccgaucaccugacagagucgaucgaucugauugcccccacugucuucuuuggcaaggagcagaacagcgcacugucaucucacgcagugaagcuuccagcucuuccuaggagggcagccaccaacaguucuugcgccaaccugaucacccccgacugccuaguggagauguacaaccucggcgacuacaaaccugaugcaucuucgggaagucgagucggcuucgguagcuucuugaaugagucggccaacuaugcagauuuggcugcguaugagcaacucuucaacaucccaccccagaauuucucagucgaauugaucaacagaggcgucaaugaucagaauugggccacugcuucccucggcgaggccaaucuggacguggaguugauuguagccgucagccacccccugccaguaguggaguuuaucacuggcgcccuaccuccaguacuacgaguacuugcucuccaaacccaacucccaucuuccucaggugauuuccaacucacuguucccgaguacuacgccaggagaguuugcaacuugaucggcuugaugggucuucguggcaucacggugcucgaguccucuggugauaccggaaucggcucggcaugcauguccaaugacggcaccaacaagccccaauucacuccuacauucccuggcaccugccccuucaucaccgcaguuggugguacucaguccuaugcuccugaaguugcuugggacggcaguuccggcggauucagcaacuacuucagccgucccugguaccagucuuucgcgguggacaacuaccucaacaaccacauuaccaaggauaccaagaaguacuauucgcaguacaccaacuucaagggccguggauucccugauguuuccgcccauaguuugaccccuuacuacgaggucgucuugacuggcaaacacuacaagucuggcggcacauccgccgccagccccgucuuugccgguauugucggucugcugaacgacgcccgucugcgcgccggcaaguccacucuuggcuuccugaacccauugcuguauagcauccuggccgaaggauucaccgauaucacugccggaaguucaaucgguuguaaugguaucaacccacagaccggaaagccaguuccugguggugguauuauccccuacgcucacuggaacgcuacugccggcugggauccuguuacuggccuugggguuccugauuucaugaaauugaaggaguugguucugucguuguaa

SEQ ID NO: 81 - DNA, 1785 bp, Phaeosphaeria nodorum
atgcagaccttcggtgcttttctcgtttccttcctcgccgccagcggcctggccgcggccgagcccttcgagaagctctttagcacccccgagggctggaagatgcagggcctcgccaccaacgagcagatcgtcaagctccagatcgccctccagcagggcgacgtggccggctttgagcagcacgtcatcgacatcagcacccccagccaccccagctacggcgctcactacggcagccacgaagagatgaagcgcatgatccagcccagcagcgagactgtcgccagcgtcagcgcctggctcaaggccgctggcatcaacgacgccgagatcgacagcgactgggtcaccttcaagaccaccgtcggcgtcgccaacaagatgctcgacaccaagttcgcctggtacgtcagcgaggaagccaagccccgcaaggtcctccgcacccttgagtacagcgtccccgacgacgtcgccgagcacatcaacctcatccagcccaccacccgcttcgccgccatccgccagaaccacgaggtcgcccacgagatcgtcggcctccagtttgccgccctcgccaacaacaccgtcaactgcgacgccaccatcaccccccagtgcctcaagaccctctacaagatcgactacaaggccgaccccaagagcggcagcaaggtcgccttcgccagctaccttgagcagtacgcccgctacaacgacctcgccctcttcgagaaggccttcctgcctgaggccgtcggccagaacttcagcgtcgtccagttctctggcggcctcaacgaccagaacaccacccaggatagcggcgaggccaacctcgacctccagtacatcgtcggcgtcagcgcccctctgcccgtcaccgagtttagcactggcggccgaggcccttgggtcgccgatctcgatcagcctgacgaggccgacagcgccaacgagccctaccttgagttcctccagggcgtcctcaagctcccccagagcgagctgccccaggtcatcagcacctcgtacggcgagaacgagcagagcgtccccaagagctacgccctcagcgtctgcaacctcttcgcccagcttggctctcgcggcgtcagcgtcatcttcagcagcggcgatagcggccctggcagcgcctgccagtctaacgacggcaagaacaccaccaagttccagccccagtaccctgccgcctgccccttcgtcactagcgtcggctctacccgctacctcaacgagactgccaccggcttcagctccggcggcttcagcgactactggaagcgccccagctaccaggacgacgccgtcaaggcctacttccaccacctcggcgagaagttcaagccctacttcaaccgccacggccgaggcttccctgacgtcgccactcagggctacggcttccgcgtctacgaccagggcaagctcaagggcctccagggcacttctgccagcgcccctgccttcgccggcgtcattggcctgctcaacgacgcccgcctcaaggccaagaagcccaccctcggctttctcaaccccctgctctacagcaacagcgacgccctcaacgacatcgtcctcggcggctccaagggctgcgacggccacgctaggtttaacggccctcccaacggcagccccgtcatcccttacgccggctggaacgccactgccggctgggaccctgttaccggcctcggcacccccaacttccccaagctcctcaaggccgccgtcccctctcgataccgcgcttaa

SEQ ID NO: 82 - DNA, 1833 bp, Trichoderma atroviride
atgcagaccttcggtgcttttctcgtttccttcctcgccgccagcggcctggccgcggccaacgctgctgtcctcctcgacagcctcgacaaggtccccgtcggctggcaggctgcttctgcccctgctcccagcagcaagatcaccctccaggtcgccctcacccagcagaacatcgaccagcttgagagcaagctcgccgccgtcagcacccccaacagcagcaactacggcaagtacctcgacgtcgacgagatcaaccagatcttcgcccccagcagcgccagcactgccgctgtcgagagctggctcaagagctacggcgtcgactacaaggtccagggcagcagcatctggttccagaccgacgtcagcacggccaacaagatgctcagcaccaacttccacacctacaccgacagcgtcggcgccaagaaggtccgcaccctccagtacagcgtccccgagactctcgccgaccacatcgacctcatcagccccaccacctacttcggcaccagcaaggccatgcgagccctcaagatccagaacgccgccagcgccgtcagccctctcgctgctcgacaagagcccagcagctgcaagggcaccatcgagttcgagaaccgcaccttcaacgtctttcagcccgactgcctccgcaccgagtacagcgtcaacggctacaagcccagcgccaagagcggcagccgaatcggcttcggcagcttcctcaaccagagcgccagcagcagcgacctcgccctcttcgagaagcacttcggcttcgccagccagggcttcagcgtcgagctgatcaacggcggcagcaacccccagcctcccaccgatgctaacgacggcgaggccaacctcgacgcccagaacatcgtcagcttcgtccagcccctgcccatcaccgagtttatcgctggcggcaccgccccctacttccccgatcctgttgagcctgccggcacccccgacgagaacgagccctaccttgagtactacgagtacctcctcagcaagagcaacaaggaactcccccaggtcatcaccaacagctacggcgacgaggaacagaccgtcccccaggcctacgccgtccgcgtctgcaacctcatcggcctcatgggcctccgcggcatcagcatccttgagagcagcggcgacgagggcgtcggcgcttcttgcctcgccaccaacagcaccaccaccccccagttcaaccccatcttccccgccacgtgcccctacgtcactagcgtcggcggcaccgtcagcttcaaccccgaggtcgcttgggacggcagcagcggcggcttcagctactacttcagccgcccctggtatcaagaggccgccgtcggcacctacctcaacaagtacgtcagcgaggaaacgaaggaatattacaagagctacgtcgacttcagcggccgaggcttccctgacgtcgccgctcactctgtcagccccgactaccccgtctttcagggcggcgagctgactccttctggcggcacttctgccgccagccccatcgtcgccagcgtcattgccctgctcaacgacgcccgactccgagccggcaagcctgccctcggctttctcaaccccctcatctacggctacgcctacaagggcttcaccgacatcacctccggccaggccgttggctgcaacggcaacaacacccagaccggcggaccccttcctggcgctggcgttatccctggcgccttctggaacgccaccaagggctgggaccccaccaccggctttggcgtccccaacttcaagaagctccttgagctggtccgctacatc

SEQ ID NO: 83 - DNA, 1803 bp, Arthroderma benhamiae
atgcagaccttcggtgcttttctcgtttccttcctcgccgccagcggcctggccgcggccaagcctactcctggcgcttcccacaaggtcatcgagcacctcgacttcgtccccgagggctggcagatggtcggcgctgctgaccctgccgccatcatcgacttttggctcgccatcgagcgcgagaaccccgagaagctctacgacaccatctacgacgtcagcacccccggacgcgcccagtacggcaagcacctcaagcgcgaggaactcgacgacctcctccgccctcgcgccgagactagcgagagcatcatcaactggctcaccaacggcggcgtcaacccccagcacattcgcgacgagggcgactgggtccgcttcagcaccaacgtcaagaccgccgagactctcatgaacacccgcttcaacgtctttaaggacaacctcaacagcgtcagcaagatccgcacccttgagtacagcgtccccgtcgccatcagcgcccacgtccagatgatccagcccaccaccctcttcggccgccagaagccccagaacagcctcatcctcaaccccctcaccaaggaccttgagagcatgagcgtcgaagagttcgccgccagccagtgccgcagcctcgtcactactgcctgcctccgcgagctgtacggcctcggcgatcgagtcacccaggcccgcgacgacaaccgaattggcgtcagcggcttcctcgaagagtacgcccagtaccgcgaccttgagctgttcctcagccgcttcgagcccagcgccaagggcttcaacttcagcgagggcctgatcgctggcggcaagaacacccagggtggccctggctctagcaccgaggccaacctcgacatgcagtacgtcgtcggcctcagccacaaggccaaggtcacctactacagcactgccggccgaggccccctcatccctgatctctcacagcccagccaggccagcaacaacaacgagccctaccttgagcagctccgctacctcgtcaagctccccaagaaccagctccccagcgtcctcaccaccagctacggcgacaccgagcagagcctccccgccagctacaccaaggccacgtgcgacctcttcgcccagctcggcactatgggcgtcagcgtcatcttcagcagcggcgacactggccctggcagctcgtgccagaccaacgacggcaagaacgccacgcgcttcaaccccatctaccccgccagctgccccttcgtcaccagcattggcggcaccgtcggcaccggccctgagcgagctgtcagctttagcagcggcggcttcagcgaccgcttccctcgccctcagtaccaggacaacgccgtcaaggactacctcaagatcctcggcaaccagtggtccggcctcttcgaccctaacggccgagccttccccgacattgccgcccagggcagcaactacgccgtctacgacaagggccgcatgaccggcgttagcggcacttctgcttccgcccctgctatggccgccatcattgcccagctcaacgacttccgcctcgccaagggcagccccgtcctcggctttctcaacccctggatctacagcaagggcttcagcggcttcaccgacatcgtcgacggcggctctaggggctgcaccggctacgacatctacagcggcctcaaggccaagaaggtcccctacgccagctggaacgccaccaagggctgggaccccgtcaccggctttggcacccccaacttccaggccctgaccaaggtcctgccctaa

SEQ ID NO: 84 - DNA, 1803 bp, Fusarium graminearum
atgcagaccttcggtgcttttctcgtttccttcctcgccgccagcggcctggccgcggccaagagctactctcaccacgccgaggcccccaagggctggaaggtcgacgatactgcccgcgtcgccagcaccggcaagcagcaggtcttttcgatcgccctgaccatgcagaacgtcgaccagcttgagagcaagctcctcgacctcagcagccccgacagcaagaactacggccagtggatgagccagaaggacgtcaccaccgccttctaccccagcaaggaagccgtcagcagcgtcaccaagtggctcaagagcaagggcgtcaagcactacaacgtcaacggcggcttcatcgacttcgccctcgacgtgaagggcgccaacgccctcctcgacagcgactaccagtactacaccaaggaaggccagaccaagctccgcaccctcagctacagcatccccgacgacgtcgccgagcacgtccagttcgtcgaccccagcaccaacttcggcggcaccctcgcctttgcccccgtcactcaccctagccgcaccctcaccgagcgcaagaacaagcccaccaagagcaccgtcgacgccagctgccagaccagcatcacccccagctgcctcaagcagatgtacaacatcggcgactacacccccaaggtcgagagcggcagcacgatcggcttcagcagcttcctcggcgagagcgctatctacagcgacgtctttctgttcgaggaaaagttcggcatccccacccagaacttcaccaccgtcctcatcaacaacggcaccgacgaccagaacaccgcccacaagaacttcggcgaggccgacctcgacgccgagaacatcgtcggcattgcccaccccctgcccttcacccagtacatcactggcggcagcccccccttcctgcccaacatcgatcagcccactgccgccgacaaccagaacgagccctacgtccccttcttccgctacctcctcagccagaaggaagtccccgccgtcgtcagcaccagctacggcgacgaagaggacagcgtcccccgcgagtacgccaccatgacctgcaacctcatcggcctgctcggcctccgcggcatcagcgtcatcttcagcagcggcgacatcggcgtcggcgctggctgtcttggccccgaccacaagaccgtcgagttcaacgccatcttccccgccacgtgcccctacctcactagcgtcggcggcacggtcgacgtcacccccgagattgcttgggagggcagcagcggcggcttcagcaagtacttccctcgccccagctaccaggacaaggccgtcaagacctacatgaagaccgtcagcaagcagaccaagaagtactacggcccctacaccaactgggagggccgaggctttcctgacgtcgccggccacagcgtcagccccaactacgaggtcatctacgccggcaagcagagcgcctctggcggcacttctgctgccgcccctgtctgggctgccatcgtcggcctgctcaacgacgcccgattccgagccggcaagcctagcctcggctggctcaaccccctcgtctacaagtacggccccaaggtcctcaccgacatcaccggcggctacgccattggctgcgacggcaacaacacccagagcggcaagcccgagcctgccggctctggcattgtccctggcgcccgatggaacgccactgccggatgggaccctgtcaccggctacggcacccccgacttcggcaagctcaaggacctcgtcctcagcttctaa

SEQ ID NO: 85 - DNA, 1872 bp, Acremonium alcalophilum
atgcagaccttcggtgcttttctcgtttccttcctcgccgccagcggcctggccgcggccgccgtcgtcattcgcgccgccgtcctccccgacgccgtcaagctgatgggcaaggccatgcccgacgacattatttccctccagttttccctgaagcagcagaacatcgaccagctggagacccgcctccgcgccgtctcggaccccagctcccccgagtacggccagtacatgagcgagtccgaggtcaacgagttctttaagccccgcgacgactcgttcgccgaggtcattgactgggtcgccgccagcggctttcaggacatccacctgacgccccaggctgccgccattaacctcgccgccaccgtcgagacggccgaccagctcctgggcgccaacttcagctggtttgacgtcgacggcacccgcaagctccgcaccctggagtacacgatccccgaccgcctcgccgaccacgtcgacctgatttcccccaccacgtacttcggccgcgcccgactggacggcccccgcgagacccccacgcgcctcgacaagcgccagcgcgaccccgtcgccgacaaggcctacttccacctcaagtgggaccgcggcaccagcaactgcgacctggtcatcacgcccccctgcctggaggccgcctacaactacaagaactacatgcccgaccccaactcgggcagccgcgtctcgttcaccagctttctggagcaggccgcccagcagagcgacctcaccaagttcctctccctgacgggcctcgaccgcctgcgcccccccagcagcaagcccgccagcttcgacacggtcctgatcaacggcggcgagacccaccagggcacgccccccaacaagacctccgaggccaacctcgacgtccagtggctggccgccgtcattaaggcccgactccccatcacccagtggattacgggcggccgcccccccttcgtccccaacctccgcctgcgccacgagaaggacaacacgaacgagccctacctggagttctttgagtacctcgtccgcctgcccgcccgcgacctcccccaggtcatctccaactcgtacgccgaggacgagcagaccgtccccgaggcctacgcccgacgcgtctgcaacctcatcggcattatgggcctgcgcggcgtcaccgtcctcacggcctccggcgactcgggcgtcggcgccccctgccgcgccaacgacggcagcgaccgcctggagttctccccccagtttcccacctcgtgcccctacatcaccgccgtcggcggcacggagggctgggaccccgaggtcgcctgggaggcctcctcgggcggcttcagccactactttctccgcccctggtaccaggccaacgccgtcgagaagtacctcgacgaggagctggaccccgccacccgcgcctactacgacggcaacggcttcgtccagtttgccggccgagcctaccccgacctgtccgcccacagctcctcgccccgctacgcctacatcgacaagctcgcccccggcctgaccggcggcacgagcgcctcctgccccgtcgtcgccggcatcgtcggcctcctgaacgacgcccgactccgccgcggcctgcccacgatgggcttcattaacccctggctgtacacgcgcggctttgaggccctccaggacgtcaccggcggccgcgcctcgggctgccagggcatcgacctccagcgcggcacccgcgtccccggcgccggcatcattccctgggcctcctggaacgccacccccggctgggaccccgccacgggcctcggcctgcccgacttctgggccatgcgcggcctcgccctgggccgcggcacctaa

SEQ ID NO: 86 - DNA, 1905 bp, Sodiomyces alkalinus
atgcagaccttcggtgcttttctcgtttccttcctcgccgccagcggcctggccgcggccgccgtcgtcattcgcgccgcccccctccccgagagcgtcaagctcgtccgcaaggccgccgccgaggacggcattaacctccagctctccctgaagcgccagaacatggaccagctggagaagttcctccgcgccgtcagcgaccccttttcccccaagtacggccagtacatgtcggacgccgaggtccacgagatcttccgccccaccgaggactcctttgaccaggtcattgactggctcaccaagtcgggcttcggcaacctgcacatcacgccccaggctgccgccattaacgtcgccaccacggtcgagaccgccgaccagctgtttggcgccaacttctcctggtttgacgtcgacggcacgcccaagctccgcaccggcgagtacacgatccccgaccgcctcgtcgagcacgtcgacctggtcagccccaccacgtacttcggccgcatgcgccccccccctcgcggcgacggcgtcaacgactggatcaccgagaactcgcccgagcagcccgcccccctgaacaagcgcgacaccaagacggagagcgaccaggcccgcgaccacccctcctgggactcgcgcacccccgactgcgccaccatcattacgcccccctgcctggagacggcctacaactacaagggctacatccccgaccccaagtccggctcgcgcgtcagcttcaccagcttcctggagcaggccgcccagcaggccgacctgaccaagttcctcagcctgacgcgcctggagggctttcgcacccccgccagcaagaagaagaccttcaagacggtcctgatcaacggcggcgagtcccacgagggcgtccacaagaagtcgaagaccagcgaggccaacctcgacgtccagtggctggccgccgtcacccagacgaagctgcccatcacccagtggattacgggcggccgcccccccttcgtccccaacctccgcatccccacccccgaggccaacacgaacgagccctacctggagttcctggagtacctctttcgcctgcccgacaaggacctcccccaggtcatcagcaactcctacgccgaggacgagcagagcgtccccgaggcctacgcccgacgcgtctgcggcctcctgggcattatgggcctccgcggcgtcaccgtcctgacggcctccggcgactcgggcgtcggcgccccctgccgcgccaacgacggctcgggccgcgaggagttcagcccccagtttcccagctcctgcccctacatcaccacggtcggcggcacccaggcctgggaccccgaggtcgcctggaagggcagcagcggcggcttctccaactactttccccgcccctggtaccaggtcgccgccgtcgagaagtacctggaggagcagctggaccccgccgcccgcgagtactacgaggagaacggcttcgtccgctttgccggccgagccttccccgacctgagcgcccacagcagcagccccaagtacgcctacgtcgacaagcgcgtccccggcctcaccggcggcacgtcggccagctgccccgtcgtcgccggcatcgtcggcctcctgaacgacgcccgactccgccgcggcctgcccacgatgggcttcattaacccctggctctacgccaagggctaccaggccctggaggacgtcaccggcggcgccgccgtcggctgccagggcatcgacattcagacgggcaagcgcgtccccggcgccggcatcattcccggcgccagctggaacgccacccccgactgggaccccgccacgggcctcggcctgcccaacttctgggccatgcgcgagctcgccctggaggactaa

SEQ ID NO: 87 - DNA, 1788 bp, Aspergillus kawachii
atgcagaccttcggtgcttttctcgtttccttcctcgccgccagcggcctggccgcggccgtcgtccatgagaagctcgctgctgtccccagcggctggcaccaccttgaggatgccggcagcgaccaccagatcagcctctcgattgccctcgcccgcaagaacctcgaccagcttgagagcaagctcaaggacctcagcacccctggcgagagccagtacggccagtggctcgaccaagaggaagtcgacaccctgttccccgtcgccagcgacaaggccgtcatcagctggctccgcagcgccaacatcacccacattgcccgccagggcagcctcgtcaacttcgccaccaccgtcgacaaggtcaacaagctcctcaacaccaccttcgcctactaccagcgcggcagctctcagcgcctccgcaccaccgagtacagcatccccgacgacctcgtcgacagcatcgacctgatcagccccaccacgttcttcggcaaggaaaagacctctgccggcctcacccagcgcagccagaaggtcgataaccacgtcgccaagcgcagcaacagcagcagctgcgccgacaccatcaccctcagctgcctcaaggaaatgtacaacttcggcaactacacccccagcgccagcagcggcagcaagctcggcttcgccagcttcctcaacgagagcgccagctacagcgacctcgccaagttcgagcgcctcttcaacctccccagccagaacttcagcgtcgagctgatcaacggcggcgtcaacgaccagaaccagagcaccgccagcctcaccgaggccgacctcgatgtcgagctgcttgtcggcgtcggccaccccctgcccgtcaccgagtttatcaccagcggcgagccccccttcatccccgaccctgatgagccttctgccgccgacaacgagaacgagccctacctccagtactacgagtacctcctcagcaagcccaacagcgccctgccccaggtcatcagcaacagctacggcgacgacgagcagaccgtccccgagtactacgccaagcgcgtctgcaacctcatcggcctcgtcggcctccgcggcatcagcgtccttgagtctagcggcgacgagggcatcggctctggctgccgaaccaccgacggcaccaacagcacccagttcaaccccatcttccccgccacgtgcccctacgtcactgccgtcggcggcaccatgagctacgcccccgagattgcttgggaggccagctccggcggcttcagcaactacttcgagcgagcctggttccagaaggaagccgtccagaactacctcgccaaccacatcaccaacgagactaagcagtactacagccagttcgccaacttcagcggccgaggcttccccgacgtcagcgcccacagcttcgagcccagctacgaggtcatcttctacggcgctcgctacggcagcggcggcacttctgctgcctgccccctgttttctgccctcgtcggcatgctcaacgacgcccgactccgagccggcaagtcgaccctcggcttcctcaaccccctgctctacagcaagggctacaaggccctcaccgacgtcaccgctggccagagcattggctgcaacggcatcgacccccagagcgacgaggctgtcgctggcgctggcatcattccctgggcccactggaacgccaccgtcggctgggaccctgtcactggccttggcctccccgacttcgagaagctccgccagctcgtcctcagcctctaa

SEQ ID NO: 88 - DNA, 1809 bp, Talaromyces stipitatus
atgcagaccttcggtgcttttctcgtttccttcctcgccgccagcggcctggccgcggccgctgctgctcttgttggccacgagtctctcgccgccctccctgtcggctgggacaaggtcagcactcctgccgctggcaccaacatccagctcagcgtcgccctcgccctccagaacatcgagcagcttgaggaccacctcaagagcgtcagcacccccggctctgccagctacggccagtacctcgacagcgacggcattgccgcccagtacggcccttctgacgccagcgtcgaggccgtcaccaactggctcaaggaagccggcgtcaccgacatctacaacaacggccagagcatccacttcgccaccagcgtcagcaaggccaacagcctcctcggcgccgacttcaactactacagcgacggctccgccaccaagctccgcaccctcgcttacagcgtccccagcgacctgaaggaagccatcgacctcgtcagccccaccacctacttcggcaagaccaccgccagccgcagcatccaggcctacaagaacaagcgagccagcaccaccagcaagagcggcagcagcagcgtccaggtcagcgcctcttgccagaccagcatcacccccgcctgcctcaagcagatgtacaacgtcggcaactacacccccagcgtcgcccacggctctcgcgttggcttcggcagcttcctcaaccagagcgccatcttcgacgacctcttcacctacgagaaggtcaacgacatccccagccagaacttcaccaaggtcatcattgccaacgccagcaacagccaggacgccagcgacggcaactacggcgaggccaacctcgacgtccagaacattgtcggcatcagccaccccctgcccgtcaccgagtttctcactggcggcagcccacccttcgtcgccagcctcgacacccccaccaaccagaacgagccctacatcccctactacgagtacctcctcagccagaagaacgaggacctcccccaggtcatcagcaacagctacggcgacgacgagcagagcgtcccctacaagtacgccatccgcgcctgcaacctcatcggcctcactggcctccgcggcatcagcgtccttgagagcagcggcgatctcggcgttggcgctggctgccgatccaacgacggcaagaacaagacccagttcgaccccatcttccccgccacgtgcccctacgtcactagcgtcggcggcacccagagcgtcacccccgagattgcttgggtcgcttccagcggcggcttcagcaactacttcccccgcacctggtatcaagagcccgccatccagacctacctcggcctcctcgacgacgagactaagacctactacagccagtacaccaacttcgagggccgaggcttccccgacgtcagcgcccattctctcacccccgactaccaggtcgtcggcggaggctaccttcagccttctggcggcacttctgccgccagccctgtctttgccggcatcattgccctgctcaacgacgcccgactcgccgctggcaagcccaccctcggctttctcaaccccttcttctacctctacggctacaagggcctcaacgacatcactggcggccagagcgtcggctgcaacggcatcaacggccagactggcgcccctgttcccggcggaggaattgtccctggcgccgcttggaacagcaccaccggatgggaccctgccaccggccttggcacccccgactttcagaagctcaaggaactcgtcctcagcttctaa

SEQ ID NO: 89 - DNA, 1800 bp, Fusarium oxysporum
atgcagaccttcggtgcttttctcgtttccttcctcgccgccagcggcctggccgcggccaagtcgttttcccaccacgccgaggccccccagggctggcaggtccagaagaccgccaaggtcgcctccaacacgcagcacgtctttagcctcgccctgaccatgcagaacgtcgaccagctggagtcgaagctcctggacctgagctcccccgacagcgccaactacggcaactggctcagccacgacgagctgacctccacgttctcgcccagcaaggaggccgtcgcctcggtcaccaagtggctgaagagcaagggcatcaagcactacaaggtcaacggcgccttcattgactttgccgccgacgtcgagaaggccaacaccctcctgggcggcgactaccagtactacacgaaggacggccagaccaagctgcgcacgctctcctactcgatccccgacgacgtcgccggccacgtccagttcgtcgaccccagcaccaacttcggcggcacggtcgcctttaaccccgtcccccacccctcccgcaccctccaggagcgcaaggtctccccctccaagtcgacggtcgacgcctcctgccagacctcgatcacgcccagctgcctgaagcagatgtacaacattggcgactacacccccgacgccaagagcggctccgagatcggcttcagcagcttcctcggccaggccgccatttacagcgacgtcttcaagtttgaggagctcttcggcatccccaagcagaactacaccacgatcctgattaacaacggcaccgacgaccagaacacggcccacggcaactttggcgaggccaacctcgacgccgagaacatcgtcggcattgcccaccccctgcccttcaagcagtacatcaccggcggcagccccccctttgtccccaacattgaccagcccacggagaaggacaaccagaacgagccctacgtccccttctttcgctacctcctgggccagaaggacctgcccgccgtcatctcgaccagctacggcgacgaggaggactccgtcccccgcgagtacgccaccctcacgtgcaacatgatcggcctcctgggcctgcgcggcatctccgtcattttctcctcgggcgacattggcgtcggctcgggctgcctcgcccccgactacaagaccgtcgagttcaacgccatctttcccgccacctgcccctacctgacgtccgtcggcggcaccgtcgacgtcacgcccgagattgcctgggagggcagctccggcggcttctccaagtactttccccgcccctcgtaccaggacaaggccatcaagaagtacatgaagaccgtctcgaaggagacgaagaagtactacggcccctacaccaactgggagggccgcggcttccccgacgtcgccggccactccgtcgcccccgactacgaggtcatctacaacggcaagcaggcccgatccggcggcaccagcgccgccgcccccgtctgggccgccatcgtcggcctcctgaacgacgcccgattcaaggccggcaagaagagcctgggctggctcaaccccctgatctacaagcacggccccaaggtcctcaccgacatcacgggcggctacgccattggctgcgacggcaacaacacccagagcggcaagcccgagcccgccggctccggcctggtccccggcgcccgatggaacgccaccgccggctgggaccccaccacgggctacggcacgcccaacttccagaagctcaaggacctcgtcctgtccctctaa

SEQ ID NO: 90 - DNA, 1788 bp, Trichoderma virens; identical to SEQ ID NO: 87

SEQ ID NO: 91 - DNA, 1770 bp, Trichoderma atroviride
atgcagaccttcggtgcttttctcgtttccttcctcgccgccagcggcctggccgcggccgctgtccttgtcgagtctctcaagcaggtccccaacggctggaacgccgtcagcacccctgaccccagcaccagcatcgtcctccagatcgccctcgcccagcagaacatcgacgagcttgagtggcgcctcgccgccgtgtctacccccaactctggcaactacggcaagtacctcgacatcggcgagatcgagggcatcttcgcccccagcaacgccagctacaaggccgtcgcttcctggctccagagccacggcgtcaagaacttcgtcaagcaggccggcagcatctggttctacaccaccgtcagcaccgccaacaagatgctcagcaccgacttcaagcactacagcgaccccgtcggcatcgagaagctccgcaccctccagtacagcatccccgaggaactcgtcggccacgtcgacctcatcagccccaccacctacttcggcaacaaccaccctgccaccgcccgcacccccaacatgaaggccatcaacgtcacctaccagatcttccaccccgactgcctcaagaccaagtacggcgtcgacggctacgccccctcacctcgatgcggcagccgaatcggcttcggcagcttcctcaacgagactgccagctacagcgacctcgcccagttcgagaagtacttcgacctccccaaccagaacctcagcaccctcctcatcaacggcgccatcgacgtccagccccccagcaacaagaacgacagcgaggccaacatggacgtccagaccatcctcaccttcgtccagcccctgcccatcaccgagttcgtcgtcgccggcatccccccctacattcccgatgccgccctccccattggcgaccccgttcagaacgagccctggcttgagtacttcgagttcctcatgagccgcaccaacgccgagctgccccaggtcattgccaacagctacggcgacgaggaacagaccgtcccccaggcctacgccgtccgcgtctgcaaccagattggcctcctcggcctccgcggcatcagcgtcattgcctctagcggcgacaccggcgtcggcatgtcttgcatggccagcaacagcaccaccccccagttcaaccccatgttccccgccagctgcccctacatcaccaccgtcggcggcacccagcacctcgacaacgagatcgcctgggagctgagcagcggcggcttcagcaactacttcacccgcccctggtatcaagaggacgccgccaagacctaccttgagcgccacgtcagcaccgagactaaggcctactacgagcgctacgccaacttcctgggccgaggctttcctgacgtcgccgccctcagcctcaaccccgactaccccgtcatcatcggcggcgagcttggccctaacggcggcacttctgctgccgcccctgtcgtcgccagcatcattgccctgctcaacgacgcccgcctctgcctcggcaagcctgccctcggctttctcaaccccctcatctaccagtacgccgacaagggcggcttcaccgacatcaccagcggccagtcttggggctgcgccggcaacaccactcagactggacctccccctcctggcgctggcgtcattcctggcgctcactggaacgccaccaagggctgggaccccgtcaccggctttggcacccccaacttcaagaagctcctcagcctcgccctcagcgtctaa

SEQ ID NO: 92 - DNA, 1758 bp, Agaricus bisporus
atgcagaccttcggtgcttttctcgtttccttcctcgccgccagcggcctggccgcggcctctcctcttgctcgacgctgggacgacttcgccgagaagcacgcctgggtcgaggttcctcgcggctgggagatggtcagcgaggcccctagcgaccacaccttcgacctccgcatcggcgtcaagagcagcggcatggaacagctcatcgagaacctcatgcagaccagcgaccccacccacagccgctacggccagcacctcagcaaggaagaactccacgacttcgtccagccccaccccgactctactggcgccgtcgaggcctggcttgaggacttcggcatcagcgacgacttcatcgaccgcaccggcagcggcaactgggtcaccgtccgagtctctgtcgcccaggccgagcgaatgctcggcaccaagtacaacgtctaccgccacagcgagagcggcgagtccgtcgtccgcaccatgagctacagcctccccagcgagctgcacagccacatcgacgtcgtcgcccccaccacctacttcggcaccatgaagtcgatgcgcgtcacctcgttcctccagcccgagatcgagcccgtcgacccctctgccaagccttctgctgctcccgccagctgcctcagcaccaccgtcattacccccgactgcctccgcgacctctacaacaccgccgactacgtccccagcgccaccagccgcaacgccattggcattgccggctacctcgaccgcagcaaccgagccgacctccagaccttcttccgccgctttcgccctgacgccgtcggcttcaactacaccaccgtccagctcaacggcggaggcgacgaccagaacgaccctggcgtcgaggccaacctcgacatccagtacgccgctggcattgccttccccacccccgccacctactggtctactggcggcagcccccccttcatccccgacacccagacccccaccaacaccaacgagccctacctcgactggatcaacttcgtcctcggccaggatgagatcccccaggtcatcagcaccagctacggcgacgacgagcagaccgtccccgaggactacgccaccagcgtctgcaacctcttcgcccagcttggctctcgcggcgtcaccgtctttttcagcagcggcgacttcggcgtcggcggtggcgactgcctcactaacgacggcagcaaccaggtcctcttccagcccgccttccctgccagctgcccctttgtcactgccgtcggcggcaccgtccgactcgaccctgagatcgccgtcagcttcagcggcggtggcttcagccgctacttcagccgccccagctaccagaaccagaccgtcgcccagttcgtcagcaacctcggcaacaccttcaacggcctctacaacaagaacggccgagcctaccccgacctcgccgctcagggcaacggcttccaggtcgtcatcgacggcatcgtccgatcggtcggcggcacttctgccagcagccctaccgtcgccggcatcttcgccctgctcaacgacttcaagctctctcgcggccagagcaccctcggcttcatcaaccccctcatctacagcagcgccacctccggcttcaacgacatccgagccggcaccaaccctggctgtggcacccgaggctttaccgccggcactggctgggaccctgtcaccggactcggcacccctgactttctccgcctccagggcctcatctaa

SEQ ID NO: 93 - DNA, 1812 bp, Magnaporthe oryzae
atgcagaccttcggtgcttttctcgtttccttcctcgccgccagcggcctggccgcggcccgcgtctttgattctctccctcacccccctcgcggctggtcctactctcacgccgctgagagcaccgagcccctcaccctccgaattgccctccgccagcagaacgccgctgcccttgagcaggtcgtcctccaggtcagcaacccccgccacgccaactacggccagcacctcacccgagatgagctgcgctcttacaccgcccctacccctcgcgctgtccgctctgtcactagctggctcgtcgacaacggcgtcgacgactacaccgtcgagcacgactgggtcaccctccgcaccactgtcggcgctgccgatcgactcctcggcgccgactttgcctggtacgctggccctggcgagactctccagctccgcactctcagctacggcgtggacgacagcgtcgcccctcacgtcgatctcgtccagcccaccacccgctttggcggccctgttggccaggccagccacatcttcaagcaggacgacttcgacgagcagcagctcaagaccctcagcgtcggcttccaggtcatggccgacctccctgctaacggccctggcagcattaaggccgcctgcaacgagagcggcgtcacccctctctgcctccgcaccctctaccgcgtcaactacaagcccgccaccaccggcaacctcgtcgccttcgccagcttccttgagcagtacgcccgctacagcgaccagcaggccttcacccagcgagtccttggccctggcgtcccgctccagaacttcagcgtcgagactgtcaacggcggagccaacgaccagcagagcaagctcgatagcggcgaggccaacctcgacctccagtacgtcatggccatgtcccaccccatccccatccttgagtacagcactggcggccgaggccccctcgtccctactctcgatcagcccaacgccaacaacagcagcaacgagccctaccttgagttcctcacctacctgctcgcccagcccgacagcgccattccccagactctcagcgtgagctacggcgaggaagaacagagcgtcccccgcgactacgccatcaaggtctgcaacatgttcatgcagctcggcgctcgcggcgtcagcgtcatgtttagcagcggcgatagcggccctggcaacgactgcgtccgagcctctgacaacgccaccttcttcggcagcaccttccctgccggctgcccctacgtcactagcgtcggcagcaccgtcggcttcgagcctgagcgagccgtcagctttagctccggcggcttcagcatctaccacgcccgacccgactaccagaacgaggtcgtccccaagtacatcgagagcatcaaggccagcggctacgagaagttcttcgacggcaacggccgaggcatccccgatgtcgctgctcagggcgctcgcttcgtcgtcatcgacaagggccgcgtcagcctcatcagcggcactagcgcttccagccccgccttcgctggcatggtcgccctcgtcaacgccgctcgcaagagcaaggatatgcccgccctcggcttcctcaaccccatgctctaccagaacgctgccgccatgaccgacatcgtcaacggcgctggcatcggctgccgcaagcagcgcaccgagtttcccaacggtgcccgcttcaacgccaccgccggatgggaccctgtcactggccttggcacccccctgttcgacaagctcctcgccgttggcgctcccggcgtccctaacgcctaa

SEQ ID NO: 94 - DNA, 1845 bp, Togninia minima
atgcagaccttcggtgcttttctcgtttccttcctcgccgccagcggcctggccgcggcctccgatgtcgtccttgagtctctccgcgaggtcccccagggctggaagcgactccgagatgccgaccccgagcagagcatcaagctccgcattgcccttgagcagcccaacctcgacctcttcgagcagaccctctacgacatcagcagccccgaccaccccaagtacggccagcacctcaagagccacgagctgcgcgacatcatggcccctcgcgaggaatccactgccgccgtcattgcctggctccaggatgctggcctcagcggcagccagatcgaggacgacagcgactggatcaacatccagaccaccgtcgcccaggccaacgacatgctcaacaccaccttcggcctcttcgcccaagagggcaccgaggtcaaccgcattcgcgccctcgcctacagcgtccccgaggaaattgtcccccacgtcaagatgatcgcccccatcatccgcttcggccagctccgccctcagatgagccacatcttcagccacgagaaggtcgaggaaacccccagcatcggcaccatcaaggccgctgccatccccagcgtcgacctcaacgtcaccgcctgcaacgccagcatcacccccgagtgcctccgcgccctctacaacgtcggcgactacgaggccgaccccagcaagaagtccctcttcggcgtctgcggctaccttgagcagtacgccaagcacgaccagctcgccaagttcgagcagacgtacgccccctacgccatcggcgccgacttcagcgtcgtcaccatcaacggcggaggcgacaaccagaccagcaccatcgacgacggcgaggccaacctcgacatgcagtacgccgtcagcatggcctacaagacccccatcacctactacagcactggcggccgaggccccctcgtccctgatctcgatcagcccgaccccaacgacgtcagcaacgagccctacctcgacttcgtcagctacctcctcaagctccccgacagcaagctcccccagaccatcaccaccagctacggcgaggacgagcagagcgtcccccgcagctacgtcgagaaggtctgcaccatgttcggcgcccttggcgcccgaggcgtcagcgtcattttcagctctggcgacaccggcgtcggcagcgcctgccagactaacgacggcaagaacaccacccgctttctgcccatcttccctgccgcctgcccctacgtcactagcgtcggcggcacccgctacgtcgatcctgaggtcgccgtcagcttcagcagcggcggcttcagcgacatcttccccacccccctgtaccagaagggcgccgtcagcggctacctcaagatcctcggcgaccgctggaagggcctctacaaccctcacggccgaggcttccctgacgtcagcggccagtctgtccgctaccacgtctttgactacggcaaggacgtcatgtacagcggcaccagcgccagcgcccccatgtttgctgctctcgtcagcctcctcaacaacgcccgcctcgccaagaagctcccccctatgggcttcctcaacccctggctctacaccgtcggcttcaacggcctcaccgacatcgtccacggcggctctactggctgcaccggcaccgatgtctacagcggcctgcctacccccttcgtcccctacgcctcttggaacgccaccgtcggctgggaccctgtcactggccttggcacccccctgttcgacaagctcctcaacctcagcacccccaacttccacctcccccacatcggcggccactaa

SEQ ID NO: 95 - DNA, 1848 bp, Bipolaris maydis
atgcagaccttcggtgcttttctcgtttccttcctcgccgccagcggcctggccgcggcctctaccacttctcacgtcgagggcgaggtcgtcgagcgccttcatggcgtccctgagggctggtcacaggtcggcgctcccaaccccgaccagaagctccgcttccgcattgccgtccgcagcgccgacagcgagctgttcgagcgcaccctcatggaagtcagcagccccagccacccccgctacggccagcacctcaagcgccacgagctgaaggacctcatcaagcctcgcgccaagagcaccagcaacatcctcaactggctccaagagagcggcatcgaggcccgcgacatccagaacgacggcgagtggatcagcttctacgcccccgtcaagcgagccgagcagatgatgagcaccaccttcaagacctaccagaacgaggcccgagccaacatcaagaagatccgcagcctcgactacagcgtccccaagcacatccgcgacgacatcgacatcatccagcccaccacgcgcttcggccagatccagcctgagcgcagccaggtctttagccaagaggaagtccccttcagcgccctcgtcgtcaacgccacgtgcaacaagaagatcacccccgactgcctcgccaacctctacaacttcaaggactacgacgccagcgacgccaacgtcacgatcggcgtcagcggcttccttgagcagtacgcccgcttcgacgacctcaagcagttcatcagcaccttccagcccaaggccgctggctccaccttccaggtcaccagcgtcaacgctggccccttcgaccagaacagcaccgcctctagcgtcgaggccaacctcgacatccagtacaccaccggcctcgtcgcccccgacatcgagactcgctacttcaccgtccccggacgcggcatcctcatccccgacctcgaccagcctaccgagagcgacaacgccaacgagccctacctcgactacttcacctacctcaacaaccttgaggacgaggaactccccgacgtcctcaccaccagctacggcgagagcgagcagagcgtccctgccgagtacgccaagaaggtctgcaacctcatcggccagctcggcgctcgcggcgtcagcgtcattttcagcagcggcgacaccggccctggcagcgcctgccagactaacgacggcaagaacaccacccgctttctgcccatcttccccgccagctgcccctacgtcactagcgtcggcggcactgtcggcgtcgagcctgagaaggccgtcagctttagcagcggcggcttcagcgacctctggccccgacctgcctaccaagagaaggccgtgagcgagtaccttgagaagctcggcgaccgctggaacggcctctacaaccctcagggccgaggcttccctgacgtcgctgctcagggccagggcttccaggtctttgacaagggccgcctcatctcggtcggcggcacatctgcttccgcccctgtctttgccagcgtcgtcgccctcctcaacaacgcccgaaaggctgccggaatgagcagcctcggcttcctcaacccctggatctacgagcagggctacaagggcctcaccgacatcgtcgctggcggctctactggctgcaccggccgctctatctacagcggcctccctgcccccctggtcccttacgcttcttggaacgccaccgagggctgggaccccgtcactggctatggcacccccgacttcaagcagctcctcaccctcgccaccgcccccaagtctggcgagcgacgagttcgacgaggcggccttggaggccaggcttaa

SEQ ID NO: 96 - protein, 6 aa, Artificial Sequence
  Sequence motif
  MISC_FEATURE (1)..(1): Xaa may be Gly, Thr, Ser or Val
XEANLD

SEQ ID NO: 97 - protein, 5 aa, Artificial Sequence
  Sequence motif
  MISC_FEATURE (1)..(1): Xaa may be Ile, Leu or Val
  MISC_FEATURE (3)..(3): Xaa may be Ser or Thr
  MISC_FEATURE (4)..(4): Xaa may be Ile or Val
XTXXG

SEQ ID NO: 98 - protein, 5 aa, Artificial Sequence
  Sequence motif
QNFSV

SEQ ID NO: 99 - protein, 8 aa, Artificial Sequence
  Peptide substrate
RGPFPIIV

SEQ ID NO: 100 - protein, 5 aa, Artificial Sequence
  Peptide substrate
AAFPA

SEQ ID NO: 101 - protein, 5 aa, Artificial Sequence
  Peptide substrate sequence
AAPPA

SEQ ID NO: 102 - protein, 525 aa, Artificial Sequence
  Enzyme polypetide sequence (not to be combined with)
MRTAAASLTLAATCLFELASALMPRAPLIPAMKAKVALPSGNATFEQYIDHNNPGLGTFPQRYWYNPEFWAGPGSPVLLFTPGESDAADYDGFLTNKTIVGRFAEEIGGAVILLEHRYWGASSPYPELTTETLQYLTLEQSIADLVHFAKTVNLPFDEIHSSNADNAPWVMTGGSYSGALAAWTASIAPGTFWAYHASSAPVQAIYDFWQYFVPVVEGMPKNCSKDLNRVVEYIDHVYESGDIERQQEIKEMFGLGALKHFDDFAAAITNGPWLWQDMNFVSGYSRFYKFCDAVENVTPGAKSVPGPEGVGLEKALQGYASWFNSTYLPGSCAEYKYWTDKDAVDCYDSYETNSPIYTDKAVNNTSNKQWTWFLCNEPLFYWQDGAPKDESTIVSRIVSAEYWQRQCHAYFPEVNGYTFGSANGKTAEDVNKWTKGWDLTNTTRLIWANGQFDPWRDASVSSKTRPGGPLQSTEQAPVHVIPGGFHCSDQWLVYGEANAGVQKVIDEEVAQIKAWVAEYPKYRKP

SEQ ID NO: 103 - protein, 844 aa, Lactobacillus helveticus
MAVKRFYKTFHPEHYDLRINVNRKNKTINGTSTITGDVIENPVFINQKFMTIDSVKVDGKNVDFDVIEKDEAIKIKTGVTGKAVIEIAYSAPLTDTMMGIYPSYYELEGKKKQIIGTQFETTFARQAFPCVDEPEAKATFSLALKWDEQDGEVALANMPEVEVDKDGYHHFEETVRMSSYLVAFAFGELQSKTTHTKDGVLIGVYATKAHKPKELDFALDIAKRAIEFYEEFYQTKYPLPQSLQLALPDFSAGAMENWGLVTYREAYLLLDPDNTSLEMKKLVATVITHELAHQWFGDLVTMKWWDNLWLNESFANMMEYLSVDGLEPDWHIWEMFQTSEAASALNRDATDGVQPIQMEINDPADIDSVFDGAIVYAKGSRMLVMVRSLLGDDALRKGLKYYFDHHKFGNATGDDLWDALSTATDLDIGKIMHSWLKQPGYPVVNAFVAEDGHLKLTQKQFFIGEGEDKGRQWQIPLNANFDAPKIMSDKEIDLGNYKVLREEAGHPLRLNVGNNSHFIVEYDKTLLDDILSDVNELDPIDKLQLLQDLRLLAEGKQISYASIVPLLVKFADSKSSLVINALYTTAAKLRQFVEPESNEEKNLKKLYDLLSKDQVARLGWEVKPGESDEDVQIRPYELSASLYAENADSIKAAHQIFTENEDNLEALNADIRPYVLINEVKNFGNAELVDKLIKEYQRTADPSYKVDLRSAVTSTKDLAAIKAIVGDFENADVVKPQDLCDWYRGLLANHYGQQAAWDWIREDWDWLDKTVGGDMEFAKFITVTAGVFHTPERLKEFKEFFEPKINVPLLSREIKMDVKVIESKVNLIEAEKDAVNDAVAKAID

(19) United States
(12) Patent Application Publication
(10) Pub. No.: US 2017/0306360 A1
(43) Pub. Date: Oct. 26, 2017

(54) METHOD FOR PRODUCING ALCOHOL BY USE OF A TRIPEPTIDYL PEPTIDASE

(71) Applicant: DANISCO US INC., Palo Alto, CA (US)

(72) Inventors: Andrei MIASNIKOV, Union City, CA (US); Maria MA, Fremont, CA (US); Scott D. POWER, Woodside, CA (US)

(73) Assignee: DANISCO US INC., Palo Alto, CA (US)

(21) Appl. No.: 15/520,584

(22) PCT Filed: Oct. 23, 2015
(86) PCT No.: PCT/US2015/057079

§ 371 Date: Apr. 20, 2017

Related U.S. Application Data
(60) Provisional application No. 62/068,294, filed on Oct. 24, 2014.

(51) Int. Cl. C12P 7/10; C12P 7/56; C12P 7/48

(52) U.S. Cl. CPC C12P 7/10; C12Y 304/14; C12P 7/48; C12P 7/56

(57) ABSTRACT

The present invention provides a method for producing an alcohol comprising: (a) admixing a tripeptidyl peptidase, predominantly having exopeptidase activity, with a feedstock or a fraction thereof before, during or after fermentation of said feedstock or a fraction thereof; and (b) recovering an alcohol. Also provided are uses of a tripeptidyl peptidase and by-products of alcohol production obtainable by the method of the invention.

### CROSS-REFERENCE TO RELATED APPLICATIONS

This application claims the benefit of priority from U.S. Provisional Application No. 62/068,294, filed Oct. 24, 2014, which is hereby incorporated by reference.

### FIELD OF THE INVENTION

The present invention relates to tripeptidyl peptidases for use in ethanol production, particularly bioethanol for biofuel production.

### BACKGROUND

Proteases (synonymous with peptidases) are enzymes that are capable of cleaving peptide bonds between amino acids in substrate peptides, oligopeptides and/or proteins.

Proteases are grouped into 7 families based on their catalytic reaction mechanism and the amino acid residue involved in the active site for catalysis. The serine proteases, aspartic acid proteases, cysteine proteases and metalloprotease are the 4 major families, whilst the threonine proteases, glutamic acid proteases and ungrouped proteases make up the remaining 3 families.

The substrate specificity of a protease is usually defined in terms of preferential cleavage of bonds between particular amino acids in a substrate. Typically, amino acid positions in a substrate peptide are defined relative to the location of the scissile bond (i.e. the position at which a protease cleaves):

NH₂— . . . P3-P2-P1*P1′-P2′-P3′ . . . —COOH

Illustrated using the hypothetical peptide above, the scissile bond is indicated by the asterisk (*) whilst amino acid residues are represented by the letter ‘P’, with the residues N-terminal to the scissile bond beginning at P1 and increasing in number when moving away from the scissile bond towards the N-terminus. Amino acid residues C-terminal to the scissile bond begin at P1′ and increase in number moving towards the C-terminal residue.

Proteases can be also generally subdivided into two broad groups based on their substrate-specificity. The first group is that of the endoproteases, which are proteolytic peptidases capable of cleaving internal peptide bonds of a peptide or protein substrate and tending to act away from the N-terminus or C-terminus. Examples of endoproteases include trypsin, chymotrypsin and pepsin. In contrast, the second group of proteases is the exopeptidases which cleave peptide bonds between amino acids located towards the C or N-terminus of a protein or peptide substrate.

Certain enzymes of the exopeptidase group may have tripeptidyl peptidase activity. Such enzymes are therefore capable of cleaving 3 amino acid fragments (tripeptides) from the unsubstituted N-terminus of substrate peptides, oligopeptides and/or proteins. Tripeptidyl peptidases are known to cleave tripeptide sequences from the N-terminus of a substrate but except bonds with proline at the P1 and/or P1′ position. Alternatively tripeptidyl peptidases may be proline-specific and only capable of cleaving substrates having a proline residue N-terminal to the scissile bond (i.e. in the P1 position).

### SUMMARY OF THE INVENTION

In a broad aspect the present invention provides a method for producing an alcohol comprising:
(a) admixing a tripeptidyl peptidase comprising one or more amino acid sequence selected from the group consisting of SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof or an amino acid sequence having at least 70% identity therewith; or a tripeptidyl peptidase expressed from one or more of the nucleotide sequences selected from the group consisting of: SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95 or a nucleotide sequence having at least 70% identity therewith, or which differs from these nucleotide sequences by the degeneracy of the genetic code, or which hybridises under medium or high stringency conditions; and
(b) recovering an alcohol.

In a first aspect the present invention provides a method for producing an alcohol comprising:
(a) admixing a tripeptidyl peptidase, predominantly having exopeptidase activity, with a feedstock or a fraction thereof before, during or after fermentation of said feedstock or a fraction; and
(b) recovering an alcohol.

In a second aspect there is provided the use of a tripeptidyl peptidase, predominantly having exopeptidase activity, in the manufacture of an alcohol for improving yield of the alcohol.

In a third aspect there is provided the use of one or more tripeptidyl peptidases(s), predominantly having exopeptidase activity, in the manufacture of an alcohol for improving an alcohol production host's ability to ferment.

In a fourth aspect there is provided a by-product of alcohol production obtainable (e.g. obtained) by the method of the invention.

### BRIEF DESCRIPTION OF THE DRAWINGS

Embodiments of the invention will now be described, by way of example only, with reference to accompanying drawings, in which:

FIG. 1 shows ethanol levels for peptidase in 200 ppm urea fermentations.

FIG. 2 shows ethanol levels in double dosed fermentations.

FIG. 3 shows glucose levels in fermentations for different proteases.

FIG. 4 shows ethanol levels in fermentations at 400 ppm urea with a comparison of proteases.

FIG. 5 shows late fermentation ethanol levels for 400 ppm urea fermentations.

FIG. 6 shows ethanol levels in fermentations with added tripeptidyl peptidase.

FIG. 7 shows total glucose release in fermentations.

FIG. 8 shows concentrations of small sugars in fermentation and different protease treatment.

FIG. 9 shows the effects of a tripeptidyl peptidase on ethanol yield by itself and combined with Fermgen® (an acid fungal endoprotease).

FIG. 10 shows double dose fermentation rate comparisons.

FIG. 11 shows a plasmid map of the expression vector pTTT-TRI083.

FIG. 12 shows a plasmid map of the expression vector pTTT-pyrG13-TRI071. The endogenous signal sequences was replaced by the secretion signal sequence from the Trichoderma reesei acidic fungal protease (Alphalase® AFP (available at Genencor Division, Food Enzymes)) and an intron from a Trichoderma reesei glycoamylase gene (TrGA1) (see lower portion of FIG. 12).

FIG. 13 shows alignments between a number of tripeptidyl peptidase amino acid sequences. The xEANLD, y′Tzx′G and QNFSV motifs are shown (boxed).

### DETAILED DESCRIPTION

A seminal finding of the present invention is that use of a tripeptidyl peptidase predominantly having exopeptidase activity during alcohol production improves alcohol yield.

In addition or alternatively a further finding was that use of a tripeptidyl peptidase predominantly having exopeptidase activity during alcohol production improves the ability of an alcohol production host to ferment.

Based on these findings, there is provided a method for producing an alcohol comprising: (a) admixing a tripeptidyl peptidase, predominantly having exopeptidase activity, with a feedstock or a fraction thereof before, during or after fermentation of said feedstock or a fraction; and (b) recovering an alcohol.

The term “alcohol” as used herein refers to any alcohol produced as a result of a biological fermentation process. The alcohol may for example be ethanol and/or butanol. Preferably, the alcohol may be a biofuel, such as bioethanol for example.

The method of the present invention comprises a step for the recovery of an alcohol.

The term “recovery of an alcohol” or “recovering an alcohol” refers to purification and/or isolation of an alcohol. Suitably, the recovery step results in an alcohol that is substantially free of other components (e.g. contaminants). Therefore, the recovery may result in an alcohol that is at least about 90% pure, suitably at least about 95% pure, more suitably at least 99% pure. Preferably the recovery may result in an alcohol that is at least about 99.9% pure.

The recovery of an alcohol may be achieved by any means known to one skilled in the art. In one embodiment the alcohol may be distilled.

The term “admixing” as used herein refers to the mixing of one or more ingredients and/or enzymes where the one or more ingredients or enzymes are added in any order and in any combination. Suitably, admixing may relate to mixing one or more ingredients and/or enzymes simultaneously or sequentially.

In one embodiment the one or more ingredients and/or enzymes may be mixed sequentially. Preferably, the one or more ingredients and/or enzymes may be mixed simultaneously.

In one embodiment a tripeptidyl peptidase for use in the methods and/or uses of the present invention may be incubated with a substrate (e.g. a protein and/or peptide substrate) at a temperature of at least about 25° C. In other words the method of the present invention may be carried out at a temperature of at least about 25° C.

Suitably the tripeptidyl peptidase may be incubated with a substrate at a temperature of at least about 30° C., suitably at least about 35° C.

In one embodiment a tripeptidyl peptidase for use in the methods and/or uses of the present invention may be incubated with a substrate at a temperature of at between about 25° C. to about 40° C., suitably at a temperature of between about 25° C. to about 35° C.

In another embodiment the tripeptidyl peptidase for use in the methods and/or uses of the present invention may be incubated with a substrate (e.g. a protein and/or peptide substrate) at a temperature of between about 40° C. to about 70° C. In other words the method of the present invention may be carried out at a temperature of between about 40° C. to about 70° C.

Suitably the tripeptidyl peptidase may be incubated with a substrate at a temperature of between about 40° C. to about 65° C., more suitably at a temperature of between about 45° C. to about 65° C.

Preferably the tripeptidyl peptidase may be incubated with a substrate at a temperature of between about 50° C. to about 60° C.

The term “tripeptidyl peptidase” refers to a protease predominantly having exopeptidase activity and that is capable of cleaving tripeptides from the N-terminus of a protein, oligopeptide and/or peptide substrate.

In one embodiment the tripeptidyl peptidase is not an endoprotease.

In another embodiment the tripeptidyl peptidase is not an enzyme which cleaves tetrapeptides from the N-terminus of a substrate.

In a further embodiment the tripeptidyl peptidase is not an enzyme which cleaves dipeptides from the N-terminus of a substrate.

In a yet further embodiment the tripeptidyl peptidase is not an enzyme which cleaves single amino acids from the N-terminus of a substrate.

A tripeptidyl peptidase can cleave protein and/or peptide substrates present in a feedstock to liberate tripeptides, surprisingly this may increase alcohol production during fermentation.

Therefore in another aspect there is provided the use of a tripeptidyl peptidase predominantly having exopeptidase activity in the manufacture of an alcohol for improving the yield of an alcohol.

A further advantage of the use of a tripeptidyl peptidase is that its use may improve an alcohol production host's ability to ferment during alcohol production.

Thus, in a further aspect there is provided the use of a tripeptidyl peptidase predominantly having exopeptidase activity in the manufacture of an alcohol for improving the alcohol production host's ability to ferment.

In one embodiment a tripeptidyl peptidase for use in accordance with the present invention may be an exo-tripeptidyl peptidase of the S53 family.

The term “exo-tripeptidyl peptidase of the S53 family” as used herein refers to a protease predominantly having exopeptidase activity as well as the ability to cleave tripeptides from the N-terminus of a protein and/or peptide substrate. The S53 family peptidases broadly encompass a class of serine proteases. Although the S53 family includes both endoproteases and exopeptidases it is intended herein that this definition refers only to those tripeptidyl peptidases predominantly having exopeptidase activity.

An “exo-tripeptidyl peptidase of the S53 family” has an activity of at least about 50 nkat per mg of protein in the “Exopeptidase Broad-Specificity Assay” (EBSA) taught herein. Suitably an “exo-tripeptidyl peptidase of the S53 family” in accordance with the present invention has an activity of between about 50-2000 nkat per mg of protein in the EBSA activity assay taught herein.

In one embodiment the tripeptidyl peptidase may be a “proline tolerant tripeptidyl peptidase”, also referred to herein as 3PP.

The term “proline tolerant tripeptidyl peptidase” as used herein relates to an exopeptidase which can cleave tripeptides from the N-terminus of a peptide, oligopeptide and/or protein substrate. A “proline tolerant tripeptidyl peptidase” is capable of cleaving peptide bonds where proline is at position P1 as well as cleaving peptide bonds where an amino acid other than proline is at P1 and/or capable of cleaving peptide bonds where proline is at position P1′ as well as cleaving peptide bonds where an amino acid other than proline is at P1′.

Advantageously the tripeptidyl peptidase for use in the present invention (e.g. a proline tolerant tripeptidyl peptidase) may have activity on a substrate having proline at P1 and/or P1′ as well as any other amino acid at P1 and/or P1′. This is highly surprising as tripeptidyl peptidases that have been documented in the art typically are inhibited when proline is at P1 or are active when proline is at P1 but inactive when an amino acid other than proline is present at position P1 in the substrate, this is sometimes referred to herein as a proline-specific tripeptidyl peptidase.

Further advantageously, a tripeptidyl peptidase (e.g. a proline tolerant tripeptidyl peptidase) having such an activity is capable of acting on a wide range of peptide and/or protein substrates and due to having such a broad substrate-specificity is not readily inhibited from cleaving substrates enriched in certain amino acids (e.g. proline and/or lysine and/or arginine and/or glycine). The use of such a proline tolerant tripeptidyl peptidase therefore may efficiently and/or rapidly breakdown protein substrates (e.g. present in a substrate for preparation of a hydrolysate).

Suitably the tripeptidyl peptidase (e.g. proline tolerant tripeptidyl peptidase) for use in the methods and/or uses of the present invention may be capable of cleaving tri-peptides from the N-terminus of peptides having proline at P1; and an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1.

Alternatively or additionally, the tripeptidyl peptidase (e.g. proline tolerant tripeptidyl peptidase) for use in the methods of the present invention may be capable of cleaving tri-peptides from the N-terminus of peptides having proline at P1′; and an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′.

In one embodiment the tripeptidyl peptidase (e.g. proline tolerant tripeptidyl peptidase) may be capable of cleaving peptide bonds where proline is at position P1 as well as cleaving peptide bonds where an amino acid other than proline is at P1.

In another embodiment the tripeptidyl peptidase (e.g. proline tolerant tripeptidyl peptidase) may be capable of cleaving peptide bonds where proline is at position P1′ as well as cleaving peptide bonds where an amino acid other than proline is at P1′.

Suitably, the tripeptidyl peptidase (e.g. proline tolerant tripeptidyl peptidase) may also be able to cleave peptide bonds where the proline present at position P1 and/or P1′ is present in its cis or trans configuration.

Suitably an “amino acid other than proline” may be an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids.

In another embodiment the “amino acid other than proline” may be an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine or valine.

Suitably, in such an embodiment synthetic amino acids may be excluded.

Preferably, the proline tolerant tripeptidyl peptidase may be able to cleave peptide bonds where proline is present at position P1 and P1′.

It is surprising that a tripeptidyl peptidase can act on a substrate having proline at position P1 and/or P1′. It is even more surprising that in addition to this activity a tripeptidyl peptidase may also have activity when an amino acid other than proline is present at position P1 and/or P1′.

In addition to having activity on any of the various substrates as described above the tripeptidyl peptidase (e.g. proline tolerant tripeptidyl peptidase) for use in the present invention may additionally be tolerant of proline at one or more positions selected from the group consisting of: P2, P2′, P3 and P3′.

Suitably the tripeptidyl peptidase (e.g. proline tolerant tripeptidyl peptidase) in addition to having the activities described above may be tolerant of proline at position P2, P2′, P3 and P3′.

This is advantageous as it allows the efficient cleavage of peptide and/or protein substrates having stretches of proline and allows cleavage of a wide range of peptide and/or protein substrates.

The tripeptidyl peptidase (e.g. proline tolerant tripeptidyl peptidase) may have a preferential activity on peptides and/or proteins having one or more of lysine, arginine or glycine in the P1 position. Without wishing to be bound by theory peptide and/or protein substrates comprising these amino acids at the P1 position may be difficult to digest for many tripeptidyl peptidases and/or proteases in generally and upon encountering such residues cleavage of the peptide and/or protein substrate by a tripeptidyl peptidase and/or protease may halt or slow. Advantageously, by using a tripeptidyl peptidase (e.g. proline tolerant tripeptidyl peptidase) of the invention it is possible to digest protein and/or peptide substrates comprising lysine, arginine and/or glycine at P1 efficiently.

Suitably the tripeptidyl peptidase (e.g. proline tolerant tripeptidyl peptidase) may have a preferential activity on peptides and/or proteins having lysine at the P1 position. Advantageously this allows the efficient cleavage of substrates having high lysine content, such as whey protein.

In one embodiment the tripeptidyl peptidase (e.g. proline tolerant tripeptidyl peptidase) may comprise a catalytic triad of the amino acids serine, aspartate and histidine.

The tripeptidyl peptidase for use in the present invention may be a thermostable tripeptidyl peptidase.

The term “thermostable” means that an enzyme retains its activity when heated to temperatures of up to about 60° C. Suitably “thermostable” may mean that an enzyme retains its activity when heated to about 65° C., more suitably about 70° C.

In another embodiment “thermostable” means that an enzyme retains its activity when heated to temperatures up to about 75° C. Suitably “thermostable” may mean that an enzyme retains its activity when heated to about 80° C., more suitably about 90° C.

Advantageously, a thermostable tripeptidyl peptidase is less prone to being denatured (e.g. when added to a feedstock before fermentation) and/or will retain its activity for a longer period of time when subjected to increased temperatures when compared to a non-thermostable variant.

The tripeptidyl peptidase for use in the present invention may have activity in a range of about pH 2 to about pH 8. Suitably, the tripeptidyl peptidase may have activity in a range of about pH 4 to about pH 8, more suitably in a range of about pH 4.5 to about pH 6.5.

Suitably the method of the present invention may be carried out at a pH of between 2 to about 7.

In one embodiment the method of the present invention may be carried out at a pH of between about 4 to about 7, e.g. 4.5 to 6.5.

Using a tripeptidyl peptidase having activity in a pH range between about pH 4 to about pH 7 is advantageous as it allows the tripeptidyl peptidase to be used with one more endoproteases having activity in this pH range.

When a tripeptidyl peptidase having activity in a pH range between about pH 4 to about pH 7 is used, suitably it may be used in combination with a neutral or an alkaline endoprotease.

Advantageously this means that changing the pH of the reaction medium comprising the protein and/or peptide substrate for hydrolysate production is not necessary between enzyme treatments. In other words it allows the tripeptidyl peptidase and the endoprotease to be added to a reaction simultaneously, which may make the process for producing the hydrolysate quicker and/or more efficient and/or more cost-effective. Moreover, this allows for a more efficient reaction as at lower pH values the substrate may precipitate out of solution and therefore not be cleaved.

Any suitable alkaline endoprotease may be used in the present invention.

In one embodiment the alkaline endoprotease may be a member of the serine protease family of enzymes (EC 3.4.21). Serine proteases possess an active site serine that initiates hydrolysis of peptide bonds of proteins. There are two broad categories of serine proteases, based on their structure: chymotrypsin-like (trypsin-like) and subtilisin-like. The prototypical subtilisin (EC No. 3.4.21.62) was initially obtained from Bacillus subtilis. Subtilisins and their homologues are members of the S8 peptidase family of the MEROPS classification scheme. Members of family S8 have a catalytic triad in the order Asp, His and Ser in their amino acid sequence.

Suitably, the alkaline endoprotease may be one or more selected from the group consisting of: a subtilisin, a bacterial neutral protease, a thermolysin, a trypsin and a chymotrypsin.

In one embodiment the subtilisin may be a subtilisin of the serine protease family.

Suitably the subtilisin may be a subtilisin obtainable (e.g. obtained) from the Bacillus genera of bacteria.

In one embodiment the subtilisin may be a FNA subtilisin e.g. as taught in US20120003718 the contents of which is incorporated herein by reference.

In another embodiment the tripeptidyl peptidase may have activity at an acidic pH (suitably, the tripeptidyl peptidase may have optimum activity at acidic pH). The tripeptidyl peptidase may have activity at a pH of less than about pH 6, more suitably less than about pH 5. Preferably, the tripeptidyl peptidase may have activity at a pH of between about 2.5 to about pH 4.0, more suitably at between about 3.0 to about 3.3.

Suitably the method of the present invention, in particular the hydrolysis step, may be carried out at a pH of between 2 to about 4, e.g. 3 to 3.3. In one embodiment the proline tolerant tripeptidyl peptidase may have activity at a pH around 2.5.

In one embodiment the proline tolerant tripeptidyl peptidase may have activity at a pH around 2.5.

In some embodiments the tripeptidyl peptidase may be used in combination with an endoprotease.

The term “endoprotease” as used herein is synonymous with the term “endopeptidase” and refers to an enzyme which is a proteolytic peptidase capable of cleaving internal peptide bonds of a peptide or protein substrate (e.g. not located towards the C or N-terminus of the peptide or protein substrate). Such endoproteases may be defined as one that tend to act away from the N-terminus or C-terminus.

Suitable endoproteases include those of animal, vegetable or microbial origin. Chemically modified or protein engineered mutants are included, as well as naturally processed proteins. The endoprotease may be a serine protease or a metalloprotease, an alkaline microbial protease, a trypsin-like protease, or a chymotrypsin-like protease. Examples of alkaline endoproteases are subtilisins, especially those derived from Bacillus, e.g., subtilisin Novo, subtilisin Carlsberg, subtilisin 309, subtilisin 147, and subtilisin 168 (see, e.g., WO 89/06279). Additional examples include those mutant proteases described in U.S. Pat. Nos. RE 34,606, 5,955,340, 5,700,676, 6,312,936, and 6,482,628, all of which are incorporated herein by reference. Examples of trypsin-like endoproteases are trypsin (e.g., of porcine or bovine origin), and Fusarium proteases (see, e.g., WO 89/06270 and WO 94/25583). Examples of useful proteases also include but are not limited to the variants described in WO 92/19729, WO 98/20115, WO 98/20116, and WO 98/34946. Commercially available protease enzymes include but are not limited to: Alcalase®, Savinase®, Primase™, Duralase™, Esperase®, BLAZE™, POLARZYME®, OVOZYME®, KANNASE®, LIQUANASE®, NEUTRASE®, RELASE®, and ESPERASE® (Novo Nordisk A/S and Novozymes A/S), Maxatase®, Maxacal™, Maxapem™, Properase®, Purafect®, Purafect OxP™, Purafect Prime™, FNA™, FN2™, FN3™, OPTICLEAN®, OPTIMASE®, PURAMAX™, EXCELLASE™, and PURAFAST™ (Danisco US Inc./DuPont Industrial Biosciences, Palo Alto, Calif., USA), BLAP™ and BLAP™ variants (Henkel Kommanditgesellschaft auf Aktien, Duesseldorf, Germany), and KAP (B. alkalophilus subtilisin; Kao Corp., Tokyo, Japan). Another exemplary proteases NprE from Bacillus amyloliquifaciens and ASP from Cellulomonas sp. strain 69B4 (Danisco US Inc./DuPont Industrial Biosciences, Palo Alto, Calif., USA). Various proteases are described in WO95/23221, WO 92/21760, WO 09/149200, WO 09/149144, WO 09/149145, WO 11/072099, WO 10/056640, WO 10/056653, WO 11/140364, WO 12/151534, U.S. Pat. Publ. No. 2008/0090747, and U.S. Pat. Nos. 5,801,039, 5,340,735, 5,500,364, 5,855,625, U.S. Pat. No. RE 34,606, 5,955,340, 5,700,676, 6,312,936, and 6,482,628, and various other patents. In some further embodiments, metalloproteases find use in the present invention, including but not limited to the neutral metalloprotease described in WO 07/044993. Suitable endoproteases include naturally occurring proteases or engineered variants specifically selected or engineered to work at relatively low temperatures.

In one embodiment the endoprotease may be one or more selected from the group consisting of: a serine protease, an aspartic acid protease, a cysteine protease, a metalloprotease, a threonine protease, a glutamic acid protease and a protease selected from the family of ungrouped proteases.

In one embodiment the endoprotease may be one or more selected from the group consisting of: an acid fungal protease, a subtilisin, a chymotrypsin, a trypsin, a pepsin, papain, bromalin, thermostable bacterial neutral metalloendopeptidase, metalloneutral endopeptidase, alkaline serine protease, fungal endoprotease or from the group of commercial protease products Alphalase® AFP, Alphalase® FP2, Alphalase® NP.

Preferably an endoprotease for use in accordance with the present invention may be an aspartic acid endoprotease.

In one embodiment, the endoprotease may be an acid endoprotease. Suitably, the endoprotease may be an acid fungal protease. Preferably, the acid fungal protease may be an aspartic acid endoprotease.

At least one example of a suitable acid fungal protease is the enzyme composition FERMGEN® (available from DuPont Industrial Biosciences—formerly Genencor (USA)).

In one embodiment a protease for use in accordance with the present invention may not be obtainable (e.g. obtained) from Nocardiopsis.

Advantageously, the use of an endoprotease in combination with a tripeptidyl peptidase can increase the efficiency of substrate cleavage. Without wishing to be bound by theory, it is believed that an endoprotease is able to cleave a peptide and/or protein substrate at multiple regions away from the C or N-terminus, thereby producing more N-terminal ends for the tripeptidyl peptidase to use as a substrate, thereby advantageously increasing reaction efficiency and/or reducing reaction times.

The term “alcohol production host” refers to any organism that has the ability to ferment a fermentable sugar source to produce an alcohol. Such an organism may also be referred to as an ethanologen or said to be ethanologenic.

As used herein, “fermentable sugars” refer to saccharides that are capable of being metabolized under fermentation conditions. These sugars typically refer to glucose, maltose and maltotriose (DP1, DP2 and DP3). In some embodiments sucrose may also be a fermentable sugar.

Suitably, the fermentable sugars may be obtainable (e.g. obtained) by the hydrolysis of starch.

As used herein, “starch” refers to any material comprised of the complex polysaccharide carbohydrates of plants, comprised of amylose and amylopectin with the formula (C6H10O5)x, wherein “X” can be any number. In particular, the term refers to any plant-based material including but not limited to grains, cereals, grasses, tubers and roots and more specifically wheat, barley, corn, rye, rice, sorghum, brans, cassava, millet, potato, sweet potato, and tapioca. “Granular starch” refers to uncooked (raw) starch, which has not been subject to gelatinization, where “starch gelatinization” means solubilisation of a starch molecule to form a viscous suspension.

Suitably the tuber may be a grain cereal tuber.

As used herein, “hydrolysis of starch” and the like refers to the cleavage of glucosidic bonds with the addition of water molecules. Thus, enzymes having “starch hydrolysis activity” catalyze the cleavage of glucosidic bonds with the addition of water molecules.

The alcohol production host may be selected from any suitable eukaryotic organism.

In one embodiment the alcohol production host may be a bacterium. Suitably, selected from the Proteobacteria, more suitably from the family Shingomonadaceae.

In a particular embodiment, the alcohol production host may be a bacterium from one or more genus selected from the group consisting of: Zymomonas, Arthrobacter, Bacillus, Clostridium, Erwinia, Escherichia, Klebsiella, Lactobacillus, Pseudomonas, Streptomyces, Thermoanaerobacter.

Suitably, the bacterium may be selected from the group consisting of Zymomonas mobilis.

In some embodiments the alcohol production host may be a fungus. The fungus for use in accordance with the present invention may be any ascomycetous fungus (e.g. an ascomycete).

Suitably, the alcohol production host may be a yeast.

Suitably the yeast may be selected from the group consisting of: Saccharomyces Kluyveromyces, Zygosaccharomyces, Issatchenkia, Kazachstania and Torulaspora.

More suitably the yeast may be one or more selected from the group consisting of: Saccharomyces cerevisiae, Saccharomyces bayanus, Saccharomyces carlsbergensis, Saccharomyces kudriavtsevii, Saccharomyces kudriavzevii and Saccharomyces pastorianus.

Suitably the yeast may be a Saccharomyces cerevisiae var. diastaticus yeast.

The term “feedstock” as used herein refers to a composition comprising at least one of the following: starch, cellulose, hemicellulose, lignocellulose, fermentable sugars or a combination thereof.

A “fraction of a feedstock” refers to any component of a feedstock that is separated out during the processing of said feedstock.

The feedstock may be a starch, a grain-based material (e.g. a cereal, wheat, barley, rye, rice, triticale, millet, milo, sorghum or corn), a tuber (e.g. potato or cassava), a root, a sugar (e.g. cane sugar, beet sugar, molasses or a sugar syrup), stillage, wet cake, DDGS, cellulosic biomass, hemicellulosic biomass, a whey protein, soy based material, lignocellulosic biomass or combinations thereof.

Lignocellulosic biomass may comprise cellulose, hemicellulose and the aromatic polymer lignin.

Hemicellulose and cellulose (including insoluble arabinoxylans) by themselves are also potential energy sources, as they consist of C5- and C6-saccharides. Mono C6-saccharides can be used as energy source by the animal, while oligo C5-saccharides can be transformed into short chain fatty acids by the micro flora present in the animal gut (van den Broek et al., 2008 Molecular Nutrition & Food Research, 52, 146-63), which short chain fatty acids can be taken up and digested by the animal's gut.

Suitably the lignocellulosic biomass may be any cellulosic, hemicellulosic or lignocellulosic material, for example agricultural residues, bioenergy crops, industrial solid waste, municipal solid waste, sludge from paper manufacture, yard waste, wood waste, forestry waste and combinations thereof.

The lignocellulosic biomass may be selected from the group consisting of corn cobs, crop residues such as corn husks, corn gluten meal (CGM), corn stover, corn fiber, grasses, beet pulp, wheat straw, wheat chaff, oat straw, wheat middlings, wheat shorts, rice bran, rice hulls, wheat bran, oat hulls, wet cake, Distillers Dried Grain (DDG), Distillers Dried Grain Solubles (DDGS), palm kernel, citrus pulp, cotton, lignin, barley straw, hay, rice straw, rice hulls, switchgrass, miscanthus, cord grass, reed canary grass, waste paper, sugar cane bagasse, sorghum bagasse, forage sorghum, sorghum stover, soybean stover, soy, components obtained from milling of trees, branches, roots, leaves, wood chips, sawdust, shrubs and bushes, vegetables, fruits and flowers.

Wet-cake, Distillers Dried Grains and Distillers Dried Grains with Solubles are products obtained after the removal of ethyl alcohol by distillation from fermentation of a grain or a grain mixture by methods employed in the grain distilling industry.

Stillage coming from the distillation (e.g. comprising water, remainings of the grain, yeast cells etc.) is separated into a “solid” part and a liquid part.

The solid part is called “wet-cake” and can be used as animal feed as such.

The liquid part is (partially) evaporated into a syrup (solubles). The liquid part is often referred to as the thin stillage.

When the wet-cake is dried it is Distillers Dried Grains (DDG).

When the wet-cake is dried together with the syrup (solubles) it is Distillers Dried Grans with Solubles (DDGS).

Wet-cake may be used in dairy operations and beef cattle feedlots.

The dried DDGS may be used in livestock, (e.g. dairy, beef and swine) feeds and poultry feeds.

Corn DDGS is a very good protein source for dairy cows.

Corn gluten meal (CGM) is a powdery by-product of the corn milling industry. CGM has utility in, for example, animal feed. It can be used as an inexpensive protein source for feed such as pet food, livestock feed and poultry feed. It is an especially good source of the amino acid cysteine but must be balanced with other proteins for lysine.

The grain-based material may be one or more selected from the group consisting of: corn, wheat, barley, oats, rye, maize, millet, rice, cassava and sorghum.

In some embodiments the use of a tripeptidyl peptidase in the methods and/or uses of the invention may increase the concentration of tripeptides in the fermentation mixture when compared to a fermentation mixture not comprising one or more tripeptidyl peptidase.

The feedstock or a portion thereof may be subjected to one or more processing steps either before, during or after fermentation.

In one embodiment the feedstock or a portion thereof may have been subjected to one or more processing steps selected from the group consisting of: milling, cooking, saccharification, fermentation and simultaneous saccharification and fermentation.

The term “milling” as used herein refers to any milling of a feedstock. For example milling may include wet milling, dry grinding or combinations thereof.

Milling refers to a process which aids in breaking up the raw material used for the preparation of the feedstock into appropriately sized particles to facilitate downstream processing of the feedstock, e.g. for facilitating the cooking process. In some methods, the milling process aids in exposing the starch.

Wet milling is a process of milling that requires wet steeping of e.g. corn kernel before processing. This is then followed by a series of unit operations carried out in order to recover starch. The grain is typically soaked or “steeped” in water with dilute sulphurous acid for 24 to 48 hours prior to being subject to a series of grinders. The downstream processes may include removal of oil (e.g. corn oil) followed by further stages to separate out fiber, protein (e.g. gluten) and starch components (e.g. such as the endosperm). This may be achieved by centrifugation, use of screens and hydroclonic separators. The starch and water remaining from this process may then be subjected to fermentation.

Dry grinding refers to a process in which a starting material, such as a grain, is ground into a flour (e.g. meal) before further processing. Typically the flour is then slurried with water to form a mash prior to being processed in downstream steps (e.g. saccharification). Ammonia may be added to the mash and serves to both control the pH and provide a nutrient source to the alcohol production host used in fermentation.

In a preferred embodiment dry grinding may be used during processing of the feedstock or a fraction thereof.

Suitably, the tripeptidyl peptidase may be admixed with the feedstock or a fraction thereof during milling or dry grinding.

Suitably, the feedstock or a fraction thereof obtained after milling or dry grinding may be subjected to liquefaction and/or saccharification and/or fermentation and/or simultaneous saccharification and fermentation. This may be with or without a cooking step, e.g. after milling and before either liquefaction or saccharification.

The feedstock or a fraction thereof may be subjected to cooking. Typically the cooking process may take place post-milling. Suitably the cooking process may take place at 90-120° C. Suitably the cooking may be carried out prior to liquefaction and/or saccharification. Suitably the cooking process may reduce bacteria levels prior to fermentation. In some embodiments one or more enzymes may be added at this stage or thereafter. Suitably, alpha-amylase may be added following the cooking process, e.g. in a liquefaction process.

In some embodiments the feedstock or a fraction thereof may not be subjected to cooking.

In such an embodiment saccharification and fermentation or SSF may be carried out on a feedstock or fraction thereof comprising granular or raw starch (e.g. starch that has been treated at temperatures below gelatinization of the starch).

In one embodiment the feedstock or a fraction thereof may be subjected to enzymatic treatment, e.g. with an alpha-amylase and/or an amyloglucosidase. In some embodiments this enzymatic treatment replaces the cooking step.

In some embodiments the feedstock or fraction thereof may undergo one or more liquefaction steps.

The term “liquefaction” as used herein refers to a process in which the starch is liquefied, usually by increasing the temperature. Liquefaction of the starch results in a significant increase in viscosity. For this reason amylases may be introduced in order to reduce the viscosity. The temperature at which the starch liquefies varies depending upon the source of the starch. Starch processing can also be carried at temperatures from about 25° C. to just below the liquefaction temperature. These types of processes are often referred to as Granular starch hydrolysis, Direct starch hydrolysis, raw starch hydrolysis, low temperature starch hydrolysis or other terms. In some cases, the starch is pretreated at temperatures below the liquefaction temperatures in order to enhance enzymatic hydrolysis or other processes for treatment of starch.

Liquefaction can be carried out at high or low temperatures with suitable temperatures being known to, and able to be selected by, those skilled in the art. For example, liquefaction may be carried out at a temperature at which the starch and/or polysaccharides present in a feedstock or a fraction thereof are liquefied (e.g. a temperature at which there is an increase in viscosity). Such a temperature will be dependent on the origin of the feedstock or fraction thereof and the starch and/or polysaccharide content therein. In some embodiments the skilled person may carry out liquefaction at a temperature below (e.g. just below) the liquefaction temperature of the starch and/or polysaccharide comprised in a feedstock or fraction thereof.

The liquefaction may be carried out at high or low temperatures such as from about 25° C. to about 95° C., e.g. from about 25° C. to about 84° C.

In one embodiment the liquefaction may be carried out at around 85° C. to about 95° C.

In other embodiments liquefaction may be carried out at a lower temperature and/or a “cold cook process” that does not involve complete liquefaction of starch may be employed.

The feedstock or a fraction thereof may also undergo saccharification. The saccharification may be separate to fermentation or simultaneously therewith.

Separate saccharification and fermentation is a process whereby starch present in a feedstock, e.g., corn, or a fraction thereof is converted to glucose and subsequently an alcohol production host (e.g. an ethanologen) converts the glucose into ethanol. Simultaneous saccharification and fermentation (SSF) is a process whereby starch present in a feedstock or a fraction thereof is converted to glucose and, at the same time and in the same reactor, an alcohol production host (e.g. ethanologen) converts the glucose into ethanol.

In some embodiments the saccharification may be carried out at low temperatures

During saccharification, typically one or more enzymes may be added to facilitate glucose breakdown. A suitable enzyme preparation for use in saccharification includes Distillase® SSF (available from DuPont Industrial Biosciences—formerly Genencor), which comprises amylase (1,4-α-D-glucan glucanohydrolase—EC 3.2.1.1), glucoamylase (1,4-α-D-glucan glucohydrolase E.C. 3.2.1.3), isoamylase, beta amylase, pullulanase, and Aspergillopepsin 1 (EC 3.4.23.18).

Alternatively or additionally one or more endoprotease and/or exopeptidase may be added during the saccharification step. Suitably, the endoprotease and/or exopeptidase may be obtainable (e.g. obtained) from Trichoderma.

In one embodiment FERMGEN™ (available from Genencor) may be added during the saccharification step.

In some embodiments cellulases and/or hemicellulases and/or further enzymes may be added during the saccharification process.

Suitably there may be also added one or more further enzymes selected from the group consisting of: endoglucanases (E.C. 3.2.1.4); cellobiohydrolases (E.C. 3.2.1.91), β-glucosidases (E.C. 3.2.1.21), cellulases (E.C. 3.2.1.74), lichenases (E.C. 3.1.1.73), lipases (E.C. 3.1.1.3), lipid acyltransferases (generally classified as E.C. 2.3.1.x), phospholipases (E.C. 3.1.1.4, E.C. 3.1.1.32 or E.C. 3.1.1.5), phytases (e.g. 6-phytase (E.C. 3.1.3.26) or a 3-phytase (E.C. 3.1.3.8), amylases, alpha-amylases (E.C. 3.2.1.1), xylanases (e.g. endo-1,4-β-d-xylanase (E.C. 3.2.1.8) or 1,4 β-xylosidase (E.C. 3.2.1.37) or E.C. 3.2.1.32, E.C. 3.1.1.72, E.C. 3.1.1.73), glucoamylases (E.C. 3.2.1.3), hemicellulases (e.g. xylanases), proteases (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)), debranching enzymes, cutinases, esterases and/or mannanases (e.g. a β-mannanase (E.C. 3.2.1.78)) transferases, glucosidases, arabinofuranosidase. The tripeptidyl peptidase may be added at one or more of the stages of processing of a feedstock or a fraction thereof.

Suitably the tripeptidyl peptidase may be added during milling.

Suitably the tripeptidyl peptidase may be added during saccharification. Preferably the tripeptidyl peptidase may be added during simultaneous saccharification and fermentation.

Suitably the tripeptidyl peptidase may be added during liquefaction.

Suitably the tripeptidyl peptidase may be added during fermentation.

In some embodiments the tripeptidyl peptidase may be used in combination with an endoprotease.

In some embodiments the tripeptidyl peptidase may be admixed with a feedstock or a fraction thereof after fermentation. Suitably thereafter the admixture may be further processed (e.g. via milling).

The method of the present invention may comprise one or a plurality of fermentations. Tripeptidyl peptidase of the present invention may be added before, during or after any of the fermentations.

In some embodiments the tripeptidyl peptidase in accordance with the present invention is admixed with a feedstock or a fraction thereof (e.g. whole stillage or DDGS) obtainable (e.g. obtained) after fermentation.

In one embodiment the feedstock or a fraction thereof obtainable (or obtained) after fermentation (e.g. whole stillage or DDGS) may be treated (or further treated) with a tripeptidyl peptidase according to the present invention. Suitably this may be used in combination with one or more additional treatments of the feedstock or fraction thereof, e.g. acid addition and/or grinding. In one embodiment, the treated feedstock or fraction thereof may then serve as a feedstock for one or more additional fermentation(s). A tripeptidyl peptidase according to the present invention may be added in the additional fermentation.

Suitably, the feedstock or fraction thereof for use in the present invention may be a fibre-containing fraction.

The method of the present invention may comprise adding an alcohol production strain before, during or after admixing a tripeptidyl peptidase with a feedstock or a fraction thereof.

The method may also comprise admixing urea with a feedstock or fraction thereof. Advantageously, use of the tripeptidyl peptidase of the present invention may reduce the amount of urea that needs to be added to the feedstock.

### Uses

In one aspect there is provided the use of a tripeptidyl peptidase, predominantly having exopeptidase activity, in the manufacture of an alcohol (preferably bioethanol) for improving the yield of an alcohol (preferably bioethanol).

The term “improving the yield of an alcohol” as used herein refers to an increase in the concentration of alcohol (e.g. bioethanol) recovered post-fermentation when a tripeptidyl peptidase has been used during the processing method when compared with the concentration of alcohol recovered post-fermentation when a tripeptidyl peptidase has not been used during the processing method.

Suitably, the yield of an alcohol may be improved by at least about 0.1% v/v, more suitably by at least about 0.3% v/v, even more suitably by at least 0.5% v/v.

In some embodiments the use of the tripeptidyl peptidase in combination with an endoprotease may improve the concentration of alcohol recovered by at least about 0.4% v/v, suitably by at least 0.6% v/v, more suitably by at least 0.8% v/v.

In a further aspect there is provided the use of a tripeptidyl peptidase, predominantly having exopeptidase activity, in the manufacture of an alcohol for improving the alcohol production host's ability to ferment.

Without wishing to be bound by theory it is believed that the tripeptidyl peptidase of the present invention may increase the concentration of tripeptides present in a feedstock or fraction thereof. One advantage of the present invention is that the tripeptides so formed may be a good amino acid source and/or energy and/or nutrient source, e.g. for the alcohol production host.

The improvement in the alcohol production host's ability to ferment may be measured by an increase in the amount of sugar (e.g. glucose) consumed during fermentation by the alcohol production host when compared to the level of sugar (e.g. glucose) consumed during fermentation by said alcohol production host not admixed with the tripeptidyl peptidase.

Suitably, the level of sugar (e.g. glucose) in the fermentation medium when measured at between about 15 hours to about 20 hours may be less than about 0.1% w/v when compared to the level of glucose consumed during fermentation by the alcohol production host not admixed with the tripeptidyl peptidase. Suitably, the level of sugar (e.g. glucose) in the fermentation medium may be less than about 0.2% w/v, suitably less than about 0.3% w/v.

The concentration of an alcohol and/or sugar (e.g. glucose) may be measured by any method known to one skilled in the art. For example high performance liquid chromatography (HPLC) analysis may be used.

Alternative end of fermentation (EOF) products include, but are not limited to, metabolites, such as citric acid, lactic acid, succinic acid, monosodium glutamate, gluconic acid, sodium gluconate, calcium gluconate, potassium gluconate, itaconic acid and other carboxylic acids, glucono delta-lactone, sodium erythorbate, lysine and other amino acids, omega-3 fatty acid, isoprene, 1,3-propanediol, ethanol, butanol, other alcohols, and other biochemicals and biomaterials.

In addition to EOF, tripeptidyl peptidases could also be used in production of sugar syrups (e.g., DP1, 2, 3, and the like, specialty syrups, oligosaccharides, and polysaccharides). Tripeptidyl peptidases may also generate peptides that may be of use for the production host; or work synergistically with another protease(s) to generate amino acids and/or peptides of potential value.

### Expression by the Alcohol Production Host

The tripeptidyl peptidase for use in the invention may be expressed and secreted by an alcohol production host.

Suitably the tripeptidyl peptidase may be heterologous to the alcohol production host.

The term “heterologous to the alcohol production host” as used herein may refer to an enzyme that is normally expressed in an alcohol production host but either the enzyme or the nucleotide sequence encoding it has been engineered in some manner such that either the enzyme and/or nucleotide sequence is different to the “native” enzyme and/or nucleotide sequence encoding said enzyme. Alternatively or additionally, a heterologous enzyme may be one that is derived from a different organism, for example an enzyme derived from an exogenous source. In other words it may refer to an enzyme that is not normally expressed in the alcohol production host.

In other embodiments the tripeptidyl peptidase may be homologous to the alcohol production host.

In some embodiments the alcohol production host may co-express the tripeptidyl peptidase with one or more enzymes selected from the group consisting of a glucoamylase, an amylase, a further starch modifying enzyme, a protease, a phytase, a cellulase, a hemicellulase, a further enzyme and combinations thereof.

Suitably, in addition to expressing the tripeptidyl peptidase the alcohol production host may additionally express one or more enzymes selected from the group consisting of: endoglucanases (E.C. 3.2.1.4); cellobiohydrolases (E.C. 3.2.1.91), β-glucosidases (E.C. 3.2.1.21), cellulases (E.C. 3.2.1.74), lichenases (E.C. 3.1.1.73), lipases (E.C. 3.1.1.3), lipid acyltransferases (generally classified as E.C. 2.3.1.x), phospholipases (E.C. 3.1.1.4, E.C. 3.1.1.32 or E.C. 3.1.1.5), phytases (e.g. 6-phytase (E.C. 3.1.3.26) or a 3-phytase (E.C. 3.1.3.8), amylases, alpha-amylases (E.C. 3.2.1.1), pullulanase, isoamylase, beta-amylase, alpha-glucosidase, xylanases (e.g. endo-1,4-β-d-xylanase (E.C. 3.2.1.8) or 1,4 β-xylosidase (E.C. 3.2.1.37) or E.C. 3.2.1.32, E.C. 3.1.1.72, E.C. 3.1.1.73), glucoamylases (E.C. 3.2.1.3), hemicellulases, proteases (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)), debranching enzymes, cutinases, esterases and/or mannanases (e.g. a β-mannanase (E.C. 3.2.1.78)) transferases, glucosidases, arabinofuranosidase.

### Activity and Assays

The tripeptidyl peptidase for use in the present invention predominantly has exopeptidase activity.

The term “exopeptidase” activity as used herein means that the tripeptidyl peptidase is capable of cleaving tri-peptides from the N-terminus of a substrate, such as a protein and/or peptide substrate.

The term “predominantly has exopeptidase activity” as used herein means that the tripeptidyl peptidase has no or substantially no endoprotease activity.

“Substantially no endoprotease activity” means that the proline tolerant tripeptidyl peptidase or exo-peptidase of the S53 family has less than about 1000 endoprotease activity in the “Endoprotease Assay” taught herein when compared to 1000 nkat of exopeptidase activity in the “Exopeptidase Broad-Specificity Assay (EBSA)” taught herein. Suitably, “substantially no endoprotease activity” means that the proline tolerant tripeptidyl peptidase has less than about 100 U endoprotease activity in the “Endoprotease Assay” taught herein when compared to 1000 nkat of exopeptidase activity in the “Exopeptidase Broad-Specificity Assay” taught herein.

Preferably the proline tolerant tripeptidyl peptidase or exo-peptidase of the S53 family may have less than about 10 U endoprotease activity in the “Endoprotease Assay” taught herein when compared to 1000 nkat of exopeptidase activity in the “Exopeptidase Broad-Specificity Assay” taught herein, more preferably less than about 1 U endoprotease activity in the “Endoprotease Assay” taught herein when compared to 1000 nkat of exopeptidase activity in the “Exopeptidase Broad-Specificity Assay” taught herein. Even more preferably the proline tolerant tripeptidyl peptidase or exo-tripeptidyl peptidase may have less than about 0.1 U endoprotease activity in the “Endoprotease Assay” taught herein when compared to 1000 nkat of exopeptidase activity in the “Exopeptidase Broad-Specificity Assay” taught herein.

### “Endoprotease Assay”

### Azoscasein Assay for Endoprotease Activity

A modified version of the endoprotease assay described by Iversen and Jørgensen, 1995 (Biotechnology Techniques 9, 573-576) is used. An enzyme sample of 50 μl is added to 250 μl of azocasein (0.25% w/v; from Sigma) in 4 times diluted McIlvaine buffer, pH 5 and incubated for 15 min at 40° C. with shaking (800 rpm). The reaction is terminated by adding 50 μl of 2 M trichloroacetic acid (TCA) (from Sigma Aldrich, Denmark) and centrifugation for 5 min at 20,000 g. To a 195 μl sample of the supernatant 65 μl of 1 M NaOH is added and absorbance at 450 nm is measured. One unit of endoprotease activity is defined as the amount which yields an increase in absorbance of 0.1 in 15 min at 40° C. at 450 nm.

### “Exopeptidase Assay”

### Part 1—“Exopeptidase Broad-Specificity Assay” (EBSA)

10 μL of the chromogenic peptide solution (10 mM H-Ala-Ala-Ala-pNA dissolved in dimethyl sulfoxide (DMSO); MW=387.82; Bachem, Switzerland) were added to 130 μl Na-acetate (20 mM, adjusted to pH 4.0 with acetic acid) in a microtiter plate and heated for 5 minutes at 40° C. 10 μL of appropriately diluted enzyme was added and the absorption was measured in a MTP reader (Versa max, Molecular Devices, Denmark) at 405 nm. One katal of proteolytic activity was defined as the amount of enzyme required to release 1 mole of p-nitroaniline per second.

In one embodiment a tripeptidyl peptidase in accordance with the present invention has an activity of at least about 50 nkat in the EBSA activity assay taught herein

Suitably a tripeptidyl peptidase in accordance with the present invention has an activity of between about 50-2000 nkat units in the EBSA activity assay taught herein

To determine if a tripeptidyl peptidase is a proline tolerant tripeptidyl peptidase the following assays may be combined with Part 1.

Part 2 (i)—P1 Proline Assay

(a) Dissolve the substrate H-Arg-Gly-Pro-Phe-Pro-Ile-Ile-Val (MW=897.12; from Schafer-N, Copenhagen in 10 times diluted McIlvain buffer, pH=4.5 at 1 mg/ml concentration.

(b) Incubate 1000 ul of the substrate solution with 10 ug of proline tolerant tripeptidyl peptidase solution at 40° C.

(c) Take 100 ul samples at seven time points (0, 30, 60, 120, 720 and 900 min), dilute with 50 ul 5% TFA, heat inactivate (10 min at 80° C.) and keep at −20° C. until LC-MS analysis;

(d) Perform LC-MC/MS analysis using an Agilent 1100 Series Capillary HPLC system (Agilent Technologies, Santa Clara, Calif.) interfaced to a LTQ Orbitrap Classic hybrid mass spectrometer (Thermo Scientific, Bremen, Germany);

(e) Load samples onto a 50 mm Fortis™ C18 column with an inner diameter of 2.1 mm and a practical size of 1.7 μm

(f) Perform separation at a flow rate of 200 μL/min using a 14 min gradient of 2-28% Solvent B (H2O/CH3CN/HCOOH (50/950/0.65 v/v/v)) into the IonMAX source—The LTQ Orbitrap Classic instrument was operated in a data-dependent MS/MS mode;

(g) Measure the peptide masses by the Orbitrap (obtain MS scans with a resolution of 60.000 at m/z 400), and select up to 2 of the most intense peptide m/z and subject to fragmentation using CID in the linear ion trap (LTQ). Enable dynamic exclusion with a list size of 500 masses, duration of 40 s, and an exclusion mass width of ±10 ppm relative to masses on the list;

(h) Use the open source program Skyline 1.4.0.4421 (available from MacCoss Lab Software, University of Washington, Department of Genome Sciences, 3720 15^{th} Ave NE Seattle, Wash., US) to access the RAW files and extract MS1 intensities to build chromatograms. Set the precursor isotopic import filter to a count of three, (M, M+1, and M+2) at a resolution of 60,000 and use the most intense charge state;

(i) Peptide sequences of the substrate and cleavage products were typed into Skyline and intensities were calculated in each sample (0, 30, 60, 120, 720 and 900 min hydrolysis).

(j) One unit of activity is defined as the amount of enzyme which in this assay will hydrolyse 50% of the substrate within 720 min while releasing Arg-Gly-Pro.

Part 2 (ii)—P1′ Proline Assay

(a) Dissolve the peptide H-Ala-Ala-Phe-Pro-Ala-NH2 (MW=474.5; from Schafer-N, Copenhagen) in 10 times diluted McIlvain buffer, pH=4.5 at 0.1 mg/ml concentration.

(b) Incubate 1000 ul of the substrate solution with 10 ug proline tolerant tripeptidyl peptidase solution at 40° C.

(c) Take 100 ul samples at seven time points (0, 30, 60, 120, 720 and 900 min), dilute with 50 ul 5% TFA, heat inactivate (10 min at 80° C.) and keep at −20° C. until LC-MS analysis;

(d) Perform LC-MC/MS analysis using a Agilent 1100 Series Capillary HPLC system (Agilent Technologies, Santa Clara, Calif.) interfaced to a LTQ Orbitrap Classic hybrid mass spectrometer (Thermo Scientific, Bremen, Germany);

(e) Load samples onto a 50 mm Fortis™ C18 column with an inner diameter of 2.1 mm and a practical size of 1.7 μm

(f) Perform separation at a flow rate of 200 μL/min using a 14 min gradient of 2-28% Solvent B (H2O/CH3CN/HCOOH (50/950/0.65 v/v/v)) into the IonMAX source—The LTQ Orbitrap Classic instrument was operated in a data-dependent MS/MS mode;

(g) Measure the peptide masses by the Orbitrap (obtain MS scans with a resolution of 60.000 at m/z 400), and select up to 2 of the most intense peptide m/z and subject to fragmentation using CID in the linear ion trap (LTQ). Enable dynamic exclusion with a list size of 500 masses, duration of 40 s, and an exclusion mass width of ±10 ppm relative to masses on the list.

(h) Use the open source program Skyline 1.4.0.4421 (available from MacCoss Lab Software, University of Washington, Department of Genome Sciences, 3720 15^{th} Ave NE Seattle, Wash., US) to access the RAW files and extract MS1 intensities to build chromatograms. Set the precursor isotopic import filter to a count of three, (M, M+1, and M+2) at a resolution of 60,000 and use the most intense charge state;

(i) Peptide sequences of the substrate as well as cleavage products were typed into Skyline and intensities were calculated in each sample.

(j) One unit of activity is defined as the amount of enzyme which in this assay will hydrolyse 50% of the substrate within 720 min while releasing Ala-Ala-Phe.

If the tripeptidyl peptidase for use in the present invention is a proline tolerant tripeptidyl peptidase as defined herein then in one embodiment the proline tolerant tripeptidyl peptidase has an activity of at least 50 nkat in Part 1 of the activity taught herein and at least 100 U activity in Part 2(i) or Part 2(ii) of the assay taught herein per mg of protein.

In one embodiment a proline tolerant tripeptidyl peptidase in accordance with the present invention has an activity of between about 50-2000 nkat in Part 1 of the activity taught herein and between about 1-500 units activity in Part 2(i) or Part 2(ii) of the assay taught herein per mg of protein. Note the protein measurement is described in Example 4.

### “P1 and P1′ Proline Activity Assay”

Suitably the tripeptidyl peptidase for use in the present invention may be able to cleave substrates having proline at position P1 and P1′. This can be assessed using the assay taught below.

In this assay a tripeptidyl peptidase is examined for its ability to hydrolyse a synthetic substrate AAPPA by LC-MS and label free quantification.

(a) Dissolve the peptide H-AAPPA-NH2 (MW=424.3, from Schafer-N, Copenhagen) in 20 mM MES buffer, pH=4.0 (1 mg/ml);

(b) Incubate 1000 ul of the H-AAPPA-NH2 solution with 200 ul proline tolerant tripeptidyl peptidase solution (40 ug/ml) (substrate/enzyme 100:0.8) at room temperature;

(c) Take 100 ul samples at seven time points (0, 5, 15, 60, 180, 720 and 1440 min), dilute with 50 ul 5% TFA, heat inactivate (10 min at 80° C.) and keep at −20° C. until LC-MS analysis;

(d) Perform Nano LC-MS/MS analyses using an Easy LC system (Thermo Scientific, Odense, DK) interfaced to a LTQ Orbitrap Classic hybrid mass spectrometer (Thermo Scientific, Bremen, Germany);

(e) Load samples onto a custom-made 2 cm trap column (100 μm i.d., 375 μm o.d., packed with Reprosil C18, 5 μm reversed phase particles (Dr. Maisch GmbH, Ammerbuch-Entringen, Germany)) connected to a 10 cm analytical column (75 μm i.d., 375 μm o.d., packed with Reprosil C18, 3 μm reversed phase particles (Dr. Maisch GmbH, Ammerbuch-Entringen, Germany)) with a steel needle;

(f) Perform separation at a flow rate of 300 nL/m in using a 10 min gradient of 0-34% Solvent B (H2O/CH3CN/TFE/HCOOH (100/800/100/1 v/v/v/v)) into the nanoelectrospray ion source (Thermo Scientific, Odense, DK)—operate the LTQ Orbitrap Classic instrument in a data-dependent MS/MS mode;

(g) Measure the peptide masses by the Orbitrap (obtain MS scans with a resolution of 60 000 at m/z 400), and select up to 2 of the most intense peptide m/z and subject to fragmentation using CID in the linear ion trap (LTQ). Enable dynamic exclusion with a list size of 500 masses, duration of 40 s, and an exclusion mass width of ±10 ppm relative to masses on the list;

(h) Use the open source program Skyline 1.4.0.4421 (available from MacCoss Lab Software, University of Washington, Department of Genome Sciences, 3720 15^{th} Ave NE Seattle, Wash., US) to access the RAW files which program can use the MS1 intensities to build chromatograms. Set the precursor isotopic import filter to a count of three, (M, M+1, and M+2) at a resolution of 60,000 and use the most intense charge state;

(i) Peptide sequences of the substrate as well as cleavage products were typed into Skyline and intensities were calculated in each sample.

(j) One unit of activity is defined as the amount of enzyme which in this assay will hydrolyse 50% of the substrate within 24 h while releasing AAP.

In one embodiment a proline tolerant tripeptidyl peptidase in accordance with the present invention has an activity of at least 50 nkat in Part 1 of the activity taught herein and at least 100 U activity in Part 2(i) or Part 2(ii) of the assay taught herein per mg of protein.

In one embodiment a proline tolerant tripeptidyl peptidase in accordance with the present invention has an activity of between about 50-2000 nkat in Part 1 of the activity taught herein and between about 1-500 units activity in Part 2(i) or Part 2(ii) of the assay taught herein per mg of protein (protein concentration is calculated as in Example 2).

In one embodiment a proline tolerant tripeptidyl peptidase for use in the present invention may have at least 10 U activity in the “P1 and P1′ Proline Activity Assay” taught herein per mg of protein.

In one embodiment a proline tolerant tripeptidyl peptidase in accordance with the present invention has an activity of between about 1 U-500 U activity in the “P1 and P1′ Proline Activity Assay” taught herein per mg of protein.

In addition to the above, the proline tolerant tripeptidyl peptidase may also have activity in accordance with Part 1 of the “Exopeptidase Activity Assay” taught above.

In one embodiment the proline tolerant tripeptidyl peptidase for use in the present invention may have at least 10 U activity in the “P1 and P1′ Proline Activity Assay” taught herein and at least 50 nkatal in Part 1 of the “Exopeptidase Activity Assay” taught herein per mg of protein.

In another embodiment a proline tolerant tripeptidyl peptidase in accordance with the present invention has an activity of between about 1 U-500 U activity in the “P1 and P1′ Proline Activity Assay” taught herein and between about 50 U-2000 U katal in Part 1 of the “Exopeptidase Activity Assay” taught herein per mg of protein.

### Amino Acid and Nucleotide Sequences

The tripeptidyl peptidase for use in accordance with the present invention may be obtainable (e.g. obtained) from any source so long as it has the activity described herein.

In one embodiment the tripeptidyl peptidase for use in accordance with the present invention may be obtainable (e.g. obtained) from Trichoderma.

Suitably from Trichoderma reesei, more suitably, Trichoderma reesei QM6A.

Suitably from Trichoderma virens, more suitably, Trichoderma virens Gv29-8.

Suitably from Trichoderma atroviride. More suitably, Trichoderma atroviride IMI 206040.

In one embodiment the tripeptidyl peptidase for use in accordance with the present invention may be obtainable (e.g. obtained) from Aspergillus.

Suitably from Aspergillus fumigatus, more suitably Aspergillus fumigatus CAE17675.

Suitably from Aspergillus kawachii, more suitably from Aspergillus kawachii IFO 4308.

Suitably from Aspergillus nidulans, more suitably from Aspergillus nidulans FGSC A4.

Suitably from Aspergillus oryzae, more suitably Aspergillus oryzae RIB40.

Suitably from Aspergillus ruber, more suitably Aspergillus ruber CBS135680.

Suitably from Aspergillus terreus, more suitably from Aspergillus terreus NIH2624.

In one embodiment the tripeptidyl peptidase for use in accordance with the present invention may be obtainable (e.g. obtained) from Bipolaris, suitably from Bipolaris maydis, more suitably Bipolaris maydis C5.

In one embodiment the tripeptidyl peptidase for use in accordance with the present invention may be obtainable (e.g. obtained) from Togninia, suitably from Togninia minima more suitably Togninia minima UCRPA7.

In one embodiment the tripeptidyl peptidase for use in accordance with the present invention may be obtainable (e.g. obtained) from Talaromyces, suitably from Talaromyces stipitatus more suitably Talaromyces stipitatus ATCC 10500.

In one embodiment the tripeptidyl peptidase for use in accordance with the present invention may be obtainable (e.g. obtained) from Arthroderma, suitably from Arthroderma benhamiae more suitably Arthroderma benhamiae CBS 112371.

In one embodiment the tripeptidyl peptidase for use in accordance with the present invention may be obtainable (e.g. obtained) from Magnaporthe, suitably from Magnaporthe oryzae more suitably Magnaporthe oryzae 70-1.

In another embodiment the tripeptidyl peptidase for use in accordance with the present invention may be obtainable (e.g. obtained) from Fusarium.

Suitably from Fusarium oxysporum, more suitably from Fusarium oxysporum f. sp. cubense race 4.

Suitably from Fusarium graminearum, more suitably Fusarium graminearum PH-1.

In a further embodiment the tripeptidyl peptidase for use in accordance with the present invention may be obtainable (e.g. obtained) from Phaeosphaeria, suitably from Phaeosphaeria nodorum more suitably Phaeosphaeria nodorum SN15.

In a yet further embodiment the proline tolerant tripeptidyl peptidase for use in accordance with the present invention may be obtainable (e.g. obtained) from Agaricus, suitably from Agaricus bisporus more suitably Agaricus bisporus var. burnettii JB137-S8.

In a yet further embodiment the tripeptidyl peptidase for use in accordance with the present invention may be obtainable (e.g. obtained) from Acremonium, suitably from Acremonium alcalophilum.

In a yet further embodiment the proline tolerant tripeptidyl peptidase for use in accordance with the present invention may be obtainable (e.g. obtained) from Sodiomyces, suitably from Sodiomyces alkalinus.

In one embodiment the tripeptidyl peptidase for use in accordance with the present invention may be obtainable (e.g. obtained) from Penicillium.

Suitably the tripeptidyl peptidase may be obtainable from Penicillium digitatum, more suitably from Penicillium digitatum Pd1.

Suitably the tripeptidyl peptidase may be obtainable from Penicillium oxalicum, more suitably from Penicillium oxalicum 114-2.

Suitably the tripeptidyl peptidase may be obtainable from Penicillium roqueforti, more suitably from Penicillium roqueforti FM164.

Suitably the tripeptidyl peptidase may be obtainable from Penicillium rubens, more suitably from Penicillium rubens Wisconsin 54-1255.

In another embodiment the tripeptidyl peptidase for use in accordance with the present invention may be obtainable (e.g. obtained) from Neosartorya.

Suitably the tripeptidyl peptidase may be obtainable from Neosartorya fischeri, more suitably from Neosartorya fischeri NRRL181.

In one embodiment the tripeptidyl peptidase (e.g. proline tolerant tripeptidyl peptidase) for use in accordance with the present invention is not obtainable (e.g. obtained) from Aspergillus niger.

| SEQ | | |
|---|---|---|
| ID | | |
| No. | Sequence | Origin |
| 1 | MAKLSTLRLASLLSLVSVQVSASVHLLESLEKLPHGWKAAETPSPSSQI | Trichoderma |
| | VLQVALTQQNIDQLESRLAAVSTPTSSTYGKYLDVDEINSIFAPSDASSS | reesei QM6a |
| | AVESWLQSHGVTSYTKQGSSIWFQTNISTANAMLSTNFHTYSDLTGAK | |
| | KVRTLKYSIPESLIGHVDLISPTTYFGTTKAMRKLKSSGVSPAADALAAR | |
| | QEPSSCKGTLVFEGETFNVFQPDCLRTEYSVDGYTPSVKSGSRIGFGS | |
| | FLNESASFADQALFEKHFNIPSQNFSVVLINGGTDLPQPPSDANDGEAN | |
| | LDAQTILTIAHPLPITEFITAGSPPYFPDPVEPAGTPNENEPYLQYYEFLL | |
| | SKSNAEIPQVITNSYGDEEQTVPRSYAVRVCNLIGLLGLRGISVLHSSGD | |
| | EGVGASCVATNSTTPQFNPIFPATCPYVTSVGGTVSFNPEVAWAGSSG | |
| | GFSYYFSRPWYQQEAVGTYLEKYVSAETKKYYGPYVDFSGRGFPDVA | |
| | AHSVSPDYPVFQGGELTPSGGTSAASPVVAAIVALLNDARLREGKPTL | |
| | GFLNPLIYLHASKGFTDITSGQSEGCNGNNTQTGSPLPGAGFIAGAHW | |
| | NATKGWDPTTGFGVPNLKKLLALVRF | |
| 2 | SVHLLESLEKLPHGWKAAETPSPSSQIVLQVALTQQNIDQLESRLAAVS | Trichoderma |
| | TPTSSTYGKYLDVDEINSIFAPSDASSSAVESWLQSHGVTSYTKQGSSI | reesei QM6a |
| | WFQTNISTANAMLSTNFHTYSDLTGAKKVRTLKYSIPESLIGHVDLISPT | |
| | TYFGTTKAMRKLKSSGVSPAADALAARQEPSSCKGTLVFEGETFNVFQ | |
| | PDCLRTEYSVDGYTPSVKSGSRIGFGSFLNESASFADQALFEKHFNIPS | |
| | QNFSVVLINGGTDLPQPPSDANDGEANLDAQTILTIAHPLPITEFITAGSP | |
| | PYFPDPVEPAGTPNENEPYLQYYEFLLSKSNAEIPQVITNSYGDEEQTV | |
| | PRSYAVRVCNLIGLLGLRGISVLHSSGDEGVGASCVATNSTTPQFNPIF | |
| | PATCPYVTSVGGTVSFNPEVAWAGSSGGFSYYFSRPWYQQEAVGTYL | |
| | EKYVSAETKKYYGPYVDFSGRGFPDVAAHSVSPDYPVFQGGELTPSG | |
| | GTSAASPVVAAIVALLNDARLREGKPTLGFLNPLIYLHASKGFTDITSGQ | |
| | SEGCNGNNTQTGSPLPGAGFIAGAHWNATKGWDPTTGFGVPNLKKLL | |
| | ALVRF | |
| 3 | EAFEKLSAVPKGWHYSSTPKGNTEVCLKIALAQKDAAGFEKTVLEMSD | Aspergillus |
| | PDHPSYGQHFTTHDEMKRMLLPRDDTVDAVRQWLENGGVTDFTQDA | oryzae RIB40 |
| | DWINFCTTVDTANKLLNAQFKWYVSDVKHIRRLRTLQYDVPESVTPHIN | |
| | TIQPTTRFGKISPKKAVTHSKPSQLDVTALAAAVVAKNISHCDSIITPTCL | |
| | KELYNIGDYQADANSGSKIAFASYLEEYARYADLENFENYLAPWAKGQ | |
| | NFSVTTFNGGLNDQNSSSDSGEANLDLQYILGVSAPLPVTEFSTGGRG | |
| | PLVPDLTQPDPNSNSNEPYLEFFQNVLKLDQKDLPQVISTSYGENEQEI | |
| | PEKYARTVCNLIAQLGSRGVSVLFSSGDSGVGEGCMTNDGTNRTHFP | |
| | PQFPAACPWVTSVGATFKTTPERGTYFSSGGFSDYWPRPEWQDEAV | |
| | SSYLETIGDTFKGLYNSSGRAFPDVAAQGMNFAVYDKGTLGEFDGTSA | |
| | SAPAFSAVIALLNDARLRAGKPTLGELNPWLYKTGRQGLQDITLGASIG | |
| | CTGRARFGGAPDGGPVVPYASWNATQGWDPVTGLGTPDFAELKKLALGN | |
| 4 | EPFEKLFSTPEGWKMQGLATNEQIVKLQIALQQGDVAGFEQHVIDISTP | Phaeosphaeria |
| | SHPSYGAHYGSHEEMKRMIQPSSETVASVSAWLKAAGINDAEIDSDWV | nodorum |
| | TFKTTVGVANKMLDTKFAWYVSEEAKPRKVLRTLEYSVPDDVAEHINLI | SN15 |
| | QPTTRFAAIRQNHEVAHEIVGLQFAALANNTVNCDATITPQCLKTLYKID | |
| | YKADPKSGSKVAFASYLEQYARYNDLALFEKAFLPEAVGQNFSVVQFS | |
| | GGLNDQNTTQDSGEANLDLQYIVGVSAPLPVTEFSTGGRGPWVADLD | |
| | QPDEADSANEPYLEFLQGVLKLPQSELPQVISTSYGENEQSVPKSYALS | |
| | VCNLFAQLGSRGVSVIFSSGDSGPGSACQSNDGKNTTKFQPQYPAAC | |
| | PFVTSVGSTRYLNETATGFSSGGFSDYWKRPSYQDDAVKAYFHHLGE | |
| | KFKPYFNRHGRGFPDVATQGYGFRVYDQGKLKGLQGTSASAPAFAGV | |
| | IGLLNDARLKAKKPTLGFLNPLLYSNSDALNDIVLGGSKGCDGHARFNG | |
| | PPNGSPVIPYAGWNATAGWDPVTGLGTPNFPKLLKAAVPSRYRA | |
| 5 | NAAVLLDSLDKVPVGWQAASAPAPSSKITLQVALTQQNIDQLESKLAAV | Trichoderma |
| | STPNSSNYGKYLDVDEINQIFAPSSASTAAVESWLKSYGVDYKVQGSSI | atroviride IMI |
| | WFQTDVSTANKMLSTNFHTYTDSVGAKKVRTLQYSVPETLADHIDLISP | 206040 |
| | TTYFGTSKAMRALKIQNAASAVSPLAARQEPSSCKGTIEFENRTFNVFQ | |
| | PDCLRTEYSVNGYKPSAKSGSRIGFGSFLNQSASSSDLALFEKHFGFA | |
| | SQGFSVELINGGSNPQPPTDANDGEANLDAQNIVSFVQPLPITEFIAGG | |
| | TAPYFPDPVEPAGTPDENEPYLEYYEYLLSKSNKELPQVITNSYGDEEQ | |
| | TVPQAYAVRVCNLIGLMGLRGISILESSGDEGVGASCLATNSTTTPQFN | |
| | PIFPATCPYVTSVGGTVSFNPEVAWDGSSGGFSYYFSRPWYQEAAVG | |
| | TYLNKYVSEETKEYYKSYVDFSGRGFPDVAAHSVSPDYPVFQGGELTP | |
| | SGGTSAASPIVASVIALLNDARLRAGKPALGFLNPLIYGYAYKGFTDITS | |
| | GQAVGCNGNNTQTGGPLPGAGVIPGAFWNATKGWDPTTGFGVPNFK | |
| | KLLELVRY | |
| 6 | KPTPGASHKVIEHLDFVPEGWQMVGAADPAAIIDFWLAIERENPEKLYD | Arthroderma |
| | TIYDVSTPGRAQYGKHLKREELDDLLRPRAETSESIINWLTNGGVNPQH | benhamiae |
| | IRDEGDWVRFSTNVKTAETLMNTRFNVFKDNLNSVSKIRTLEYSVPVAI | CBS 112371 |
| | SAHVQMIQPTTLFGRQKPQNSLILNPLTKDLESMSVEEFAASQCRSLVT | |
| | TACLRELYGLGDRVTQARDDNRIGVSGFLEEYAQYRDLELFLSRFEPS | |
| | AKGFNFSEGLIAGGKNTQGGPGSSTEANLDMQYVVGLSHKAKVTYYST | |
| | AGRGPLIPDLSQPSQASNNNEPYLEQLRYLVKLPKNQLPSVLTTSYGDT | |
| | EQSLPASYTKATCDLFAQLGTMGVSVIFSSGDTGPGSSCQTNDGKNAT | |
| | RFNPIYPASCPFVTSIGGTVGTGPERAVSFSSGGFSDRFPRPQYQDNA | |
| | VKDYLKILGNQWSGLFDPNGRAFPDIAAQGSNYAVYDKGRMTGVSGT | |
| | SASAPAMAAIIAQLNDFRLAKGSPVLGFLNPWIYSKGFSGFTDIVDGGS | |
| | RGCTGYDIYSGLKAKKVPYASWNATKGWDPVTGFGTPNFQALTKVLP | |
| 7 | KSYSHHAEAPKGWKVDDTARVASTGKQQVFSIALTMQNVDQLESKLLD | Fusarium |
| | LSSPDSKNYGQWMSQKDVTTAFYPSKEAVSSVTKWLKSKGVKHYNVN | graminearum |
| | GGFIDFALDVKGANALLDSDYQYYTKEGQTKLRTLSYSIPDDVAEHVQF | PH-1 |
| | VDPSTNFGGTLAFAPVTHPSRTLTERKNKPTKSTVDASCQTSITPSCLK | |
| | QMYNIGDYTPKVESGSTIGFSSFLGESAIYSDVFLFEEKFGIPTQNFTTV | |
| | LINNGTDDQNTAHKNFGEADLDAENIVGIAHPLPFTQYITGGSPPFLPNI | |
| | DQPTAADNQNEPYVPFFRYLLSQKEVPAVVSTSYGDEEDSVPREYAT | |
| | MTCNLIGLLGLRGISVIFSSGDIGVGAGCLGPDHKTVEFNAIFPATCPYL | |
| | TSVGGTVDVTPEIAWEGSSGGFSKYFPRPSYQDKAVKTYMKTVSKQT | |
| | KKYYGPYTNWEGRGFPDVAGHSVSPNYEVIYAGKQSASGGTSAAAPV | |
| | WAAIVGLLNDARFRAGKPSLGWLNPLVYKYGPKVLTDITGGYAIGCDG | |
| | NNTQSGKPEPAGSGIVPGARWNATAGWDPVTGYGTPDFGKLKDLVLSF | |
| 8 | AVVIRAAVLPDAVKLMGKAMPDDIISLQFSLKQQNIDQLETRLRAVSDPS | Acremonium |
| | SPEYGQYMSESEVNEFFKPRDDSFAEVIDWVAASGFQDIHLTPQAAAI | alcalophilum |
| | NLAATVETADQLLGANFSWFDVDGTRKLRTLEYTIPDRLADHVDLISPT | |
| | TYFGRARLDGPRETPTRLDKRQRDPVADKAYFHLKWDRGTSNCDLVIT | |
| | PPCLEAAYNYKNYMPDPNSGSRVSFTSFLEQAAQQSDLTKFLSLTGLD | |
| | RLRPPSSKPASFDTVLINGGETHQGTPPNKTSEANLDVQWLAAVIKARL | |
| | PITQWITGGRPPFVPNLRLRHEKDNTNEPYLEFFEYLVRLPARDLPQVIS | |
| | NSYAEDEQTVPEAYARRVCNLIGIMGLRGVTVLTASGDSGVGAPCRAN | |
| | DGSDRLEFSPQFPTSCPYITAVGGTEGWDPEVAWEASSGGFSHYFLR | |
| | PWYQANAVEKYLDEELDPATRAYYDGNGFVQFAGRAYPDLSAHSSSP | |
| | RYAYIDKLAPGLTGGTSASCPVVAGIVGLLNDARLRRGLPTMGFINPWL | |
| | YTRGFEALQDVTGGRASGCQGIDLQRGTRVPGAGIIPWASWNATPGW | |
| | DPATGLGLPDFWAMRGLALGRGT | |
| 9 | AVVIRAAPLPESVKLVRKAAAEDGINLQLSLKRQNMDQLEKFLRAVSDP | Sodiomyces |
| | FSPKYGQYMSDAEVHEIFRPTEDSFDQVIDWLTKSGFGNLHITPQAAAI | alkalinus |
| | NVATTVETADQLFGANFSWFDVDGTPKLRTGEYTIPDRLVEHVDLVSP | |
| | TTYFGRMRPPPRGDGVNDWITENSPEQPAPLNKRDTKTESDQARDHP | |
| | SWDSRTPDCATIITPPCLETAYNYKGYIPDPKSGSRVSFTSFLEQAAQQ | |
| | ADLTKFLSLTRLEGFRTPASKKKTFKTVLINGGESHEGVHKKSKTSEAN | |
| | LDVQWLAAVTQTKLPITQWITGGRPPFVPNLRIPTPEANTNEPYLEFLEY | |
| | LFRLPDKDLPQVISNSYAEDEQSVPEAYARRVCGLLGIMGLRGVTVLTA | |
| | SGDSGVGAPCRANDGSGREEFSPQFPSSCPYITTVGGTQAWDPEVA | |
| | WKGSSGGFSNYFPRPWYQVAAVEKYLEEQLDPAAREYYEENGFVRFA | |
| | GRAFPDLSAHSSSPKYAYVDKRVPGLTGGTSASCPVVAGIVGLLNDAR | |
| | LRRGLPTMGFINPWLYAKGYQALEDVTGGAAVGCQGIDIQTGKRVPGA | |
| | GIIPGASWNATPDWDPATGLGLPNFWAMRELALED | |
| 10 | VVHEKLAAVPSGWHHLEDAGSDHQISLSIALARKNLDQLESKLKDLSTP | Aspergillus |
| | GESQYGQWLDQEEVDTLFPVASDKAVISWLRSANITHIARQGSLVNFA | kawachii IFO |
| | TTVDKVNKLLNTTFAYYQRGSSQRLRTTEYSIPDDLVDSIDLISPTTFFG | 4308 |
| | KEKTSAGLTQRSQKVDNHVAKRSNSSSCADTITLSCLKEMYNFGNYTP | |
| | SASSGSKLGFASFLNESASYSDLAKFERLFNLPSQNFSVELINGGVNDQ | |
| | NQSTASLTEADLDVELLVGVGHPLPVTEFITSGEPPFIPDPDEPSAADN | |
| | ENEPYLQYYEYLLSKPNSALPQVISNSYGDDEQTVPEYYAKRVCNLIGL | |
| | VGLRGISVLESSGDEGIGSGCRTTDGTNSTQFNPIFPATCPYVTAVGGT | |
| | MSYAPEIAWEASSGGFSNYFERAWFQKEAVQNYLANHITNETKQYYS | |
| | QFANFSGRGFPDVSAHSFEPSYEVIFYGARYGSGGTSAACPLFSALVG | |
| | MLNDARLRAGKSTLGFLNPLLYSKGYKALTDVTAGQSIGCNGIDPQSDE | |
| | AVAGAGIIPWAHWNATVGWDPVTGLGLPDFEKLRQLVLSL | |
| 11 | AAALVGHESLAALPVGWDKVSTPAAGTNIQLSVALALQNIEQLEDHLKS | Talaromyces |
| | VSTPGSASYGQYLDSDGIAAQYGPSDASVEAVTNWLKEAGVTDIYNNG | stipitatus |
| | QSIHFATSVSKANSLLGADFNYYSDGSATKLRTLAYSVPSDLKEAIDLVS | ATCC 10500 |
| | PTTYFGKTTASRSIQAYKNKRASTTSKSGSSSVQVSASCQTSITPACLK | |
| | QMYNVGNYTPSVAHGSRVGFGSFLNQSAIFDDLFTYEKVNDIPSQNFT | |
| | KVIIANASNSQDASDGNYGEANLDVQNIVGISHPLPVTEFLTGGSPPFVA | |
| | SLDTPTNQNEPYIPYYEYLLSQKNEDLPQVISNSYGDDEQSVPYKYAIR | |
| | ACNLIGLTGLRGISVLESSGDLGVGAGCRSNDGKNKTQFDPIFPATCPY | |
| | VTSVGGTQSVTPEIAWVASSGGFSNYFPRTWYQEPAIQTYLGLLDDET | |
| | KTYYSQYTNFEGRGFPDVSAHSLTPDYQVVGGGYLQPSGGTSAASPV | |
| | FAGIIALLNDARLAAGKPTLGFLNPFFYLYGYKGLNDITGGQSVGCNGIN | |
| | GQTGAPVPGGGIVPGAAWNSTTGWDPATGLGTPDFQKLKELVLSF | |
| 12 | KSFSHHAEAPQGWQVQKTAKVASNTQHVFSLALTMQNVDQLESKLLD | Fusarium |
| | LSSPDSANYGNWLSHDELTSTFSPSKEAVASVTKWLKSKGIKHYKVNG | oxysporum f. |
| | AFIDFAADVEKANTLLGGDYQYYTKDGQTKLRTLSYSIPDDVAGHVQFV | sp. cubense |
| | DPSTNFGGTVAFNPVPHPSRTLQERKVSPSKSTVDASCQTSITPSCLK | race 4 |
| | QMYNIGDYTPDAKSGSEIGFSSFLGQAAIYSDVFKFEELFGIPKQNYTTI | |
| | LINNGTDDQNTAHGNFGEANLDAENIVGIAHPLPFKQYITGGSPPFVPNI | |
| | DQPTEKDNQNEPYVPFFRYLLGQKDLPAVISTSYGDEEDSVPREYATL | |
| | TCNMIGLLGLRGISVIFSSGDIGVGSGCLAPDYKTVEFNAIFPATCPYLTS | |
| | VGGTVDVTPEIAWEGSSGGFSKYFPRPSYQDKAIKKYMKTVSKETKKY | |
| | YGPYTNWEGRGFPDVAGHSVAPDYEVIYNGKQARSGGTSAAAPVWA | |
| | AIVGLLNDARFKAGKKSLGWLNPLIYKHGPKVLTDITGGYAIGCDGNNT | |
| | QSGKPEPAGSGLVPGARWNATAGWDPTTGYGTPNFQKLKDLVLSL | |
| 13 | SVLVESLEKLPHGWKAASAPSPSSQITLQVALTQQNIDQLESRLAAVST | Trichoderma |
| | PNSKTYGNYLDLDEINEIFAPSDASSAAVESWLHSHGVTKYTKQGSSIW | virens |
| | FQTEVSTANAMLSTNFHTYSDAAGVKKLRTLQYSIPESLVGHVDLISPTT | Gy29-8 |
| | YFGTSNAMRALRSKSVASVAQSVAARQEPSSCKGTLVFEGRTFNVFQ | |
| | PDCLRTEYNVNGYTPSAKSGSRIGFGSFLNQSASFSDLALFEKHFGFS | |
| | SQNFSVVLINGGTDLPQPPSDDNDGEANLDVQNILTIAHPLPITEFITAG | |
| | SPPYFPDPVEPAGTPDENEPYLQYFEYLLSKPNRDLPQVITNSYGDEE | |
| | QTVPQAYAVRVCNLIGLMGLRGISILESSGDEGVGASCVATNSTTPQFN | |
| | PIFPATCPYVTSVGGTVNFNPEVAWDGSSGGFSYYFSRPWYQEEAVG | |
| | NYLEKHVSAETKKYYGPYVDFSGRGFPDVAAHSVSPDYPVFQGGQLT | |
| | PSGGTSAASPVVASIIALLNDARLREGKPTLGFLNPLIYQYAYKGFTDITS | |
| | GQSDGCNGNNTQTDAPLPGAGVVLGAHWNATKGWDPTTGFGVPNFK | |
| | KLLELIRYI | |
| 14 | AVLVESLKQVPNGWNAVSTPDPSTSIVLQIALAQQNIDELEWRLAAVST | Trichoderma |
| | PNSGNYGKYLDIGEIEGIFAPSNASYKAVASWLQSHGVKNFVKQAGSI | atroviride IMI |
| | WFYTTVSTANKMLSTDFKHYSDPVGIEKLRTLQYSIPEELVGHVDLISPT | 206040 |
| | TYFGNNHPATARTPNMKAINVTYQIFHPDCLKTKYGVDGYAPSPRCGS | |
| | RIGFGSFLNETASYSDLAQFEKYFDLPNQNLSTLLINGAIDVQPPSNKND | |
| | SEANMDVQTILTFVQPLPITEFVVAGIPPYIPDAALPIGDPVQNEPWLEY | |
| | FEFLMSRTNAELPQVIANSYGDEEQTVPQAYAVRVCNQIGLLGLRGISVI | |
| | ASSGDTGVGMSCMASNSTTPQFNPMFPASCPYITTVGGTQHLDNEIA | |
| | WELSSGGFSNYFTRPWYQEDAAKTYLERHVSTETKAYYERYANFLGR | |
| | GFPDVAALSLNPDYPVIIGGELGPNGGTSAAAPVVASIIALLNDARLCLG | |
| | KPALGFLNPLIYQYADKGGFTDITSGQSWGCAGNTTQTGPPPPGAGVI | |
| | PGAHWNATKGWDPVTGFGTPNFKKLLSLALSV | |
| 15 | SPLARRWDDFAEKHAWVEVPRGWEMVSEAPSDHTFDLRIGVKSSGM | Agaricus |
| | EQLIENLMQTSDPTHSRYGQHLSKEELHDFVQPHPDSTGAVEAWLEDF | bisporus var. |
| | GISDDFIDRTGSGNWVTVRVSVAQAERMLGTKYNVYRHSESGESVVR | burnettii |
| | TMSYSLPSELHSHIDVVAPTTYFGTMKSMRVTSFLQPEIEPVDPSAKPS | JB137-S8 |
| | AAPASCLSTTVITPDCLRDLYNTADYVPSATSRNAIGIAGYLDRSNRADL | |
| | QTFFRRFRPDAVGFNYTTVQLNGGGDDQNDPGVEANLDIQYAAGIAFP | |
| | TPATYWSTGGSPPFIPDTQTPTNTNEPYLDWINFVLGQDEIPQVISTSY | |
| | GDDEQTVPEDYATSVCNLFAQLGSRGVTVFFSSGDFGVGGGDCLTND | |
| | GSNQVLFQPAFPASCPFVTAVGGTVRLDPEIAVSFSGGGFSRYFSRPS | |
| | YQNQTVAQFVSNLGNTFNGLYNKNGRAYPDLAAQGNGFQVVIDGIVRS | |
| | VGGTSASSPTVAGIFALLNDFKLSRGQSTLGFINPLIYSSATSGFNDIRA | |
| | GTNPGCGTRGFTAGTGWDPVTGLGTPDFLRLQGLI | |
| 16 | RVFDSLPHPPRGWSYSHAAESTEPLTLRIALRQQNAAALEQVVLQVSN | Magnaporthe |
| | PRHANYGQHLTRDELRSYTAPTPRAVRSVTSWLVDNGVDDYTVEHDW | oryzae 70-15 |
| | VTLRTTVGAADRLLGADFAWYAGPGETLQLRTLSYGVDDSVAPHVDLV | |
| | QPTTRFGGPVGQASHIFKQDDFDEQQLKTLSVGFQVMADLPANGPGSI | |
| | KAACNESGVTPLCLRTLYRVNYKPATTGNLVAFASFLEQYARYSDQQA | |
| | FTQRVLGPGVPLQNFSVETVNGGANDQQSKLDSGEANLDLQYVMAMS | |
| | HPIPILEYSTGGRGPLVPTLDQPNANNSSNEPYLEFLTYLLAQPDSAIPQ | |
| | TLSVSYGEEEQSVPRDYAIKVCNMFMQLGARGVSVMFSSGDSGPGND | |
| | CVRASDNATFFGSTFPAGCPYVTSVGSTVGFEPERAVSFSSGGFSIYH | |
| | ARPDYQNEVVPKYIESIKASGYEKFFDGNGRGIPDVAAQGARFVVIDKG | |
| | RVSLISGTSASSPAFAGMVALVNAARKSKDMPALGFLNPMLYQNAAAM | |
| | TDIVNGAGIGCRKQRTEFPNGARFNATAGWDPVTGLGTPLFDKLLAVG | |
| | APGVPNA | |
| 17 | SDVVLESLREVPQGWKRLRDADPEQSIKLRIALEQPNLDLFEQTLYDIS | Togninia |
| | SPDHPKYGQHLKSHELRDIMAPREESTAAVIAWLQDAGLSGSQIEDDS | minima |
| | DWINIQTTVAQANDMLNTTFGLFAQEGTEVNRIRALAYSVPEEIVPHVK | UCRPA7 |
| | MIAPIIRFGQLRPQMSHIFSHEKVEETPSIGTIKAAAIPSVDLNVTACNASI | |
| | TPECLRALYNVGDYEADPSKKSLFGVCGYLEQYAKHDQLAKFEQTYAP | |
| | YAIGADFSVVTINGGGDNQTSTIDDGEANLDMQYAVSMAYKTPITYYST | |
| | GGRGPLVPDLDQPDPNDVSNEPYLDFVSYLLKLPDSKLPQTITTSYGED | |
| | EQSVPRSYVEKVCTMFGALGARGVSVIFSSGDTGVGSACQTNDGKNT | |
| | TRFLPIFPAACPYVTSVGGTRYVDPEVAVSFSSGGFSDIFPTPLYQKGA | |
| | VSGYLKILGDRWKGLYNPHGRGFPDVSGQSVRYHVFDYGKDVMYSGT | |
| | SASAPMFAALVSLLNNARLAKKLPPMGFLNPWLYTVGFNGLTDIVHGG | |
| | STGCTGTDVYSGLPTPFVPYASWNATVGWDPVTGLGTPLFDKLLNLST | |
| | PNFHLPHIGGH | |
| 18 | STTSHVEGEVVERLHGVPEGWSQVGAPNPDQKLRFRIAVRSADSELFE | Bipolaris |
| | RTLMEVSSPSHPRYGQHLKRHELKDLIKPRAKSTSNILNWLQESGIEAR | maydis C5 |
| | DIQNDGEWISFYAPVKRAEQMMSTTFKTYQNEARANIKKIRSLDYSVPK | |
| | HIRDDIDIIQPTTRFGQIQPERSQVFSQEEVPFSALVVNATCNKKITPDCL | |
| | ANLYNFKDYDASDANVTIGVSGFLEQYARFDDLKQFISTFQPKAAGSTF | |
| | QVTSVNAGPFDQNSTASSVEANLDIQYTTGLVAPDIETRYFTVPGRGILI | |
| | PDLDQPTESDNANEPYLDYFTYLNNLEDEELPDVLTTSYGESEQSVPA | |
| | EYAKKVCNLIGQLGARGVSVIFSSGDTGPGSACQTNDGKNTTRFLPIFP | |
| | ASCPYVTSVGGTVGVEPEKAVSFSSGGFSDLWPRPAYQEKAVSEYLE | |
| | KLGDRWNGLYNPQGRGFPDVAAQGQGFQVFDKGRLISVGGTSASAP | |
| | VFASVVALLNNARKAAGMSSLGFLNPWIYEQGYKGLTDIVAGGSTGCT | |
| | GRSIYSGLPAPLVPYASWNATEGWDPVTGYGTPDFKQLLTLATAPKSG | |
| | ERRVRRGGLGGQA | |
| 19 | MLSSFLSQGAAVSLALLSLLPSPVAAEIFEKLSGVPNGWRYANNPHGN | Aspergillus |
| | EVIRLQIALQQHDVAGFEQAVMDMSTPGHADYGKHFRTHDEMKRMLL | kawachii IFO |
| | PSDTAVDSVRDWLESAGVHNIQVDADWVKFHTTVNKANALLDADFKW | 4308 |
| | YVSEAKHIRRLRTLQYSIPDALVSHINMIQPTTRFGQIQPNRATMRSKPK | |
| | HADETFLTAATLAQNTSHCDSIITPHCLKQLYNIGDYQADPKSGSKVGF | |
| | ASYLEEYARYADLERFEQHLAPNAIGQNFSVVQFNGGLNDQLSLSDSG | |
| | EANLDLQYILGVSAPVPVTEYSTGGRGELVPDLSSPDPNDNSNEPYLD | |
| | FLQGILKLDNSDLPQVISTSYGEDEQTIPVPYARTVCNLYAQLGSRGVS | |
| | VIFSSGDSGVGAACLTNDGTNRTHFPPQFPASCPWVTSVGATSKTSPE | |
| | QAVSFSSGGFSDLWPRPSYQQAAVQTYLTQHLGNKFSGLFNASGRAF | |
| | PDVAAQGVNYAVYDKGMLGQFDGTSCSAPTFSGVIALLNDARLRAGLP | |
| | VMGFLNPFLYGVGSESGALNDIVNGGSLGCDGRNRFGGTPNGSPVVP | |
| | FASWNATTGWDPVSGLGTPDFAKLRGVALGEAKAYGN | |
| 20 | MAATGRFTAFWNVASVPALIGILPLAGSHLRAVLCPVCIWRHSKAVCAP | Aspergillus |
| | DTLQAMRAFTRVTAISLAGFSCFAAAAAAAFESLRAVPDGWIYESTPDP | nidulans |
| | NQPLRLRIALKQHNVAGFEQALLDMSTPGHSSYGQHFGSYHEMKQLLL | FGSC A4 |
| | PTEEASSSVRDWLSAAGVEFEQDADWINFRTTVDQANALLDADFLWYT | |
| | TTGSTGNPTRILRTLSYSVPSELAGYVNMIQPTTRFGGTHANRATVRAK | |
| | PIFLETNRQLINAISSGSLEHCEKAITPSCLADLYNTEGYKASNRSGSKV | |
| | AFASFLEEYARYDDLAEFEETYAPYAIGQNFSVISINGGLNDQDSTADS | |
| | GEANLDLQYIIGVSSPLPVTEFTTGGRGKLIPDLSSPDPNDNTNEPFLDF | |
| | LEAVLKLDQKDLPQVISTSYGEDEQTIPEPYARSVCNLYAQLGSRGVSV | |
| | LFSSGDSGVGAACQTNDGKNTTHFPPQFPASCPWVTAVGGTNGTAPE | |
| | SGVYFSSGGFSDYWARPAYQNAAVESYLRKLGSTQAQYFNRSGRAFP | |
| | DVAAQAQNFAVVDKGRVGLFDGTSCSSPVFAGIVALLNDVRLKAGLPV | |
| | LGFLNPWLYQDGLNGLNDIVDGGSTGCDGNNRFNGSPNGSPVIPYAG | |
| | WNATEGWDPVTGLGTPDFAKLKALVLDA | |
| 21 | MLSFVRRGALSLALVSLLTSSVAAEVFEKLHVVPEGWRYASTPNPKQPI | Aspergillus |
| | RLQIALQQHDVTGFEQSLLEMSTPDHPNYGKHFRTHDEMKRMLLPNE | ruber CBS |
| | NAVHAVREWLQDAGISDIEEDADWVRFHTTVDQANDLLDANFLWYAH | 135680 |
| | KSHRNTARLRTLEYSIPDSIAPQVNVIQPTTRFGQIRANRATHSSKPKG | |
| | GLDELAISQAATADDDSICDQITTPHCLRKLYNVNGYKADPASGSKIGFA | |
| | SFLEEYARYSDLVLFEENLAPFAEGENFTVVMYNGGKNDQNSKSDSGE | |
| | ANLDLQYIVGMSAGAPVTEFSTAGRAPVIPDLDQPDPSAGTNEPYLEFL | |
| | QNVLHMDQEHLPQVISTSYGENEQTIPEKYARTVCNMYAQLGSRGVSV | |
| | IFSSGDSGVGSACMTNDGTNRTHFPPQFPASCPWVTSVGATEKMAPE | |
| | QATYFSSGGFSDLFPRPKYQDAAVSSYLQTLGSRYQGLYNGSNRAFP | |
| | DVSAQGTNFAVYDKGRLGQFDGTSCSAPAFSGIIALLNDVRLQNNKPV | |
| | LGFLNPWLYGAGSKGLNDVVHGGSTGCDGQERFAGKANGSPVVPYA | |
| | SWNATQGWDPVTGLGTPDFGKLKDLALSA | |
| 22 | MLPSLVNNGALSLAVLSLLTSSVAGEVFEKLSAVPKGWHFSHAAQADA | Aspergillus |
| | PINLKIALKQHDVEGFEQALLDMSTPGHENYGKHFHEHDEMKRMLLPS | terreus |
| | DSAVDAVQTANLTSAGITDYDLDADWINLRTTVEHANALLDTQFGWYEN | NIH2624 |
| | EVRHITRLRTLQYSIPETVAAHINMVQPTTRFGQIRPDRATFHAHHTSDA | |
| | RILSALAAASNSTSCDSVITPKCLKDLYKVGDYEADPDSGSQVAFASYL | |
| | EEYARYADMVKFQNSLAPYAKGQNFSVVLYNGGVNDQSSSADSGEAN | |
| | LDLQTIMGLSAPLPITEYITGGRGKLIPDLSQPNPNDNSNEPYLEFLQNIL | |
| | KLDQDELPQVISTSYGEDEQTIPRGYAESVCNMLAQLGSRGVSVVFSS | |
| | GDSGVGAACQTNDGRNQTHFNPQFPASCPWVTSVGATTKTNPEQAV | |
| | YFSSGGFSDFWKRPKYQDEAVAAYLDTLGDKFAGLFNKGGRAFPDVA | |
| | AQGMNYAIYDKGTLGRLDGTSCSAPAFSAIISLLNDARLREGKPTMGFL | |
| | NPWLYGEGREALNDVVVGGSKGCDGRDRFGGKPNGSPVVPFASWNA | |
| | TQGWDPVTGLGTPNFAKMLELAP | |
| 23 | MIASLFNRRALTLALLSLFASSATADVFESLSAVPQGWRYSRTPSANQP | Penicillium |
| | LKLQIALAQGDVAGFEAAVIDMSTPDHPSYGNHFNTHEEMKRMLQPSA | digitatum |
| | ESVDSIRNWLESAGISKIEQDADWMTFYTTVKTANELLAANFQFYINGV | Pd1 |
| | KKIERLRTLKYSVPDALVSHINMIQPTTRFGQLRAQRAILHTEVKDNDEA | |
| | FRSNAMSANPDCNSIITPQCLKDLYSIGDYEADPTNGNKVAFASYLEEY | |
| | ARYSDLALFEKNIAPFAKGQNFSVVQYNGGGNDQQSSSGSSEANLDL | |
| | QYIVGVSSPVPVTEFSTGGRGELVPDLDQPNPNDNNNEPYLEFLQNVL | |
| | KLHKKDLPQVISTSYGEDEQSVPEKYARAVCNLYSQLGSRGVSVIFSSG | |
| | DSGVGAACQTNDGRNATHFPPQFPAACPWVTSVGATTHTAPERAVYF | |
| | SSGGFSDLWDRPTWQEDAVSEYLENLGDRWSGLFNPKGRAFPDVAA | |
| | QGENYAIYDKGSLISVDGTSCSAPAFAGVIALLNDARIKANRPPMGFLN | |
| | PWLYSEGRSGLNDIVNGGSTGCDGHGRFSGPTNGGTSIPGASWNATK | |
| | GWDPVSGLGSPNFAAMRKLANAE | |
| 24 | MHVPLLNQGALSLAVVSLLASTVSAEVFDKLVAVPEGWRFSRTPSGDQ | Penicillium |
| | PIRLQVALTQGDVEGFEKAVLDMSTPDHPNYGKHFKSHEEVKRMLQPA | oxalicum |
| | GESVEAIHQWLEKAGITHIQQDADWMTFYTTVEKANNLLDANFQYYLN | 114-2 |
| | ENKQVERLRTLEYSVPDELVSHINLVTPTTRFGQLHAEGVTLHGKSKDV | |
| | DEQFRQAATSPSSDCNSAITPQCLKDLYKVGDYKASASNGNKVAFTSY | |
| | LEQYARYSDLALFEQNIAPYAQGQNFTVIQYNGGLNDQSSPADSSEAN | |
| | LDLQYIIGTSSPVPVTEFSTGGRGPLVPDLDQPDINDNNNEPYLDFLQN | |
| | VIKMSDKDLPQVISTSYGEDEQSVPASYARSVCNLIAQLGGRGVSVIFS | |
| | SGDSGVGSACQTNDGKNTTRFPAQFPAACPWVTSVGATTGISPERGV | |
| | FFSSGGFSDLWSRPSWQSHAVKAYLHKLGKRQDGLFNREGRAFPDVS | |
| | AQGENYAIYAKGRLGKVDGTSCSAPAFAGLVSLLNDARIKAGKSSLGFL | |
| | NPWLYSHPDALNDITVGGSTGCDGNARFGGRPNGSPVVPYASWNATE | |
| | GWDPVTGLGTPNFQKLLKSAVKQK | |
| 25 | MIASLFSRGALSLAVLSLLASSAAADVFESLSAVPQGWRYSRRPRADQ | Penicillium |
| | PLKLQIALTQGDTAGFEEAVMEMSTPDHPSYGHHFTTHEEMKRMLQPS | roqueforti |
| | AESAESIRDWLEGAGITRIEQDADWMTFYTTVETANELLAANFQFYVSN | FM164 |
| | VRHIERLRTLKYSVPKALVPHINMIQPTTRFGQLRAHRGILHGQVKESDE | |
| | AFRSNAVSAQPDCNSIITPQCLKDIYNIGDYQANDTNGNKVGFASYLEE | |
| | YARYSDLALFEKNIAPSAKGQNFSVTRYNGGLNDQSSSGSSSEANLDL | |
| | QYIVGVSSPVPVTEFSVGGRGELVPDLDQPDPNDNNNEPYLEFLQNVL | |
| | KLDKKDLPQVISTSYGEDEQSIPEKYARSVCNLYSQLGSRGVSVIFSSG | |
| | DSGVGSACLTNDGRNATRFPPQFPAACPWVTSVGATTHTAPEQAVYF | |
| | SSGGFSDLWARPKWQEEAVSEYLEILGNRWSGLFNPKGRAFPDVTAQ | |
| | GRNYAIYDKGSLTSVDGTSCSAPAFAGVVALLNDARLKVNKPPMGFLN | |
| | PWLYSTGRAGLKDIVDGGSTGCDGKSRFGGANNGGPSIPGASWNATK | |
| | GWDPVSGLGSPNFATMRKLANAE | |
| 26 | MIASLFNRGALSLAVLSLLASSASADVFESLSAVPQGWRYSRRPRADQ | Penicillium |
| | PLKLQIALAQGDTAGFEEAVMDMSTPDHPSYGNHFHTHEEMKRMLQP | rubens |
| | SAESADSIRDWLESAGINRIEQDADWMTFYTTVETANELLAANFQFYAN | Wisconsin |
| | SAKHIERLRTLQYSVPEALMPHINMIQPTTRFGQLRVQGAILHTQVKETD | 54-1255 |
| | EAFRSNAVSTSPDCNSIITPQCLKNMYNVGDYQADDDNGNKVGFASYL | |
| | EEYARYSDLELFEKNVAP FAKGQNFSVIQYNGGLNDQHSSASSSEANL | |
| | DLQYIVGVSSPVPVTEFSVGGRGELVPDLDQPDPNDNNNEPYLEFLQN | |
| | VLKMEQQDLPQVISTSYGENEQSVPEKYARTVCNLFSQLGSRGVSVIF | |
| | ASGDSGVGAACQTNDGRNATRFPAQFPAACPWVTSVGATTHTAPEKA | |
| | VYFSSGGFSDLWDRPKWQEDAVSDYLDTLGDRWSGLFNPKGRAFPD | |
| | VSAQGQNYAIYDKGSLTSVDGTSCSAPAFAGVIALLNDARLKANKPPM | |
| | GFLNPWLYSTGRDGLNDIVHGGSTGCDGNARFGGPGNGSPRVPGAS | |
| | WNATKGWDPVSGLGSPNFATMRKLANGE | |
| 27 | MLSSTLYAGLLCSLAAPALGVVHEKLSAVPSGWTLVEDASESDTTTLSI | Neosartorya |
| | ALARQNLDQLESKLTTLATPGNAEYGKWLDQSDIESLFPTASDDAVIQW | fischeri |
| | LKDAGVTQVSRQGSLVNFATTVGTANKLFDTKFSYYRNGASQKLRTTQ | NRRL 181 |
| | YSIPDSLTESIDLIAPTVFFGKEQDSALPPHAVKLPALPRRAATNSSCAN | |
| | LITPDCLVEMYNLGDYKPDASSGSRVGFGSFLNQSANYADLAAYEQLF | |
| | NIPPQNFSVELINGGANDQNWATASLGEANLDVELIVAVSHALPVVEFIT | |
| | GGSPPFVPNVDEPTAADNQNEPYLQYYEYLLSKPNSHLPQVISNSYGD | |
| | DEQTVPEYYARRVCNLIGLMGLRGITVLESSGDTGIGSACMSNDGTNT | |
| | PQFTPTFPGTCPFITAVGGTQSYAPEVAWDASSGGFSNYFSRPWYQY | |
| | FAVENYLNNHITKDTKKYYSQYTNFKGRGFPDVSAHSLTPDYEVVLTGK | |
| | HYKSGGTSAACPVFAGIVGLLNDARLRAGKSTLGFLNPLLYSILAEGFT | |
| | DITAGSSIGCNGINPQTGKPVPGGGIIPYAHWNATAGWDPVTGLGVPD | |
| | FMKLKELVLSL | |
| 28 | MLSSTLYAGWLLSLAAPALCVVQEKLSAVPSGWTLIEDASESDTITLSIA | Aspergillus |
| | LARQNLDQLESKLTTLATPGNPEYGKWLDQSDIESLFPTASDDAVLQW | fumigatus |
| | LKAAGITQVSRQGSLVNFATTVGTANKLFDTKFSYYRNGASQKLRTTQ | CAE17675 |
| | YSIPDHLTESIDLIAPTVFFGKEQNSALSSHAVKLPALPRRAATNSSCAN | |
| | LITPDCLVEMYNLGDYKPDASSGSRVGFGSFLNESANYADLAAYEQLF | |
| | NIPPQNFSVELINRGVNDQNWATASLGEANLDVELIVAVSHPLPVVEFIT | |
| | GALPPVLRVLALQTQLPSSSGDFQLTVPEYYARRVCNLIGLMGLRGITV | |
| | LESSGDTGIGSACMSNDGTNKPQFTPTFPGTCPFITAVGGTQSYAPEV | |
| | AWDGSSGGFSNYFSRPWYQSFAVDNYLNNHITKDTKKYYSQYTNFKG | |
| | RGFPDVSAHSLTPYYEVVLTGKHYKSGGTSAASPVFAGIVGLLNDARL | |
| | RAGKSTLGFLNPLLYSILAEGFTDITAGSSIGCNGINPQTGKPVPGGGIIP | |
| | YAHWNATAGWDPVTGLGVPDFMKLKELVLSL | |
| 29 | QEPSSCKGTLVFEGETFNVFQPDCLRTEYSVDGYTPSVKSGSRIGFGS | Trichoderma |
| | FLNESASFADQALFEKHFNIPSQNFSVVLINGGTDLPQPPSDANDGEAN | reesei QM6a |
| | LDAQTILTIAHPLPITEFITAGSPPYFPDPVEPAGTPNENEPYLQYYEFLL | |
| | SKSNAEIPQVITNSYGDEEQTVPRSYAVRVCNLIGLLGLRGISVLHSSGD | |
| | EGVGASCVATNSTTPQFNPIFPATCPYVTSVGGTVSFNPEVAWAGSSG | |
| | GFSYYFSRPWYQQEAVGTYLEKYVSAETKKYYGPYVDFSGRGFPDVA | |
| | AHSVSPDYPVFQGGELTPSGGTSAASPVVAAIVALLNDARLREGKPTL | |
| | GFLNPLIYLHASKGFTDITSGQSEGCNGNNTQTGSPLPGAGFIAGAHW | |
| | NATKGWDPTTGFGVPNLKKLLALVRF | |
| 30 | CDSIITPTCLKELYNIGDYQADANSGSKIAFASYLEEYARYADLENFENY | Aspergillus |
| | LAPWAKGQNFSVTTFNGGLNDQNSSSDSGEANLDLQYILGVSAPLPVT | oryzae RIB40 |
| | EFSTGGRGPLVPDLTQPDPNSNSNEPYLEFFQNVLKLDQKDLPQVIST | |
| | SYGENEQEIPEKYARTVCNLIAQLGSRGVSVLFSSGDSGVGEGCMTND | |
| | GTNRTHFPPQFPAACPWVTSVGATFKTTPERGTYFSSGGFSDYWPRP | |
| | EWQDEAVSSYLETIGDTFKGLYNSSGRAFPDVAAQGMNFAVYDKGTL | |
| | GEFDGTSASAPAFSAVIALLNDARLRAGKPTLGFLNPWLYKTGRQGLQ | |
| | DITLGASIGCTGRARFGGAPDGGPVVPYASWNATQGWDPVTGLGTPD | |
| | FAELKKLA | |
| 31 | CDATITPQCLKTLYKIDYKADPKSGSKVAFASYLEQYARYNDLALFEKAF | Phaeosphaer |
| | LPEAVGQNFSVVQFSGGLNDQNTTQDSGEANLDLQYIVGVSAPLPVTE | ia nodorum |
| | FSTGGRGPWVADLDQPDEADSANEPYLEFLQGVLKLPQSELPQVISTS | SN15 |
| | YGENEQSVPKSYALSVCNLFAQLGSRGVSVIFSSGDSGPGSACQSND | |
| | GKNTTKFQPQYPAACPFVTSVGSTRYLNETATGFSSGGFSDYWKRPS | |
| | YQDDAVKAYFHHLGEKFKPYFNRHGRGFPDVATQGYGFRVYDQGKLK | |
| | GLQGTSASAPAFAGVIGLLNDARLKAKKPTLGFLNPLLYSNSDALNDIVL | |
| | GGSKGCDGHARFNGPPNGSPVIPYAGWNATAGWDPVTGLGTPNFPK | |
| | LLKAA | |
| 32 | VFQPDCLRTEYSVNGYKPSAKSGSRIGFGSFLNQSASSSDLALFEKHF | Trichoderma |
| | GFASQGFSVELINGGSNPQPPTDANDGEANLDAQNIVSFVQPLPITEFI | atroviride IMI |
| | AGGTAPYFPDPVEPAGTPDENEPYLEYYEYLLSKSNKELPQVITNSYGD | 206040 |
| | EEQTVPQAYAVRVCNLIGLMGLRGISILESSGDEGVGASCLATNSTTTP | |
| | QFNPIFPATCPYVTSVGGTVSFNPEVAWDGSSGGFSYYFSRPWYQEA | |
| | AVGTYLNKYVSEETKEYYKSYVDFSGRGFPDVAAHSVSPDYPVFQGG | |
| | ELTPSGGTSAASPIVASVIALLNDARLRAGKPALGFLNPLIYGYAYKGFT | |
| | DITSGQAVGCNGNNTQTGGPLPGAGVIPGAFWNATKGWDPTTGFGVP | |
| | NFKKLLELV | |
| 33 | CRSLVTTACLRELYGLGDRVTQARDDNRIGVSGFLEEYAQYRDLELFLS | Arthroderma |
| | RFEPSAKGFNFSEGLIAGGKNTQGGPGSSTEANLDMQYVVGLSHKAK | benhamiae |
| | VTYYSTAGRGPLIPDLSQPSQASNNNEPYLEQLRYLVKLPKNQLPSVLT | CBS 112371 |
| | TSYGDTEQSLPASYTKATCDLFAQLGTMGVSVIFSSGDTGPGSSCQTN | |
| | DGKNATRFNPIYPASCPFVTSIGGTVGTGPERAVSFSSGGFSDRFPRP | |
| | QYQDNAVKDYLKILGNQWSGLFDPNGRAFPDIAAQGSNYAVYDKGRM | |
| | TGVSGTSASAPAMAAIIAQLNDFRLAKGSPVLGFLNPWIYSKGFSGFTDI | |
| | VDGGSRGCTGYDIYSGLKAKKVPYASWNATKGWDPVTGFGTPNFQAL | |
| | TKVL | |
| 34 | CQTSITPSCLKQMYNIGDYTPKVESGSTIGFSSFLGESAIYSDVFLFEEK | Fusarium |
| | FGIPTQNFTTVLINNGTDDQNTAHKNFGEADLDAENIVGIAHPLPFTQYI | graminearum |
| | TGGSPPFLPNIDQPTAADNQNEPYVPFFRYLLSQKEVPAVVSTSYGDE | PH-1 |
| | EDSVPREYATMTCNLIGLLGLRGISVIFSSGDIGVGAGCLGPDHKTVEF | |
| | NAIFPATCPYLTSVGGTVDVTPEIAWEGSSGGFSKYFPRPSYQDKAVK | |
| | TYMKTVSKQTKKYYGPYTNWEGRGFPDVAGHSVSPNYEVIYAGKQSA | |
| | SGGTSAAAPVWAAIVGLLNDARFRAGKPSLGWLNPLVYKYGPKVLTDI | |
| | TGGYAIGCDGNNTQSGKPEPAGSGIVPGARWNATAGWDPVTGYGTP | |
| | DFGKLKDLVLS | |
| 35 | CDLVITPPCLEAAYNYKNYMPDPNSGSRVSFTSFLEQAAQQSDLTKFLS | Acremonium |
| | LTGLDRLRPPSSKPASFDTVLINGGETHQGTPPNKTSEANLDVQWLAA | alcalophilum |
| | VIKARLPITQWITGGRPPFVPNLRLRHEKDNTNEPYLEFFEYLVRLPARD | |
| | LPQVISNSYAEDEQTVPEAYARRVCNLIGIMGLRGVTVLTASGDSGVGA | |
| | PCRANDGSDRLEFSPQFPTSCPYITAVGGTEGWDPEVAWEASSGGFS | |
| | HYFLRPWYQANAVEKYLDEELDPATRAYYDGNGFVQFAGRAYPDLSA | |
| | HSSSPRYAYIDKLAPGLTGGTSASCPVVAGIVGLLNDARLRRGLPTMGF | |
| | INPWLYTRGFEALQDVTGGRASGCQGIDLQRGTRVPGAGIIPWASWNA | |
| | TPGWDPATGLGLPDFWAMRGL | |
| 36 | CATIITPPCLETAYNYKGYIPDPKSGSRVSFTSFLEQAAQQADLTKFLSL | Sodiomyces |
| | TRLEGFRTPASKKKTFKTVLINGGESHEGVHKKSKTSEANLDVQWLAA | alkalinus |
| | VTQTKLPITQWITGGRPPFVPNLRIPTPEANTNEPYLEFLEYLFRLPDKD | |
| | LPQVISNSYAEDEQSVPEAYARRVCGLLGIMGLRGVTVLTASGDSGVG | |
| | APCRANDGSGREEFSPQFPSSCPYITTVGGTQAWDPEVAWKGSSGG | |
| | FSNYFPRPWYQVAAVEKYLEEQLDPAAREYYEENGFVRFAGRAFPDL | |
| | SAHSSSPKYAYVDKRVPGLTGGTSASCPVVAGIVGLLNDARLRRGLPT | |
| | MGFINPWLYAKGYQALEDVTGGAAVGCQGIDIQTGKRVPGAGIIPGAS | |
| | WNATPDWDPATGLGLPNFWAMRELA | |
| 37 | CADTITLSCLKEMYNFGNYTPSASSGSKLGFASFLNESASYSDLAKFER | Aspergillus |
| | LFNLPSQNFSVELINGGVNDQNQSTASLTEADLDVELLVGVGHPLPVTE | kawachii IFO |
| | FITSGEPPFIPDPDEPSAADNENEPYLQYYEYLLSKPNSALPQVISNSYG | 4308 |
| | DDEQTVPEYYAKRVCNLIGLVGLRGISVLESSGDEGIGSGCRTTDGTNS | |
| | TQFNPIFPATCPYVTAVGGTMSYAPEIAWEASSGGFSNYFERAWFQKE | |
| | AVQNYLANHITNETKQYYSQFANFSGRGFPDVSAHSFEPSYEVIFYGA | |
| | RYGSGGTSAACPLFSALVGMLNDARLRAGKSTLGFLNPLLYSKGYKAL | |
| | TDVTAGQSIGCNGIDPQSDEAVAGAGIIPWAHWNATVGWDPVTGLGLP | |
| | DFEKLRQLVLS | |
| 38 | CQTSITPACLKQMYNVGNYTPSVAHGSRVGFGSFLNQSAIFDDLFTYE | Talaromyces |
| | KVNDIPSQNFTKVIIANASNSQDASDGNYGEANLDVQNIVGISHPLPVTE | stipitatus |
| | FLTGGSPPFVASLDTPTNQNEPYIPYYEYLLSQKNEDLPQVISNSYGDD | ATCC 10500 |
| | EQSVPYKYAIRACNLIGLTGLRGISVLESSGDLGVGAGCRSNDGKNKTQ | |
| | FDPIFPATCPYVTSVGGTQSVTPEIAWVASSGGFSNYFPRTANYQEPAI | |
| | QTYLGLLDDETKTYYSQYTNFEGRGFPDVSAHSLTPDYQVVGGGYLQ | |
| | PSGGTSAASPVFAGIIALLNDARLAAGKPTLGFLNPFFYLYGYKGLNDIT | |
| | GGQSVGCNGINGQTGAPVPGGGIVPGAAWNSTTGWDPATGLGTPDF | |
| | QKLKELVLS | |
| 39 | CQTSITPSCLKQMYNIGDYTPDAKSGSEIGFSSFLGQAAIYSDVFKFEEL | Fusarium |
| | FGIPKQNYTTILINNGTDDQNTAHGNFGEANLDAENIVGIAHPLPFKQYIT | oxysporum f. |
| | GGSPPFVPNIDQPTEKDNQNEPYVPFFRYLLGQKDLPAVISTSYGDEE | sp. cubense |
| | DSVPREYATLTCNMIGLLGLRGISVIFSSGDIGVGSGCLAPDYKTVEFNA | race 4 |
| | IFPATCPYLTSVGGTVDVTPEIAWEGSSGGFSKYFPRPSYQDKAIKKYM | |
| | KTVSKETKKYYGPYTNWEGRGFPDVAGHSVAPDYEVIYNGKQARSGG | |
| | TSAAAPVWAAIVGLLNDARFKAGKKSLGWLNPLIYKHGPKVLTDITGGY | |
| | AIGCDGNNTQSGKPEPAGSGLVPGARWNATAGWDPTTGYGTPNFQK | |
| | LKDLVLS | |
| 40 | VFQPDCLRTEYNVNGYTPSAKSGSRIGFGSFLNQSASFSDLALFEKHF | Trichoderma |
| | GFSSQNFSVVLINGGTDLPQPPSDDNDGEANLDVQNILTIAHPLPITEFIT | virens |
| | AGSPPYFPDPVEPAGTPDENEPYLQYFEYLLSKPNRDLPQVITNSYGD | Gv29-8 |
| | EEQTVPQAYAVRVCNLIGLMGLRGISILESSGDEGVGASCVATNSTTPQ | |
| | FNPIFPATCPYVTSVGGTVNFNPEVAWDGSSGGFSYYFSRPWYQEEA | |
| | VGNYLEKHVSAETKKYYGPYVDFSGRGFPDVAAHSVSPDYPVFQGGQ | |
| | LTPSGGTSAASPVVASIIALLNDARLREGKPTLGFLNPLIYQYAYKGFTDI | |
| | TSGQSDGCNGNNTQTDAPLPGAGVVLGAHWNATKGWDPTTGFGVPN | |
| | FKKLLELI | |
| 41 | QIFHPDCLKTKYGVDGYAPSPRCGSRIGFGSFLNETASYSDLAQFEKYF | Trichoderma |
| | DLPNQNLSTLLINGAIDVQPPSNKNDSEANMDVQTILTFVQPLPITEFVV | atroviride IMI |
| | AGIPPYIPDAALPIGDPVQNEPWLEYFEFLMSRTNAELPQVIANSYGDE | 206040 |
| | EQTVPQAYAVRVCNQIGLLGLRGISVIASSGDTGVGMSCMASNSTTPQ | |
| | FNPMFPASCPYITTVGGTQHLDNEIAWELSSGGFSNYFTRPWYQEDAA | |
| | KTYLERHVSTETKAYYERYANFLGRGFPDVAALSLNPDYPVIIGGELGP | |
| | NGGTSAAAPVVASIIALLNDARLCLGKPALGFLNPLIYQYADKGGFTDIT | |
| | SGQSWGCAGNTTQTGPPPPGAGVIPGAHWNATKGWDPVTGFGTPNF | |
| | KKLLSLALS | |
| 42 | TVITPDCLRDLYNTADYVPSATSRNAIGIAGYLDRSNRADLQTFFRRFRP | Agaricus |
| | DAVGFNYTTVQLNGGGDDQNDPGVEANLDIQYAAGIAFPTPATYWSTG | bisporus var. |
| | GSPPFIPDTQTPTNTNEPYLDWINFVLGQDEIPQVISTSYGDDEQTVPE | burnettii |
| | DYATSVCNLFAQLGSRGVTVFFSSGDFGVGGGDCLTNDGSNQVLFQP | JB137-S8 |
| | AFPASCPFVTAVGGTVRLDPEIAVSFSGGGFSRYFSRPSYQNQTVAQF | |
| | VSNLGNTFNGLYNKNGRAYPDLAAQGNGFQVVIDGIVRSVGGTSASSP | |
| | TVAGIFALLNDFKLSRGQSTLGFINPLIYSSATSGFNDIRAGTNPGCGTR | |
| | GFTAGTGWDPVTGLGTPDFLRLQ | |
| 43 | GVTPLCLRTLYRVNYKPATTGNLVAFASFLEQYARYSDQQAFTQRVLG | Magnaporthe |
| | PGVPLQNFSVETVNGGANDQQSKLDSGEANLDLQYVMAMSHPIPILEY | oryzae 70-15 |
| | STGGRGPLVPTLDQPNANNSSNEPYLEFLTYLLAQPDSAIPQTLSVSYG | |
| | EEEQSVPRDYAIKVCNMFMQLGARGVSVMFSSGDSGPGNDCVRASD | |
| | NATFFGSTFPAGCPYVTSVGSTVGFEPERAVSFSSGGFSIYHARPDYQ | |
| | NEVVPKYIESIKASGYEKFFDGNGRGIPDVAAQGARFVVIDKGRVSLISG | |
| | TSASSPAFAGMVALVNAARKSKDMPALGFLNPMLYQNAAAMTDIVNGA | |
| | GIGCRKQRTEFPNGARFNATAGWDPVTGLGTPLFDKLLA | |
| 44 | CNASITPECLRALYNVGDYEADPSKKSLFGVCGYLEQYAKHDQLAKFE | Togninia |
| | QTYAPYAIGADFSVVTINGGGDNUSTIDDGEANLDMQYAVSMAYKTPI | minima |
| | TYYSTGGRGPLVPDLDQPDPNDVSNEPYLDFVSYLLKLPDSKLPQTITT | UCRPA7 |
| | SYGEDEQSVPRSYVEKVCTMFGALGARGVSVIFSSGDTGVGSACQTN | |
| | DGKNTTRFLPIFPAACPYVTSVGGTRYVDPEVAVSFSSGGFSDIFPTPL | |
| | YQKGAVSGYLKILGDRWKGLYNPHGRGFPDVSGQSVRYHVFDYGKDV | |
| | MYSGTSASAPMFAALVSLLNNARLAKKLPPMGFLNPWLYTVGFNGLTD | |
| | IVHGGSTGCTGTDVYSGLPTPFVPYASWNATVGWDPVTGLGTPLFDKL | |
| | LNL | |
| 45 | CNKKITPDCLANLYNFKDYDASDANVTIGVSGFLEQYARFDDLKQFISTF | Bipolaris |
| | QPKAAGSTFQVTSVNAGPFDQNSTASSVEANLDIQYTTGLVAPDIETRY | maydis C5 |
| | FTVPGRGILIPDLDQPTESDNANEPYLDYFTYLNNLEDEELPDVLTTSYG | |
| | ESEQSVPAEYAKKVCNLIGQLGARGVSVIFSSGDTGPGSACQTNDGKN | |
| | TTRFLPIFPASCPYVTSVGGTVGVEPEKAVSFSSGGFSDLWPRPAYQE | |
| | KAVSEYLEKLGDRWNGLYNPQGRGFPDVAAQGQGFQVFDKGRLISVG | |
| | GTSASAPVFASVVALLNNARKAAGMSSLGFLNPWIYEQGYKGLTDIVA | |
| | GGSTGCTGRSIYSGLPAPLVPYASWNATEGWDPVTGYGTPDFKQLLTL | |
| | AT | |
| 46 | CDSIITPHCLKQLYNIGDYQADPKSGSKVGFASYLEEYARYADLERFEQ | Aspergillus |
| | HLAPNAIGQNFSVVQFNGGLNDQLSLSDSGEANLDLQYILGVSAPVPVT | kawachii IFO |
| | EYSTGGRGELVPDLSSPDPNDNSNEPYLDFLQGILKLDNSDLPQVISTS | 4308 |
| | YGEDEQTIPVPYARTVCNLYAQLGSRGVSVIFSSGDSGVGAACLTNDG | |
| | TNRTHFPPQFPASCPWVTSVGATSKTSPEQAVSFSSGGFSDLWPRPS | |
| | YQQAAVQTYLTQHLGNKFSGLFNASGRAFPDVAAQGVNYAVYDKGML | |
| | GQFDGTSCSAPTFSGVIALLNDARLRAGLPVMGFLNPFLYGVGSESGA | |
| | LNDIVNGGSLGCDGRNRFGGTPNGSPVVPFASWNATTGWDPVSGLG | |
| | TPDFAKLRGV | |
| 47 | CEKAITPSCLADLYNTEGYKASNRSGSKVAFASFLEEYARYDDLAEFEE | Aspergillus |
| | TYAPYAIGQNFSVISINGGLNDQDSTADSGEANLDLQYIIGVSSPLPVTE | nidulans |
| | FTTGGRGKLIPDLSSPDPNDNTNEPFLDFLEAVLKLDQKDLPQVISTSY | FGSC A4 |
| | GEDEQTIPEPYARSVCNLYAQLGSRGVSVLFSSGDSGVGAACQTNDG | |
| | KNTTHFPPQFPASCPWVTAVGGTNGTAPESGVYFSSGGFSDYWARPA | |
| | YQNAAVESYLRKLGSTQAQYFNRSGRAFPDVAAQAQNFAVVDKGRVG | |
| | LFDGTSCSSPVFAGIVALLNDVRLKAGLPVLGFLNPWLYQDGLNGLNDI | |
| | VDGGSTGCDGNNRFNGSPNGSPVIPYAGWNATEGWDPVTGLGTPDF | |
| | AKLKALVL | |
| 48 | CDQITTPHCLRKLYNVNGYKADPASGSKIGFASFLEEYARYSDLVLFEE | Aspergillus |
| | NLAPFAEGENFTVVMYNGGKNDQNSKSDSGEANLDLQYIVGMSAGAP | ruber CBS |
| | VTEFSTAGRAPVIPDLDQPDPSAGTNEPYLEFLQNVLHMDQEHLPQVIS | 135680 |
| | TSYGENEQTIPEKYARTVCNMYAQLGSRGVSVIFSSGDSGVGSACMTN | |
| | DGTNRTHFPPQFPASCPWVTSVGATEKMAPEQATYFSSGGFSDLFPR | |
| | PKYQDAAVSSYLQTLGSRYQGLYNGSNRAFPDVSAQGTNFAVYDKGR | |
| | LGQFDGTSCSAPAFSGIIALLNDVRLQNNKPVLGFLNPWLYGAGSKGL | |
| | NDVVHGGSTGCDGQERFAGKANGSPVVPYASWNATQGWDPVTGLG | |
| | TPDFGKLKDLAL | |
| 49 | CDSVITPKCLKDLYKVGDYEADPDSGSQVAFASYLEEYARYADMVKFQ | Aspergillus |
| | NSLAPYAKGQNFSVVLYNGGVNDQSSSADSGEANLDLQTIMGLSAPLP | terreus |
| | ITEYITGGRGKLIPDLSQPNPNDNSNEPYLEFLQNILKLDQDELPQVISTS | NIH2624 |
| | YGEDEQTIPRGYAESVCNMLAQLGSRGVSVVFSSGDSGVGAACQTND | |
| | GRNQTHFNPQFPASCPWVTSVGATTKTNPEQAVYFSSGGFSDFWKR | |
| | PKYQDEAVAAYLDTLGDKFAGLFNKGGRAFPDVAAQGMNYAIYDKGTL | |
| | GRLDGTSCSAPAFSAIISLLNDARLREGKPTMGFLNPWLYGEGREALN | |
| | DVVVGGSKGCDGRDRFGGKPNGSPVVPFASWNATQGWDPVTGLGT | |
| | PNFAKMLELA | |
| 50 | CNSIITPQCLKDLYSIGDYEADPTNGNKVAFASYLEEYARYSDLALFEKN | Penicillium |
| | IAPFAKGQNFSVVQYNGGGNDQQSSSGSSEANLDLQYIVGVSSPVPVT | digitatum |
| | EFSTGGRGELVPDLDQPNPNDNNNEPYLEFLQNVLKLHKKDLPQVIST | Pd1 |
| | SYGEDEQSVPEKYARAVCNLYSQLGSRGVSVIFSSGDSGVGAACQTN | |
| | DGRNATHFPPQFPAACPWVTSVGATTHTAPERAVYFSSGGFSDLWDR | |
| | PTANQEDAVSEYLENLGDRWSGLFNPKGRAFPDVAAQGENYAIYDKGS | |
| | LISVDGTSCSAPAFAGVIALLNDARIKANRPPMGFLNPWLYSEGRSGLN | |
| | DIVNGGSTGCDGHGRFSGPTNGGTSIPGASWNATKGWDPVSGLGSP | |
| | NFAAMRKLA | |
| 51 | CNSAITPQCLKDLYKVGDYKASASNGNKVAFTSYLEQYARYSDLALFE | Penicillium |
| | QNIAPYAQGQNFTVIQYNGGLNDQSSPADSSEANLDLQYIIGTSSPVPV | oxalicum |
| | TEFSTGGRGPLVPDLDQPDINDNNNEPYLDFLQNVIKMSDKDLPQVIST | 114-2 |
| | SYGEDEQSVPASYARSVCNLIAQLGGRGVSVIFSSGDSGVGSACQTND | |
| | GKNTTRFPAQFPAACPWVTSVGATTGISPERGVFFSSGGFSDLWSRP | |
| | SWQSHAVKAYLHKLGKRQDGLFNREGRAFPDVSAQGENYAIYAKGRL | |
| | GKVDGTSCSAPAFAGLVSLLNDARIKAGKSSLGFLNPWLYSHPDALNDI | |
| | TVGGSTGCDGNARFGGRPNGSPVVPYASWNATEGWDPVTGLGTPNF | |
| | QKLLKSAV | |
| 52 | CNSIITPQCLKDIYNIGDYQANDTNGNKVGFASYLEEYARYSDLALFEKN | Penicillium |
| | IAPSAKGQNFSVTRYNGGLNDQSSSGSSSEANLDLQYIVGVSSPVPVT | roqueforti |
| | EFSVGGRGELVPDLDQPDPNDNNNEPYLEFLQNVLKLDKKDLPQVIST | FM164 |
| | SYGEDEQSIPEKYARSVCNLYSQLGSRGVSVIFSSGDSGVGSACLTND | |
| | GRNATRFPPQFPAACPWVTSVGATTHTAPEQAVYFSSGGFSDLWARP | |
| | KWQEEAVSEYLEILGNRWSGLFNPKGRAFPDVTAQGRNYAIYDKGSLT | |
| | SVDGTSCSAPAFAGVVALLNDARLKVNKPPMGFLNPWLYSTGRAGLK | |
| | DIVDGGSTGCDGKSRFGGANNGGPSIPGASWNATKGWDPVSGLGSP | |
| | NFATMRKLA | |
| 53 | CNSIITPQCLKNMYNVGDYQADDDNGNKVGFASYLEEYARYSDLELFE | Penicillium |
| | KNVAPFAKGQNFSVIQYNGGLNDQHSSASSSEANLDLQYIVGVSSPVP | rubens |
| | VTEFSVGGRGELVPDLDQPDPNDNNNEPYLEFLQNVLKMEQQDLPQVI | Wisconsin |
| | STSYGENEQSVPEKYARTVCNLFSQLGSRGVSVIFASGDSGVGAACQT | 54-1255 |
| | NDGRNATRFPAQFPAACPWVTSVGATTHTAPEKAVYFSSGGFSDLWD | |
| | RPKWQEDAVSDYLDTLGDRWSGLFNPKGRAFPDVSAQGQNYAIYDKG | |
| | SLTSVDGTSCSAPAFAGVIALLNDARLKANKPPMGFLNPWLYSTGRDG | |
| | LNDIVHGGSTGCDGNARFGGPGNGSPRVPGASWNATKGWDPVSGLG | |
| | SPNFATMRKLA | |
| 54 | CANLITPDCLVEMYNLGDYKPDASSGSRVGFGSFLNQSANYADLAAYE | Neosartorya |
| | QLFNIPPQNFSVELINGGANDQNWATASLGEANLDVELIVAVSHALPVV | fischeri |
| | EFITGGSPPFVPNVDEPTAADNQNEPYLQYYEYLLSKPNSHLPQVISNS | NRRL 181 |
| | YGDDEQTVPEYYARRVCNLIGLMGLRGITVLESSGDTGIGSACMSNDG | |
| | TNTPQFTPTFPGTCPFITAVGGTQSYAPEVAWDASSGGFSNYFSRPW | |
| | YQYFAVENYLNNHITKDTKKYYSQYTNFKGRGFPDVSAHSLTPDYEVV | |
| | LTGKHYKSGGTSAACPVFAGIVGLLNDARLRAGKSTLGFLNPLLYSILAE | |
| | GFTDITAGSSIGCNGINPQTGKPVPGGGIIPYAHWNATAGWDPVTGLG | |
| | VPDFMKLKELVLS | |
| 55 | CANLITPDCLVEMYNLGDYKPDASSGSRVGFGSFLNESANYADLAAYE | Aspergillus |
| | QLFNIPPQNFSVELINRGVNDQNWATASLGEANLDVELIVAVSHPLPVV | fumigatus |
| | EFITGALPPVLRVLALQTQLPSSSGDFQLTVPEYYARRVCNLIGLMGLR | CAE17675 |
| | GITVLESSGDTGIGSACMSNDGTNKPQFTPTFPGTCPFITAVGGTQSYA | |
| | PEVAWDGSSGGFSNYFSRPWYQSFAVDNYLNNHITKDTKKYYSQYTN | |
| | FKGRGFPDVSAHSLTPYYEVVLTGKHYKSGGTSAASPVFAGIVGLLND | |
| | ARLRAGKSTLGFLNPLLYSILAEGFTDITAGSSIGCNGINPQTGKPVPGG | |
| | GIIPYAHWNATAGWDPVTGLGVPDFMKLKELVLS | |
| 56 | ATGGCAAAGTTGAGCACTCTCCGGCTTGCGAGCCTTCTTTCCCTTGT | Trichoderma |
| | CAGTGTGCAGGTATCTGCCTCTGTCCATCTATTGGAGAGTCTGGAG | reesei QM6a |
| | AAGCTGCCTCATGGATGGAAAGCAGCTGAAACCCCGAGCCCTTCGT | |
| | CTCAAATCGTCTTGCAGGTTGCTCTGACGCAGCAGAACATTGACCA | |
| | GCTTGAATCGAGGCTCGCAGCTGTATCCACACCCACTTCTAGCACC | |
| | TACGGCAAATACTTGGATGTAGACGAGATCAACAGCATCTTCGCTCC | |
| | AAGTGATGCTAGCAGTTCTGCCGTCGAGTCTTGGCTTCAGTCCCAC | |
| | GGAGTGACGAGTTACACCAAGCAAGGCAGCAGCATTTGGTTTCAAA | |
| | CAAACATCTCCACTGCAAATGCGATGCTCAGCACCAATTTCCACACG | |
| | TACAGCGATCTCACCGGCGCGAAGAAGGTGCGCACTCTCAAGTACT | |
| | CGATCCCGGAGAGCCTCATCGGCCATGTCGATCTCATCTCTCCCAC | |
| | GACCTATTTTGGCACGACAAAGGCCATGAGGAAGTTGAAATCCAGT | |
| | GGCGTGAGCCCAGCCGCTGATGCTCTAGCCGCTCGCCAAGAACCT | |
| | TCCAGCTGCAAAGGAACTCTAGTCTTTGAGGGAGAAACGTTCAATGT | |
| | CTTTCAGCCAGACTGTCTCAGGACCGAGTATAGTGTTGATGGATACA | |
| | CCCCGTCTGTCAAGTCTGGCAGCAGAATTGGGTTTGGTTCCTTTCTC | |
| | AATGAGAGCGCAAGCTTCGCAGATCAAGCACTCTTTGAGAAGCACT | |
| | TCAACATCCCCAGTCAAAACTTCTCCGTTGTCCTGATCAACGGTGGA | |
| | ACGGATCTCCCTCAGCCGCCTTCTGACGCCAACGATGGCGAAGCCA | |
| | ACCTGGACGCTCAAACCATTTTGACCATCGCACATCCTCTCCCCATC | |
| | ACCGAATTCATCACCGCCGGCAGTCCGCCATACTTCCCCGATCCAG | |
| | TTGAACCTGCGGGAACACCCAACGAGAACGAGCCTTATTTACAGTA | |
| | TTACGAATTTCTGTTGTCCAAGTCCAACGCTGAAATTCCGCAAGTCA | |
| | TTACCAACTCCTACGGCGACGAGGAGCAAACTGTGCCGCGGTCATA | |
| | TGCCGTTCGAGTTTGCAATCTGATTGGTCTGCTAGGACTACGCGGT | |
| | ATCTCTGTCCTTCATTCCTCGGGCGACGAGGGTGTGGGCGCCTCTT | |
| | GCGTTGCTACCAACAGCACCACGCCTCAGTTTAACCCCATCTTTCCT | |
| | GTAGGTCTTCTACGTCAACACTTCCAGACAACCATTTTCTCCTACTA | |
| | ACCACTCTACCCTACTCTCTGTTCACATAGGCTACATGTCCTTATGTT | |
| | ACAAGTGTTGGCGGAACCGTGAGCTTCAATCCCGAGGTTGCCTGGG | |
| | CTGGTTCATCTGGAGGTTTCAGCTACTACTTCTCTAGACCCTGGTAC | |
| | CAGCAGGAAGCTGTGGGTACTTACCTTGAGAAATATGTCAGTGCTG | |
| | AGACAAAGAAATACTATGGACCTTATGTCGATTTCTCCGGACGAGGT | |
| | TTCCCCGATGTTGCAGCCCACAGCGTCAGCCCCGAGTGAGTTCTAT | |
| | TCCTACCTATGCAAATCATAGAATGTATGCTAACTCGCCATGAAGCT | |
| | ATCCTGTGTTTCAGGGCGGTGAACTCACCCCAAGCGGAGGCACTTC | |
| | AGCAGCCTCTCCTGTCGTAGCAGCCATCGTGGCGCTGTTGAACGAT | |
| | GCCCGTCTCCGCGAAGGAAAACCCACGCTTGGATTTCTCAATCCGC | |
| | TGATTTACCTACACGCCTCCAAAGGGTTCACCGACATCACCTCGGG | |
| | CCAATCTGAAGGGTGCAACGGCAATAACACCCAGACGGGCAGTCCT | |
| | CTCCCAGGAGTATGCAGAACATCAAGAAGCCTTCTATCAGACGCCA | |
| | ATGCTAACTTGTGGATAGGCCGGCTTCATTGCAGGCGCACACTGGA | |
| | ACGCGACCAAGGGATGGGACCCGACGACTGGATTTGGTGTTCCAAA | |
| | CCTCAAAAAGCTCCTCGCACTTGTCCGGTTCTAA | |
| 57 | ATGTTCTTCAGTCGTGGAGCGCTTTCGCTCGCAGTGCTTTCACTGCT | Aspergillus |
| | CAGCTCCTCCGCCGCAGGGGAGGCTTTTGAGAAGCTGTCTGCCGTT | oryzae RIB40 |
| | CCAAAGGGATGGCACTATTCTAGTACCCCTAAAGGCAACACTGAGG | |
| | TTTGTCTGAAGATCGCCCTCGCGCAGAAGGATGCTGCTGGGTTCGA | |
| | AAAGACCGTCTTGGAGATGTCGGATCCCGACCACCCCAGCTACGGC | |
| | CAGCACTTCACCACCCACGACGAGATGAAGCGCATGCTTCTTCCCA | |
| | GAGATGACACCGTTGATGCCGTTCGACAATGGCTCGAAAACGGCGG | |
| | CGTGACCGACTTTACCCAGGATGCCGACTGGATCAACTTCTGTACT | |
| | ACCGTCGATACCGCGAACAAACTCTTGAATGCCCAGTTCAAATGGTA | |
| | CGTCAGCGATGTGAAGCACATCCGCCGTCTCAGAACACTGCAGTAC | |
| | GACGTCCCCGAGTCGGTCACCCCTCACATCAACACCATCCAACCGA | |
| | CCACCCGTTTTGGCAAGATTAGCCCCAAGAAGGCCGTTACCCACAG | |
| | CAAGCCCTCCCAGTTGGACGTGACCGCCCTTGCTGCCGCTGTCGTT | |
| | GCAAAGAACATCTCGCACTGTGATTCTATCATTACCCCCACCTGTCT | |
| | GAAGGAGCTTTACAACATTGGTGATTACCAGGCCGATGCAAACTCG | |
| | GGCAGCAAGATCGCCTTCGCCAGCTATCTGGAGGAGTACGCGCGC | |
| | TACGCTGACCTGGAGAACTTTGAGAACTACCTTGCTCCCTGGGCTA | |
| | AGGGCCAGAACTTCTCCGTTACCACCTTCAACGGCGGTCTCAATGA | |
| | TCAGAACTCCTCGTCCGATAGCGGTGAGGCCAACCTGGACCTGCAG | |
| | TACATTCTTGGTGTCAGCGCTCCACTGCCCGTTACTGAATTCAGCAC | |
| | CGGAGGCCGTGGTCCCCTCGTTCCTGATCTGACCCAGCCGGATCC | |
| | CAACTCTAACAGCAATGAGCCGTACCTTGAGTTCTTCCAGAATGTGT | |
| | TGAAGCTCGACCAGAAGGACCTCCCCCAGGTCATCTCGACCTCCTA | |
| | TGGAGAGAACGAACAGGAAATCCCCGAAAAGTACGCTCGCACCGTC | |
| | TGCAACCTGATCGCTCAGCTTGGCAGCCGCGGTGTCTCCGTTCTCT | |
| | TCTCCTCCGGTGACTCTGGTGTTGGCGAGGGCTGCATGACCAACGA | |
| | CGGCACCAACCGGACTCACTTCCCACCCCAGTTCCCCGCCGCTTGC | |
| | CCGTGGGTCACCTCCGTCGGCGCCACCTTCAAGACCACTCCCGAG | |
| | CGCGGCACCTACTTCTCCTCGGGCGGTTTCTCCGACTACTGGCCCC | |
| | GTCCCGAATGGCAGGATGAGGCCGTGAGCAGCTACCTCGAGACGA | |
| | TCGGCGACACTTTCAAGGGCCTCTACAACTCCTCCGGCCGTGCTTT | |
| | CCCCGACGTCGCAGCCCAGGGCATGAACTTCGCCGTCTACGACAA | |
| | GGGCACCTTGGGCGAGTTCGACGGCACCTCCGCCTCCGCCCCGGC | |
| | CTTCAGCGCCGTCATCGCTCTCCTGAACGATGCCCGTCTCCGCGCC | |
| | GGCAAGCCCACTCTCGGCTTCCTGAACCCCTGGTTGTACAAGACCG | |
| | GCCGCCAGGGTCTGCAAGATATCACCCTCGGTGCTAGCATTGGCTG | |
| | CACCGGTCGCGCTCGCTTCGGCGGCGCCCCTGACGGTGGTCCCGT | |
| | CGTGCCTTACGCTAGCTGGAACGCTACCCAGGGCTGGGATCCCGT | |
| | CACTGGTCTCGGAACTCCCGATTTCGCCGAGCTCAAGAAGCTTGCC | |
| | CTTGGCAACTAA | |
| 58 | ATGGCGCCCATCCTCTCGTTCCTTGTTGGCTCTCTCCTGGCGGTTC | Phaeosphaeria |
| | GCGCTCTTGCTGAGCCATTTGAGAAGCTGTTCAGCACCCCGGAAGG | nodorum |
| | ATGGAAGATGCAAGGTCTTGCTACCAATGAGCAGATCGTCAAGCTC | SN15 |
| | CAGATTGCTCTTCAGCAAGGCGATGTTGCAGGTTTCGAGCAACATG | |
| | TGATTGACATCTCAACGCCTAGCCACCCGAGCTATGGTGCTCACTAT | |
| | GGCTCGCATGAGGAGATGAAGAGGATGATCCAGCCAAGCAGCGAG | |
| | ACAGTCGCTTCTGTGTCTGCATGGCTGAAGGCCGCCGGTATCAACG | |
| | ACGCTGAGATTGACAGCGACTGGGTCACCTTCAAGACGACCGTTGG | |
| | CGTTGCCAACAAGATGCTCGACACCAAGTTCGCTTGGTACGTGAGC | |
| | GAGGAGGCCAAGCCCCGCAAGGTCCTTCGCACACTCGAGTACTCT | |
| | GTACCAGATGATGTTGCAGAACACATCAACTTGATCCAGCCCACTAC | |
| | TCGGTTTGCTGCGATCCGCCAAAACCACGAGGTTGCGCACGAGATT | |
| | GTTGGTCTTCAGTTCGCTGCTCTTGCCAACAACACCGTTAACTGCGA | |
| | TGCCACCATCACTCCCCAGTGCTTGAAGACTCTTTACAAGATTGACT | |
| | ACAAGGCCGATCCCAAGAGTGGTTCCAAGGTCGCTTTTGCTTCGTA | |
| | TTTGGAGCAGTACGCGCGTTACAATGACCTCGCCCTCTTCGAGAAG | |
| | GCCTTCCTCCCCGAAGCAGTTGGCCAGAACTTCTCTGTCGTCCAGT | |
| | TCAGCGGCGGTCTCAACGACCAGAACACCACGCAAGACAGTGGCG | |
| | AGGCCAACTTGGACTTGCAGTACATTGTCGGTGTCAGCGCTCCTCT | |
| | TCCCGTCACCGAGTTCAGCACCGGTGGTCGCGGCCCATGGGTCGC | |
| | TGACCTAGACCAACCTGACGAGGCGGACAGCGCCAACGAGCCCTA | |
| | CCTTGAATTCCTTCAGGGTGTGCTCAAACTTCCCCAGTCTGAGCTAC | |
| | CTCAGGTCATCTCCACATCCTATGGCGAGAATGAGCAGAGTGTACC | |
| | TAAGTCATACGCTCTCTCCGTCTGCAACTTGTTCGCCCAACTCGGTT | |
| | CCCGTGGCGTCTCCGTCATCTTCTCTTCTGGTGACAGCGGCCCTGG | |
| | ATCCGCATGCCAGAGCAACGACGGCAAGAACACGACCAAGTTCCAG | |
| | CCTCAGTACCCCGCTGCCTGCCCCTTTGTCACCTCGGTTGGATCGA | |
| | CTCGCTACCTCAACGAGACCGCAACCGGCTTCTCATCTGGTGGTTT | |
| | CTCCGACTACTGGAAGCGCCCATCGTACCAGGACGATGCTGTTAAG | |
| | GCGTATTTCCACCACCTCGGTGAGAAATTCAAGCCATACTTCAACCG | |
| | CCACGGCCGTGGATTCCCCGACGTTGCAACCCAGGGATATGGCTTC | |
| | CGCGTCTACGACCAGGGCAAGCTCAAGGGTCTCCAAGGTACTTCTG | |
| | CCTCCGCGCCTGCATTCGCCGGTGTGATTGGTCTCCTCAACGACGC | |
| | GCGATTGAAGGCGAAGAAGCCTACCTTGGGATTCCTAAACCCACTG | |
| | CTTTACTCTAACTCAGACGCGCTAAATGACATTGTTCTCGGTGGAAG | |
| | CAAGGGATGCGATGGTCATGCTCGCTTTAACGGGCCGCCAAATGGC | |
| | AGCCCAGTAATCCCATATGCGGGATGGAACGCGACTGCTGGGTGG | |
| | GATCCAGTGACTGGTCTTGGAACGCCGAACTTCCCCAAGCTTCTTA | |
| | AGGCTGCGGTGCCTAGCCGGTACAGGGCGTGA | |
| 59 | ATGGCGAAACTGACAGCTCTTGCCGGTCTCCTGACCCTTGCCAGCG | Trichoderma |
| | TGCAGGCAAATGCCGCCGTGCTCTTGGACAGCCTCGACAAGGTGC | atroviride IMI |
| | CTGTTGGATGGCAGGCTGCTTCGGCCCCGGCCCCGTCATCCAAGA | 206040 |
| | TCACCCTCCAAGTTGCCCTCACGCAGCAGAACATTGATCAGTTGGA | |
| | ATCAAAGCTCGCTGCCGTCTCCACGCCCAACTCCAGCAACTATGGA | |
| | AAGTACCTGGATGTCGATGAGATTAACCAAATCTTCGCTCCCAGCAG | |
| | CGCCAGCACCGCTGCTGTTGAGTCCTGGCTCAAGTCGTACGGCGT | |
| | GGACTACAAGGTGCAGGGCAGCAGCATCTGGTTCCAGACGGATGT | |
| | CTCCACGGCCAACAAGATGCTCAGCACAAACTTCCACACTTACACC | |
| | GACTCGGTTGGTGCCAAGAAAGTGCGAACTCTCCAGTACTCGGTCC | |
| | CCGAGACCCTGGCCGACCACATCGATCTGATTTCGCCCACAACCTA | |
| | CTTTGGCACGTCCAAGGCCATGCGGGCGTTGAAGATCCAGAACGC | |
| | GGCCTCTGCCGTCTCGCCCCTGGCTGCTCGTCAGGAGCCCTCCAG | |
| | CTGCAAGGGCACAATTGAGTTTGAGAACCGCACATTCAACGTCTTC | |
| | CAGCCCGACTGTCTCAGGACCGAGTACAGCGTCAACGGATACAAGC | |
| | CCTCAGCCAAGTCCGGTAGCAGGATTGGCTTCGGCTCTTTCCTGAA | |
| | CCAGAGCGCCAGCTCCTCAGATCTCGCTCTGTTCGAGAAGCACTTT | |
| | GGCTTTGCCAGCCAGGGCTTCTCCGTCGAGCTCATCAATGGCGGAT | |
| | CAAACCCCCAGCCGCCCACAGACGCCAATGACGGCGAGGCCAACC | |
| | TGGACGCCCAGAACATTGTGTCGTTTGTGCAGCCTCTGCCCATCAC | |
| | CGAGTTTATTGCTGGAGGAACTGCGCCGTACTTCCCAGACCCCGTT | |
| | GAGCCGGCTGGAACTCCCGATGAGAACGAGCCTTACCTCGAGTACT | |
| | ACGAGTACCTGCTCTCCAAGTCAAACAAGGAGCTTCCCCAAGTCAT | |
| | CACCAACTCCTACGGTGATGAGGAGCAGACTGTTCCCCAGGCATAT | |
| | GCCGTCCGCGTGTGCAACCTCATTGGATTGATGGGCCTTCGTGGTA | |
| | TCTCTATCCTCGAGTCATCCGGTGATGAGGGTGTTGGTGCCTCTTG | |
| | TCTCGCTACCAACAGCACCACCACTCCCCAGTTCAACCCCATCTTCC | |
| | CGGCTACATGCCCCTATGTCACCAGTGTTGGTGGAACCGTCAGCTT | |
| | CAACCCCGAGGTTGCCTGGGACGGCTCATCCGGAGGCTTCAGCTA | |
| | CTACTTCTCAAGACCTTGGTACCAGGAGGCCGCAGTCGGCACATAC | |
| | CTTAACAAGTATGTCAGCGAGGAGACCAAGGAATACTACAAGTCGT | |
| | ATGTCGACTTTTCCGGACGTGGCTTCCCCGATGTTGCAGCTCACAG | |
| | CGTGAGCCCCGATTACCCCGTGTTCCAAGGCGGCGAGCTTACCCC | |
| | CAGCGGCGGTACTTCTGCGGCCTCTCCCATCGTGGCCAGTGTTATT | |
| | GCCCTCCTGAACGATGCTCGTCTCCGTGCAGGCAAGCCTGCTCTCG | |
| | GATTCTTGAACCCTCTGATCTACGGATATGCCTACAAGGGCTTTACC | |
| | GATATCACGAGTGGCCAAGCTGTCGGCTGCAACGGCAACAACACTC | |
| | AAACTGGAGGCCCTCTTCCTGGTGCGGGTGTTATTCCAGGTGCTTT | |
| | CTGGAACGCGACCAAGGGCTGGGATCCTACAACTGGATTCGGTGTC | |
| | CCCAACTTCAAGAAGCTGCTTGAGCTTGTCCGATACATTTAG | |
| 60 | ATGCGTCTTCTCAAATTTGTGTGCCTGTTGGCATCAGTTGCCGCCGC | Arthroderma |
| | AAAGCCTACTCCAGGGGCGTCACACAAGGTCATTGAACATCTTGAC | benhamiae |
| | TTTGTTCCAGAAGGATGGCAGATGGTTGGTGCCGCGGACCCTGCTG | CBS 112371 |
| | CTATCATTGATTTCTGGCTTGCCATCGAGCGCGAAAACCCAGAAAAG | |
| | CTCTACGACACCATCTATGACGTCTCCACCCCTGGACGCGCACAAT | |
| | ATGGCAAACATTTGAAGCGTGAGGAATTGGATGACTTACTACGCCC | |
| | AAGGGCAGAGACGAGTGAGAGCATCATCAACTGGCTCACCAATGGT | |
| | GGAGTCAACCCACAACATATTCGGGATGAAGGGGACTGGGTCAGAT | |
| | TCTCTACCAATGTCAAGACTGCCGAAACGTTGATGAATACCCGCTTC | |
| | AACGTCTTCAAGGACAACCTAAATTCCGTTTCAAAAATTCGAACTTTG | |
| | GAGTATTCCGTCCCTGTAGCTATATCAGCTCATGTCCAAATGATCCA | |
| | GCCAACTACCTTATTTGGACGACAGAAGCCACAGAACAGTTTGATCC | |
| | TAAACCCCTTGACCAAGGATCTAGAATCCATGTCCGTTGAAGAATTT | |
| | GCTGCTTCTCAGTGCAGGTCCTTAGTGACTACTGCCTGCCTTCGAG | |
| | AATTGTACGGACTTGGTGACCGTGTCACTCAGGCTAGGGATGACAA | |
| | CCGTATTGGAGTATCCGGCTTTTTGGAGGAGTACGCCCAATACCGC | |
| | GATCTTGAGCTCTTCCTCTCTCGCTTTGAGCCATCCGCCAAAGGATT | |
| | TAATTTCAGTGAAGGCCTTATTGCCGGAGGAAAGAACACTCAGGGT | |
| | GGTCCTGGAAGCTCTACTGAGGCCAACCTTGATATGCAATATGTCG | |
| | TCGGTCTGTCCCACAAGGCAAAGGTCACCTATTACTCCACCGCTGG | |
| | CCGTGGCCCATTAATTCCCGATCTATCTCAGCCAAGCCAAGCTTCAA | |
| | ACAACAACGAACCATACCTTGAACAGCTGCGGTACCTCGTAAAGCT | |
| | CCCCAAGAACCAGCTTCCATCTGTATTGACAACTTCCTATGGAGACA | |
| | CAGAACAGAGCTTGCCCGCCAGCTATACCAAAGCCACTTGCGACCT | |
| | CTTTGCTCAGCTAGGAACTATGGGTGTGTCTGTTATCTTCAGCAGTG | |
| | GTGATACCGGGCCCGGAAGCTCATGCCAGACCAACGATGGCAAGA | |
| | ATGCGACTCGCTTCAACCCTATCTACCCAGCTTCTTGCCCGTTTGTG | |
| | ACCTCCATCGGTGGAACCGTTGGTACCGGTCCTGAGCGTGCAGTTT | |
| | CATTCTCCTCTGGTGGCTTCTCAGACAGGTTCCCCCGCCCACAATAT | |
| | CAGGATAACGCTGTTAAAGACTACCTGAAAATTTTGGGCAACCAGTG | |
| | GAGCGGATTGTTTGACCCCAACGGCCGTGCTTTCCCAGATATCGCA | |
| | GCTCAGGGATCAAATTATGCTGTCTATGACAAGGGAAGGATGACTG | |
| | GAGTCTCCGGCACCAGTGCATCCGCCCCTGCCATGGCTGCCATCAT | |
| | TGCCCAGCTTAACGATTTCCGACTGGCAAAGGGCTCTCCTGTGCTG | |
| | GGATTCTTGAACCCATGGATATATTCCAAGGGTTTCTCTGGCTTTAC | |
| | AGATATTGTTGATGGCGGTTCCAGGGGTTGCACTGGTTACGATATAT | |
| | ACAGCGGCTTGAAAGCGAAGAAGGTTCCCTACGCAAGCTGGAATGC | |
| | AACTAAGGGATGGGACCCAGTAACGGGATTTGGTACTCCCAACTTC | |
| | CAAGCTCTCACTAAAGTGCTGCCCTAA | |
| 61 | ATGTATATCACCTCATCCCGCCTCGTGCTGGCCTTAGCGGCACTTC | Fusarium |
| | CGACAGCATTTGGTAAATCATACTCCCACCATGCCGAAGCACCAAA | graminearum |
| | GGGATGGAAGGTCGACGACACCGCTCGTGTTGCCTCCACCGGTAA | PH-1 |
| | ACAACAGGTCTTCAGCATCGCACTGACCATGCAAAATGTTGATCAGC | |
| | TCGAGTCCAAGCTCCTTGACCTCTCCAGCCCCGACAGCAAGAACTA | |
| | TGGCCAGTGGATGTCTCAAAAGGACGTAACAACTGCTTTCTATCCTT | |
| | CGAAAGAAGCTGTTTCCAGTGTGACAAAGTGGCTCAAGTCCAAGGG | |
| | TGTCAAGCACTACAACGTCAACGGTGGTTTCATTGACTTTGCTCTCG | |
| | ATGTCAAGGGTGCCAATGCGCTACTTGATAGTGACTATCAATACTAC | |
| | ACCAAAGAGGGCCAGACCAAGTTGCGAACTCTGTCTTACTCTATCC | |
| | CTGATGATGTAGCCGAACACGTTCAGTTCGTCGACCCAAGCACCAA | |
| | CTTTGGCGGCACACTGGCTTTCGCCCCTGTCACTCACCCATCGCGT | |
| | ACTCTAACCGAGCGCAAGAACAAGCCCACCAAGAGCACAGTCGATG | |
| | CTTCATGCCAAACCAGCATCACACCCTCATGCTTGAAGCAGATGTAC | |
| | AACATTGGTGACTACACTCCCAAGGTCGAGTCTGGAAGCACTATTG | |
| | GTTTCAGCAGCTTCCTTGGCGAGTCCGCCATCTACTCCGATGTTTTC | |
| | CTGTTTGAGGAGAAGTTTGGAATTCCCACGCAGAACTTTACCACTGT | |
| | TCTCATCAACAACGGCACTGATGACCAGAACACTGCTCACAAGAACT | |
| | TTGGCGAGGCTGACTTGGATGCCGAGAACATTGTTGGAATTGCCCA | |
| | CCCTCTTCCCTTCACCCAGTACATCACTGGCGGTTCACCACCTTTTC | |
| | TTCCCAACATCGATCAGCCAACTGCTGCCGATAACCAGAACGAGCC | |
| | TTATGTGCCTTTCTTCCGCTACCTTCTATCGCAGAAGGAAGTCCCTG | |
| | CAGTTGTCTCTACCTCGTATGGTGACGAAGAAGATAGCGTCCCTCG | |
| | CGAATATGCTACCATGACCTGCAACCTGATTGGTCTTCTCGGACTTC | |
| | GAGGAATCAGTGTCATCTTCTCCTCTGGCGATATCGGCGTTGGTGC | |
| | TGGATGTCTCGGCCCTGACCACAAGACTGTCGAGTTCAACGCCATC | |
| | TTCCCTGCCACCTGCCCTTACCTCACCTCCGTCGGCGGTACCGTTG | |
| | ATGTCACCCCCGAAATCGCCTGGGAAGGTTCTTCTGGTGGTTTCAG | |
| | CAAGTACTTCCCCCGACCCAGCTACCAGGACAAGGCTGTCAAGACG | |
| | TACATGAAGACTGTCTCCAAGCAGACAAAGAAGTACTACGGCCCTTA | |
| | CACCAACTGGGAAGGCCGAGGCTTCCCTGATGTTGCTGGCCACAGT | |
| | GTCTCTCCCAACTATGAGGTTATCTATGCTGGTAAGCAGAGTGCAAG | |
| | CGGAGGTACCAGTGCTGCTGCTCCTGTTTGGGCTGCCATTGTCGGT | |
| | CTGCTCAACGATGCCCGTTTCAGAGCTGGGAAGCCAAGCTTGGGAT | |
| | GGTTGAACCCTCTTGTTTACAAGTATGGACCAAAGGTGTTGACTGAC | |
| | ATCACTGGTGGTTACGCCATTGGATGTGATGGCAACAACACCCAGT | |
| | CCGGAAAGCCTGAGCCTGCAGGATCCGGTATTGTGCCCGGTGCCA | |
| | GATGGAATGCCACTGCCGGATGGGATCCTGTCACTGGTTATGGTAC | |
| | ACCCGACTTTGGAAAGTTGAAGGATTTGGTTCTTAGCTTCTAA | |
| 62 | ATGCGTTCCTCCGGTCTTTACGCAGCACTGCTGTGCTCTCTGGCCG | Aspergillus |
| | CATCGACCAACGCAGTTGTTCATGAGAAGCTCGCCGCGGTCCCCTC | kawachii IFO |
| | GGGCTGGCACCATCTCGAAGATGCTGGCTCCGATCACCAGATTAGC | 4308 |
| | CTGTCGATCGCATTGGCACGCAAGAACCTCGATCAGCTTGAATCCA | |
| | AGCTGAAAGACTTGTCCACACCAGGTGAATCGCAGTATGGCCAGTG | |
| | GCTGGATCAAGAGGAAGTCGACACACTGTTCCCAGTGGCCAGCGA | |
| | CAAGGCCGTGATCAGCTGGTTGCGCAGCGCCAACATCACCCATATT | |
| | GCCCGGCAGGGCAGCTTGGTGAACTTTGCGACCACCGTCGACAAG | |
| | GTGAACAAGCTTCTCAACACCACTTTTGCTTACTACCAAAGAGGTTC | |
| | TTCCCAGAGACTGCGCACGACAGAGTACTCCATTCCCGATGATCTG | |
| | GTCGACTCGATCGACCTCATCTCCCCGACAACCTTTTTCGGCAAGG | |
| | AAAAGACCAGTGCTGGCCTGACCCAGCGGTCGCAGAAAGTCGACA | |
| | ACCATGTGGCCAAACGCTCCAACAGCTCGTCCTGCGCCGATACCAT | |
| | CACGTTATCCTGCCTGAAGGAGATGTACAACTTTGGCAACTACACTC | |
| | CCAGCGCCTCGTCAGGAAGCAAGCTGGGATTCGCCAGCTTCCTGAA | |
| | CGAGTCCGCCTCGTATTCCGATCTTGCCAAGTTCGAGAGACTGTTC | |
| | AACTTGCCGTCTCAGAACTTCTCCGTGGAGCTGATCAACGGCGGCG | |
| | TCAATGACCAGAACCAATCGACGGCTTCTCTGACCGAGGCTGACCT | |
| | CGATGTGGAATTGCTCGTTGGCGTAGGTCATCCTCTTCCGGTGACC | |
| | GAGTTTATCACTTCTGGCGAACCTCCTTTCATTCCCGACCCCGATGA | |
| | GCCGAGTGCCGCCGATAATGAGAATGAGCCTTACCTTCAGTACTAC | |
| | GAGTACCTCCTCTCCAAGCCCAACTCGGCCCTGCCCCAAGTGATTT | |
| | CCAACTCCTACGGTGACGACGAACAGACCGTTCCAGAATACTACGC | |
| | CAAGCGAGTCTGCAACCTGATCGGACTGGTCGGCCTGCGCGGCAT | |
| | CAGCGTCCTGGAATCATCCGGTGACGAAGGAATTGGATCTGGCTGC | |
| | CGCACCACCGACGGCACTAACAGCACCCAATTCAATCCCATCTTCC | |
| | CCGCCACCTGTCCCTACGTGACCGCCGTAGGAGGCACCATGTCCTA | |
| | CGCGCCCGAAATTGCCTGGGAAGCCAGTTCCGGTGGTTTCAGCAAC | |
| | TACTTCGAGCGAGCCTGGTTCCAGAAGGAAGCCGTGCAGAACTACC | |
| | TGGCGAACCACATCACCAACGAGACGAAGCAGTATTACTCACAATT | |
| | CGCTAACTTTAGCGGTCGCGGATTTCCCGATGTTTCGGCCCATAGC | |
| | TTTGAGCCTTCGTACGAAGTTATCTTCTACGGCGCCCGTTACGGCTC | |
| | CGGCGGTACTTCCGCCGCATGTCCTCTGTTCTCTGCGCTAGTGGGC | |
| | ATGTTGAACGATGCTCGTCTGCGGGCGGGCAAGTCCACGCTTGGTT | |
| | TCTTGAACCCCCTGCTGTACAGTAAGGGGTACAAGGCGCTGACAGA | |
| | TGTCACGGCGGGACAATCGATCGGGTGCAATGGCATTGATCCGCA | |
| | GAGTGATGAGGCTGTTGCGGGCGCGGGCATTATCCCGTGGGCGCA | |
| | TTGGAATGCCACAGTCGGATGGGATCCGGTGACGGGATTGGGACTT | |
| | CCTGATTTTGAGAAGTTGAGGCAGTTGGTGCTGTCGTTGTAG | |
| 63 | ATGAGTCGAAATCTCCTCGTTGGTGCTGGCCTGTTGGCCCTCGCCC | Talaromyces |
| | AATTGAGCGGTCAAGCTCTCGCTGCCGCTGCCCTCGTCGGCCATGA | stipitatus |
| | ATCCCTAGCTGCGCTGCCAGTTGGCTGGGATAAGGTCAGCACGCCA | ATCC 10500 |
| | GCTGCAGGGACGAACATTCAATTGTCCGTCGCCCTCGCTCTGCAAA | |
| | ACATCGAGCAGCTGGAAGACCACTTGAAGTCTGTGTCAACCCCCGG | |
| | TTCTGCCAGCTACGGTCAGTACCTGGATTCCGACGGTATTGCCGCT | |
| | CAATACGGTCCCAGCGACGCATCCGTTGAGGCTGTCACCAACTGGC | |
| | TGAAGGAGGCCGGTGTCACTGACATCTACAACAACGGCCAGTCGAT | |
| | TCACTTCGCAACCAGTGTCAGCAAGGCCAACAGCTTGCTCGGGGCC | |
| | GATTTCAACTACTATTCTGATGGTAGTGCGACCAAGTTGCGTACCTT | |
| | AGCTTATTCCGTTCCCAGTGACCTCAAAGAGGCCATCGACCTTGTCT | |
| | CGCCCACCACCTATTTCGGCAAGACCACTGCTTCTCGTAGCATCCA | |
| | GGCTTACAAGAACAAGCGCGCCTCTACTACTTCCAAGTCTGGATCG | |
| | AGCTCTGTGCAAGTATCTGCTTCCTGCCAGACCAGCATCACTCCTG | |
| | CCTGCTTGAAACAGATGTACAATGTTGGCAACTACACACCCAGCGT | |
| | CGCTCACGGCAGTCGTGTCGGATTCGGTAGCTTCTTGAATCAATCT | |
| | GCCATCTTTGACGACTTGTTCACCTACGAAAAGGTCAATGATATTCC | |
| | ATCACAGAATTTCACTAAGGTGATTATTGCAAATGCATCCAACAGCC | |
| | AAGATGCCAGCGATGGCAACTACGGCGAAGCCAACCTTGACGTGCA | |
| | AAACATTGTCGGCATCTCTCATCCTCTCCCCGTGACTGAATTCCTCA | |
| | CTGGTGGCTCACCTCCCTTCGTTGCTAGCCTCGACACCCCTACCAA | |
| | CCAGAACGAGCCATATATTCCTTACTACGAATATCTTTTGTCTCAGAA | |
| | GAACGAGGATCTCCCCCAGGTCATTTCCAACTCTTACGGAGACGAC | |
| | GAGCAGTCTGTGCCGTACAAGTATGCCATCCGTGCATGCAACCTGA | |
| | TCGGCCTGACAGGTTTACGAGGTATCTCGGTCTTGGAATCCAGCGG | |
| | TGATCTCGGCGTTGGAGCCGGCTGTCGCAGCAACGATGGCAAGAA | |
| | CAAGACTCAATTTGACCCCATCTTCCCTGCCACTTGCCCCTACGTTA | |
| | CCTCTGTTGGTGGTACCCAATCCGTTACCCCTGAAATTGCCTGGGT | |
| | CGCCAGCTCCGGTGGTTTCAGCAACTACTTCCCTCGTACCTGGTAC | |
| | CAGGAACCCGCAATTCAGACCTATCTCGGACTCCTTGACGATGAGA | |
| | CCAAGACATACTATTCTCAATACACCAACTTTGAAGGCCGTGGTTTC | |
| | CCCGATGTTTCCGCCCACAGCTTGACCCCTGATTACCAGGTCGTCG | |
| | GTGGTGGCTATCTCCAGCCAAGCGGTGGTACTTCCGCTGCTTCTCC | |
| | TGTCTTTGCCGGCATCATTGCGCTTTTGAACGACGCTCGTCTCGCT | |
| | GCTGGCAAGCCCACTCTTGGCTTCTTGAACCCGTTCTTCTACCTTTA | |
| | TGGATACAAGGGTTTAAACGATATCACTGGAGGACAGTCAGTGGGT | |
| | TGCAACGGTATCAACGGCCAAACTGGGGCTCCTGTTCCCGGTGGTG | |
| | GCATTGTTCCTGGAGCGGCCTGGAACTCTACTACTGGCTGGGACCC | |
| | AGCCACTGGTCTCGGAACACCCGACTTCCAGAAGTTGAAAGAACTC | |
| | GTACTTAGCTTTTAA | |
| 64 | ATGTATATCTCCTCCCAAAATCTGGTACTCGCCTTATCGGCGCTGCC | Fusarium |
| | TTCAGCATTTGGCAAATCCTTCTCTCACCATGCTGAAGCTCCTCAAG | oxysporum f. |
| | GCTGGCAAGTCCAAAAGACTGCCAAAGTCGCTTCCAACACGCAGCA | sp. cubense |
| | TGTCTTCAGTCTTGCACTAACCATGCAAAACGTGGATCAGCTCGAAT | race 4 |
| | CCAAGCTTCTTGACCTCTCCAGCCCCGACAGCGCCAACTACGGTAA | |
| | CTGGCTCTCCCACGATGAGCTCACAAGCACTTTCTCTCCTTCCAAG | |
| | GAGGCGGTGGCTAGTGTGACAAAGTGGCTCAAGTCAAAGGGCATC | |
| | AAGCACTACAAGGTCAACGGTGCTTTCATTGACTTTGCTGCTGATGT | |
| | TGAGAAGGCCAATACGCTTCTCGGAGGTGATTACCAGTACTACACT | |
| | AAGGATGGTCAGACGAAGCTGAGAACGCTGTCTTACTCCATTCCTG | |
| | ATGATGTCGCCGGTCACGTTCAATTTGTTGATCCTAGCACAAACTTC | |
| | GGTGGCACCGTTGCGTTCAACCCTGTGCCTCACCCCTCGCGCACC | |
| | CTCCAAGAGCGCAAGGTCTCTCCCTCCAAGAGCACCGTTGATGCTT | |
| | CATGCCAGACAAGCATCACCCCTTCTTGCCTCAAGCAGATGTACAA | |
| | CATTGGAGACTACACTCCCGATGCCAAGTCTGGAAGTGAGATTGGT | |
| | TTCAGCAGCTTTCTCGGCCAGGCTGCTATTTACTCTGATGTCTTCAA | |
| | GTTTGAGGAGCTGTTTGGTATTCCTAAGCAGAACTACACCACTATTC | |
| | TGATCAACAATGGCACCGATGATCAGAATACTGCGCATGGAAACTTT | |
| | GGAGAGGCTAACCTTGATGCTGAGAACATTGTTGGAATCGCTCATC | |
| | CTCTTCCTTTCAAGCAGTACATTACTGGAGGTTCACCACCTTTCGTT | |
| | CCCAACATCGATCAGCCCACCGAGAAGGATAACCAGAACGAGCCCT | |
| | ACGTGCCTTTCTTCCGTTACCTCTTGGGCCAGAAGGATCTCCCAGC | |
| | CGTCATCTCCACTTCCTACGGCGATGAAGAAGACAGCGTTCCTCGT | |
| | GAGTATGCTACACTCACCTGCAACATGATCGGTCTTCTCGGTCTCC | |
| | GTGGCATCAGTGTCATCTTCTCTTCCGGTGACATCGGTGTCGGTTC | |
| | CGGCTGCCTTGCTCCCGACTACAAGACCGTCGAGTTCAATGCCATC | |
| | TTCCCCGCCACATGCCCCTACCTCACCTCCGTCGGCGGTACCGTCG | |
| | ACGTCACCCCCGAGATCGCCTGGGAGGGATCCTCCGGCGGATTCA | |
| | GCAAGTACTTCCCCCGACCCAGCTACCAGGACAAGGCCATCAAGAA | |
| | GTACATGAAGACAGTCTCCAAGGAGACCAAGAAGTACTACGGCCCT | |
| | TACACCAACTGGGAGGGCCGAGGTTTCCCTGATGTCGCTGGACACA | |
| | GTGTTGCGCCTGACTACGAGGTTATCTACAATGGTAAGCAGGCTCG | |
| | AAGTGGAGGTACCAGCGCTGCTGCCCCTGTTTGGGCTGCTATCGTT | |
| | GGTCTGTTGAACGATGCCCGCTTCAAGGCTGGTAAGAAGAGCTTGG | |
| | GATGGTTGAACCCTCTTATCTACAAGCATGGACCCAAGGTCTTGACT | |
| | GACATCACCGGTGGCTATGCTATTGGATGTGACGGTAACAACACTC | |
| | AGTCTGGAAAGCCCGAGCCCGCTGGATCTGGTCTTGTTCCCGGTGC | |
| | TCGATGGAACGCCACAGCTGGATGGGATCCTACCACTGGCTATGGA | |
| | ACTCCCAACTTCCAGAAGTTGAAGGACTTGGTTCTCAGCTTGTAA | |
| 65 | ATGCCTAAGTCCACAGCGCTTCGGCTTGTTAGCCTCCTTTCCCTGG | Trichoderma |
| | CCAGTGTGCCGATATCTGCCTCCGTCCTTGTGGAAAGTCTCGAAAA | virens |
| | GCTGCCTCACGGATGGAAAGCTGCTTCGGCTCCTAGCCCTTCCTCC | Gv29-8 |
| | CAGATAACCCTACAAGTCGCTCTTACGCAGCAGAACATCGATCAGC | |
| | TGGAATCGAGGCTCGCGGCTGTATCCACACCAAATTCCAAGACATA | |
| | CGGAAATTATCTGGATCTTGATGAGATCAATGAGATCTTCGCGCCAA | |
| | GCGATGCCAGCAGCGCAGCCGTGGAGTCTTGGCTCCATTCTCACG | |
| | GTGTGACAAAATACACGAAGCAAGGCAGCAGTATCTGGTTCCAAAC | |
| | CGAAGTTTCTACAGCAAATGCAATGTTGAGCACAAACTTCCACACTT | |
| | ACAGTGATGCTGCTGGCGTTAAGAAGTTGCGAACTCTTCAGTATTCA | |
| | ATTCCGGAGAGTCTTGTGGGCCATGTCGATCTCATCTCACCCACGA | |
| | CCTACTTTGGCACCTCTAACGCTATGAGAGCTTTGAGATCTAAAAGC | |
| | GTGGCTTCAGTTGCTCAAAGTGTGGCAGCCCGCCAAGAACCTTCTA | |
| | GCTGCAAGGGAACTCTGGTTTTCGAAGGAAGAACGTTCAATGTCTT | |
| | CCAACCAGATTGTCTTAGGACAGAGTACAATGTCAATGGATACACTC | |
| | CATCAGCCAAGTCTGGTAGTAGAATAGGATTTGGTTCCTTCTTAAAC | |
| | CAAAGTGCAAGCTTTTCAGACCTCGCACTCTTTGAAAAACACTTTGG | |
| | GTTTTCCAGCCAAAATTTCTCCGTCGTTCTGATCAATGGTGGAACGG | |
| | ACCTGCCCCAACCACCCTCTGACGACAACGATGGCGAGGCCAATTT | |
| | GGATGTCCAAAACATTTTGACAATCGCACACCCTCTGCCCATCACTG | |
| | AATTCATCACTGCCGGAAGCCCGCCGTACTTCCCAGATCCCGTTGA | |
| | ACCTGCAGGAACTCCCGATGAGAACGAGCCTTACTTGCAGTACTTT | |
| | GAGTATCTGTTGTCGAAGCCCAACAGAGATCTTCCTCAGGTCATTAC | |
| | CAACTCTTACGGTGATGAGGAGCAAACAGTACCTCAGGCTTATGCT | |
| | GTCCGAGTGTGCAACCTAATTGGATTGATGGGACTGCGTGGTATCA | |
| | GTATCCTCGAGTCCTCCGGCGATGAGGGAGTGGGTGCTTCCTGCG | |
| | TTGCTACCAACAGCACCACTCCTCAATTTAACCCCATTTTCCCGGCA | |
| | ACATGCCCCTATGTCACTAGCGTAGGTGGAACTGTGAACTTCAACC | |
| | CAGAAGTTGCCTGGGACGGTTCATCTGGAGGTTTCAGCTACTATTTC | |
| | TCCAGGCCATGGTACCAAGAGGAAGCAGTTGGAAACTACCTAGAGA | |
| | AGCATGTCAGCGCCGAAACAAAGAAGTACTACGGGCCTTATGTCGA | |
| | TTTCTCTGGACGTGGCTTCCCTGATGTTGCAGCTCACAGCGTGAGC | |
| | CCCGATTATCCTGTGTTCCAAGGCGGCCAGCTCACTCCTAGCGGAG | |
| | GCACTTCTGCGGCTTCTCCCGTCGTAGCCAGTATCATTGCCCTTCT | |
| | GAACGATGCACGCCTCCGTGAAGGCAAGCCCACACTTGGGTTCCTG | |
| | AACCCGCTGATTTACCAATATGCTTACAAGGGTTTCACGGATATCAC | |
| | ATCCGGCCAGTCTGATGGCTGCAATGGCAACAACACCCAAACGGAT | |
| | GCCCCTCTTCCTGGAGCTGGCGTTGTCCTAGGAGCACACTGGAATG | |
| | CGACCAAAGGATGGGATCCTACGACAGGATTTGGTGTCCCTAACTT | |
| | TAAGAAGCTACTCGAGCTGATCCGATATATATAG | |
| 66 | ATGGCTAAACTGACGGCACTTCGGCTCGTCAGCCTTCTTTGCCTTG | Trichoderma |
| | CGGCTGCGCAGGCCTCTGCTGCTGTGCTCGTGGAAAGCCTCAAAC | atroviride IMI |
| | AAGTGCCCAACGGGTGGAATGCAGTCTCGACCCCAGACCCTTCGAC | 206040 |
| | ATCGATTGTCTTGCAAATCGCCCTCGCGCAACAGAATATCGATGAAT | |
| | TGGAATGGCGTCTCGCGGCTGTATCCACGCCCAACTCTGGCAATTA | |
| | TGGCAAATACCTGGATATTGGAGAGATTGAAGGAATTTTCGCCCCAA | |
| | GCAATGCCTCTTACAAAGCCGTGGCATCGTGGCTCCAGTCTCATGG | |
| | GGTGAAGAACTTCGTCAAACAAGCCGGCAGTATTTGGTTCTACACTA | |
| | CTGTCTCTACCGCAAACAAGATGCTTAGCACAGATTTCAAACACTAT | |
| | AGCGATCCTGTTGGCATTGAGAAGCTGCGTACTCTTCAGTACTCGAT | |
| | CCCAGAAGAACTAGTCGGCCATGTTGATCTCATCTCGCCTACAACAT | |
| | ATTTTGGAAACAACCACCCCGCGACAGCGAGAACACCCAACATGAA | |
| | GGCCATTAACGTAACCTACCAAATCTTTCACCCAGACTGCCTTAAAA | |
| | CGAAATACGGCGTTGATGGCTATGCCCCATCTCCAAGATGTGGCAG | |
| | CAGGATTGGTTTTGGCTCATTCCTCAACGAAACTGCCAGTTATTCGG | |
| | ATCTTGCGCAGTTTGAGAAGTACTTTGACCTTCCCAACCAAAACCTT | |
| | TCCACCTTATTGATCAATGGCGCAATCGACGTTCAGCCACCTTCCAA | |
| | CAAAAACGACAGCGAGGCCAACATGGACGTTCAGACCATCTTGACC | |
| | TTTGTCCAACCTCTTCCTATTACTGAGTTTGTTGTTGCCGGAATCCC | |
| | GCCGTATATTCCTGATGCGGCTTTGCCGATCGGCGACCCTGTCCAA | |
| | AACGAGCCGTGGCTGGAATACTTTGAGTTTTTGATGTCCAGGACCA | |
| | ACGCAGAGCTTCCCCAGGTCATTGCCAACTCATACGGTGACGAGGA | |
| | ACAAACGGTACCACAGGCGTATGCCGTCCGAGTATGCAACCAGATT | |
| | GGGCTGTTGGGCCTTCGCGGTATATCCGTTATCGCATCATCTGGCG | |
| | ATACGGGTGTTGGAATGTCTTGTATGGCTTCGAACAGCACTACTCCT | |
| | CAGTTTAACCCCATGTTCCCGGCTTCGTGTCCTTATATCACCACTGT | |
| | CGGTGGAACTCAGCACCTTGATAATGAGATTGCTTGGGAGCTTTCAT | |
| | CGGGAGGCTTCAGTAACTATTTCACAAGGCCATGGTATCAAGAAGA | |
| | CGCAGCCAAAACATATCTTGAACGTCATGTCAGCACCGAGACAAAG | |
| | GCATATTACGAACGTTACGCCAATTTCTTGGGACGCGGCTTTCCCG | |
| | ACGTTGCAGCACTTAGTCTCAACCCCGATTATCCAGTGATTATTGGC | |
| | GGAGAACTTGGTCCCAATGGAGGCACTTCTGCGGCCGCACCCGTC | |
| | GTCGCTAGTATTATTGCACTCTTGAACGATGCACGCCTTTGCCTAGG | |
| | CAAACCTGCCCTTGGGTTCTTGAACCCCCTGATCTATCAATATGCTG | |
| | ATAAGGGTGGCTTCACGGATATCACGTCCGGCCAGTCTTGGGGCTG | |
| | TGCCGGAAATACCACTCAGACGGGGCCTCCTCCCCCTGGAGCTGG | |
| | TGTCATTCCGGGGGCACACTGGAATGCGACCAAGGGATGGGATCC | |
| | TGTAACAGGATTTGGAACCCCGAACTTCAAGAAATTACTCTCACTGG | |
| | CCCTGTCCGTCTAA | |
| 67 | ATGTTTTGGCGTCCAGCTTTTGTCCTTCTTCTCGCTCAGCTTGTCAC | Agaricus |
| | TGCTAGTCCTTTAGCTCGACGCTGGGATGATTTCGCAGAAAAACATG | bisporus var. |
| | CCTGGGTTGAAGTTCCTCGCGGGTGGGAAATGGTCTCCGAGGCTC | burnettii |
| | CCAGTGACCATACCTTTGATCTTCGCATTGGAGTAAAGTCAAGTGGC | JB137-S8 |
| | ATGGAGCAGCTCATTGAAAACTTGATGCAAACCAGCGATCCTACTCA | |
| | TTCCAGATATGGTCAACATCTTAGTAAAGAAGAGCTCCATGATTTCG | |
| | TTCAGCCTCATCCTGATTCTACCGGAGCGGTCGAAGCATGGCTTGA | |
| | AGATTTCGGTATCTCCGATGATTTCATTGATCGTACTGGAAGTGGCA | |
| | ACTGGGTTACTGTTCGAGTTTCAGTAGCCCAGGCTGAACGTATGCTT | |
| | GGTACCAAGTATAACGTCTACCGCCATTCTGAATCAGGGGAATCGG | |
| | TTGTACGAACAATGTCTTATTCGCTTCCCAGCGAACTTCACTCCCAC | |
| | ATAGATGTTGTCGCACCCACCACTTATTTCGGCACGATGAAAAGCAT | |
| | GCGGGTGACCAGCTTCTTACAGCCGGAAATAGAGCCTGTTGACCCA | |
| | AGCGCTAAACCATCGGCTGCTCCAGCTTCCTGTTTGAGTACCACTG | |
| | TCATAACCCCCGATTGCCTCCGTGACCTTTATAATACGGCTGACTAC | |
| | GTTCCTTCCGCCACTTCACGGAATGCCATTGGTATTGCTGGGTACTT | |
| | GGATCGTTCAAATCGTGCAGATCTTCAGACTTTCTTCCGACGCTTCC | |
| | GGCCCGATGCCGTTGGCTTCAATTACACGACTGTCCAACTAAATGG | |
| | CGGAGGAGACGACCAGAATGATCCCGGTGTAGAGGCCAACCTCGA | |
| | TATTCAATACGCCGCTGGTATTGCTTTCCCCACACCAGCTACATACT | |
| | GGAGTACTGGCGGCTCTCCACCTTTCATTCCAGATACTCAAACCCC | |
| | GACAAACACCAATGAGCCCTACCTGGATTGGATCAATTTTGTCCTAG | |
| | GCCAGGACGAGATTCCACAGGTGATTTCAACGTCCTATGGTGACGA | |
| | CGAGCAAACAGTTCCTGAAGATTACGCTACTAGCGTGTGTAATCTCT | |
| | TCGCGCAACTCGGCAGCCGTGGCGTTACAGTATTCTTCTCCAGCGG | |
| | TGACTTTGGTGTTGGTGGTGGAGATTGCCTCACGAATGATGGCTCA | |
| | AACCAAGTCCTTTTCCAGCCGGCTTTCCCCGCTTCCTGCCCATTCGT | |
| | AACAGCTGTTGGCGGAACTGTCAGGCTTGATCCTGAGATTGCTGTC | |
| | AGTTTCTCTGGAGGAGGCTTTTCCCGTTACTTCTCCAGGCCATCGTA | |
| | CCAGAATCAAACTGTGGCTCAATTTGTTTCTAATCTTGGGAATACATT | |
| | CAACGGACTCTACAATAAAAATGGAAGGGCCTACCCAGATCTTGCA | |
| | GCACAGGGCAATGGCTTCCAAGTTGTTATAGACGGCATCGTCCGTT | |
| | CGGTTGGAGGGACCAGCGCCAGCTCTCCGACGGTTGCCGGTATCT | |
| | TTGCGCTTTTGAATGACTTCAAGCTCTCAAGAGGCCAGTCGACACTC | |
| | GGATTTATCAACCCACTTATATACTCCTCCGCTACATCCGGCTTCAA | |
| | TGACATCAGGGCGGGTACAAACCCTGGTTGTGGTACTCGCGGATTT | |
| | ACCGCTGGTACTGGTTGGGATCCGGTCACTGGTCTGGGCACTCCC | |
| | GATTTTTTGAGGCTTCAGGGACTTATTTAA | |
| 68 | ATGCTCGACCGTATCCTTCTCCCCCTCGGCCTCCTGGCCTCCCTTG | Magnaporthe |
| | CCACCGCTCGTGTCTTTGACAGCCTACCCCACCCTCCCCGAGGCTG | oryzae 70-15 |
| | GTCATACTCGCACGCGGCGGAATCGACGGAGCCGCTGACCCTGCG | |
| | CATCGCCCTCCGCCAGCAAAATGCCGCCGCCCTGGAGCAGGTGGT | |
| | GCTGCAGGTCTCGAACCCCAGGCACGCCAATTACGGCCAGCACCT | |
| | GACGCGCGACGAGCTGCGCAGCTACACGGCGCCCACGCCGCGGG | |
| | CCGTCCGCAGCGTGACGTCGTGGCTGGTCGACAACGGCGTCGACG | |
| | ACTACACGGTCGAGCACGACTGGGTGACGCTGCGCACGACGGTCG | |
| | GGGCCGCGGACAGGCTGCTCGGCGCAGACTTTGCCTGGTATGCCG | |
| | GCCCGGGCGAGACGCTGCAGCTGCGGACGCTCTCGTACGGCGTC | |
| | GACGACTCGGTGGCGCCGCACGTCGACCTCGTGCAGCCCACGACG | |
| | CGGTTTGGCGGTCCCGTCGGGCAGGCGTCGCACATCTTCAAGCAG | |
| | GACGACTTTGACGAGCAGCAGCTCAAGACCTTGTCGGTGGGGTTCC | |
| | AGGTCATGGCTGACCTGCCGGCCAACGGGCCTGGGTCGATCAAGG | |
| | CGGCATGTAACGAGTCTGGCGTGACGCCCCTGTGCCTGCGAACTCT | |
| | GTACAGGGTCAACTACAAGCCGGCAACCACGGGGAACCTGGTCGC | |
| | TTTCGCGTCGTTCCTGGAGCAGTACGCCAGGTACAGTGATCAGCAG | |
| | GCATTCACTCAGCGGGTCCTTGGCCCTGGTGTTCCGTTGCAGAACT | |
| | TTTCGGTCGAAACGGTCAACGGTGGAGCCAATGACCAGCAGAGCAA | |
| | ACTTGACAGCGGCGAGGCGAACCTCGATCTGCAGTACGTCATGGCA | |
| | ATGAGCCACCCTATTCCAATTTTGGAGTACAGCACTGGAGGCAGAG | |
| | GACCCCTCGTCCCAACTCTGGACCAGCCCAACGCCAACAACAGCAG | |
| | CAATGAGCCTTACCTGGAGTTCCTGACGTACCTCCTGGCCCAACCC | |
| | GACTCAGCCATCCCTCAGACCCTGTCGGTGTCGTATGGCGAGGAG | |
| | GAACAGTCGGTGCCGCGCGACTACGCCATCAAGGTTTGCAACATGT | |
| | TCATGCAGCTCGGCGCCCGCGGCGTGTCGGTTATGTTTTCGTCGG | |
| | GCGACTCGGGCCCGGGTAATGACTGTGTTCGAGCCTCGGACAACG | |
| | CAACCTTTTTTGGCTCAACATTCCCCGCAGGCTGCCCCTACGTCAC | |
| | GTCGGTGGGCTCCACCGTCGGCTTCGAGCCGGAGCGCGCCGTCTC | |
| | CTTTTCCTCGGGCGGCTTCAGCATTTACCACGCTCGCCCCGACTAC | |
| | CAAAACGAAGTGGTCCCCAAGTACATTGAATCGATCAAGGCTTCGG | |
| | GCTACGAAAAGTTCTTTGACGGCAACGGCCGCGGAATTCCCGACGT | |
| | GGCTGCCCAGGGCGCCCGCTTCGTCGTCATCGACAAGGGCCGCGT | |
| | TTCTCTAATCTCGGGGACCAGCGCCAGCTCACCTGCGTTTGCTGGC | |
| | ATGGTGGCGCTCGTCAACGCCGCCCGCAAGTCAAAGGACATGCCG | |
| | GCCTTGGGCTTCCTCAACCCCATGCTGTACCAGAACGCCGCGGCCA | |
| | TGACGGACATTGTCAACGGCGCTGGCATCGGCTGCAGGAAGCAAC | |
| | GTACAGAATTCCCGAATGGCGCCAGGTTCAACGCCACGGCCGGCT | |
| | GGGATCCCGTCACAGGGCTGGGGACGCCGTTGTTTGACAAGCTGC | |
| | TGGCTGTTGGCGCACCTGGAGTTCCCAACGCGTGA | |
| 69 | ATGCGTAGCCAGTTGCTCTTCTGCACAGCATTTGCTGCTCTCCAGTC | Togninia |
| | GCTTGTGGAGGGCAGCGATGTGGTGTTGGAGTCATTGCGAGAGGT | minima |
| | CCCTCAGGGCTGGAAGAGGCTTCGAGATGCGGACCCCGAGCAGTC | UCRPA7 |
| | CATCAAGCTGCGCATTGCGCTTGAGCAGCCTAACCTGGACCTGTTC | |
| | GAGCAGACCCTCTACGACATCTCGTCACCGGATCACCCAAAATATG | |
| | GCCAGCATCTCAAGAGCCACGAGTTACGGGATATTATGGCACCTCG | |
| | CGAGGAGTCAACTGCTGCTGTCATCGCTTGGCTGCAAGACGCTGG | |
| | GCTTTCTGGCTCGCAGATTGAGGACGACAGCGACTGGATCAACATC | |
| | CAGACGACAGTCGCCCAAGCCAACGACATGCTGAACACGACTTTCG | |
| | GTCTCTTCGCCCAGGAAGGCACCGAGGTCAATCGAATTCGAGCTCT | |
| | GGCATATTCCGTGCCTGAGGAGATCGTCCCTCACGTCAAGATGATT | |
| | GCTCCCATCATCCGCTTCGGTCAGTTGAGACCTCAGATGAGCCACA | |
| | TCTTCTCGCATGAGAAAGTCGAGGAGACCCCGTCTATTGGCACCAT | |
| | CAAGGCCGCCGCTATCCCATCTGTGGATCTTAACGTCACCGCTTGC | |
| | AATGCCAGCATCACCCCCGAGTGCCTCCGAGCGCTTTACAACGTTG | |
| | GTGATTACGAGGCGGACCCATCGAAGAAGTCTCTTTTCGGAGTCTG | |
| | TGGCTACTTGGAGCAATATGCCAAGCACGATCAGCTGGCCAAGTTT | |
| | GAGCAGACCTACGCTCCGTATGCTATCGGTGCCGACTTCAGCGTCG | |
| | TGACCATCAATGGCGGAGGCGACAACCAGACCAGTACGATCGATGA | |
| | TGGAGAAGCCAACCTGGATATGCAGTATGCTGTCAGCATGGCATAC | |
| | AAGACGCCAATCACATACTATTCAACTGGGGGTCGAGGACCTCTTG | |
| | TTCCAGATCTCGACCAACCTGATCCCAACGACGTCTCAAACGAGCC | |
| | GTACCTTGATTTTGTGAGCTACCTTCTCAAGCTGCCCGACTCCAAAT | |
| | TGCCGCAGACCATCACAACTTCGTACGGAGAGGATGAGCAATCCGT | |
| | TCCACGCTCCTACGTGGAGAAGGTCTGCACCATGTTCGGCGCGCTC | |
| | GGTGCCCGAGGCGTGTCTGTGATCTTCTCCTCTGGTGATACCGGTG | |
| | TCGGCTCAGCGTGCCAGACCAACGACGGCAAGAACACCACCCGCT | |
| | TCCTGCCTATATTCCCTGCTGCGTGCCCTTATGTGACCTCGGTTGGA | |
| | GGCACTCGCTATGTCGACCCGGAAGTCGCTGTGTCCTTCTCGTCTG | |
| | GAGGCTTCTCGGACATCTTCCCTACGCCACTCTACCAGAAGGGCGC | |
| | TGTCTCTGGCTACCTGAAGATCCTCGGCGATCGCTGGAAGGGCCTC | |
| | TATAACCCTCACGGCCGCGGTTTCCCTGACGTCTCCGGACAGAGTG | |
| | TCAGATACCACGTCTTCGACTACGGCAAGGACGTCATGTACTCTGG | |
| | CACAAGTGCCTCTGCACCGATGTTCGCCGCGCTTGTCTCGCTGCTG | |
| | AACAACGCCCGTCTCGCAAAGAAGTTGCCGCCCATGGGATTCCTGA | |
| | ATCCCTGGCTGTATACCGTTGGTTTTAACGGGCTGACGGATATTGTG | |
| | CACGGTGGATCTACTGGGTGCACTGGCACAGACGTGTACAGCGGC | |
| | CTGCCCACACCTTTCGTTCCGTATGCGTCTTGGAACGCAACCGTGG | |
| | GATGGGACCCCGTTACTGGACTTGGCACGCCTCTCTTTGATAAGCT | |
| | GCTCAATTTGAGCACGCCAAACTTCCACTTGCCGCACATTGGCGGT | |
| | CACTAG | |
| 70 | ATGAAGTACAACACACTCCTCACCGGCCTGCTGGCTGTTGCCCATG | Bipolaris |
| | GCAGTGCCGTTTCCGCTTCAACTACTTCACATGTCGAGGGTGAAGT | maydis C5 |
| | TGTCGAGCGACTTCATGGCGTTCCTGAGGGTTGGAGTCAAGTGGGC | |
| | GCCCCCAATCCAGACCAGAAGCTGCGCTTTCGCATCGCAGTACGCT | |
| | CGGTGAGTAATTGCTTTTGTGAACCCATGTTTGAATCTTGCGGTGCT | |
| | TTTTACTGAACATAACAGGCGGATAGCGAGCTGTTTGAGAGGACGC | |
| | TTATGGAGGTTTCTTCTCCCAGCCATCCTCGCTACGGACAGCACCTA | |
| | AAGCGACACGAACTCAAGGACCTCATCAAACCGCGCGCCAAGTCAA | |
| | CTTCAAACATCCTGAACTGGCTGCAAGAGTCTGGAATTGAGGCCAG | |
| | AGATATCCAGAACGATGGCGAGTGGATCAGCTTCTATGCTCCGGTT | |
| | AAACGTGCCGAGCAAATGATGAGCACTACATTCAAGACCTATCAGAA | |
| | CGAGGCCCGAGCGAATATCAAGAAGATCCGCTCTCTAGACTACTCG | |
| | GTGCCGAAGCACATTCGAGATGACATCGACATCATCCAGCCTACGA | |
| | CTCGCTTCGGCCAGATCCAACCGGAGCGTAGCCAAGTCTTTAGTCA | |
| | AGAAGAGGTCCCATTCTCAGCGCTTGTTGTCAATGCGACGTGTAAC | |
| | AAGAAAATCACTCCCGACTGCCTCGCCAACCTCTACAACTTCAAAGA | |
| | CTATGATGCCAGCGATGCCAATGTCACTATCGGAGTCAGCGGCTTC | |
| | CTGGAGCAATATGCTCGCTTTGACGACTTGAAGCAATTCATCAGCAC | |
| | TTTCCAACCAAAAGCAGCTGGTTCCACATTCCAAGTTACATCTGTCA | |
| | ATGCAGGGCCTTTTGACCAGAACTCGACAGCCAGCAGTGTTGAAGC | |
| | CAATCTTGACATTCAGTACACAACAGGTCTTGTTGCGCCCGACATTG | |
| | AAACCCGCTACTTCACTGTTCCCGGTCGCGGTATCCTGATCCCTGA | |
| | TCTGGACCAGCCTACGGAGAGCGACAACGCTAATGAGCCGTATCTG | |
| | GATTACTTTACATATCTTAATAACCTCGAAGACGAAGAACTCCCCGA | |
| | CGTGCTGACCACATCTTACGGCGAGAGCGAGCAGAGTGTACCCGC | |
| | CGAATATGCAAAAAAGGTGTGCAATTTGATCGGCCAGTTGGGTGCT | |
| | CGTGGTGTGTCCGTCATCTTCTCCAGCGGTGATACTGGCCCTGGCT | |
| | CTGCATGCCAAACCAATGATGGAAAAAACACGACACGTTTCTTGCCC | |
| | ATCTTCCCTGCTTCTTGCCCCTACGTCACTTCGGTTGGCGGCACTGT | |
| | TGGTGTTGAGCCCGAAAAGGCTGTCAGCTTCTCTTCGGGCGGCTTT | |
| | TCTGACCTATGGCCTCGACCCGCTTATCAAGAGAAGGCCGTATCAG | |
| | AATATCTTGAAAAGCTCGGAGACCGCTGGAACGGGCTTTACAACCC | |
| | TCAAGGACGCGGATTTCCTGATGTAGCTGCTCAGGGCCAAGGCTTC | |
| | CAGGTGTTTGACAAGGGCAGGCTGATTTCGGTCGGAGGAACGAGC | |
| | GCTTCAGCTCCTGTTTTCGCATCCGTAGTCGCACTCCTGAACAATGC | |
| | TCGCAAGGCTGCCGGCATGTCTTCACTCGGCTTCTTGAACCCATGG | |
| | ATCTACGAGCAAGGCTACAAGGGCTTGACCGATATCGTTGCTGGAG | |
| | GCTCGACAGGATGCACAGGAAGATCCATCTATTCAGGCCTCCCAGC | |
| | ACCACTCGTGCCGTATGCTTCTTGGAATGCCACCGAAGGATGGGAT | |
| | CCGGTGACGGGCTATGGTACACCTGATTTCAAGCAATTGCTCACCC | |
| | TCGCGACGGCACCCAAGTCTGGCGAGCGTCGCGTTCGTCGTGGCG | |
| | GTCTCGGTGGCCAGGCTTAG | |
| 71 | ATGTTATCTTCCTTCCTTAGCCAGGGAGCAGCCGTATCCCTCGCGTT | Aspergillus |
| | ATTGTCGCTGCTCCCTTCGCCTGTAGCCGCGGAGATCTTCGAGAAG | kawachii IFO |
| | CTGTCCGGCGTCCCCAATGGCTGGAGATACGCCAACAATCCTCACG | 4308 |
| | GCAACGAGGTCATTCGCCTTCAAATCGCCCTTCAGCAGCACGATGT | |
| | TGCCGGTTTCGAACAAGCCGTGATGGACATGTCCACCCCCGGTCAC | |
| | GCCGACTATGGAAAGCATTTCCGCACACATGATGAGATGAAGCGCA | |
| | TGCTGCTCCCCAGCGACACTGCCGTCGACTCAGTTCGCGACTGGCT | |
| | GGAATCCGCCGGAGTCCACAATATCCAGGTCGACGCCGACTGGGT | |
| | CAAGTTCCATACCACCGTCAACAAGGCCAATGCCCTGCTGGATGCC | |
| | GACTTCAAGTGGTATGTCAGCGAGGCCAAGCACATTCGTCGTCTAC | |
| | GCACCCTGCAATACTCCATCCCCGACGCCCTGGTCTCGCACATCAA | |
| | CATGATCCAGCCCACCACTCGCTTTGGCCAGATCCAGCCGAACCGT | |
| | GCCACCATGCGCAGCAAGCCCAAGCACGCCGACGAGACATTCCTG | |
| | ACCGCAGCCACCTTGGCCCAGAACACCTCCCACTGCGACTCCATCA | |
| | TCACGCCGCACTGTCTGAAGCAGCTCTACAACATCGGTGACTACCA | |
| | GGCCGACCCCAAGTCCGGTAGCAAGGTCGGCTTCGCCAGCTACCT | |
| | CGAAGAATACGCCCGGTATGCCGATCTCGAAAGGTTCGAGCAGCAC | |
| | CTGGCTCCCAACGCCATCGGCCAGAACTTCAGCGTCGTTCAATTCA | |
| | ACGGCGGCCTCAACGACCAGCTTTCATTGAGCGACAGCGGCGAAG | |
| | CCAACCTCGACCTGCAGTACATCCTGGGCGTCAGCGCTCCCGTCCC | |
| | GGTCACTGAATACAGCACTGGCGGACGCGGCGAACTGGTCCCCGA | |
| | CCTGAGCTCCCCGGACCCCAACGACAACAGCAACGAGCCCTACCT | |
| | CGACTTCCTCCAGGGTATTCTCAAACTCGACAATTCCGACCTCCCCC | |
| | AAGTCATCTCTACCTCCTACGGCGAAGACGAACAGACCATCCCCGT | |
| | CCCCTACGCCCGCACAGTCTGCAATCTCTACGCCCAACTCGGCAGC | |
| | CGCGGTGTCTCCGTGATCTTCTCGAGCGGCGACTCCGGCGTCGGC | |
| | GCCGCCTGCCTCACCAACGACGGCACCAACCGCACCCACTTCCCT | |
| | CCTCAATTCCCGGCCTCCTGCCCCTGGGTAACCTCCGTCGGTGCCA | |
| | CCAGCAAAACCTCCCCGGAGCAAGCCGTCTCCTTCTCCTCAGGAGG | |
| | CTTCTCCGACCTCTGGCCCCGCCCCTCCTACCAACAGGCTGCCGTC | |
| | CAAACCTACCTCACCCAGCACCTGGGCAACAAGTTCTCAGGCCTCT | |
| | TCAACGCCTCCGGCCGCGCCTTCCCCGACGTCGCCGCGCAGGGCG | |
| | TCAACTACGCCGTCTACGACAAGGGCATGCTTGGCCAGTTCGATGG | |
| | AACCAGTTGCTCCGCGCCGACGTTCAGTGGTGTCATTGCCTTGTTG | |
| | AATGACGCCAGACTGAGGGCGGGTTTGCCCGTTATGGGATTCCTGA | |
| | ACCCGTTCCTCTATGGAGTTGGTAGTGAGAGTGGCGCGTTGAATGA | |
| | TATTGTCAACGGCGGGAGCCTGGGTTGTGATGGTAGGAATCGATTT | |
| | GGAGGCACGCCCAATGGAAGTCCCGTTGTGCCGTTTGCTAGTTGGA | |
| | ATGCGACCACCGGGTGGGATCCGGTTTCTGGGCTGGGAACGCCGG | |
| | ATTTTGCGAAGTTGAGGGGTGTGGCGTTGGGTGAAGCTAAGGCGTA | |
| | TGGTAATTAA | |
| 72 | AUGGCAGCGACUGGACGAUUCACUGCCUUCUGGAAUGUCGCGAG | Aspergillus |
| | CGUGCCCGCCUUGAUUGGCAUUCUCCCCCUUGCUGGAUCUCAUU | nidulans |
| | UAAGAGCUGUCCUUUGCCCUGUCUGUAUCUGGCGUCACUCGAAG | FGSC A4 |
| | GCCGUUUGUGCACCAGACACUUUGCAAGCCAUGCGCGCCUUCAC | |
| | CCGUGUAACGGCCAUCUCCCUGGCCGGUUUCUCCUGCUUCGCUG | |
| | CUGCGGCGGCUGCGGCUUUUGAGAGCCUGCGAGCUGUCCCUGAC | |
| | GGCUGGAUCUACGAGAGCACCCCCGACCCUAACCAACCGCUGCG | |
| | UCUACGCAUCGCGCUGAAACAGCACAAUGUCGCCGGCUUCGAGCA | |
| | GGCACUGCUGGAUAUGUCCACACCCGGUCACUCCAGCUACGGGC | |
| | AGCAUUUCGGCUCCUACCACGAGAUGAAGCAGCUGCUUCUCCCUA | |
| | CCGAGGAGGCGUCCUCCUCGGUGCGAGACUGGCUCUCGGCGGC | |
| | GGGCGUUGAGUUCGAACAGGACGCCGACUGGAUCAACUUCCGCA | |
| | CGACCGUCGACCAGGCUAACGCCCUCCUCGACGCCGAUUUCCUC | |
| | UGGUACACAACGACCGGCUCGACGGGCAACCCGACGCGGAUCCU | |
| | CCGAACCCUCUCCUACAGCGUUCCCAGCGAGCUCGCUGGAUACG | |
| | UCAACAUGAUCCAGCCGACUACGCGUUUCGGCGGCACGCAUGCC | |
| | AACCGGGCCACCGUUCGCGCGAAGCCGAUCUUCCUCGAGACCAA | |
| | CCGGCAGCUCAUCAACGCCAUCUCCUCUGGCUCGCUCGAGCACU | |
| | GCGAGAAGGCCAUCACCCCAUCGUGCCUGGCGGAUCUGUACAAC | |
| | ACUGAAGGGUACAAGGCGUCCAACCGCAGCGGGAGCAAGGUGGC | |
| | CUUUGCCUCCUUCCUCGAAGAGUACGCGCGCUACGACGAUCUCG | |
| | CCGAGUUCGAGGAGACCUACGCUCCCUAUGCGAUCGGGCAGAAC | |
| | UUCUCGGUUAUCUCCAUCAACGGCGGCCUCAACGACCAGGACUCC | |
| | ACGGCCGACAGCGGCGAGGCGAACCUCGACCUGCAGUACAUCAU | |
| | CGGCGUCUCGUCGCCGCUACCUGUGACCGAGUUCACAACCGGUG | |
| | GCCGCGGCAAGCUCAUUCCUGACCUCUCCUCCCCCGACCCGAAU | |
| | GACAACACCAACGAGCCUUUCCUUGACUUCCUUGAGGCCGUCCUC | |
| | AAGCUCGAUCAGAAAGACCUGCCCCAGGUCAUCUCGACCUCCUAC | |
| | GGCGAGGACGAGCAGACAAUCCCUGAGCCGUACGCCCGCUCCGU | |
| | CUGCAACCUGUACGCUCAGCUCGGUUCCCGCGGCGUGUCUGUGC | |
| | UCUUCUCCUCGGGUGACUCUGGCGUCGGCGCCGCCUGCCAGACC | |
| | AACGAUGGCAAAAACACGACGCACUUCCCGCCGCAGUUCCCGGCC | |
| | UCUUGCCCCUGGGUGACCGCCGUCGGCGGCACGAACGGCACAGC | |
| | GCCCGAAUCCGGUGUAUACUUCUCCAGCGGCGGGUUCUCCGACU | |
| | ACUGGGCGCGCCCGGCGUACCAGAACGCCGCGGUUGAGUCAUAC | |
| | CUGCGCAAACUCGGUAGCACACAGGCGCAGUACUUCAACCGCAGC | |
| | GGACGCGCCUUCCCGGACGUCGCAGCGCAGGCGCAGAACUUCGC | |
| | UGUCGUCGACAAGGGCCGUGUCGGUCUCUUCGACGGAACGAGCU | |
| | GCAGUUCGCCUGUAUUUGCGGGCAUCGUGGCGUUGCUCAACGAC | |
| | GUGCGUCUGAAGGCAGGCCUGCCCGUGCUGGGAUUCCUCAACCC | |
| | UUGGCUCUACCAGGAUGGCCUGAACGGGCUCAACGAUAUCGUGG | |
| | AUGGAGGGAGCACCGGCUGCGACGGGAACAACCGGUUUAACGGA | |
| | UCGCCAAAUGGGAGCCCCGUAAUCCCGUAUGCGGGUUGGAACGC | |
| | GACGGAGGGGUGGGAUCCUGUGACGGGGCUGGGAACGCCGGAU | |
| | UUCGCGAAGCUGAAAGCGCUCGUGCUUGAUGCUUAG | |
| 73 | ATGTTGTCATTTGTTCGTCGGGGAGCTCTCTCCCTCGCTCTCGTTTC | Aspergillus |
| | GCTGTTGACCTCGTCTGTCGCCGCCGAGGTCTTCGAGAAGCTGCAT | ruber CBS |
| | GTTGTGCCCGAAGGTTGGAGATATGCCTCCACTCCTAACCCCAAAC | 135680 |
| | AACCCATTCGTCTTCAGATCGCTCTGCAGCAGCACGATGTCACCGG | |
| | TTTCGAACAGTCCCTCTTGGAGATGTCGACTCCCGACCATCCCAACT | |
| | ACGGAAAACACTTCCGCACCCACGATGAGATGAAGCGCATGCTTCT | |
| | CCCCAATGAAAATGCCGTTCACGCCGTCCGCGAATGGCTGCAAGAC | |
| | GCCGGAATCAGCGACATCGAAGAAGACGCCGATTGGGTCCGTTTCC | |
| | ACACCACCGTGGACCAGGCCAACGACCTCCTCGACGCCAACTTCCT | |
| | CTGGTACGCGCACAAGAGCCATCGTAACACGGCGCGTCTCCGCAC | |
| | TCTCGAGTACTCGATCCCAGACTCTATTGCGCCGCAGGTCAACGTG | |
| | ATCCAGCCAACCACGCGATTCGGACAGATCCGTGCCAACCGGGCTA | |
| | CGCATAGCAGCAAGCCCAAGGGTGGGCTTGACGAGTTGGCTATCTC | |
| | GCAGGCAGCTACGGCGGATGATGATAGCATTTGTGACCAGATCACC | |
| | ACCCCACACTGTCTGCGGAAGCTGTACAATGTCAATGGCTACAAGG | |
| | CCGATCCCGCTAGTGGTAGCAAGATCGGTTTTGCTAGTTTCCTGGA | |
| | GGAATACGCGCGGTACTCTGATCTGGTACTGTTCGAGGAGAACCTG | |
| | GCACCGTTTGCGGAGGGTGAGAACTTTACTGTCGTCATGTACAACG | |
| | GCGGCAAGAATGACCAGAACTCCAAGAGCGACAGCGGCGAGGCCA | |
| | ACCTCGATCTGCAGTACATCGTGGGAATGAGCGCGGGCGCGCCCG | |
| | TGACCGAGTTCAGCACCGCCGGTCGCGCACCCGTCATCCCGGACC | |
| | TGGACCAGCCCGACCCCAGCGCCGGTACCAACGAGCCGTACCTCG | |
| | AGTTCCTGCAGAACGTGCTACACATGGACCAGGAGCACCTGCCGCA | |
| | GGTGATCTCTACTTCCTACGGTGAGAACGAACAGACCATCCCCGAA | |
| | AAGTACGCCCGCACCGTTTGCAACATGTACGCGCAGCTGGGCAGC | |
| | CGCGGTGTGTCGGTGATTTTCTCGTCGGGCGACTCCGGCGTCGGC | |
| | TCTGCCTGTATGACCAACGACGGTACAAACCGCACCCACTTCCCCC | |
| | CGCAGTTCCCGGCGTCCTGCCCCTGGGTGACATCGGTCGGGGCCA | |
| | CTGAGAAGATGGCCCCCGAGCAAGCGACATATTTCTCCTCGGGCG | |
| | GCTTCTCTGACCTCTTCCCGCGCCCAAAGTACCAGGACGCTGCTGT | |
| | CAGCAGCTACCTTCAGACCCTCGGATCCCGGTACCAGGGCTTGTAC | |
| | AACGGTTCCAACCGTGCATTCCCTGACGTCTCGGCGCAGGGTACCA | |
| | ACTTTGCTGTGTACGACAAGGGCCGTCTAGGCCAGTTCGATGGTAC | |
| | TTCTTGCTCTGCTCCCGCGTTTAGCGGTATCATCGCCTTGCTCAACG | |
| | ACGTCCGTCTCCAGAACAACAAGCCCGTCCTGGGCTTCTTGAACCC | |
| | CTGGTTGTATGGCGCTGGGAGCAAGGGCCTGAACGACGTCGTGCA | |
| | CGGTGGCAGTACAGGATGCGATGGACAGGAGCGGTTTGCAGGAAA | |
| | GGCCAATGGAAGCCCCGTCGTGCCGTACGCTAGCTGGAATGCTAC | |
| | GCAAGGCTGGGATCCAGTCACTGGCCTTGGAACGCCGGATTTCGG | |
| | CAAGTTGAAGGATTTGGCTCTGTCGGCTTAA | |
| 74 | AUGUUGCCCUCUCUUGUAAACAACGGGGCGCUGUCCCUGGCUGU | Aspergillus |
| | GCUUUCGCUGCUCACCUCGUCCGUCGCCGGCGAGGUGUUUGAGA | terreus |
| | AGCUGUCGGCCGUGCCGAAAGGAUGGCACUUCUCCCACGCUGCC | NIH2624 |
| | CAGGCCGACGCCCCCAUCAACCUGAAGAUCGCCCUGAAGCAGCAU | |
| | GAUGUCGAGGGCUUCGAGCAGGCCCUGCUGGACAUGUCCACCCC | |
| | GGGCCACGAGAACUACGGCAAGCACUUCCACGAGCACGACGAGAU | |
| | GAAACGCAUGCUGCUCCCCAGCGACUCCGCCGUCGACGCCGUCC | |
| | AGACCUGGCUGACCUCCGCCGGCAUCACCGACUACGACCUCGAC | |
| | GCCGACUGGAUCAACCUGCGCACCACCGUCGAGCACGCCAACGC | |
| | CCUGCUGGACACGCAGUUCGGCUGGUACGAGAACGAAGUGCGCC | |
| | ACAUCACGCGCCUGCGCACCCUGCAAUACUCCAUCCCCGAGACCG | |
| | UCGCCGCGCACAUCAACAUGGUGCAGCCGACCACGCGCUUUGGC | |
| | CAGAUCCGGCCCGACCGCGCGACCUUCCACGCGCACCACACCUC | |
| | CGACGCGCGCAUCCUGUCCGCCCUGGCCGCCGCCAGCAACAGCA | |
| | CCAGCUGCGACUCAGUCAUCACCCCCAAGUGCCUCAAGGACCUCU | |
| | ACAAGGUCGGCGACUACGAGGCCGACCCGGACUCGGGCAGCCAG | |
| | GUCGCCUUCGCCAGCUACCUCGAGGAAUACGCCCGCUACGCCGA | |
| | CAUGGUCAAGUUCCAGAACUCGCUCGCCCCCUACGCCAAGGGCCA | |
| | GAACUUCUCGGUCGUCCUGUACAACGGCGGCGUCAACGACCAGU | |
| | CGUCCAGCGCCGACUCCGGCGAGGCCAACCUCGACCUGCAGACC | |
| | AUCAUGGGCCUCAGCGCGCCGCUCCCCAUCACCGAGUACAUCACC | |
| | GGCGGCCGCGGCAAGCUCAUCCCCGAUCUCAGCCAGCCCAACCC | |
| | CAACGACAACAGCAACGAGCCCUACCUCGAGUUCCUCCAGAACAU | |
| | CCUCAAGCUGGACCAGGACGAGCUGCCGCAGGUGAUCUCGACCU | |
| | CCUACGGCGAGGACGAGCAGACAAUCCCCCGUGGCUACGCCGAA | |
| | UCCGUCUGCAACAUGCUGGCCCAGCUCGGCAGCCGCGGCGUGUC | |
| | GGUGGUCUUCUCGUCAGGCGAUUCGGGCGUCGGCGCCGCCUGC | |
| | CAGACCAACGACGGCCGCAACCAAACCCACUUCAACCCGCAGUUC | |
| | CCGGCCAGCUGCCCGUGGGUGACGUCGGUCGGGGCCACGACCAA | |
| | GACCAACCCGGAGCAGGCGGUGUACUUCUCGUCGGGCGGGUUCU | |
| | CGGACUUCUGGAAGCGCCCGAAGUACCAGGACGAGGCGGUGGCC | |
| | GCGUACCUGGACACGCUGGGCGACAAGUUCGCGGGGCUGUUCAA | |
| | CAAGGGCGGGCGCGCGUUCCCGGACGUCGCGGCGCAGGGCAUG | |
| | AACUACGCCAUCUACGACAAGGGCACGCUGGGCCGGCUGGACGG | |
| | CACCUCGUGCUCGGCGCCGGCCUUCUCGGCCAUCAUCUCGCUGC | |
| | UGAACGAUGCGCGCCUGCGCGAGGGUAAGCCGACCAUGGGCUUC | |
| | UUGAACCCGUGGCUGUAUGGUGAGGGCCGCGAGGCGCUGAAUGA | |
| | UGUUGUCGUGGGUGGGAGCAAGGGCUGUGAUGGGCGCGACCGG | |
| | UUUGGCGGCAAGCCCAAUGGGAGCCCUGUCGUGCCUUUUGCUAG | |
| | CUGGAAUGCUACGCAGGGCUGGGACCCGGUUACUGGGCUGGGGA | |
| | CGCCGAACUUUGCGAAGAUGUUGGAGCUGGCGCCAUAG | |
| 75 | ATGATTGCATCATTATTCAACCGTAGGGCATTGACGCTCGCTTTATT | Penicillium |
| | GTCACTTTTTGCATCCTCTGCCACAGCCGATGTTTTTGAGAGTTTGT | digitatum |
| | CTGCTGTTCCTCAGGGATGGAGATATTCTCGCACACCGAGTGCTAA | Pd1 |
| | TCAGCCCTTGAAGCTACAGATTGCTCTGGCTCAGGGAGATGTTGCT | |
| | GGGTTCGAGGCAGCTGTGATCGATATGTCAACCCCCGACCACCCCA | |
| | GTTACGGGAACCACTTCAACACCCACGAGGAAATGAAGCGGATGCT | |
| | GCAGCCTAGCGCGGAGTCCGTAGACTCGATCCGTAACTGGCTCGA | |
| | AAGTGCCGGTATTTCCAAGATCGAACAGGACGCTGACTGGATGACC | |
| | TTCTATACCACCGTGAAGACAGCGAATGAGCTGCTGGCAGCCAACT | |
| | TCCAGTTCTACATCAATGGAGTCAAGAAAATAGAGCGTCTCCGCACA | |
| | CTCAAGTACTCTGTCCCGGACGCTTTGGTGTCCCACATTAACATGAT | |
| | CCAGCCAACCACCCGTTTCGGCCAGCTGCGCGCCCAGCGCGCCAT | |
| | TTTACACACCGAGGTCAAGGATAACGACGAGGCTTTCCGCTCAAAT | |
| | GCCATGTCCGCTAATCCGGACTGCAACAGCATCATCACTCCCCAGT | |
| | GTCTCAAGGATTTGTACAGTATCGGTGACTATGAGGCCGACCCCAC | |
| | CAATGGGAACAAGGTCGCGTTTGCCAGCTACCTAGAGGAGTATGCC | |
| | CGATACTCCGATCTCGCATTATTTGAGAAAAACATCGCCCCCTTTGC | |
| | CAAGGGACAGAATTTCTCCGTTGTCCAGTATAACGGCGGTGGTAAT | |
| | GATCAACAATCGAGCAGTGGCAGTAGTGAGGCGAATCTTGACTTGC | |
| | AGTACATCGTTGGAGTCAGCTCTCCTGTTCCCGTTACAGAGTTTAGC | |
| | ACTGGAGGTCGCGGTGAACTTGTTCCGGATCTCGACCAGCCGAATC | |
| | CCAATGACAACAACAACGAGCCATACCTTGAATTCCTCCAGAACGTG | |
| | CTCAAGTTGCACAAGAAGGACCTCCCCCAGGTGATTTCCACCTCTTA | |
| | TGGCGAGGACGAGCAGAGCGTTCCAGAGAAGTACGCCCGCGCCGT | |
| | TTGCAACCTGTACTCCCAACTCGGTAGCCGTGGTGTGTCCGTAATC | |
| | TTTTCATCCGGCGACTCTGGCGTTGGCGCCGCGTGTCAGACGAACG | |
| | ACGGCCGGAACGCGACCCACTTCCCACCCCAGTTCCCGGCCGCCT | |
| | GCCCCTGGGTGACATCAGTCGGTGCGACAACCCACACTGCGCCCG | |
| | AACGAGCCGTTTACTTCTCATCTGGCGGTTTCTCCGATCTCTGGGAT | |
| | CGCCCTACGTGGCAAGAAGATGCTGTGAGTGAGTACCTCGAGAACC | |
| | TGGGCGACCGCTGGTCTGGCCTCTTCAACCCTAAGGGCCGTGCCTT | |
| | CCCCGACGTCGCAGCCCAGGGTGAAAACTACGCCATCTACGATAAG | |
| | GGTTCTTTGATCAGCGTCGATGGCACCTCTTGCTCGGCACCTGCGT | |
| | TTGCCGGAGTCATCGCCCTCCTCAACGACGCCCGCATCAAGGCCAA | |
| | TAGACCACCCATGGGCTTCCTCAACCCTTGGCTGTACTCTGAAGGC | |
| | CGCAGCGGCCTAAACGACATTGTCAACGGCGGTAGCACTGGCTGC | |
| | GACGGTCATGGCCGCTTCTCCGGCCCCACTAACGGTGGTACGTCG | |
| | ATTCCAGGTGCCAGCTGGAACGCTACTAAGGGCTGGGACCCTGTCT | |
| | CCGGTCTTGGATCGCCCAACTTTGCTGCCATGCGCAAACTCGCCAA | |
| | CGCTGAGTAG | |
| 76 | ATGCATGTTCCTCTGTTGAACCAAGGCGCGCTGTCGCTGGCCGTCG | Penicillium |
| | TCTCGCTGTTGGCCTCCACGGTCTCGGCCGAAGTATTCGACAAGCT | oxalicum |
| | TGTCGCTGTCCCTGAAGGATGGCGATTCTCCCGCACTCCCAGTGGA | 114-2 |
| | GACCAGCCCATCCGACTGCAGGTTGCCCTCACACAGGGTGACGTT | |
| | GAGGGCTTCGAGAAGGCCGTTCTGGACATGTCAACTCCCGACCACC | |
| | CCAACTATGGCAAGCACTTCAAGTCACACGAGGAAGTTAAGCGCAT | |
| | GCTGCAGCCTGCAGGCGAGTCCGTCGAAGCCATCCACCAGTGGCT | |
| | CGAGAAGGCCGGCATCACCCACATTCAACAGGATGCCGACTGGAT | |
| | GACCTTCTACACCACCGTTGAGAAGGCCAACAACCTGCTGGATGCC | |
| | AACTTCCAGTACTACCTCAACGAGAACAAGCAGGTCGAGCGTCTGC | |
| | GCACCTTGGAGTACTCGGTTCCTGACGAGCTCGTCTCGCACATTAA | |
| | CCTTGTCACCCCGACCACTCGCTTCGGCCAGCTGCACGCCGAGGG | |
| | TGTGACGCTGCACGGCAAGTCTAAGGACGTCGACGAGCAATTCCGC | |
| | CAGGCTGCTACTTCCCCTAGCAGCGACTGCAACAGTGCTATCACCC | |
| | CGCAGTGCCTCAAGGACCTGTACAAGGTCGGCGACTACAAGGCCA | |
| | GTGCCTCCAATGGCAACAAGGTCGCCTTCACCAGCTACCTGGAGCA | |
| | GTACGCCCGGTACTCGGACCTGGCTCTGTTTGAGCAGAACATTGCC | |
| | CCCTATGCTCAGGGCCAGAACTTCACCGTTATCCAGTACAACGGTG | |
| | GTCTGAACGACCAGAGCTCGCCTGCGGACAGCAGCGAGGCCAACC | |
| | TGGATCTCCAGTACATTATCGGAACGAGCTCTCCCGTCCCCGTGAC | |
| | TGAGTTCAGCACCGGTGGTCGTGGTCCCTTGGTCCCCGACTTGGAC | |
| | CAGCCTGACATCAACGACAACAACAACGAGCCTTACCTCGACTTCTT | |
| | GCAGAATGTCATCAAGATGAGCGACAAGGATCTTCCCCAGGTTATC | |
| | TCCACCTCGTACGGTGAGGACGAGCAGAGCGTCCCCGCAAGCTAC | |
| | GCTCGTAGCGTCTGCAACCTCATCGCTCAGCTCGGCGGCCGTGGT | |
| | GTCTCCGTGATCTTCTCATCTGGTGATTCCGGTGTGGGCTCTGCCT | |
| | GTCAGACCAACGACGGCAAGAACACCACTCGCTTCCCCGCTCAGTT | |
| | CCCCGCCGCCTGCCCCTGGGTGACCTCTGTTGGTGCTACTACCGG | |
| | TATCTCCCCCGAGCGCGGTGTCTTCTTCTCCTCCGGTGGCTTCTCC | |
| | GACCTCTGGAGCCGCCCCTCGTGGCAAAGCCACGCCGTCAAGGCC | |
| | TACCTTCACAAGCTTGGCAAGCGTCAAGACGGTCTCTTCAACCGCG | |
| | AAGGCCGTGCGTTCCCCGACGTGTCAGCCCAGGGTGAGAACTACG | |
| | CTATCTACGCGAAGGGTCGTCTCGGCAAGGTTGACGGCACTTCCTG | |
| | CTCGGCTCCCGCTTTCGCCGGTCTGGTTTCTCTGCTGAACGACGCT | |
| | CGCATCAAGGCGGGCAAGTCCAGCCTCGGCTTCCTGAACCCCTGG | |
| | TTGTACTCGCACCCCGATGCCTTGAACGACATCACCGTCGGTGGAA | |
| | GCACCGGCTGCGACGGCAACGCTCGCTTCGGTGGTCGTCCCAACG | |
| | GCAGTCCCGTCGTCCCTTACGCTAGCTGGAACGCTACTGAGGGCTG | |
| | GGACCCCGTCACCGGTCTGGGTACTCCCAACTTCCAGAAGCTGCTC | |
| | AAGTCTGCCGTTAAGCAGAAGTAA | |
| 77 | ATGATTGCATCCCTATTTAGTCGTGGAGCATTGTCGCTCGCGGTCTT | Penicillium |
| | GTCGCTTCTCGCGTCCTCTGCTGCAGCCGATGTATTTGAGAGTTTGT | roqueforti |
| | CTGCTGTTCCTCAAGGATGGAGATATTCTCGCAGGCCGCGTGCTGA | FM164 |
| | TCAGCCCTTGAAGTTACAGATCGCTCTGACACAGGGGGATACTGCC | |
| | GGCTTCGAAGAGGCTGTGATGGAGATGTCAACCCCCGATCACCCTA | |
| | GCTACGGGCACCACTTCACCACCCACGAAGAAATGAAGCGGATGCT | |
| | ACAGCCCAGTGCGGAGTCCGCGGAGTCAATCCGTGACTGGCTCGA | |
| | AGGCGCGGGTATTACCAGGATCGAACAGGATGCAGATTGGATGACC | |
| | TTCTACACCACCGTGGAGACGGCAAATGAGCTGCTGGCAGCCAATT | |
| | TCCAGTTCTACGTCAGTAATGTCAGGCACATTGAGCGTCTTCGCACA | |
| | CTCAAGTACTCAGTCCCGAAGGCTCTGGTGCCACACATCAACATGA | |
| | TCCAGCCAACCACCCGTTTCGGCCAGCTGCGCGCCCATCGGGGCA | |
| | TATTACACGGCCAGGTCAAGGAATCCGACGAGGCTTTCCGCTCAAA | |
| | CGCCGTGTCCGCTCAGCCGGATTGCAACAGTATCATCACTCCTCAG | |
| | TGTCTCAAGGATATATATAATATCGGTGATTACCAGGCCAATGATAC | |
| | CAATGGGAACAAGGTCGGGTTTGCCAGCTACCTAGAGGAGTATGCA | |
| | CGATACTCCGATCTGGCACTATTTGAGAAAAATATCGCGCCCTCTGC | |
| | CAAGGGCCAGAACTTCTCCGTCACCAGGTACAACGGCGGTCTTAAT | |
| | GATCAAAGTTCCAGCGGTAGCAGCAGCGAGGCGAACCTGGACTTG | |
| | CAGTACATTGTTGGAGTCAGCTCTCCTGTTCCCGTCACCGAATTTAG | |
| | CGTTGGCGGCCGTGGTGAACTTGTTCCCGATCTCGACCAGCCTGAT | |
| | CCCAATGATAACAACAACGAGCCATACCTTGAATTCCTCCAGAACGT | |
| | GCTCAAGCTGGACAAAAAGGACCTTCCCCAGGTGATTTCTACCTCC | |
| | TATGGTGAGGACGAGCAGAGCATTCCCGAGAAGTACGCCCGCAGT | |
| | GTTTGCAACTTGTACTCGCAGCTCGGTAGCCGTGGTGTATCCGTCA | |
| | TTTTCTCATCTGGCGACTCCGGCGTTGGGTCCGCGTGCCTGACGAA | |
| | CGACGGCAGGAACGCGACCCGCTTCCCACCCCAGTTCCCCGCCGC | |
| | CTGCCCGTGGGTGACATCAGTCGGCGCGACAACCCATACCGCGCC | |
| | CGAACAGGCCGTGTACTTCTCGTCCGGCGGCTTTTCCGATCTCTGG | |
| | GCTCGCCCGAAATGGCAAGAGGAGGCCGTGAGTGAGTACCTCGAG | |
| | ATCCTGGGTAACCGCTGGTCTGGCCTCTTCAACCCTAAGGGTCGTG | |
| | CCTTCCCCGATGTCACAGCCCAAGGTCGCAATTACGCTATATACGAT | |
| | AAGGGCTCGTTGACCAGCGTCGACGGCACCTCCTGCTCGGCACCT | |
| | GCCTTCGCCGGAGTCGTCGCCCTCCTCAACGACGCTCGCCTCAAA | |
| | GTCAACAAACCACCAATGGGCTTCCTTAATCCTTGGCTGTACTCGAC | |
| | AGGGCGCGCCGGCCTAAAGGACATTGTCGATGGCGGCAGCACGGG | |
| | TTGCGATGGCAAGAGCCGCTTCGGTGGTGCCAATAACGGTGGTCC | |
| | GTCGATCCCAGGTGCTAGCTGGAACGCTACTAAGGGTTGGGACCCT | |
| | GTTTCTGGTCTCGGGTCGCCCAACTTTGCTACCATGCGCAAGCTTG | |
| | CGAACGCTGAGTAG | |
| 78 | AUGAUUGCAUCUCUAUUUAACCGUGGAGCAUUGUCGCUCGCGGU | Penicillium |
| | AUUGUCGCUUCUCGCGUCUUCGGCUUCCGCUGAUGUAUUUGAGA | rubens |
| | GUUUGUCUGCUGUUCCUCAAGGAUGGAGAUAUUCUCGCAGACCG | Wisconsin |
| | CGUGCUGAUCAGCCCCUGAAGCUACAGAUUGCUCUGGCACAAGG | 54-1255 |
| | GGAUACUGCCGGAUUCGAAGAGGCUGUGAUGGACAUGUCAACCC | |
| | CUGAUCACCCCAGCUACGGGAACCACUUCCACACCCACGAGGAAA | |
| | UGAAGCGGAUGCUGCAGCCCAGCGCGGAGUCCGCAGACUCGAUC | |
| | CGUGACUGGCUUGAAAGUGCGGGUAUCAAUAGAAUUGAACAGGAU | |
| | GCCGACUGGAUGACAUUCUACACCACCGUCGAGACGGCAAAUGAG | |
| | CUGCUGGCAGCCAAUUUCCAGUUCUAUGCCAACAGUGCCAAGCAC | |
| | AUUGAGCGUCUUCGCACACUCCAGUACUCCGUCCCGGAGGCUCU | |
| | GAUGCCACACAUCAACAUGAUCCAGCCAACCACUCGUUUCGGCCA | |
| | GCUGCGCGUCCAGGGGGCCAUAUUGCACACCCAGGUCAAGGAAA | |
| | CCGACGAGGCUUUCCGCUCAAACGCCGUGUCCACUUCACCGGAC | |
| | UGCAACAGUAUCAUCACUCCUCAGUGUCUCAAGAAUAUGUACAAU | |
| | GUGGGUGACUACCAGGCCGACGACGACAAUGGGAACAAGGUCGG | |
| | AUUUGCCAGCUACCUAGAGGAGUAUGCACGGUACUCCGAUUUGG | |
| | AACUAUUUGAGAAAAAUGUCGCACCCUUCGCCAAGGGCCAGAACU | |
| | UCUCCGUCAUCCAGUAUAACGGCGGUCUUAACGAUCAACACUCGA | |
| | GUGCUAGCAGCAGCGAGGCGAACCUUGACUUACAGUACAUUGUU | |
| | GGAGUUAGCUCUCCUGUUCCAGUUACAGAGUUUAGCGUUGGCGG | |
| | UCGUGGUGAACUUGUUCCCGAUCUUGACCAGCCUGAUCCCAAUG | |
| | AUAACAACAACGAGCCAUACCUUGAAUUCCUCCAGAACGUGCUCA | |
| | AGAUGGAACAACAGGACCUCCCCCAGGUGAUUUCCACCUCUUAUG | |
| | GCGAGAACGAGCAGAGUGUUCCCGAGAAAUACGCCCGCACCGUAU | |
| | GCAACUUGUUCUCGCAGCUUGGCAGCCGUGGUGUGUCCGUCAUC | |
| | UUCGCAUCUGGCGACUCCGGCGUUGGCGCCGCGUGCCAGACGAA | |
| | UGACGGCAGGAACGCGACCCGCUUCCCGGCCCAGUUCCCUGCUG | |
| | CCUGCCCAUGGGUGACAUCGGUCGGCGCGACAACCCACACCGCG | |
| | CCCGAGAAGGCCGUGUACUUCUCGUCCGGUGGCUUCUCCGAUCU | |
| | UUGGGAUCGCCCGAAAUGGCAAGAAGACGCCGUGAGUGACUACC | |
| | UCGACACCCUGGGCGACCGCUGGUCCGGCCUCUUCAAUCCUAAG | |
| | GGCCGUGCCUUCCCCGACGUCUCAGCCCAAGGUCAAAACUACGC | |
| | CAUAUACGAUAAGGGCUCGUUGACCAGCGUCGACGGCACCUCGU | |
| | GCUCGGCACCCGCCUUCGCCGGUGUCAUCGCCCUCCUCAACGAC | |
| | GCCCGCCUCAAGGCCAACAAACCACCCAUGGGCUUCCUCAAUCCC | |
| | UGGCUGUACUCGACAGGCCGUGACGGCCUGAACGACAUUGUUCA | |
| | UGGCGGCAGCACUGGCUGUGAUGGCAACGCCCGCUUCGGCGGCC | |
| | CCGGUAACGGCAGUCCGAGGGUUCCAGGUGCCAGCUGGAACGCU | |
| | ACUAAGGGCUGGGACCCUGUUUCUGGUCUUGGAUCACCCAACUU | |
| | UGCUACCAUGCGCAAGCUCGCGAACGGUGAGUAG | |
| 79 | AUGCUGUCCUCGACUCUCUACGCAGGGUUGCUCUGCUCCCUCGC | Neosartorya |
| | AGCCCCAGCCCUUGGUGUGGUGCACGAGAAGCUCUCAGCUGUUC | fischeri |
| | CUAGUGGCUGGACACUCGUCGAGGAUGCAUCGGAGAGCGACACG | NRRL 181 |
| | ACCACUCUCUCAAUUGCCCUUGCUCGGCAGAACCUCGACCAGCUC | |
| | GAGUCCAAGUUGACCACACUGGCGACCCCAGGGAACGCGGAGUA | |
| | CGGCAAGUGGCUGGACCAGUCCGACAUUGAGUCCCUAUUUCCUA | |
| | CUGCAAGCGAUGACGCUGUUAUCCAAUGGCUCAAGGAUGCCGGG | |
| | GUCACCCAAGUGUCUCGUCAGGGCAGCUUGGUGAACUUUGCCAC | |
| | CACUGUGGGAACGGCGAACAAGCUCUUUGACACCAAGUUCUCCUA | |
| | CUACCGCAAUGGUGCUUCCCAGAAACUGCGUACCACGCAGUACUC | |
| | CAUUCCCGAUAGCCUGACAGAGUCGAUCGAUCUGAUUGCCCCCAC | |
| | UGUCUUCUUUGGCAAGGAGCAAGACAGCGCACUGCCACCUCACG | |
| | CAGUGAAGCUUCCAGCCCUUCCCAGGAGGGCAGCCACCAACAGUU | |
| | CuUGCGCCAACCUGAUCACUCCCGACUGCCUAGUGGAGAUGUACA | |
| | ACCUCGGCGACUACAAGCCUGAUGCAUCUUCGGGCAGUCGAGUC | |
| | GGCUUUGGUAGCUUCUUGAAUCAGUCAGCCAACUAUGCAGAUCU | |
| | GGCUGCUUAUGAGCAACUGUUCAACAUCCCACCCCAGAAUUUCUC | |
| | AGUCGAAUUGAUUAACGGAGGCGCCAAUGAUCAGAAUUGGGCCAC | |
| | UGCUUCCCUCGGCGAGGCCAAUCUGGACGUGGAGUUGAUUGUAG | |
| | CCGUCAGCCACGCCCUGCCAGUAGUGGAGUUUAUCACUGGCGGU | |
| | UCACCUCCGUUUGUUCCCAAUGUCGACGAGCCAACCGCUGCGGA | |
| | CAACCAGAAUGAGCCCUACCUCCAGUACUACGAGUACUUGCUCUC | |
| | CAAACCCAACUCCCAUCUUCCUCAGGUGAUUUCCAACUCGUAUGG | |
| | UGACGAUGAACAGACUGUUCCCGAGUACUACGCCAGGAGAGUUU | |
| | GCAACUUGAUCGGCUUGAUGGGUCUUCGUGGUAUCACUGUGCUC | |
| | GAGUCCUCUGGUGAUACCGGAAUCGGCUCGGCGUGCAUGUCCAA | |
| | UGACGGCACCAACACGCCUCAGUUCACUCCUACAUUCCCUGGCAC | |
| | CUGCCCCUUCAUCACCGCAGUUGGUGGUACACAGUCCUAUGCUC | |
| | CUGAAGUUGCCUGGGACGCCAGCUCGGGUGGAUUCAGCAACUAC | |
| | UUCAGCCGUCCCUGGUACCAGUAUUUCGCGGUGGAGAACUACCU | |
| | CAAUAAUCACAUUACCAAGGACACCAAGAAGUACUAUUCGCAGUAC | |
| | ACCAACUUCAAGGGCCGUGGAUUCCCUGAUGUUUCUGCCCAUAG | |
| | CUUGACCCCUGACUACGAGGUCGUCCUAACUGGCAAACAUUACAA | |
| | GUCCGGUGGCACAUCGGCCGCCUGCCCCGUCUUUGCUGGUAUCG | |
| | UCGGCCUGUUGAAUGACGCCCGUCUGCGCGCCGGCAAGUCCACC | |
| | CUUGGCUUCCUGAACCCAUUGCUGUAUAGCAUACUCGCGGAAGG | |
| | AUUCACCGAUAUCACUGCCGGAAGUUCUAUCGGUUGUAAUGGUAU | |
| | CAACCCACAGACCGGAAAGCCAGUCCCCGGUGGUGGUAUCAUCCC | |
| | CUACGCUCACUGGAACGCUACUGCCGGCUGGGAUCCUGUUACAG | |
| | GUCUUGGGGUUCCUGAUUUCAUGAAGUUGAAGGAGUUGGUUUUG | |
| | UCGUUGUAA | |
| 80 | AUGCUGUCCUCGACUCUCUACGCAGGGUGGCUCCUCUCCCUCGC | Aspergillus |
| | AGCCCCAGCCCUUUGUGUGGUGCAGGAGAAGCUCUCAGCUGUUC | fumigatus |
| | CUAGUGGCUGGACACUCAUCGAGGAUGCAUCGGAGAGCGACACG | CAE17675 |
| | AUCACUCUCUCAAUUGCCCUUGCUCGGCAGAACCUCGACCAGCUU | |
| | GAGUCCAAGCUGACCACGCUGGCGACCCCAGGGAACCCGGAGUA | |
| | CGGCAAGUGGCUGGACCAGUCCGACAUUGAGUCCCUAUUUCCUA | |
| | CUGCAAGCGAUGAUGCUGUUCUCCAAUGGCUCAAGGCGGCCGGG | |
| | AUUACCCAAGUGUCUCGUCAGGGCAGCUUGGUGAACUUCGCCAC | |
| | CACUGUGGGAACAGCGAACAAGCUCUUUGACACCAAGUUCUCUUA | |
| | CUACCGCAAUGGUGCUUCCCAGAAACUGCGUACCACGCAGUACUC | |
| | CAUCCCCGAUCACCUGACAGAGUCGAUCGAUCUGAUUGCCCCCAC | |
| | UGUCUUCUUUGGCAAGGAGCAGAACAGCGCACUGUCAUCUCACG | |
| | CAGUGAAGCUUCCAGCUCUUCCUAGGAGGGCAGCCACCAACAGUU | |
| | CuUGCGCCAACCUGAUCACCCCCGACUGCCUAGUGGAGAUGUACA | |
| | ACCUCGGCGACUACAAACCUGAUGCAUCUUCGGGAAGUCGAGUC | |
| | GGCUUCGGUAGCUUCUUGAAUGAGUCGGCCAACUAUGCAGAUUU | |
| | GGCUGCGUAUGAGCAACUCUUCAACAUCCCACCCCAGAAUUUCUC | |
| | AGUCGAAUUGAUCAACAGAGGCGUCAAUGAUCAGAAUUGGGCCAC | |
| | UGCUUCCCUCGGCGAGGCCAAUCUGGACGUGGAGUUGAUUGUAG | |
| | CCGUCAGCCACCCCCUGCCAGUAGUGGAGUUUAUCACUGGCGCC | |
| | CUACCUCCAGUACUACGAGUACUUGCUCUCCAAACCCAACUCCCA | |
| | UCUUCCUCAGGUGAUUUCCAACUCACUGUUCCCGAGUACUACGCC | |
| | AGGAGAGUUUGCAACUUGAUCGGCUUGAUGGGUCUUCGUGGCAU | |
| | CACGGUGCUCGAGUCCUCUGGUGAUACCGGAAUCGGCUCGGCAU | |
| | GCAUGUCCAAUGACGGCACCAACAAGCCCCAAUUCACUCCUACAU | |
| | UCCCUGGCACCUGCCCCUUCAUCACCGCAGUUGGUGGUACUCAG | |
| | UCCUAUGCUCCUGAAGUUGCUUGGGACGGCAGUUCCGGCGGAUU | |
| | CAGCAACUACUUCAGCCGUCCCUGGUACCAGUCUUUCGCGGUGG | |
| | ACAACUACCUCAACAACCACAUUACCAAGGAUACCAAGAAGUACUA | |
| | UUCGCAGUACACCAACUUCAAGGGCCGUGGAUUCCCUGAUGUUU | |
| | CCGCCCAUAGUUUGACCCCUUACUACGAGGUCGUCUUGACUGGC | |
| | AAACACUACAAGUCUGGCGGCACAUCCGCCGCCAGCCCCGUCUUU | |
| | GCCGGUAUUGUCGGUCUGCUGAACGACGCCCGUCUGCGCGCCGG | |
| | CAAGUCCACUCUUGGCUUCCUGAACCCAUUGCUGUAUAGCAUCCU | |
| | GGCCGAAGGAUUCACCGAUAUCACUGCCGGAAGUUCAAUCGGUU | |
| | GUAAUGGUAUCAACCCACAGACCGGAAAGCCAGUUCCUGGUGGU | |
| | GGUAUUAUCCCCUACGCUCACUGGAACGCUACUGCCGGCUGGGA | |
| | UCCUGUUACUGGCCUUGGGGUUCCUGAUUUCAUGAAAUUGAAGG | |
| | AGUUGGUUCUGUCGUUGUAA | |
| 81 | ATGCAGACCTTCGGTGCTTTTCTCGTTTCCTTCCTCGCCGCCAGCG | Phaeosphaeria |
| | GCCTGGCCGCGGCCGAGCCCTTCGAGAAGCTCTTTAGCACCCCCG | nodorum |
| | AGGGCTGGAAGATGCAGGGCCTCGCCACCAACGAGCAGATCGTCA | SN15 |
| | AGCTCCAGATCGCCCTCCAGCAGGGCGACGTGGCCGGCTTTGAGC | |
| | AGCACGTCATCGACATCAGCACCCCCAGCCACCCCAGCTACGGCG | |
| | CTCACTACGGCAGCCACGAAGAGATGAAGCGCATGATCCAGCCCA | |
| | GCAGCGAGACTGTCGCCAGCGTCAGCGCCTGGCTCAAGGCCGCTG | |
| | GCATCAACGACGCCGAGATCGACAGCGACTGGGTCACCTTCAAGAC | |
| | CACCGTCGGCGTCGCCAACAAGATGCTCGACACCAAGTTCGCCTG | |
| | GTACGTCAGCGAGGAAGCCAAGCCCCGCAAGGTCCTCCGCACCCT | |
| | TGAGTACAGCGTCCCCGACGACGTCGCCGAGCACATCAACCTCATC | |
| | CAGCCCACCACCCGCTTCGCCGCCATCCGCCAGAACCACGAGGTC | |
| | GCCCACGAGATCGTCGGCCTCCAGTTTGCCGCCCTCGCCAACAACA | |
| | CCGTCAACTGCGACGCCACCATCACCCCCCAGTGCCTCAAGACCCT | |
| | CTACAAGATCGACTACAAGGCCGACCCCAAGAGCGGCAGCAAGGT | |
| | CGCCTTCGCCAGCTACCTTGAGCAGTACGCCCGCTACAACGACCTC | |
| | GCCCTCTTCGAGAAGGCCTTCCTGCCTGAGGCCGTCGGCCAGAAC | |
| | TTCAGCGTCGTCCAGTTCTCTGGCGGCCTCAACGACCAGAACACCA | |
| | CCCAGGATAGCGGCGAGGCCAACCTCGACCTCCAGTACATCGTCG | |
| | GCGTCAGCGCCCCTCTGCCCGTCACCGAGTTTAGCACTGGCGGCC | |
| | GAGGCCCTTGGGTCGCCGATCTCGATCAGCCTGACGAGGCCGACA | |
| | GCGCCAACGAGCCCTACCTTGAGTTCCTCCAGGGCGTCCTCAAGCT | |
| | CCCCCAGAGCGAGCTGCCCCAGGTCATCAGCACCTCGTACGGCGA | |
| | GAACGAGCAGAGCGTCCCCAAGAGCTACGCCCTCAGCGTCTGCAA | |
| | CCTCTTCGCCCAGCTTGGCTCTCGCGGCGTCAGCGTCATCTTCAGC | |
| | AGCGGCGATAGCGGCCCTGGCAGCGCCTGCCAGTCTAACGACGGC | |
| | AAGAACACCACCAAGTTCCAGCCCCAGTACCCTGCCGCCTGCCCCT | |
| | TCGTCACTAGCGTCGGCTCTACCCGCTACCTCAACGAGACTGCCAC | |
| | CGGCTTCAGCTCCGGCGGCTTCAGCGACTACTGGAAGCGCCCCAG | |
| | CTACCAGGACGACGCCGTCAAGGCCTACTTCCACCACCTCGGCGA | |
| | GAAGTTCAAGCCCTACTTCAACCGCCACGGCCGAGGCTTCCCTGAC | |
| | GTCGCCACTCAGGGCTACGGCTTCCGCGTCTACGACCAGGGCAAG | |
| | CTCAAGGGCCTCCAGGGCACTTCTGCCAGCGCCCCTGCCTTCGCC | |
| | GGCGTCATTGGCCTGCTCAACGACGCCCGCCTCAAGGCCAAGAAG | |
| | CCCACCCTCGGCTTTCTCAACCCCCTGCTCTACAGCAACAGCGACG | |
| | CCCTCAACGACATCGTCCTCGGCGGCTCCAAGGGCTGCGACGGCC | |
| | ACGCTAGGTTTAACGGCCCTCCCAACGGCAGCCCCGTCATCCCTTA | |
| | CGCCGGCTGGAACGCCACTGCCGGCTGGGACCCTGTTACCGGCCT | |
| | CGGCACCCCCAACTTCCCCAAGCTCCTCAAGGCCGCCGTCCCCTCT | |
| | CGATACCGCGCTTAA | |
| 82 | ATGCAGACCTTCGGTGCTTTTCTCGTTTCCTTCCTCGCCGCCAGCG | Trichoderma |
| | GCCTGGCCGCGGCCAACGCTGCTGTCCTCCTCGACAGCCTCGACA | atroviride IMI |
| | AGGTCCCCGTCGGCTGGCAGGCTGCTTCTGCCCCTGCTCCCAGCA | 206040 |
| | GCAAGATCACCCTCCAGGTCGCCCTCACCCAGCAGAACATCGACCA | |
| | GCTTGAGAGCAAGCTCGCCGCCGTCAGCACCCCCAACAGCAGCAA | |
| | CTACGGCAAGTACCTCGACGTCGACGAGATCAACCAGATCTTCGCC | |
| | CCCAGCAGCGCCAGCACTGCCGCTGTCGAGAGCTGGCTCAAGAGC | |
| | TACGGCGTCGACTACAAGGTCCAGGGCAGCAGCATCTGGTTCCAGA | |
| | CCGACGTCAGCACGGCCAACAAGATGCTCAGCACCAACTTCCACAC | |
| | CTACACCGACAGCGTCGGCGCCAAGAAGGTCCGCACCCTCCAGTA | |
| | CAGCGTCCCCGAGACTCTCGCCGACCACATCGACCTCATCAGCCCC | |
| | ACCACCTACTTCGGCACCAGCAAGGCCATGCGAGCCCTCAAGATCC | |
| | AGAACGCCGCCAGCGCCGTCAGCCCTCTCGCTGCTCGACAAGAGC | |
| | CCAGCAGCTGCAAGGGCACCATCGAGTTCGAGAACCGCACCTTCAA | |
| | CGTCTTTCAGCCCGACTGCCTCCGCACCGAGTACAGCGTCAACGGC | |
| | TACAAGCCCAGCGCCAAGAGCGGCAGCCGAATCGGCTTCGGCAGC | |
| | TTCCTCAACCAGAGCGCCAGCAGCAGCGACCTCGCCCTCTTCGAGA | |
| | AGCACTTCGGCTTCGCCAGCCAGGGCTTCAGCGTCGAGCTGATCAA | |
| | CGGCGGCAGCAACCCCCAGCCTCCCACCGATGCTAACGACGGCGA | |
| | GGCCAACCTCGACGCCCAGAACATCGTCAGCTTCGTCCAGCCCCTG | |
| | CCCATCACCGAGTTTATCGCTGGCGGCACCGCCCCCTACTTCCCCG | |
| | ATCCTGTTGAGCCTGCCGGCACCCCCGACGAGAACGAGCCCTACC | |
| | TTGAGTACTACGAGTACCTCCTCAGCAAGAGCAACAAGGAACTCCC | |
| | CCAGGTCATCACCAACAGCTACGGCGACGAGGAACAGACCGTCCC | |
| | CCAGGCCTACGCCGTCCGCGTCTGCAACCTCATCGGCCTCATGGG | |
| | CCTCCGCGGCATCAGCATCCTTGAGAGCAGCGGCGACGAGGGCGT | |
| | CGGCGCTTCTTGCCTCGCCACCAACAGCACCACCACCCCCCAGTTC | |
| | AACCCCATCTTCCCCGCCACGTGCCCCTACGTCACTAGCGTCGGCG | |
| | GCACCGTCAGCTTCAACCCCGAGGTCGCTTGGGACGGCAGCAGCG | |
| | GCGGCTTCAGCTACTACTTCAGCCGCCCCTGGTATCAAGAGGCCGC | |
| | CGTCGGCACCTACCTCAACAAGTACGTCAGCGAGGAAACGAAGGAA | |
| | TATTACAAGAGCTACGTCGACTTCAGCGGCCGAGGCTTCCCTGACG | |
| | TCGCCGCTCACTCTGTCAGCCCCGACTACCCCGTCTTTCAGGGCGG | |
| | CGAGCTGACTCCTTCTGGCGGCACTTCTGCCGCCAGCCCCATCGTC | |
| | GCCAGCGTCATTGCCCTGCTCAACGACGCCCGACTCCGAGCCGGC | |
| | AAGCCTGCCCTCGGCTTTCTCAACCCCCTCATCTACGGCTACGCCT | |
| | ACAAGGGCTTCACCGACATCACCTCCGGCCAGGCCGTTGGCTGCA | |
| | ACGGCAACAACACCCAGACCGGCGGACCCCTTCCTGGCGCTGGCG | |
| | TTATCCCTGGCGCCTTCTGGAACGCCACCAAGGGCTGGGACCCCA | |
| | CCACCGGCTTTGGCGTCCCCAACTTCAAGAAGCTCCTTGAGCTGGT | |
| | CCGCTACATC | |
| 83 | ATGCAGACCTTCGGTGCTTTTCTCGTTTCCTTCCTCGCCGCCAGCG | Arthroderma |
| | GCCTGGCCGCGGCCAAGCCTACTCCTGGCGCTTCCCACAAGGTCA | benhamiae |
| | TCGAGCACCTCGACTTCGTCCCCGAGGGCTGGCAGATGGTCGGCG | CBS 112371 |
| | CTGCTGACCCTGCCGCCATCATCGACTTTTGGCTCGCCATCGAGCG | |
| | CGAGAACCCCGAGAAGCTCTACGACACCATCTACGACGTCAGCACC | |
| | CCCGGACGCGCCCAGTACGGCAAGCACCTCAAGCGCGAGGAACTC | |
| | GACGACCTCCTCCGCCCTCGCGCCGAGACTAGCGAGAGCATCATC | |
| | AACTGGCTCACCAACGGCGGCGTCAACCCCCAGCACATTCGCGAC | |
| | GAGGGCGACTGGGTCCGCTTCAGCACCAACGTCAAGACCGCCGAG | |
| | ACTCTCATGAACACCCGCTTCAACGTCTTTAAGGACAACCTCAACAG | |
| | CGTCAGCAAGATCCGCACCCTTGAGTACAGCGTCCCCGTCGCCATC | |
| | AGCGCCCACGTCCAGATGATCCAGCCCACCACCCTCTTCGGCCGC | |
| | CAGAAGCCCCAGAACAGCCTCATCCTCAACCCCCTCACCAAGGACC | |
| | TTGAGAGCATGAGCGTCGAAGAGTTCGCCGCCAGCCAGTGCCGCA | |
| | GCCTCGTCACTACTGCCTGCCTCCGCGAGCTGTACGGCCTCGGCG | |
| | ATCGAGTCACCCAGGCCCGCGACGACAACCGAATTGGCGTCAGCG | |
| | GCTTCCTCGAAGAGTACGCCCAGTACCGCGACCTTGAGCTGTTCCT | |
| | CAGCCGCTTCGAGCCCAGCGCCAAGGGCTTCAACTTCAGCGAGGG | |
| | CCTGATCGCTGGCGGCAAGAACACCCAGGGTGGCCCTGGCTCTAG | |
| | CACCGAGGCCAACCTCGACATGCAGTACGTCGTCGGCCTCAGCCA | |
| | CAAGGCCAAGGTCACCTACTACAGCACTGCCGGCCGAGGCCCCCT | |
| | CATCCCTGATCTCTCACAGCCCAGCCAGGCCAGCAACAACAACGAG | |
| | CCCTACCTTGAGCAGCTCCGCTACCTCGTCAAGCTCCCCAAGAACC | |
| | AGCTCCCCAGCGTCCTCACCACCAGCTACGGCGACACCGAGCAGA | |
| | GCCTCCCCGCCAGCTACACCAAGGCCACGTGCGACCTCTTCGCCC | |
| | AGCTCGGCACTATGGGCGTCAGCGTCATCTTCAGCAGCGGCGACA | |
| | CTGGCCCTGGCAGCTCGTGCCAGACCAACGACGGCAAGAACGCCA | |
| | CGCGCTTCAACCCCATCTACCCCGCCAGCTGCCCCTTCGTCACCAG | |
| | CATTGGCGGCACCGTCGGCACCGGCCCTGAGCGAGCTGTCAGCTT | |
| | TAGCAGCGGCGGCTTCAGCGACCGCTTCCCTCGCCCTCAGTACCA | |
| | GGACAACGCCGTCAAGGACTACCTCAAGATCCTCGGCAACCAGTGG | |
| | TCCGGCCTCTTCGACCCTAACGGCCGAGCCTTCCCCGACATTGCCG | |
| | CCCAGGGCAGCAACTACGCCGTCTACGACAAGGGCCGCATGACCG | |
| | GCGTTAGCGGCACTTCTGCTTCCGCCCCTGCTATGGCCGCCATCAT | |
| | TGCCCAGCTCAACGACTTCCGCCTCGCCAAGGGCAGCCCCGTCCT | |
| | CGGCTTTCTCAACCCCTGGATCTACAGCAAGGGCTTCAGCGGCTTC | |
| | ACCGACATCGTCGACGGCGGCTCTAGGGGCTGCACCGGCTACGAC | |
| | ATCTACAGCGGCCTCAAGGCCAAGAAGGTCCCCTACGCCAGCTGG | |
| | AACGCCACCAAGGGCTGGGACCCCGTCACCGGCTTTGGCACCCCC | |
| | AACTTCCAGGCCCTGACCAAGGTCCTGCCCTAA | |
| 84 | ATGCAGACCTTCGGTGCTTTTCTCGTTTCCTTCCTCGCCGCCAGCG | Fusarium |
| | GCCTGGCCGCGGCCAAGAGCTACTCTCACCACGCCGAGGCCCCCA | graminearum |
| | AGGGCTGGAAGGTCGACGATACTGCCCGCGTCGCCAGCACCGGCA | PH-1 |
| | AGCAGCAGGTCTTTTCGATCGCCCTGACCATGCAGAACGTCGACCA | |
| | GCTTGAGAGCAAGCTCCTCGACCTCAGCAGCCCCGACAGCAAGAA | |
| | CTACGGCCAGTGGATGAGCCAGAAGGACGTCACCACCGCCTTCTAC | |
| | CCCAGCAAGGAAGCCGTCAGCAGCGTCACCAAGTGGCTCAAGAGC | |
| | AAGGGCGTCAAGCACTACAACGTCAACGGCGGCTTCATCGACTTCG | |
| | CCCTCGACGTGAAGGGCGCCAACGCCCTCCTCGACAGCGACTACC | |
| | AGTACTACACCAAGGAAGGCCAGACCAAGCTCCGCACCCTCAGCTA | |
| | CAGCATCCCCGACGACGTCGCCGAGCACGTCCAGTTCGTCGACCC | |
| | CAGCACCAACTTCGGCGGCACCCTCGCCTTTGCCCCCGTCACTCAC | |
| | CCTAGCCGCACCCTCACCGAGCGCAAGAACAAGCCCACCAAGAGC | |
| | ACCGTCGACGCCAGCTGCCAGACCAGCATCACCCCCAGCTGCCTC | |
| | AAGCAGATGTACAACATCGGCGACTACACCCCCAAGGTCGAGAGCG | |
| | GCAGCACGATCGGCTTCAGCAGCTTCCTCGGCGAGAGCGCTATCTA | |
| | CAGCGACGTCTTTCTGTTCGAGGAAAAGTTCGGCATCCCCACCCAG | |
| | AACTTCACCACCGTCCTCATCAACAACGGCACCGACGACCAGAACA | |
| | CCGCCCACAAGAACTTCGGCGAGGCCGACCTCGACGCCGAGAACA | |
| | TCGTCGGCATTGCCCACCCCCTGCCCTTCACCCAGTACATCACTGG | |
| | CGGCAGCCCCCCCTTCCTGCCCAACATCGATCAGCCCACTGCCGC | |
| | CGACAACCAGAACGAGCCCTACGTCCCCTTCTTCCGCTACCTCCTC | |
| | AGCCAGAAGGAAGTCCCCGCCGTCGTCAGCACCAGCTACGGCGAC | |
| | GAAGAGGACAGCGTCCCCCGCGAGTACGCCACCATGACCTGCAAC | |
| | CTCATCGGCCTGCTCGGCCTCCGCGGCATCAGCGTCATCTTCAGCA | |
| | GCGGCGACATCGGCGTCGGCGCTGGCTGTCTTGGCCCCGACCACA | |
| | AGACCGTCGAGTTCAACGCCATCTTCCCCGCCACGTGCCCCTACCT | |
| | CACTAGCGTCGGCGGCACGGTCGACGTCACCCCCGAGATTGCTTG | |
| | GGAGGGCAGCAGCGGCGGCTTCAGCAAGTACTTCCCTCGCCCCAG | |
| | CTACCAGGACAAGGCCGTCAAGACCTACATGAAGACCGTCAGCAAG | |
| | CAGACCAAGAAGTACTACGGCCCCTACACCAACTGGGAGGGCCGA | |
| | GGCTTTCCTGACGTCGCCGGCCACAGCGTCAGCCCCAACTACGAG | |
| | GTCATCTACGCCGGCAAGCAGAGCGCCTCTGGCGGCACTTCTGCT | |
| | GCCGCCCCTGTCTGGGCTGCCATCGTCGGCCTGCTCAACGACGCC | |
| | CGATTCCGAGCCGGCAAGCCTAGCCTCGGCTGGCTCAACCCCCTC | |
| | GTCTACAAGTACGGCCCCAAGGTCCTCACCGACATCACCGGCGGCT | |
| | ACGCCATTGGCTGCGACGGCAACAACACCCAGAGCGGCAAGCCCG | |
| | AGCCTGCCGGCTCTGGCATTGTCCCTGGCGCCCGATGGAACGCCA | |
| | CTGCCGGATGGGACCCTGTCACCGGCTACGGCACCCCCGACTTCG | |
| | GCAAGCTCAAGGACCTCGTCCTCAGCTTCTAA | |
| 85 | ATGCAGACCTTCGGTGCTTTTCTCGTTTCCTTCCTCGCCGCCAGCG | Acremonium |
| | GCCTGGCCGCGGCCGCCGTCGTCATTCGCGCCGCCGTCCTCCCCG | alcalophilum |
| | ACGCCGTCAAGCTGATGGGCAAGGCCATGCCCGACGACATTATTTC | |
| | CCTCCAGTTTTCCCTGAAGCAGCAGAACATCGACCAGCTGGAGACC | |
| | CGCCTCCGCGCCGTCTCGGACCCCAGCTCCCCCGAGTACGGCCAG | |
| | TACATGAGCGAGTCCGAGGTCAACGAGTTCTTTAAGCCCCGCGACG | |
| | ACTCGTTCGCCGAGGTCATTGACTGGGTCGCCGCCAGCGGCTTTCA | |
| | GGACATCCACCTGACGCCCCAGGCTGCCGCCATTAACCTCGCCGC | |
| | CACCGTCGAGACGGCCGACCAGCTCCTGGGCGCCAACTTCAGCTG | |
| | GTTTGACGTCGACGGCACCCGCAAGCTCCGCACCCTGGAGTACAC | |
| | GATCCCCGACCGCCTCGCCGACCACGTCGACCTGATTTCCCCCACC | |
| | ACGTACTTCGGCCGCGCCCGACTGGACGGCCCCCGCGAGACCCCC | |
| | ACGCGCCTCGACAAGCGCCAGCGCGACCCCGTCGCCGACAAGGCC | |
| | TACTTCCACCTCAAGTGGGACCGCGGCACCAGCAACTGCGACCTG | |
| | GTCATCACGCCCCCCTGCCTGGAGGCCGCCTACAACTACAAGAACT | |
| | ACATGCCCGACCCCAACTCGGGCAGCCGCGTCTCGTTCACCAGCTT | |
| | TCTGGAGCAGGCCGCCCAGCAGAGCGACCTCACCAAGTTCCTCTC | |
| | CCTGACGGGCCTCGACCGCCTGCGCCCCCCCAGCAGCAAGCCCGC | |
| | CAGCTTCGACACGGTCCTGATCAACGGCGGCGAGACCCACCAGGG | |
| | CACGCCCCCCAACAAGACCTCCGAGGCCAACCTCGACGTCCAGTG | |
| | GCTGGCCGCCGTCATTAAGGCCCGACTCCCCATCACCCAGTGGATT | |
| | ACGGGCGGCCGCCCCCCCTTCGTCCCCAACCTCCGCCTGCGCCAC | |
| | GAGAAGGACAACACGAACGAGCCCTACCTGGAGTTCTTTGAGTACC | |
| | TCGTCCGCCTGCCCGCCCGCGACCTCCCCCAGGTCATCTCCAACTC | |
| | GTACGCCGAGGACGAGCAGACCGTCCCCGAGGCCTACGCCCGACG | |
| | CGTCTGCAACCTCATCGGCATTATGGGCCTGCGCGGCGTCACCGTC | |
| | CTCACGGCCTCCGGCGACTCGGGCGTCGGCGCCCCCTGCCGCGC | |
| | CAACGACGGCAGCGACCGCCTGGAGTTCTCCCCCCAGTTTCCCAC | |
| | CTCGTGCCCCTACATCACCGCCGTCGGCGGCACGGAGGGCTGGGA | |
| | CCCCGAGGTCGCCTGGGAGGCCTCCTCGGGCGGCTTCAGCCACTA | |
| | CTTTCTCCGCCCCTGGTACCAGGCCAACGCCGTCGAGAAGTACCTC | |
| | GACGAGGAGCTGGACCCCGCCACCCGCGCCTACTACGACGGCAAC | |
| | GGCTTCGTCCAGTTTGCCGGCCGAGCCTACCCCGACCTGTCCGCC | |
| | CACAGCTCCTCGCCCCGCTACGCCTACATCGACAAGCTCGCCCCC | |
| | GGCCTGACCGGCGGCACGAGCGCCTCCTGCCCCGTCGTCGCCGG | |
| | CATCGTCGGCCTCCTGAACGACGCCCGACTCCGCCGCGGCCTGCC | |
| | CACGATGGGCTTCATTAACCCCTGGCTGTACACGCGCGGCTTTGAG | |
| | GCCCTCCAGGACGTCACCGGCGGCCGCGCCTCGGGCTGCCAGGG | |
| | CATCGACCTCCAGCGCGGCACCCGCGTCCCCGGCGCCGGCATCAT | |
| | TCCCTGGGCCTCCTGGAACGCCACCCCCGGCTGGGACCCCGCCAC | |
| | GGGCCTCGGCCTGCCCGACTTCTGGGCCATGCGCGGCCTCGCCCT | |
| | GGGCCGCGGCACCTAA | |
| 86 | ATGCAGACCTTCGGTGCTTTTCTCGTTTCCTTCCTCGCCGCCAGCG | Sodiomyces |
| | GCCTGGCCGCGGCCGCCGTCGTCATTCGCGCCGCCCCCCTCCCCG | alkalinus |
| | AGAGCGTCAAGCTCGTCCGCAAGGCCGCCGCCGAGGACGGCATTA | |
| | ACCTCCAGCTCTCCCTGAAGCGCCAGAACATGGACCAGCTGGAGAA | |
| | GTTCCTCCGCGCCGTCAGCGACCCCTTTTCCCCCAAGTACGGCCAG | |
| | TACATGTCGGACGCCGAGGTCCACGAGATCTTCCGCCCCACCGAG | |
| | GACTCCTTTGACCAGGTCATTGACTGGCTCACCAAGTCGGGCTTCG | |
| | GCAACCTGCACATCACGCCCCAGGCTGCCGCCATTAACGTCGCCAC | |
| | CACGGTCGAGACCGCCGACCAGCTGTTTGGCGCCAACTTCTCCTG | |
| | GTTTGACGTCGACGGCACGCCCAAGCTCCGCACCGGCGAGTACAC | |
| | GATCCCCGACCGCCTCGTCGAGCACGTCGACCTGGTCAGCCCCAC | |
| | CACGTACTTCGGCCGCATGCGCCCCCCCCCTCGCGGCGACGGCGT | |
| | CAACGACTGGATCACCGAGAACTCGCCCGAGCAGCCCGCCCCCCT | |
| | GAACAAGCGCGACACCAAGACGGAGAGCGACCAGGCCCGCGACCA | |
| | CCCCTCCTGGGACTCGCGCACCCCCGACTGCGCCACCATCATTAC | |
| | GCCCCCCTGCCTGGAGACGGCCTACAACTACAAGGGCTACATCCC | |
| | CGACCCCAAGTCCGGCTCGCGCGTCAGCTTCACCAGCTTCCTGGA | |
| | GCAGGCCGCCCAGCAGGCCGACCTGACCAAGTTCCTCAGCCTGAC | |
| | GCGCCTGGAGGGCTTTCGCACCCCCGCCAGCAAGAAGAAGACCTT | |
| | CAAGACGGTCCTGATCAACGGCGGCGAGTCCCACGAGGGCGTCCA | |
| | CAAGAAGTCGAAGACCAGCGAGGCCAACCTCGACGTCCAGTGGCT | |
| | GGCCGCCGTCACCCAGACGAAGCTGCCCATCACCCAGTGGATTAC | |
| | GGGCGGCCGCCCCCCCTTCGTCCCCAACCTCCGCATCCCCACCCC | |
| | CGAGGCCAACACGAACGAGCCCTACCTGGAGTTCCTGGAGTACCTC | |
| | TTTCGCCTGCCCGACAAGGACCTCCCCCAGGTCATCAGCAACTCCT | |
| | ACGCCGAGGACGAGCAGAGCGTCCCCGAGGCCTACGCCCGACGC | |
| | GTCTGCGGCCTCCTGGGCATTATGGGCCTCCGCGGCGTCACCGTC | |
| | CTGACGGCCTCCGGCGACTCGGGCGTCGGCGCCCCCTGCCGCGC | |
| | CAACGACGGCTCGGGCCGCGAGGAGTTCAGCCCCCAGTTTCCCAG | |
| | CTCCTGCCCCTACATCACCACGGTCGGCGGCACCCAGGCCTGGGA | |
| | CCCCGAGGTCGCCTGGAAGGGCAGCAGCGGCGGCTTCTCCAACTA | |
| | CTTTCCCCGCCCCTGGTACCAGGTCGCCGCCGTCGAGAAGTACCT | |
| | GGAGGAGCAGCTGGACCCCGCCGCCCGCGAGTACTACGAGGAGAA | |
| | CGGCTTCGTCCGCTTTGCCGGCCGAGCCTTCCCCGACCTGAGCGC | |
| | CCACAGCAGCAGCCCCAAGTACGCCTACGTCGACAAGCGCGTCCC | |
| | CGGCCTCACCGGCGGCACGTCGGCCAGCTGCCCCGTCGTCGCCG | |
| | GCATCGTCGGCCTCCTGAACGACGCCCGACTCCGCCGCGGCCTGC | |
| | CCACGATGGGCTTCATTAACCCCTGGCTCTACGCCAAGGGCTACCA | |
| | GGCCCTGGAGGACGTCACCGGCGGCGCCGCCGTCGGCTGCCAGG | |
| | GCATCGACATTCAGACGGGCAAGCGCGTCCCCGGCGCCGGCATCA | |
| | TTCCCGGCGCCAGCTGGAACGCCACCCCCGACTGGGACCCCGCCA | |
| | CGGGCCTCGGCCTGCCCAACTTCTGGGCCATGCGCGAGCTCGCCC | |
| | TGGAGGACTAA | |
| 87 | ATGCAGACCTTCGGTGCTTTTCTCGTTTCCTTCCTCGCCGCCAGCG | Aspergillus |
| | GCCTGGCCGCGGCCGTCGTCCATGAGAAGCTCGCTGCTGTCCCCA | kawachii IFO |
| | GCGGCTGGCACCACCTTGAGGATGCCGGCAGCGACCACCAGATCA | 4308 |
| | GCCTCTCGATTGCCCTCGCCCGCAAGAACCTCGACCAGCTTGAGAG | |
| | CAAGCTCAAGGACCTCAGCACCCCTGGCGAGAGCCAGTACGGCCA | |
| | GTGGCTCGACCAAGAGGAAGTCGACACCCTGTTCCCCGTCGCCAG | |
| | CGACAAGGCCGTCATCAGCTGGCTCCGCAGCGCCAACATCACCCA | |
| | CATTGCCCGCCAGGGCAGCCTCGTCAACTTCGCCACCACCGTCGA | |
| | CAAGGTCAACAAGCTCCTCAACACCACCTTCGCCTACTACCAGCGC | |
| | GGCAGCTCTCAGCGCCTCCGCACCACCGAGTACAGCATCCCCGAC | |
| | GACCTCGTCGACAGCATCGACCTGATCAGCCCCACCACGTTCTTCG | |
| | GCAAGGAAAAGACCTCTGCCGGCCTCACCCAGCGCAGCCAGAAGG | |
| | TCGATAACCACGTCGCCAAGCGCAGCAACAGCAGCAGCTGCGCCG | |
| | ACACCATCACCCTCAGCTGCCTCAAGGAAATGTACAACTTCGGCAA | |
| | CTACACCCCCAGCGCCAGCAGCGGCAGCAAGCTCGGCTTCGCCAG | |
| | CTTCCTCAACGAGAGCGCCAGCTACAGCGACCTCGCCAAGTTCGAG | |
| | CGCCTCTTCAACCTCCCCAGCCAGAACTTCAGCGTCGAGCTGATCA | |
| | ACGGCGGCGTCAACGACCAGAACCAGAGCACCGCCAGCCTCACCG | |
| | AGGCCGACCTCGATGTCGAGCTGCTTGTCGGCGTCGGCCACCCCC | |
| | TGCCCGTCACCGAGTTTATCACCAGCGGCGAGCCCCCCTTCATCCC | |
| | CGACCCTGATGAGCCTTCTGCCGCCGACAACGAGAACGAGCCCTA | |
| | CCTCCAGTACTACGAGTACCTCCTCAGCAAGCCCAACAGCGCCCTG | |
| | CCCCAGGTCATCAGCAACAGCTACGGCGACGACGAGCAGACCGTC | |
| | CCCGAGTACTACGCCAAGCGCGTCTGCAACCTCATCGGCCTCGTCG | |
| | GCCTCCGCGGCATCAGCGTCCTTGAGTCTAGCGGCGACGAGGGCA | |
| | TCGGCTCTGGCTGCCGAACCACCGACGGCACCAACAGCACCCAGT | |
| | TCAACCCCATCTTCCCCGCCACGTGCCCCTACGTCACTGCCGTCGG | |
| | CGGCACCATGAGCTACGCCCCCGAGATTGCTTGGGAGGCCAGCTC | |
| | CGGCGGCTTCAGCAACTACTTCGAGCGAGCCTGGTTCCAGAAGGAA | |
| | GCCGTCCAGAACTACCTCGCCAACCACATCACCAACGAGACTAAGC | |
| | AGTACTACAGCCAGTTCGCCAACTTCAGCGGCCGAGGCTTCCCCGA | |
| | CGTCAGCGCCCACAGCTTCGAGCCCAGCTACGAGGTCATCTTCTAC | |
| | GGCGCTCGCTACGGCAGCGGCGGCACTTCTGCTGCCTGCCCCCTG | |
| | TTTTCTGCCCTCGTCGGCATGCTCAACGACGCCCGACTCCGAGCCG | |
| | GCAAGTCGACCCTCGGCTTCCTCAACCCCCTGCTCTACAGCAAGGG | |
| | CTACAAGGCCCTCACCGACGTCACCGCTGGCCAGAGCATTGGCTG | |
| | CAACGGCATCGACCCCCAGAGCGACGAGGCTGTCGCTGGCGCTGG | |
| | CATCATTCCCTGGGCCCACTGGAACGCCACCGTCGGCTGGGACCC | |
| | TGTCACTGGCCTTGGCCTCCCCGACTTCGAGAAGCTCCGCCAGCTC | |
| | GTCCTCAGCCTCTAA | |
| 88 | ATGCAGACCTTCGGTGCTTTTCTCGTTTCCTTCCTCGCCGCCAGCG | Talaromyces |
| | GCCTGGCCGCGGCCGCTGCTGCTCTTGTTGGCCACGAGTCTCTCG | stipitatus |
| | CCGCCCTCCCTGTCGGCTGGGACAAGGTCAGCACTCCTGCCGCTG | ATCC 10500 |
| | GCACCAACATCCAGCTCAGCGTCGCCCTCGCCCTCCAGAACATCGA | |
| | GCAGCTTGAGGACCACCTCAAGAGCGTCAGCACCCCCGGCTCTGC | |
| | CAGCTACGGCCAGTACCTCGACAGCGACGGCATTGCCGCCCAGTA | |
| | CGGCCCTTCTGACGCCAGCGTCGAGGCCGTCACCAACTGGCTCAA | |
| | GGAAGCCGGCGTCACCGACATCTACAACAACGGCCAGAGCATCCA | |
| | CTTCGCCACCAGCGTCAGCAAGGCCAACAGCCTCCTCGGCGCCGA | |
| | CTTCAACTACTACAGCGACGGCTCCGCCACCAAGCTCCGCACCCTC | |
| | GCTTACAGCGTCCCCAGCGACCTGAAGGAAGCCATCGACCTCGTCA | |
| | GCCCCACCACCTACTTCGGCAAGACCACCGCCAGCCGCAGCATCC | |
| | AGGCCTACAAGAACAAGCGAGCCAGCACCACCAGCAAGAGCGGCA | |
| | GCAGCAGCGTCCAGGTCAGCGCCTCTTGCCAGACCAGCATCACCC | |
| | CCGCCTGCCTCAAGCAGATGTACAACGTCGGCAACTACACCCCCAG | |
| | CGTCGCCCACGGCTCTCGCGTTGGCTTCGGCAGCTTCCTCAACCAG | |
| | AGCGCCATCTTCGACGACCTCTTCACCTACGAGAAGGTCAACGACA | |
| | TCCCCAGCCAGAACTTCACCAAGGTCATCATTGCCAACGCCAGCAA | |
| | CAGCCAGGACGCCAGCGACGGCAACTACGGCGAGGCCAACCTCGA | |
| | CGTCCAGAACATTGTCGGCATCAGCCACCCCCTGCCCGTCACCGAG | |
| | TTTCTCACTGGCGGCAGCCCACCCTTCGTCGCCAGCCTCGACACCC | |
| | CCACCAACCAGAACGAGCCCTACATCCCCTACTACGAGTACCTCCT | |
| | CAGCCAGAAGAACGAGGACCTCCCCCAGGTCATCAGCAACAGCTAC | |
| | GGCGACGACGAGCAGAGCGTCCCCTACAAGTACGCCATCCGCGCC | |
| | TGCAACCTCATCGGCCTCACTGGCCTCCGCGGCATCAGCGTCCTTG | |
| | AGAGCAGCGGCGATCTCGGCGTTGGCGCTGGCTGCCGATCCAACG | |
| | ACGGCAAGAACAAGACCCAGTTCGACCCCATCTTCCCCGCCACGTG | |
| | CCCCTACGTCACTAGCGTCGGCGGCACCCAGAGCGTCACCCCCGA | |
| | GATTGCTTGGGTCGCTTCCAGCGGCGGCTTCAGCAACTACTTCCCC | |
| | CGCACCTGGTATCAAGAGCCCGCCATCCAGACCTACCTCGGCCTCC | |
| | TCGACGACGAGACTAAGACCTACTACAGCCAGTACACCAACTTCGA | |
| | GGGCCGAGGCTTCCCCGACGTCAGCGCCCATTCTCTCACCCCCGA | |
| | CTACCAGGTCGTCGGCGGAGGCTACCTTCAGCCTTCTGGCGGCAC | |
| | TTCTGCCGCCAGCCCTGTCTTTGCCGGCATCATTGCCCTGCTCAAC | |
| | GACGCCCGACTCGCCGCTGGCAAGCCCACCCTCGGCTTTCTCAAC | |
| | CCCTTCTTCTACCTCTACGGCTACAAGGGCCTCAACGACATCACTG | |
| | GCGGCCAGAGCGTCGGCTGCAACGGCATCAACGGCCAGACTGGCG | |
| | CCCCTGTTCCCGGCGGAGGAATTGTCCCTGGCGCCGCTTGGAACA | |
| | GCACCACCGGATGGGACCCTGCCACCGGCCTTGGCACCCCCGACT | |
| | TTCAGAAGCTCAAGGAACTCGTCCTCAGCTTCTAA | |
| 89 | ATGCAGACCTTCGGTGCTTTTCTCGTTTCCTTCCTCGCCGCCAGCG | Fusarium |
| | GCCTGGCCGCGGCCAAGTCGTTTTCCCACCACGCCGAGGCCCCCC | oxysporum f. |
| | AGGGCTGGCAGGTCCAGAAGACCGCCAAGGTCGCCTCCAACACGC | sp. cubense |
| | AGCACGTCTTTAGCCTCGCCCTGACCATGCAGAACGTCGACCAGCT | race 4 |
| | GGAGTCGAAGCTCCTGGACCTGAGCTCCCCCGACAGCGCCAACTA | |
| | CGGCAACTGGCTCAGCCACGACGAGCTGACCTCCACGTTCTCGCC | |
| | CAGCAAGGAGGCCGTCGCCTCGGTCACCAAGTGGCTGAAGAGCAA | |
| | GGGCATCAAGCACTACAAGGTCAACGGCGCCTTCATTGACTTTGCC | |
| | GCCGACGTCGAGAAGGCCAACACCCTCCTGGGCGGCGACTACCAG | |
| | TACTACACGAAGGACGGCCAGACCAAGCTGCGCACGCTCTCCTACT | |
| | CGATCCCCGACGACGTCGCCGGCCACGTCCAGTTCGTCGACCCCA | |
| | GCACCAACTTCGGCGGCACGGTCGCCTTTAACCCCGTCCCCCACC | |
| | CCTCCCGCACCCTCCAGGAGCGCAAGGTCTCCCCCTCCAAGTCGA | |
| | CGGTCGACGCCTCCTGCCAGACCTCGATCACGCCCAGCTGCCTGA | |
| | AGCAGATGTACAACATTGGCGACTACACCCCCGACGCCAAGAGCG | |
| | GCTCCGAGATCGGCTTCAGCAGCTTCCTCGGCCAGGCCGCCATTTA | |
| | CAGCGACGTCTTCAAGTTTGAGGAGCTCTTCGGCATCCCCAAGCAG | |
| | AACTACACCACGATCCTGATTAACAACGGCACCGACGACCAGAACA | |
| | CGGCCCACGGCAACTTTGGCGAGGCCAACCTCGACGCCGAGAACA | |
| | TCGTCGGCATTGCCCACCCCCTGCCCTTCAAGCAGTACATCACCGG | |
| | CGGCAGCCCCCCCTTTGTCCCCAACATTGACCAGCCCACGGAGAA | |
| | GGACAACCAGAACGAGCCCTACGTCCCCTTCTTTCGCTACCTCCTG | |
| | GGCCAGAAGGACCTGCCCGCCGTCATCTCGACCAGCTACGGCGAC | |
| | GAGGAGGACTCCGTCCCCCGCGAGTACGCCACCCTCACGTGCAAC | |
| | ATGATCGGCCTCCTGGGCCTGCGCGGCATCTCCGTCATTTTCTCCT | |
| | CGGGCGACATTGGCGTCGGCTCGGGCTGCCTCGCCCCCGACTACA | |
| | AGACCGTCGAGTTCAACGCCATCTTTCCCGCCACCTGCCCCTACCT | |
| | GACGTCCGTCGGCGGCACCGTCGACGTCACGCCCGAGATTGCCTG | |
| | GGAGGGCAGCTCCGGCGGCTTCTCCAAGTACTTTCCCCGCCCCTC | |
| | GTACCAGGACAAGGCCATCAAGAAGTACATGAAGACCGTCTCGAAG | |
| | GAGACGAAGAAGTACTACGGCCCCTACACCAACTGGGAGGGCCGC | |
| | GGCTTCCCCGACGTCGCCGGCCACTCCGTCGCCCCCGACTACGAG | |
| | GTCATCTACAACGGCAAGCAGGCCCGATCCGGCGGCACCAGCGCC | |
| | GCCGCCCCCGTCTGGGCCGCCATCGTCGGCCTCCTGAACGACGCC | |
| | CGATTCAAGGCCGGCAAGAAGAGCCTGGGCTGGCTCAACCCCCTG | |
| | ATCTACAAGCACGGCCCCAAGGTCCTCACCGACATCACGGGCGGC | |
| | TACGCCATTGGCTGCGACGGCAACAACACCCAGAGCGGCAAGCCC | |
| | GAGCCCGCCGGCTCCGGCCTGGTCCCCGGCGCCCGATGGAACGC | |
| | CACCGCCGGCTGGGACCCCACCACGGGCTACGGCACGCCCAACTT | |
| | CCAGAAGCTCAAGGACCTCGTCCTGTCCCTCTAA | |
| 90 | ATGCAGACCTTCGGTGCTTTTCTCGTTTCCTTCCTCGCCGCCAGCG | Trichoderma |
| | GCCTGGCCGCGGCCGTCGTCCATGAGAAGCTCGCTGCTGTCCCCA | virens |
| | GCGGCTGGCACCACCTTGAGGATGCCGGCAGCGACCACCAGATCA | Gv29-8 |
| | GCCTCTCGATTGCCCTCGCCCGCAAGAACCTCGACCAGCTTGAGAG | |
| | CAAGCTCAAGGACCTCAGCACCCCTGGCGAGAGCCAGTACGGCCA | |
| | GTGGCTCGACCAAGAGGAAGTCGACACCCTGTTCCCCGTCGCCAG | |
| | CGACAAGGCCGTCATCAGCTGGCTCCGCAGCGCCAACATCACCCA | |
| | CATTGCCCGCCAGGGCAGCCTCGTCAACTTCGCCACCACCGTCGA | |
| | CAAGGTCAACAAGCTCCTCAACACCACCTTCGCCTACTACCAGCGC | |
| | GGCAGCTCTCAGCGCCTCCGCACCACCGAGTACAGCATCCCCGAC | |
| | GACCTCGTCGACAGCATCGACCTGATCAGCCCCACCACGTTCTTCG | |
| | GCAAGGAAAAGACCTCTGCCGGCCTCACCCAGCGCAGCCAGAAGG | |
| | TCGATAACCACGTCGCCAAGCGCAGCAACAGCAGCAGCTGCGCCG | |
| | ACACCATCACCCTCAGCTGCCTCAAGGAAATGTACAACTTCGGCAA | |
| | CTACACCCCCAGCGCCAGCAGCGGCAGCAAGCTCGGCTTCGCCAG | |
| | CTTCCTCAACGAGAGCGCCAGCTACAGCGACCTCGCCAAGTTCGAG | |
| | CGCCTCTTCAACCTCCCCAGCCAGAACTTCAGCGTCGAGCTGATCA | |
| | ACGGCGGCGTCAACGACCAGAACCAGAGCACCGCCAGCCTCACCG | |
| | AGGCCGACCTCGATGTCGAGCTGCTTGTCGGCGTCGGCCACCCCC | |
| | TGCCCGTCACCGAGTTTATCACCAGCGGCGAGCCCCCCTTCATCCC | |
| | CGACCCTGATGAGCCTTCTGCCGCCGACAACGAGAACGAGCCCTA | |
| | CCTCCAGTACTACGAGTACCTCCTCAGCAAGCCCAACAGCGCCCTG | |
| | CCCCAGGTCATCAGCAACAGCTACGGCGACGACGAGCAGACCGTC | |
| | CCCGAGTACTACGCCAAGCGCGTCTGCAACCTCATCGGCCTCGTCG | |
| | GCCTCCGCGGCATCAGCGTCCTTGAGTCTAGCGGCGACGAGGGCA | |
| | TCGGCTCTGGCTGCCGAACCACCGACGGCACCAACAGCACCCAGT | |
| | TCAACCCCATCTTCCCCGCCACGTGCCCCTACGTCACTGCCGTCGG | |
| | CGGCACCATGAGCTACGCCCCCGAGATTGCTTGGGAGGCCAGCTC | |
| | CGGCGGCTTCAGCAACTACTTCGAGCGAGCCTGGTTCCAGAAGGAA | |
| | GCCGTCCAGAACTACCTCGCCAACCACATCACCAACGAGACTAAGC | |
| | AGTACTACAGCCAGTTCGCCAACTTCAGCGGCCGAGGCTTCCCCGA | |
| | CGTCAGCGCCCACAGCTTCGAGCCCAGCTACGAGGTCATCTTCTAC | |
| | GGCGCTCGCTACGGCAGCGGCGGCACTTCTGCTGCCTGCCCCCTG | |
| | TTTTCTGCCCTCGTCGGCATGCTCAACGACGCCCGACTCCGAGCCG | |
| | GCAAGTCGACCCTCGGCTTCCTCAACCCCCTGCTCTACAGCAAGGG | |
| | CTACAAGGCCCTCACCGACGTCACCGCTGGCCAGAGCATTGGCTG | |
| | CAACGGCATCGACCCCCAGAGCGACGAGGCTGTCGCTGGCGCTGG | |
| | CATCATTCCCTGGGCCCACTGGAACGCCACCGTCGGCTGGGACCC | |
| | TGTCACTGGCCTTGGCCTCCCCGACTTCGAGAAGCTCCGCCAGCTC | |
| | GTCCTCAGCCTCTAA | |
| 91 | ATGCAGACCTTCGGTGCTTTTCTCGTTTCCTTCCTCGCCGCCAGCG | Trichoderma |
| | GCCTGGCCGCGGCCGCTGTCCTTGTCGAGTCTCTCAAGCAGGTCC | atroviride IMI |
| | CCAACGGCTGGAACGCCGTCAGCACCCCTGACCCCAGCACCAGCA | 206040 |
| | TCGTCCTCCAGATCGCCCTCGCCCAGCAGAACATCGACGAGCTTGA | |
| | GTGGCGCCTCGCCGCCGTGTCTACCCCCAACTCTGGCAACTACGG | |
| | CAAGTACCTCGACATCGGCGAGATCGAGGGCATCTTCGCCCCCAG | |
| | CAACGCCAGCTACAAGGCCGTCGCTTCCTGGCTCCAGAGCCACGG | |
| | CGTCAAGAACTTCGTCAAGCAGGCCGGCAGCATCTGGTTCTACACC | |
| | ACCGTCAGCACCGCCAACAAGATGCTCAGCACCGACTTCAAGCACT | |
| | ACAGCGACCCCGTCGGCATCGAGAAGCTCCGCACCCTCCAGTACA | |
| | GCATCCCCGAGGAACTCGTCGGCCACGTCGACCTCATCAGCCCCA | |
| | CCACCTACTTCGGCAACAACCACCCTGCCACCGCCCGCACCCCCAA | |
| | CATGAAGGCCATCAACGTCACCTACCAGATCTTCCACCCCGACTGC | |
| | CTCAAGACCAAGTACGGCGTCGACGGCTACGCCCCCTCACCTCGAT | |
| | GCGGCAGCCGAATCGGCTTCGGCAGCTTCCTCAACGAGACTGCCA | |
| | GCTACAGCGACCTCGCCCAGTTCGAGAAGTACTTCGACCTCCCCAA | |
| | CCAGAACCTCAGCACCCTCCTCATCAACGGCGCCATCGACGTCCAG | |
| | CCCCCCAGCAACAAGAACGACAGCGAGGCCAACATGGACGTCCAG | |
| | ACCATCCTCACCTTCGTCCAGCCCCTGCCCATCACCGAGTTCGTCG | |
| | TCGCCGGCATCCCCCCCTACATTCCCGATGCCGCCCTCCCCATTGG | |
| | CGACCCCGTTCAGAACGAGCCCTGGCTTGAGTACTTCGAGTTCCTC | |
| | ATGAGCCGCACCAACGCCGAGCTGCCCCAGGTCATTGCCAACAGC | |
| | TACGGCGACGAGGAACAGACCGTCCCCCAGGCCTACGCCGTCCGC | |
| | GTCTGCAACCAGATTGGCCTCCTCGGCCTCCGCGGCATCAGCGTCA | |
| | TTGCCTCTAGCGGCGACACCGGCGTCGGCATGTCTTGCATGGCCA | |
| | GCAACAGCACCACCCCCCAGTTCAACCCCATGTTCCCCGCCAGCTG | |
| | CCCCTACATCACCACCGTCGGCGGCACCCAGCACCTCGACAACGA | |
| | GATCGCCTGGGAGCTGAGCAGCGGCGGCTTCAGCAACTACTTCAC | |
| | CCGCCCCTGGTATCAAGAGGACGCCGCCAAGACCTACCTTGAGCG | |
| | CCACGTCAGCACCGAGACTAAGGCCTACTACGAGCGCTACGCCAAC | |
| | TTCCTGGGCCGAGGCTTTCCTGACGTCGCCGCCCTCAGCCTCAACC | |
| | CCGACTACCCCGTCATCATCGGCGGCGAGCTTGGCCCTAACGGCG | |
| | GCACTTCTGCTGCCGCCCCTGTCGTCGCCAGCATCATTGCCCTGCT | |
| | CAACGACGCCCGCCTCTGCCTCGGCAAGCCTGCCCTCGGCTTTCTC | |
| | AACCCCCTCATCTACCAGTACGCCGACAAGGGCGGCTTCACCGACA | |
| | TCACCAGCGGCCAGTCTTGGGGCTGCGCCGGCAACACCACTCAGA | |
| | CTGGACCTCCCCCTCCTGGCGCTGGCGTCATTCCTGGCGCTCACTG | |
| | GAACGCCACCAAGGGCTGGGACCCCGTCACCGGCTTTGGCACCCC | |
| | CAACTTCAAGAAGCTCCTCAGCCTCGCCCTCAGCGTCTAA | |
| 92 | ATGCAGACCTTCGGTGCTTTTCTCGTTTCCTTCCTCGCCGCCAGCG | Agaricus |
| | GCCTGGCCGCGGCCTCTCCTCTTGCTCGACGCTGGGACGACTTCG | bisporus var. |
| | CCGAGAAGCACGCCTGGGTCGAGGTTCCTCGCGGCTGGGAGATGG | burnettii |
| | TCAGCGAGGCCCCTAGCGACCACACCTTCGACCTCCGCATCGGCG | JB137-S8 |
| | TCAAGAGCAGCGGCATGGAACAGCTCATCGAGAACCTCATGCAGAC | |
| | CAGCGACCCCACCCACAGCCGCTACGGCCAGCACCTCAGCAAGGA | |
| | AGAACTCCACGACTTCGTCCAGCCCCACCCCGACTCTACTGGCGCC | |
| | GTCGAGGCCTGGCTTGAGGACTTCGGCATCAGCGACGACTTCATCG | |
| | ACCGCACCGGCAGCGGCAACTGGGTCACCGTCCGAGTCTCTGTCG | |
| | CCCAGGCCGAGCGAATGCTCGGCACCAAGTACAACGTCTACCGCC | |
| | ACAGCGAGAGCGGCGAGTCCGTCGTCCGCACCATGAGCTACAGCC | |
| | TCCCCAGCGAGCTGCACAGCCACATCGACGTCGTCGCCCCCACCA | |
| | CCTACTTCGGCACCATGAAGTCGATGCGCGTCACCTCGTTCCTCCA | |
| | GCCCGAGATCGAGCCCGTCGACCCCTCTGCCAAGCCTTCTGCTGCT | |
| | CCCGCCAGCTGCCTCAGCACCACCGTCATTACCCCCGACTGCCTCC | |
| | GCGACCTCTACAACACCGCCGACTACGTCCCCAGCGCCACCAGCC | |
| | GCAACGCCATTGGCATTGCCGGCTACCTCGACCGCAGCAACCGAG | |
| | CCGACCTCCAGACCTTCTTCCGCCGCTTTCGCCCTGACGCCGTCGG | |
| | CTTCAACTACACCACCGTCCAGCTCAACGGCGGAGGCGACGACCA | |
| | GAACGACCCTGGCGTCGAGGCCAACCTCGACATCCAGTACGCCGC | |
| | TGGCATTGCCTTCCCCACCCCCGCCACCTACTGGTCTACTGGCGGC | |
| | AGCCCCCCCTTCATCCCCGACACCCAGACCCCCACCAACACCAACG | |
| | AGCCCTACCTCGACTGGATCAACTTCGTCCTCGGCCAGGATGAGAT | |
| | CCCCCAGGTCATCAGCACCAGCTACGGCGACGACGAGCAGACCGT | |
| | CCCCGAGGACTACGCCACCAGCGTCTGCAACCTCTTCGCCCAGCTT | |
| | GGCTCTCGCGGCGTCACCGTCTTTTTCAGCAGCGGCGACTTCGGC | |
| | GTCGGCGGTGGCGACTGCCTCACTAACGACGGCAGCAACCAGGTC | |
| | CTCTTCCAGCCCGCCTTCCCTGCCAGCTGCCCCTTTGTCACTGCCG | |
| | TCGGCGGCACCGTCCGACTCGACCCTGAGATCGCCGTCAGCTTCA | |
| | GCGGCGGTGGCTTCAGCCGCTACTTCAGCCGCCCCAGCTACCAGA | |
| | ACCAGACCGTCGCCCAGTTCGTCAGCAACCTCGGCAACACCTTCAA | |
| | CGGCCTCTACAACAAGAACGGCCGAGCCTACCCCGACCTCGCCGC | |
| | TCAGGGCAACGGCTTCCAGGTCGTCATCGACGGCATCGTCCGATC | |
| | GGTCGGCGGCACTTCTGCCAGCAGCCCTACCGTCGCCGGCATCTT | |
| | CGCCCTGCTCAACGACTTCAAGCTCTCTCGCGGCCAGAGCACCCTC | |
| | GGCTTCATCAACCCCCTCATCTACAGCAGCGCCACCTCCGGCTTCA | |
| | ACGACATCCGAGCCGGCACCAACCCTGGCTGTGGCACCCGAGGCT | |
| | TTACCGCCGGCACTGGCTGGGACCCTGTCACCGGACTCGGCACCC | |
| | CTGACTTTCTCCGCCTCCAGGGCCTCATCTAA | |
| 93 | ATGCAGACCTTCGGTGCTTTTCTCGTTTCCTTCCTCGCCGCCAGCG | Magnaporthe |
| | GCCTGGCCGCGGCCCGCGTCTTTGATTCTCTCCCTCACCCCCCTCG | oryzae 70-15 |
| | CGGCTGGTCCTACTCTCACGCCGCTGAGAGCACCGAGCCCCTCAC | |
| | CCTCCGAATTGCCCTCCGCCAGCAGAACGCCGCTGCCCTTGAGCA | |
| | GGTCGTCCTCCAGGTCAGCAACCCCCGCCACGCCAACTACGGCCA | |
| | GCACCTCACCCGAGATGAGCTGCGCTCTTACACCGCCCCTACCCCT | 114123 |
| | CGCGCTGTCCGCTCTGTCACTAGCTGGCTCGTCGACAACGGCGTC | |
| | GACGACTACACCGTCGAGCACGACTGGGTCACCCTCCGCACCACT | |
| | GTCGGCGCTGCCGATCGACTCCTCGGCGCCGACTTTGCCTGGTAC | |
| | GCTGGCCCTGGCGAGACTCTCCAGCTCCGCACTCTCAGCTACGGC | |
| | GTGGACGACAGCGTCGCCCCTCACGTCGATCTCGTCCAGCCCACC | |
| | ACCCGCTTTGGCGGCCCTGTTGGCCAGGCCAGCCACATCTTCAAGC | |
| | AGGACGACTTCGACGAGCAGCAGCTCAAGACCCTCAGCGTCGGCT | |
| | TCCAGGTCATGGCCGACCTCCCTGCTAACGGCCCTGGCAGCATTAA | |
| | GGCCGCCTGCAACGAGAGCGGCGTCACCCCTCTCTGCCTCCGCAC | |
| | CCTCTACCGCGTCAACTACAAGCCCGCCACCACCGGCAACCTCGTC | |
| | GCCTTCGCCAGCTTCCTTGAGCAGTACGCCCGCTACAGCGACCAGC | |
| | AGGCCTTCACCCAGCGAGTCCTTGGCCCTGGCGTCCCGCTCCAGA | |
| | ACTTCAGCGTCGAGACTGTCAACGGCGGAGCCAACGACCAGCAGA | |
| | GCAAGCTCGATAGCGGCGAGGCCAACCTCGACCTCCAGTACGTCAT | |
| | GGCCATGTCCCACCCCATCCCCATCCTTGAGTACAGCACTGGCGGC | |
| | CGAGGCCCCCTCGTCCCTACTCTCGATCAGCCCAACGCCAACAACA | |
| | GCAGCAACGAGCCCTACCTTGAGTTCCTCACCTACCTGCTCGCCCA | |
| | GCCCGACAGCGCCATTCCCCAGACTCTCAGCGTGAGCTACGGCGA | |
| | GGAAGAACAGAGCGTCCCCCGCGACTACGCCATCAAGGTCTGCAA | |
| | CATGTTCATGCAGCTCGGCGCTCGCGGCGTCAGCGTCATGTTTAGC | |
| | AGCGGCGATAGCGGCCCTGGCAACGACTGCGTCCGAGCCTCTGAC | |
| | AACGCCACCTTCTTCGGCAGCACCTTCCCTGCCGGCTGCCCCTACG | |
| | TCACTAGCGTCGGCAGCACCGTCGGCTTCGAGCCTGAGCGAGCCG | |
| | TCAGCTTTAGCTCCGGCGGCTTCAGCATCTACCACGCCCGACCCGA | |
| | CTACCAGAACGAGGTCGTCCCCAAGTACATCGAGAGCATCAAGGCC | |
| | AGCGGCTACGAGAAGTTCTTCGACGGCAACGGCCGAGGCATCCCC | |
| | GATGTCGCTGCTCAGGGCGCTCGCTTCGTCGTCATCGACAAGGGC | |
| | CGCGTCAGCCTCATCAGCGGCACTAGCGCTTCCAGCCCCGCCTTC | |
| | GCTGGCATGGTCGCCCTCGTCAACGCCGCTCGCAAGAGCAAGGAT | |
| | ATGCCCGCCCTCGGCTTCCTCAACCCCATGCTCTACCAGAACGCTG | |
| | CCGCCATGACCGACATCGTCAACGGCGCTGGCATCGGCTGCCGCA | |
| | AGCAGCGCACCGAGTTTCCCAACGGTGCCCGCTTCAACGCCACCG | |
| | CCGGATGGGACCCTGTCACTGGCCTTGGCACCCCCCTGTTCGACAA | |
| | GCTCCTCGCCGTTGGCGCTCCCGGCGTCCCTAACGCCTAA | |
| 94 | ATGCAGACCTTCGGTGCTTTTCTCGTTTCCTTCCTCGCCGCCAGCG | Togninia |
| | GCCTGGCCGCGGCCTCCGATGTCGTCCTTGAGTCTCTCCGCGAGG | minima |
| | TCCCCCAGGGCTGGAAGCGACTCCGAGATGCCGACCCCGAGCAGA | UCRPA7 |
| | GCATCAAGCTCCGCATTGCCCTTGAGCAGCCCAACCTCGACCTCTT | |
| | CGAGCAGACCCTCTACGACATCAGCAGCCCCGACCACCCCAAGTAC | |
| | GGCCAGCACCTCAAGAGCCACGAGCTGCGCGACATCATGGCCCCT | |
| | CGCGAGGAATCCACTGCCGCCGTCATTGCCTGGCTCCAGGATGCT | |
| | GGCCTCAGCGGCAGCCAGATCGAGGACGACAGCGACTGGATCAAC | |
| | ATCCAGACCACCGTCGCCCAGGCCAACGACATGCTCAACACCACCT | |
| | TCGGCCTCTTCGCCCAAGAGGGCACCGAGGTCAACCGCATTCGCG | |
| | CCCTCGCCTACAGCGTCCCCGAGGAAATTGTCCCCCACGTCAAGAT | |
| | GATCGCCCCCATCATCCGCTTCGGCCAGCTCCGCCCTCAGATGAGC | |
| | CACATCTTCAGCCACGAGAAGGTCGAGGAAACCCCCAGCATCGGCA | |
| | CCATCAAGGCCGCTGCCATCCCCAGCGTCGACCTCAACGTCACCG | |
| | CCTGCAACGCCAGCATCACCCCCGAGTGCCTCCGCGCCCTCTACAA | |
| | CGTCGGCGACTACGAGGCCGACCCCAGCAAGAAGTCCCTCTTCGG | |
| | CGTCTGCGGCTACCTTGAGCAGTACGCCAAGCACGACCAGCTCGC | |
| | CAAGTTCGAGCAGACGTACGCCCCCTACGCCATCGGCGCCGACTT | |
| | CAGCGTCGTCACCATCAACGGCGGAGGCGACAACCAGACCAGCAC | |
| | CATCGACGACGGCGAGGCCAACCTCGACATGCAGTACGCCGTCAG | |
| | CATGGCCTACAAGACCCCCATCACCTACTACAGCACTGGCGGCCGA | |
| | GGCCCCCTCGTCCCTGATCTCGATCAGCCCGACCCCAACGACGTCA | |
| | GCAACGAGCCCTACCTCGACTTCGTCAGCTACCTCCTCAAGCTCCC | |
| | CGACAGCAAGCTCCCCCAGACCATCACCACCAGCTACGGCGAGGA | |
| | CGAGCAGAGCGTCCCCCGCAGCTACGTCGAGAAGGTCTGCACCAT | |
| | GTTCGGCGCCCTTGGCGCCCGAGGCGTCAGCGTCATTTTCAGCTCT | |
| | GGCGACACCGGCGTCGGCAGCGCCTGCCAGACTAACGACGGCAAG | |
| | AACACCACCCGCTTTCTGCCCATCTTCCCTGCCGCCTGCCCCTACG | |
| | TCACTAGCGTCGGCGGCACCCGCTACGTCGATCCTGAGGTCGCCG | |
| | TCAGCTTCAGCAGCGGCGGCTTCAGCGACATCTTCCCCACCCCCCT | |
| | GTACCAGAAGGGCGCCGTCAGCGGCTACCTCAAGATCCTCGGCGA | |
| | CCGCTGGAAGGGCCTCTACAACCCTCACGGCCGAGGCTTCCCTGA | |
| | CGTCAGCGGCCAGTCTGTCCGCTACCACGTCTTTGACTACGGCAAG | |
| | GACGTCATGTACAGCGGCACCAGCGCCAGCGCCCCCATGTTTGCT | |
| | GCTCTCGTCAGCCTCCTCAACAACGCCCGCCTCGCCAAGAAGCTCC | |
| | CCCCTATGGGCTTCCTCAACCCCTGGCTCTACACCGTCGGCTTCAA | |
| | CGGCCTCACCGACATCGTCCACGGCGGCTCTACTGGCTGCACCGG | |
| | CACCGATGTCTACAGCGGCCTGCCTACCCCCTTCGTCCCCTACGCC | |
| | TCTTGGAACGCCACCGTCGGCTGGGACCCTGTCACTGGCCTTGGC | |
| | ACCCCCCTGTTCGACAAGCTCCTCAACCTCAGCACCCCCAACTTCC | |
| | ACCTCCCCCACATCGGCGGCCACTAA | |
| 95 | ATGCAGACCTTCGGTGCTTTTCTCGTTTCCTTCCTCGCCGCCAGCG | Bipolaris |
| | GCCTGGCCGCGGCCTCTACCACTTCTCACGTCGAGGGCGAGGTCG | maydis C5 |
| | TCGAGCGCCTTCATGGCGTCCCTGAGGGCTGGTCACAGGTCGGCG | |
| | CTCCCAACCCCGACCAGAAGCTCCGCTTCCGCATTGCCGTCCGCAG | |
| | CGCCGACAGCGAGCTGTTCGAGCGCACCCTCATGGAAGTCAGCAG | |
| | CCCCAGCCACCCCCGCTACGGCCAGCACCTCAAGCGCCACGAGCT | |
| | GAAGGACCTCATCAAGCCTCGCGCCAAGAGCACCAGCAACATCCTC | |
| | AACTGGCTCCAAGAGAGCGGCATCGAGGCCCGCGACATCCAGAAC | |
| | GACGGCGAGTGGATCAGCTTCTACGCCCCCGTCAAGCGAGCCGAG | |
| | CAGATGATGAGCACCACCTTCAAGACCTACCAGAACGAGGCCCGAG | |
| | CCAACATCAAGAAGATCCGCAGCCTCGACTACAGCGTCCCCAAGCA | |
| | CATCCGCGACGACATCGACATCATCCAGCCCACCACGCGCTTCGGC | |
| | CAGATCCAGCCTGAGCGCAGCCAGGTCTTTAGCCAAGAGGAAGTCC | |
| | CCTTCAGCGCCCTCGTCGTCAACGCCACGTGCAACAAGAAGATCAC | |
| | CCCCGACTGCCTCGCCAACCTCTACAACTTCAAGGACTACGACGCC | |
| | AGCGACGCCAACGTCACGATCGGCGTCAGCGGCTTCCTTGAGCAG | |
| | TACGCCCGCTTCGACGACCTCAAGCAGTTCATCAGCACCTTCCAGC | |
| | CCAAGGCCGCTGGCTCCACCTTCCAGGTCACCAGCGTCAACGCTG | |
| | GCCCCTTCGACCAGAACAGCACCGCCTCTAGCGTCGAGGCCAACC | |
| | TCGACATCCAGTACACCACCGGCCTCGTCGCCCCCGACATCGAGAC | |
| | TCGCTACTTCACCGTCCCCGGACGCGGCATCCTCATCCCCGACCTC | |
| | GACCAGCCTACCGAGAGCGACAACGCCAACGAGCCCTACCTCGAC | |
| | TACTTCACCTACCTCAACAACCTTGAGGACGAGGAACTCCCCGACG | |
| | TCCTCACCACCAGCTACGGCGAGAGCGAGCAGAGCGTCCCTGCCG | |
| | AGTACGCCAAGAAGGTCTGCAACCTCATCGGCCAGCTCGGCGCTC | |
| | GCGGCGTCAGCGTCATTTTCAGCAGCGGCGACACCGGCCCTGGCA | |
| | GCGCCTGCCAGACTAACGACGGCAAGAACACCACCCGCTTTCTGCC | |
| | CATCTTCCCCGCCAGCTGCCCCTACGTCACTAGCGTCGGCGGCACT | |
| | GTCGGCGTCGAGCCTGAGAAGGCCGTCAGCTTTAGCAGCGGCGGC | |
| | TTCAGCGACCTCTGGCCCCGACCTGCCTACCAAGAGAAGGCCGTG | |
| | AGCGAGTACCTTGAGAAGCTCGGCGACCGCTGGAACGGCCTCTAC | |
| | AACCCTCAGGGCCGAGGCTTCCCTGACGTCGCTGCTCAGGGCCAG | |
| | GGCTTCCAGGTCTTTGACAAGGGCCGCCTCATCTCGGTCGGCGGC | |
| | ACATCTGCTTCCGCCCCTGTCTTTGCCAGCGTCGTCGCCCTCCTCA | |
| | ACAACGCCCGAAAGGCTGCCGGAATGAGCAGCCTCGGCTTCCTCA | |
| | ACCCCTGGATCTACGAGCAGGGCTACAAGGGCCTCACCGACATCGT | |
| | CGCTGGCGGCTCTACTGGCTGCACCGGCCGCTCTATCTACAGCGG | |
| | CCTCCCTGCCCCCCTGGTCCCTTACGCTTCTTGGAACGCCACCGAG | |
| | GGCTGGGACCCCGTCACTGGCTATGGCACCCCCGACTTCAAGCAG | |
| | CTCCTCACCCTCGCCACCGCCCCCAAGTCTGGCGAGCGACGAGTT | |
| | CGACGAGGCGGCCTTGGAGGCCAGGCTTAA | |

The at least one tripeptidyl peptidase may:
(a) comprise the amino acid sequence SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof;
(b) comprise an amino acid having at least 70% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof;
(c) be encoded by a nucleotide sequence comprising the sequence SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95;
(d) be encoded by a nucleotide sequence comprising at least about 70% sequence identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95;
(e) be encoded by a nucleotide sequence which hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95 under medium stringency conditions; or

(f) be encoded by a nucleotide sequence which differs from SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95 due to degeneracy of the genetic code.

The tripeptidyl peptidase may be expressed as a polypeptide sequence which undergoes further post-transcriptional and/or post-translational modification.

In one embodiment the tripeptidyl peptidase may comprise the amino acid sequence SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28 or a functional fragment thereof.

In another embodiment the tripeptidyl peptidase comprise an amino acid having at least 70% identity to SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28 or a functional fragment thereof.

In one embodiment the tripeptidyl peptidase may comprise the amino acid sequence SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18 or a functional fragment thereof.

In another embodiment the tripeptidyl peptidase comprise an amino acid having at least 70% identity to SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18 or a functional fragment thereof.

In another embodiment the tripeptidyl peptidase may be a “mature” tripeptidyl peptidase which has undergone post-transcriptional and/or post-translational modification (e.g. post-translational cleavage). Suitably such modification may lead to an activation of the enzyme. Suitably the tripeptidyl peptidase may comprise the amino acid sequence SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof.

In another embodiment the tripeptidyl peptidase comprise an amino acid having at least 70% identity to SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof.

In a yet further embodiment the tripeptidyl peptidase may comprise the amino acid sequence SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45 or a functional fragment thereof.

In another embodiment the tripeptidyl peptidase comprise an amino acid having at least 70% identity to SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45 or a functional fragment thereof.

The term “functional fragment” is a portion of an amino acid sequence that retains its peptidase enzyme activity. In other words it if a portion of an amino acid sequence which retains tripeptidyl peptidase activity as defined herein e.g. retains tripeptidyl peptidase activity as measured using the EBSA assay taught herein.

In one embodiment, a functional fragment of a tripeptidyl peptidase may be a functional fragment of a proline tolerant tripeptidyl peptidase.

A functional fragment of a proline tolerant tripeptidyl peptidase is a portion of a proline tolerant tripeptidyl peptidase predominantly having exopeptidase activity wherein said proline tolerant tripeptidyl peptidase is capable of cleaving tri-peptides from the N-terminus of peptides having
(i) (A) Proline at P1; and
   (B) An amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; and/or
(ii) (a′) Proline at P1′; and
   (b′) An amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′. Alternatively or additionally a functional fragment of a proline tolerant tripeptidyl peptidase is a portion of a proline tolerant tripeptidyl peptidase predominantly having exopeptidase activity and capable of cleaving tri-peptides from the N-terminus of peptides having proline at P1 and P1′.

The “portion” is any portion that still has the activity as defined above, suitably a portion may be at least 50 amino acids in length, more suitably at least 100. In other embodiments the portion may be about 150 or about 200 amino acids in length.

In one embodiment the functional fragment may be portion of a tripeptidyl peptidase following post transcriptional and/or post-translational modification (e.g. cleavage). Suitably the functional fragment may comprise a sequence shown as: SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54 or SEQ ID No. 55.

The tripeptidyl peptidase may comprise one or more amino acid sequence selected from SEQ ID No. 1, or a functional fragment thereof.

The tripeptidyl peptidase may comprise an amino acid having at least 70% identity to SEQ ID No. 1 or a functional fragment thereof.

The tripeptidyl peptidase may comprise one or more amino acid sequence selected from SEQ ID No. 2, or a functional fragment thereof.

The tripeptidyl peptidase may comprise an amino acid having at least 70% identity to SEQ ID No. 2 or a functional fragment thereof.

The proline tolerant tripeptidyl peptidase may comprise one or more amino acid sequence selected from SEQ ID No. 3 or a functional fragment thereof.

The tripeptidyl peptidase may comprise an amino acid having at least 70% identity to SEQ ID No. 3 or a functional fragment thereof.

The tripeptidyl peptidase may comprise one or more amino acid sequence selected from SEQ ID No. 4 or a functional fragment thereof.

The tripeptidyl peptidase may comprise an amino acid having at least 70% identity to SEQ ID No. 4 or a functional fragment thereof.

The tripeptidyl peptidase may comprise one or more amino acid sequence selected from SEQ ID No. 5 or a functional fragment thereof.

The tripeptidyl peptidase may comprise an amino acid having at least 70% identity to SEQ ID No. 5 or a functional fragment thereof.

The tripeptidyl peptidase may comprise one or more amino acid sequence selected from SEQ ID No. 6 or a functional fragment thereof.

The tripeptidyl peptidase may comprise an amino acid having at least 70% identity to SEQ ID No. 6 or a functional fragment thereof.

The tripeptidyl peptidase may comprise one or more amino acid sequence selected from SEQ ID No. 7 or a functional fragment thereof.

The tripeptidyl peptidase may comprise an amino acid having at least 70% identity to SEQ ID No. 7 or a functional fragment thereof.

The tripeptidyl peptidase may comprise one or more amino acid sequence selected from SEQ ID No. 8 or a functional fragment thereof.

The tripeptidyl peptidase may comprise an amino acid having at least 70% identity to SEQ ID No. 8 or a functional fragment thereof.

The tripeptidyl peptidase may comprise one or more amino acid sequence selected from SEQ ID No. 9 or a functional fragment thereof.

The tripeptidyl peptidase may comprise an amino acid having at least 70% identity to SEQ ID No. 9 or a functional fragment thereof.

The tripeptidyl peptidase may comprise one or more amino acid sequence selected from SEQ ID No. 10 or a functional fragment thereof.

The tripeptidyl peptidase may comprise an amino acid having at least 70% identity to SEQ ID No. 10 or a functional fragment thereof.

The tripeptidyl peptidase may comprise one or more amino acid sequence selected from SEQ ID No. 11 or a functional fragment thereof.

The tripeptidyl peptidase may comprise an amino acid having at least 70% identity to SEQ ID No. 11 or a functional fragment thereof.

The tripeptidyl peptidase may comprise one or more amino acid sequence selected from SEQ ID No. 12 or a functional fragment thereof.

The tripeptidyl peptidase may comprise an amino acid having at least 70% identity to SEQ ID No. 12 or a functional fragment thereof.

The tripeptidyl peptidase may comprise one or more amino acid sequence selected from SEQ ID No. 13 or a functional fragment thereof.

The tripeptidyl peptidase may comprise an amino acid having at least 70% identity to SEQ ID No. 13 or a functional fragment thereof.

The tripeptidyl peptidase may comprise one or more amino acid sequence selected from SEQ ID No. 14 or a functional fragment thereof.

The tripeptidyl peptidase may comprise an amino acid having at least 70% identity to SEQ ID No. 14 or a functional fragment thereof.

The tripeptidyl peptidase may comprise one or more amino acid sequence selected from SEQ ID No. 15 or a functional fragment thereof.

The tripeptidyl peptidase may comprise an amino acid having at least 70% identity to SEQ ID No. 15 or a functional fragment thereof.

The tripeptidyl peptidase may comprise one or more amino acid sequence selected from SEQ ID No. 16 or a functional fragment thereof.

The tripeptidyl peptidase may comprise an amino acid having at least 70% identity to SEQ ID No. 16 or a functional fragment thereof.

The tripeptidyl peptidase may comprise one or more amino acid sequence selected from SEQ ID No. 17 or a functional fragment thereof.

The tripeptidyl peptidase may comprise an amino acid having at least 70% identity to SEQ ID No. 17 or a functional fragment thereof.

The tripeptidyl peptidase may comprise one or more amino acid sequence selected from SEQ ID No. 18 or a functional fragment thereof.

The tripeptidyl peptidase may comprise an amino acid having at least 70% identity to SEQ ID No. 18 or a functional fragment thereof.

The tripeptidyl peptidase may comprise one or more amino acid sequence selected from SEQ ID No. 19 or a functional fragment thereof.

The tripeptidyl peptidase may comprise an amino acid having at least 70% identity to SEQ ID No. 19 or a functional fragment thereof.

The tripeptidyl peptidase may comprise one or more amino acid sequence selected from SEQ ID No. 20 or a functional fragment thereof.

The tripeptidyl peptidase may comprise an amino acid having at least 70% identity to SEQ ID No. 20 or a functional fragment thereof.

The tripeptidyl peptidase may comprise one or more amino acid sequence selected from SEQ ID No. 21 or a functional fragment thereof.

The tripeptidyl peptidase may comprise an amino acid having at least 70% identity to SEQ ID No. 21 or a functional fragment thereof.

The tripeptidyl peptidase may comprise one or more amino acid sequence selected from SEQ ID No. 22 or a functional fragment thereof.

The tripeptidyl peptidase may comprise an amino acid having at least 70% identity to SEQ ID No. 22 or a functional fragment thereof.

The tripeptidyl peptidase may comprise one or more amino acid sequence selected from SEQ ID No. 23 or a functional fragment thereof.

The tripeptidyl peptidase may comprise an amino acid having at least 70% identity to SEQ ID No. 23 or a functional fragment thereof.

The tripeptidyl peptidase may comprise one or more amino acid sequence selected from SEQ ID No. 24 or a functional fragment thereof.

The tripeptidyl peptidase may comprise an amino acid having at least 70% identity to SEQ ID No. 24 or a functional fragment thereof.

The tripeptidyl peptidase may comprise one or more amino acid sequence selected from SEQ ID No. 25 or a functional fragment thereof.

The tripeptidyl peptidase may comprise an amino acid having at least 70% identity to SEQ ID No. 25 or a functional fragment thereof.

The tripeptidyl peptidase may comprise one or more amino acid sequence selected from SEQ ID No. 26 or a functional fragment thereof.

The tripeptidyl peptidase may comprise an amino acid having at least 70% identity to SEQ ID No. 26 or a functional fragment thereof.

The tripeptidyl peptidase may comprise one or more amino acid sequence selected from SEQ ID No. 27 or a functional fragment thereof.

The tripeptidyl peptidase may comprise an amino acid having at least 70% identity to SEQ ID No. 27 or a functional fragment thereof.

The tripeptidyl peptidase may comprise one or more amino acid sequence selected from SEQ ID No. 28, or a functional fragment thereof.

The tripeptidyl peptidase may comprise an amino acid having at least 70% identity to SEQ ID No. 28 or a functional fragment thereof.

The tripeptidyl peptidase may comprise one or more amino acid sequence selected from SEQ ID No. 29, or a functional fragment thereof.

The tripeptidyl peptidase may comprise an amino acid having at least 70% identity to SEQ ID No. 29 or a functional fragment thereof.

The tripeptidyl peptidase may comprise one or more amino acid sequence selected from SEQ ID No. 30 or a functional fragment thereof.

The tripeptidyl peptidase may comprise an amino acid having at least 70% identity to SEQ ID No. 30 or a functional fragment thereof.

The tripeptidyl peptidase may comprise one or more amino acid sequence selected from SEQ ID No. 31 or a functional fragment thereof.

The tripeptidyl peptidase may comprise an amino acid having at least 70% identity to SEQ ID No. 31 or a functional fragment thereof.

The tripeptidyl peptidase may comprise one or more amino acid sequence selected from SEQ ID No. 32 or a functional fragment thereof.

The tripeptidyl peptidase may comprise an amino acid having at least 70% identity to SEQ ID No. 32 or a functional fragment thereof.

The tripeptidyl peptidase may comprise one or more amino acid sequence selected from SEQ ID No. 33 or a functional fragment thereof.

The tripeptidyl peptidase may comprise an amino acid having at least 70% identity to SEQ ID No. 33 or a functional fragment thereof.

The tripeptidyl peptidase may comprise one or more amino acid sequence selected from SEQ ID No. 34 or a functional fragment thereof.

The tripeptidyl peptidase may comprise an amino acid having at least 70% identity to SEQ ID No. 34 or a functional fragment thereof.

The tripeptidyl peptidase may comprise one or more amino acid sequence selected from SEQ ID No. 35 or a functional fragment thereof.

The tripeptidyl peptidase may comprise an amino acid having at least 70% identity to SEQ ID No. 35 or a functional fragment thereof.

The tripeptidyl peptidase may comprise one or more amino acid sequence selected from SEQ ID No. 36 or a functional fragment thereof.

The tripeptidyl peptidase may comprise an amino acid having at least 70% identity to SEQ ID No. 36 or a functional fragment thereof.

The tripeptidyl peptidase may comprise one or more amino acid sequence selected from SEQ ID No. 37 or a functional fragment thereof.

The tripeptidyl peptidase may comprise an amino acid having at least 70% identity to SEQ ID No. 37 or a functional fragment thereof.

The tripeptidyl peptidase may comprise one or more amino acid sequence selected from SEQ ID No. 38 or a functional fragment thereof.

The tripeptidyl peptidase may comprise an amino acid having at least 70% identity to SEQ ID No. 38 or a functional fragment thereof.

The tripeptidyl peptidase may comprise one or more amino acid sequence selected from SEQ ID No. 39 or a functional fragment thereof.

The tripeptidyl peptidase may comprise an amino acid having at least 70% identity to SEQ ID No. 39 or a functional fragment thereof.

The tripeptidyl peptidase may comprise one or more amino acid sequence selected from SEQ ID No. 40 or a functional fragment thereof.

The tripeptidyl peptidase may comprise an amino acid having at least 70% identity to SEQ ID No. 40 or a functional fragment thereof.

The tripeptidyl peptidase may comprise one or more amino acid sequence selected from SEQ ID No. 41 or a functional fragment thereof.

The tripeptidyl peptidase may comprise an amino acid having at least 70% identity to SEQ ID No. 41 or a functional fragment thereof.

The tripeptidyl peptidase may comprise one or more amino acid sequence selected from SEQ ID No. 42 or a functional fragment thereof.

The tripeptidyl peptidase may comprise an amino acid having at least 70% identity to SEQ ID No. 42 or a functional fragment thereof.

The tripeptidyl peptidase may comprise one or more amino acid sequence selected from SEQ ID No. 43 or a functional fragment thereof.

The tripeptidyl peptidase may comprise an amino acid having at least 70% identity to SEQ ID No. 43 or a functional fragment thereof.

The tripeptidyl peptidase may comprise one or more amino acid sequence selected from SEQ ID No. 44 or a functional fragment thereof.

The tripeptidyl peptidase may comprise an amino acid having at least 70% identity to SEQ ID No. 44 or a functional fragment thereof.

The tripeptidyl peptidase may comprise one or more amino acid sequence selected from SEQ ID No. 45 or a functional fragment thereof.

The tripeptidyl peptidase may comprise an amino acid having at least 70% identity to SEQ ID No. 45 or a functional fragment thereof.

The tripeptidyl peptidase may comprise one or more amino acid sequence selected from SEQ ID No. 46 or a functional fragment thereof.

The tripeptidyl peptidase may comprise an amino acid having at least 70% identity to SEQ ID No. 46 or a functional fragment thereof.

The tripeptidyl peptidase may comprise one or more amino acid sequence selected from SEQ ID No. 47 or a functional fragment thereof.

The tripeptidyl peptidase may comprise an amino acid having at least 70% identity to SEQ ID No. 47 or a functional fragment thereof.

The tripeptidyl peptidase may comprise one or more amino acid sequence selected from SEQ ID No. 48 or a functional fragment thereof.

The tripeptidyl peptidase may comprise an amino acid having at least 70% identity to SEQ ID No. 48 or a functional fragment thereof.

The tripeptidyl peptidase may comprise one or more amino acid sequence selected from SEQ ID No. 49 or a functional fragment thereof.

The tripeptidyl peptidase may comprise an amino acid having at least 70% identity to SEQ ID No. 49 or a functional fragment thereof.

The tripeptidyl peptidase may comprise one or more amino acid sequence selected from SEQ ID No. 50 or a functional fragment thereof.

The tripeptidyl peptidase may comprise an amino acid having at least 70% identity to SEQ ID No. 50 or a functional fragment thereof.

The tripeptidyl peptidase may comprise one or more amino acid sequence selected from SEQ ID No. 51 or a functional fragment thereof.

The tripeptidyl peptidase may comprise an amino acid having at least 70% identity to SEQ ID No. 51 or a functional fragment thereof.

The tripeptidyl peptidase may comprise one or more amino acid sequence selected from SEQ ID No. 52 or a functional fragment thereof.

The tripeptidyl peptidase may comprise an amino acid having at least 70% identity to SEQ ID No. 52 or a functional fragment thereof.

The tripeptidyl peptidase may comprise one or more amino acid sequence selected from SEQ ID No. 53 or a functional fragment thereof.

The tripeptidyl peptidase may comprise an amino acid having at least 70% identity to SEQ ID No. 53 or a functional fragment thereof.

The tripeptidyl peptidase may comprise one or more amino acid sequence selected from SEQ ID No. 54 or a functional fragment thereof.

The tripeptidyl peptidase may comprise an amino acid having at least 70% identity to SEQ ID No. 54 or a functional fragment thereof.

The tripeptidyl peptidase may comprise one or more amino acid sequence selected from SEQ ID No. 55 or a functional fragment thereof.

The tripeptidyl peptidase may comprise an amino acid having at least 70% identity to SEQ ID No. 55 or a functional fragment thereof.

Suitably the tripeptidyl peptidase may comprise an amino acid having at least 80% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof.

Suitably the tripeptidyl peptidase may comprise an amino acid having at least 85% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof.

Suitably the tripeptidyl peptidase may comprise an amino acid having at least 90% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof.

Suitably the tripeptidyl peptidase may comprise an amino acid having at least 95% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof.

Suitably the tripeptidyl peptidase may comprise an amino acid having at least 97% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof.

Suitably the tripeptidyl peptidase may comprise an amino acid having at least 99% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof.

In one embodiment the tripeptidyl peptidase may comprise an amino acid sequence selected from one more of the group consisting of: SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54 and SEQ ID No. 55.

In some embodiments, the tripeptidyl peptidase may comprise an amino acid sequence selected from one more of the group consisting of: SEQ ID No. 1, SEQ ID No. 2, SEQ ID No. 3, SEQ ID No. 29 and SEQ ID No. 30, or a sequence having at least 70% identity thereto. Suitably a sequence having at least 80% thereto or at least 90% thereto.

In some embodiments it may be suitable that the tripeptidyl peptidase may comprise an amino acid sequence selected from the group consisting of SEQ ID No. 3, SEQ ID No. 4, SEQ ID No. 30 and SEQ ID No. 31, or a sequence having at least 70% identity thereto. Suitably a sequence having at least 80% thereto or at least 90% thereto.

Advantageously these particular amino acid sequences may be particularly suited to cleaving peptide and/or protein substrates enriched in lysine, arginine and/or glycine. Particularly where lysine, arginine and/or glycine are present at the P1 position.

In other embodiments, the tripeptidyl peptidase may comprise an amino acid sequence selected from one more of the group consisting of: SEQ ID No. 1, SEQ ID No. 2, SEQ ID No. 5, SEQ ID No. 6, SEQ ID No. 7, SEQ ID No. 29, SEQ ID No. 32, SEQ ID No. 33 and SEQ ID No. 34, or a sequence having at least 70% identity thereto. Suitably a sequence having at least 80% thereto or at least 90% thereto.

Suitably the proline tolerant tripeptidyl peptidase may have the sequence SEQ ID No. 1, SEQ ID No. 2 or SEQ ID No. 29.

The tripeptidyl peptidase may comprise one or more of the sequence motifs selected from the group consisting of: xEANLD, y′Tzx′G and QNFSV.

Suitably, the tripeptidyl peptidase may comprise xEANLD.

x may be one or more amino acid selected from the group consisting of: G, T, S and V.

In another embodiment the proline tolerant tripeptidyl peptidase may comprise y′Tzx′G.

y′ may be one or more amino acid selected from the group consisting of: I, L and V.

z may be one or more amino acid selected from the group consisting of: S and T.

x′ may be one or more amino acid selected from the group consisting of: I and V.

In another embodiment the tripeptidyl peptidase may comprise the sequence motif QNFSV.

In a further embodiment the tripeptidyl peptidase may comprise the sequence motifs xEANLD and y′Tzx′G or xEANLD and QNFSV.

In a yet further embodiment the tripeptidyl peptidase may comprise the sequence motifs y′Tzx′G and QNFSV.

Suitably the tripeptidyl peptidase may comprise the sequence motifs xEANLD, y′Tzx′G and QNFSV.

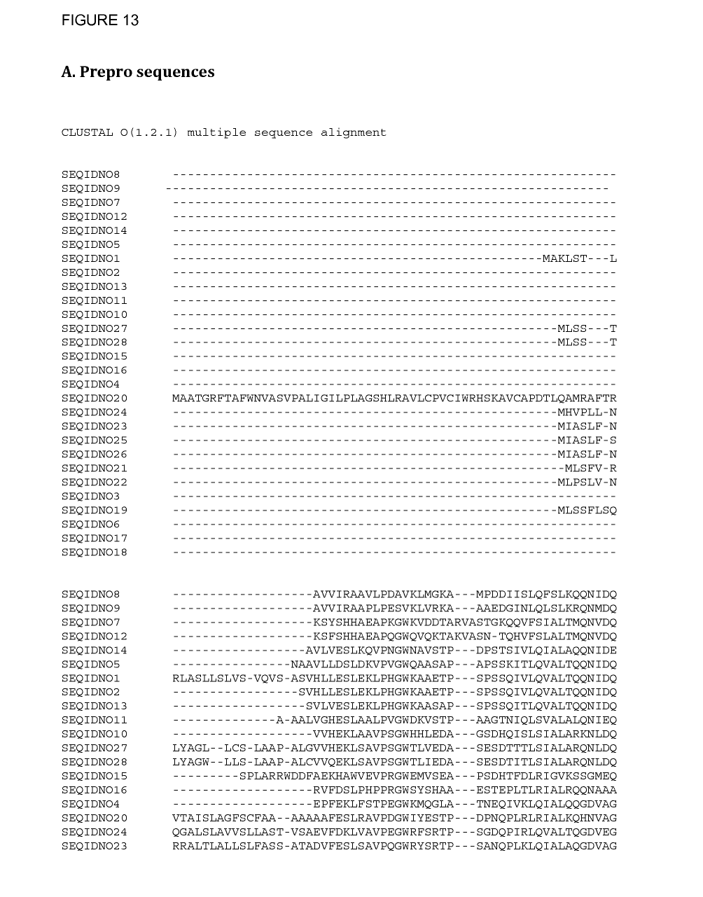

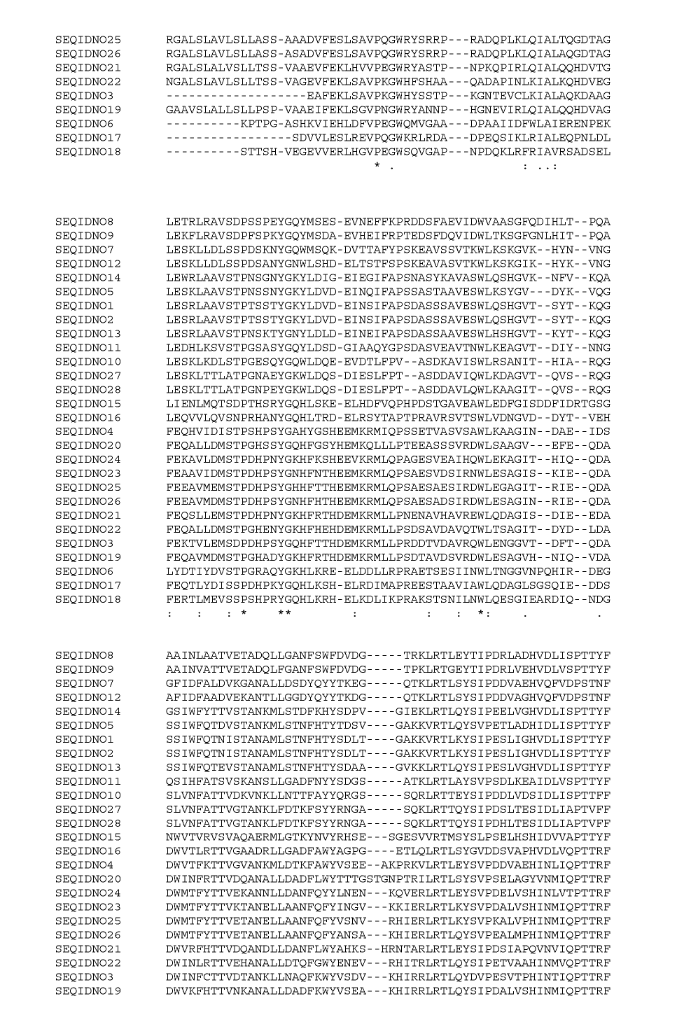

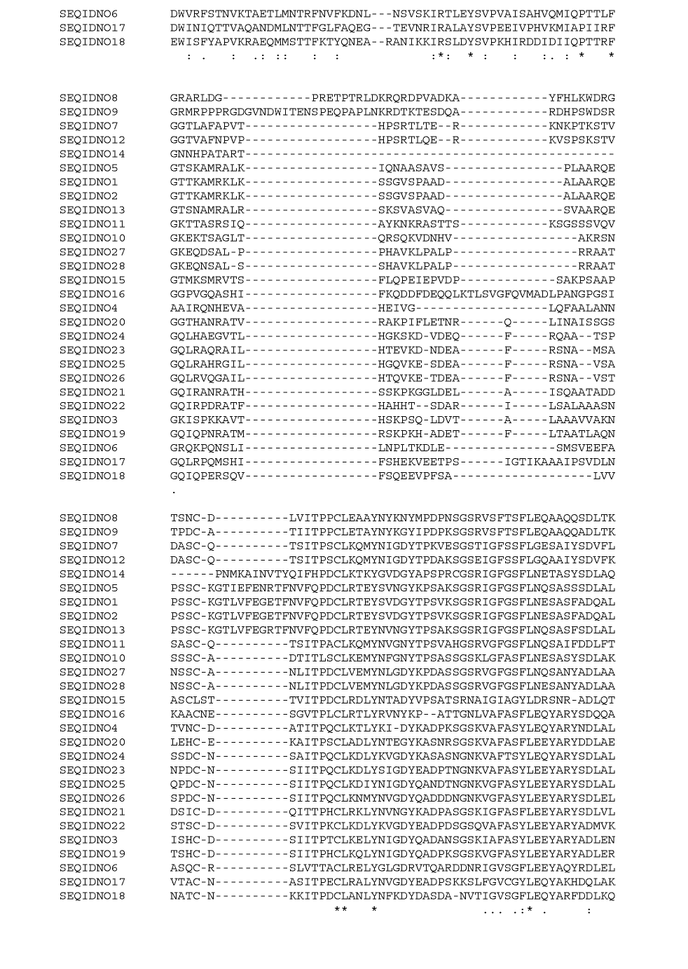

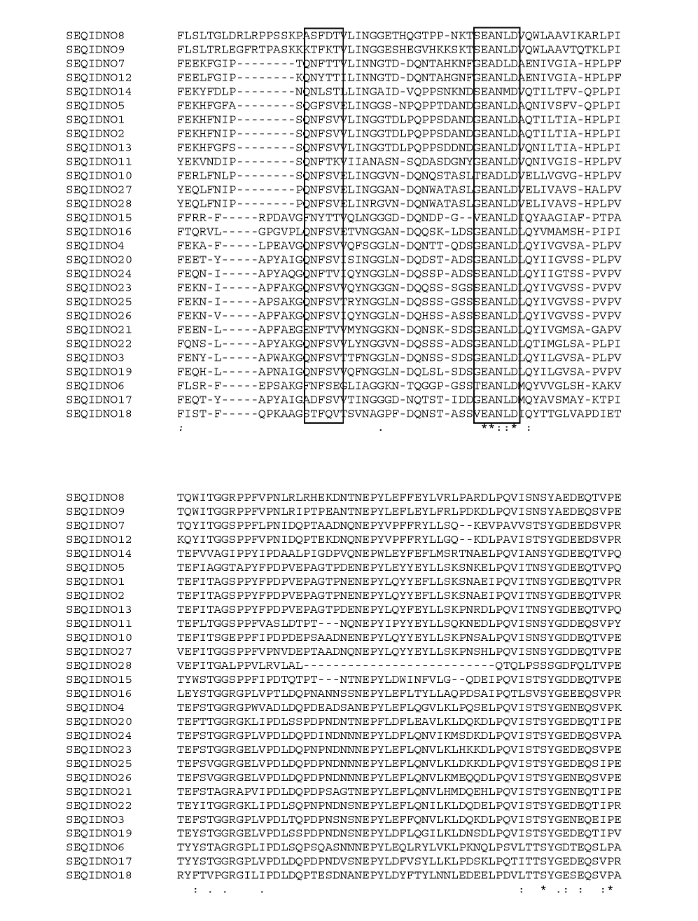

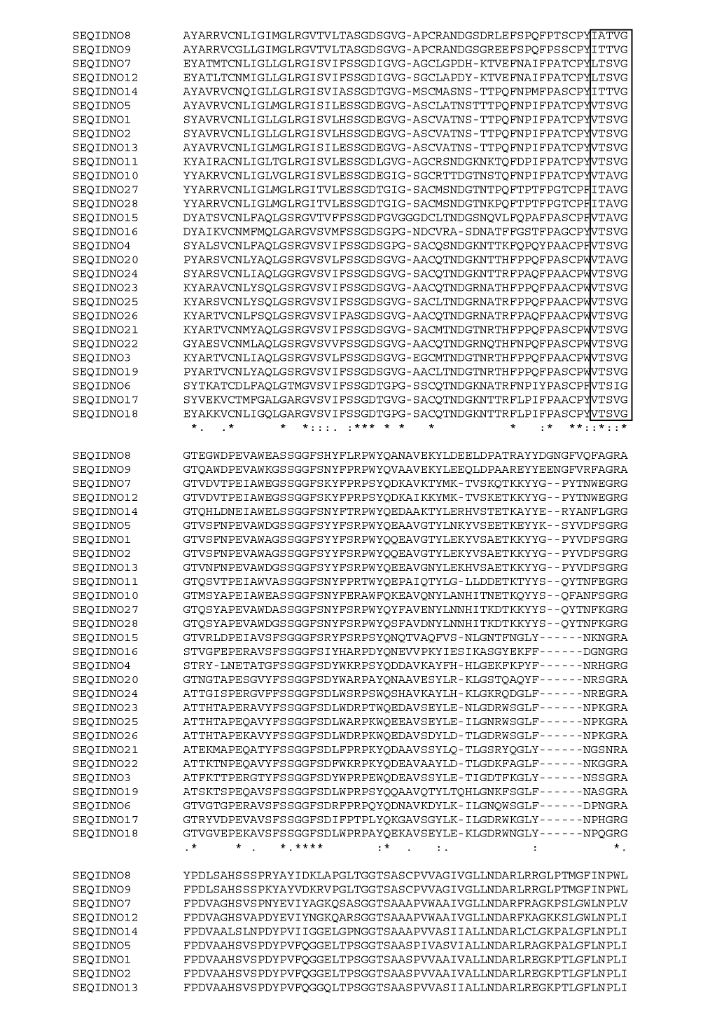

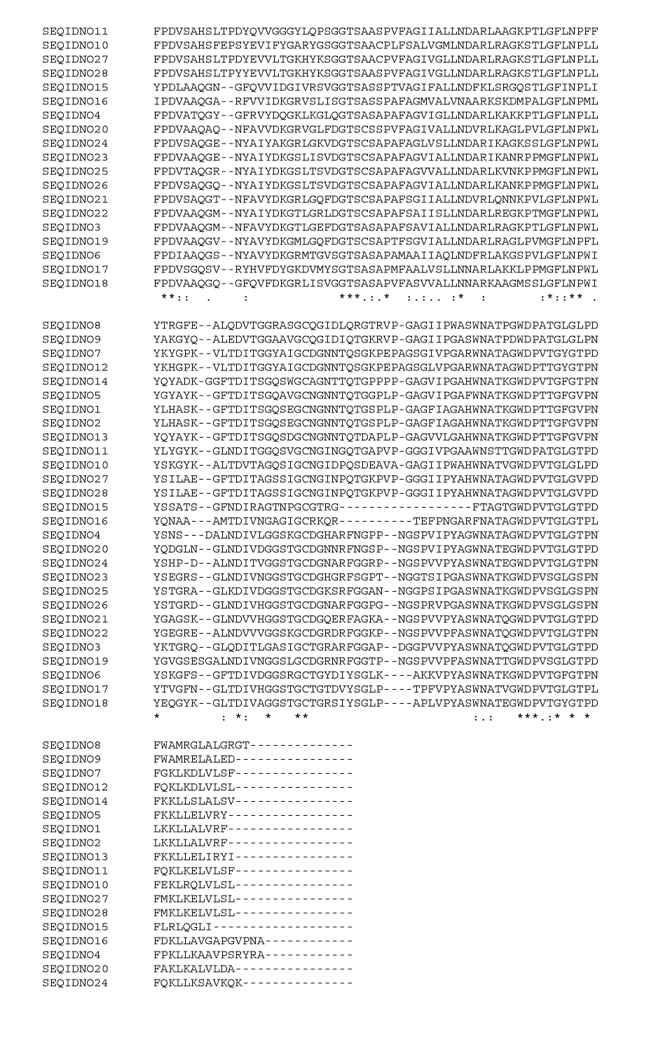

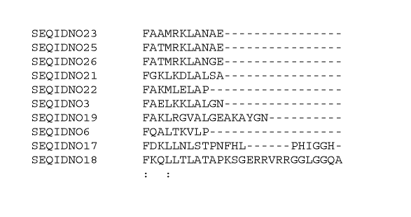

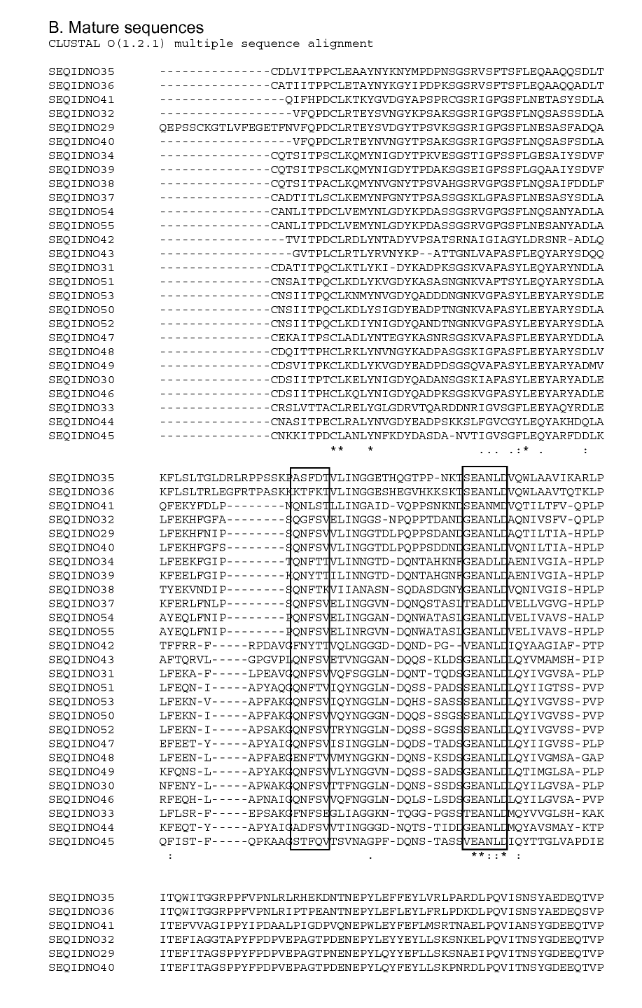

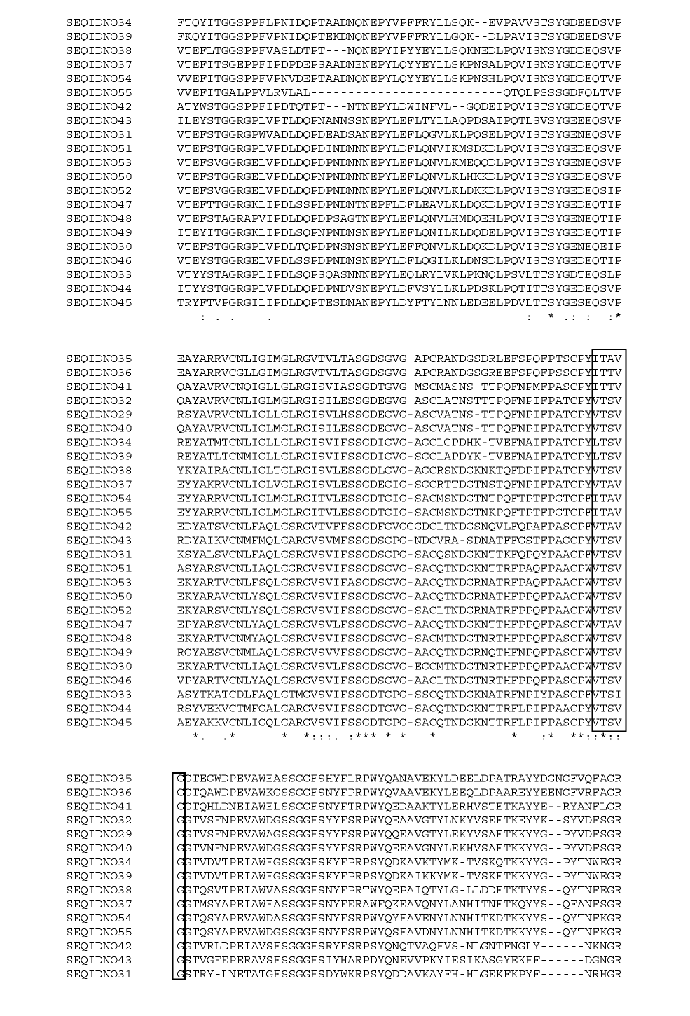

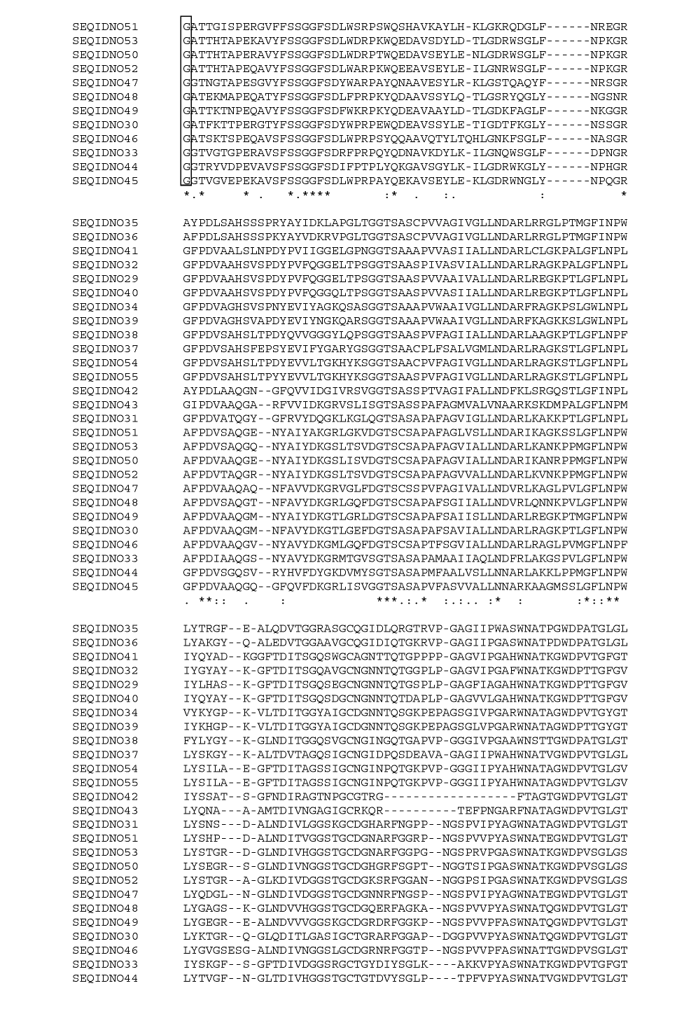

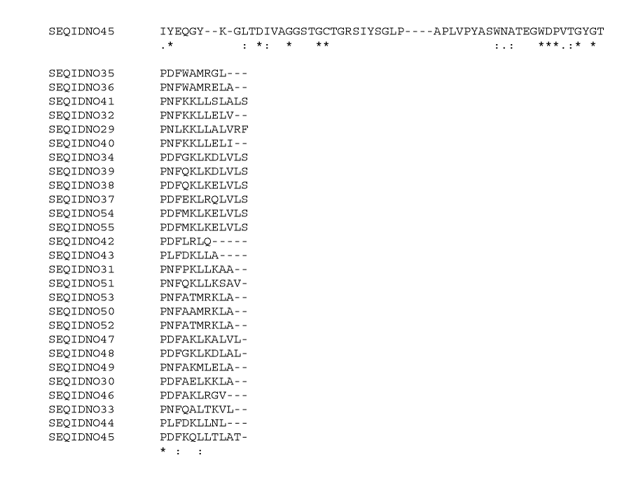

One or more of the motifs are present in the tripeptidyl peptidases for use in the present invention. FIG. 13 indicates the positioning of these motifs.

In one embodiment the tripeptidyl peptidase may be encoded by a nucleotide sequence shown as SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80 or a nucleotide sequence having at least 70% identity thereto. Suitably a sequence having at least 80% thereto or at least 90% thereto.

Preferably the tripeptidyl peptidase may be encoded by a nucleotide sequence having at least 95% sequence identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79 or SEQ ID No. 80, more preferably at least 99% identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79 or SEQ ID No. 80.

In another embodiment the tripeptidyl peptidase may be encoded by a nucleotide sequence which hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80 under medium stringency conditions. Suitably, a nucleotide sequence which hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80 under high stringency conditions

In a further embodiment, the tripeptidyl peptidase may be encoded by a nucleotide sequence which differs from SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80 due to degeneracy of the genetic code.

In one embodiment the nucleotide sequence comprising a nucleotide sequence shown as SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95 may be a DNA, cDNA, synthetic DNA and/or RNA sequence.

Preferably the sequence is a DNA sequence, more preferably a cDNA sequence coding for the tripeptidyl peptidase of the present invention.

In one aspect, preferably the amino acid and/or nucleotide sequence for use in the present invention is in an isolated form. The term “isolated” means that the sequence is at least substantially free from at least one other component with which the sequence is naturally associated in nature and as found in nature. The amino acid and/or nucleotide sequence for use in the present invention may be provided in a form that is substantially free of one or more contaminants with which the substance might otherwise be associated. Thus, for example it may be substantially free of one or more potentially contaminating polypeptides and/or nucleic acid molecules.

In one aspect, preferably the amino acid and/or nucleotide sequence for use in the present invention is in a purified form. The term “purified” means that a given component is present at a high level. The component is desirably the predominant component present in a composition. Preferably, it is present at a level of at least about 90%, or at least about 95% or at least about 98%, said level being determined on a dry weight/dry weight basis with respect to the total composition under consideration.

### Enzymes

In one embodiment the enzyme for use in the present invention may be a proline tolerant tripeptidyl peptidase comprising SEQ ID No. 1, a functional fragment thereof or a sequence having at least 70% identity to SEQ ID No. 1. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 2, a functional fragment thereof or a sequence having at least 70% identity to SEQ ID No. 2. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 3, a functional fragment thereof or a sequence having at least 70% identity to SEQ ID No. 3. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 4, a functional fragment thereof or a sequence having at least 70% identity to SEQ ID No. 4. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 5, a functional fragment thereof or a sequence having at least 70% identity to SEQ ID No. 5. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 6, a functional fragment thereof or a sequence having at least 70% identity to SEQ ID No. 6. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 7, a functional fragment thereof or a sequence having at least 70% identity to SEQ ID No. 7. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 8, a functional fragment thereof or a sequence having at least 70% identity to SEQ ID No. 8. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 9, a functional fragment thereof or a sequence having at least 70% identity to SEQ ID No. 9. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 10, a functional fragment thereof or a sequence having at least 70% identity to SEQ ID No. 10. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 11, a functional fragment thereof or a sequence having at least 70% identity to SEQ ID No. 11. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 12, a functional fragment thereof or a sequence having at least 70% identity to SEQ ID No. 12. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 13, a functional fragment thereof or a sequence having at least 70% identity to SEQ ID No. 13. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 14, a functional fragment thereof or a sequence having at least 70% identity to SEQ ID No. 14. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 15, a functional fragment thereof or a sequence having at least 70% identity to SEQ ID No. 15. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 16, a functional fragment thereof or a sequence having at least 70% identity to SEQ ID No. 16. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 17, a functional fragment thereof or a sequence having at least 70% identity to SEQ ID No. 17. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 18, a functional fragment thereof or a sequence having at least 70% identity to SEQ ID No. 18. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 19, a functional fragment thereof or a sequence having at least 70% identity to SEQ ID No. 19. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 20, a functional fragment thereof or a sequence having at least 70% identity to SEQ ID No. 20. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 21, a functional fragment thereof or a sequence having at least 70% identity to SEQ ID No. 21. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 22, a functional fragment thereof or a sequence having at least 70% identity to SEQ ID No. 22. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 23, a functional fragment thereof or a sequence having at least 70% identity to SEQ ID No. 23. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 24, a functional fragment thereof or a sequence having at least 70% identity to SEQ ID No. 24. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 25, a functional fragment thereof or a sequence having at least 70% identity to SEQ ID No. 25. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 26, a functional fragment thereof or a sequence having at least 70% identity to SEQ ID No. 26. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 27, a functional fragment thereof or a sequence having at least 70% identity to SEQ ID No. 27. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 28, a functional fragment thereof or a sequence having at least 70% identity to SEQ ID No. 28. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 29, a functional fragment thereof or a sequence having at least 70% identity to SEQ ID No. 29. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 30, a functional fragment thereof or a sequence having at least 70% identity to SEQ ID No. 30. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 31, a functional fragment thereof or a sequence having at least 70% identity to SEQ ID No. 31. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 32, a functional fragment thereof or a sequence having at least 70% identity to SEQ ID No. 32. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 33, a functional fragment thereof or a sequence having at least 70% identity to SEQ ID No. 33. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 34, a functional fragment thereof or a sequence having at least 70% identity to SEQ ID No. 34. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 35, a functional fragment thereof or a sequence having at least 70% identity to SEQ ID No. 35. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 36, a functional fragment thereof or a sequence having at least 70% identity to SEQ ID No. 36. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 37, a functional fragment thereof or a sequence having at least 70% identity to SEQ ID No. 37. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 38, a functional fragment thereof or a sequence having at least 70% identity to SEQ ID No. 38. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 39, a functional fragment thereof or a sequence having at least 70% identity to SEQ ID No. 39. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 40, a functional fragment thereof or a sequence having at least 70% identity to SEQ ID No. 40. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 41, a functional fragment thereof or a sequence having at least 70% identity to SEQ ID No. 41. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 42, a functional fragment thereof or a sequence having at least 70% identity to SEQ ID No. 42. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 43, a functional fragment thereof or a sequence having at least 70% identity to SEQ ID No. 43. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 44, a functional fragment thereof or a sequence having at least 70% identity to SEQ ID No. 44. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 45, a functional fragment thereof or a sequence having at least 70% identity to SEQ ID No. 45. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 46, a functional fragment thereof or a sequence having at least 70% identity to SEQ ID No. 46. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 47, a functional fragment thereof or a sequence having at least 70% identity to SEQ ID No. 47. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 48, a functional fragment thereof or a sequence having at least 70% identity to SEQ ID No. 48. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 49, a functional fragment thereof or a sequence having at least 70% identity to SEQ ID No. 49. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 50, a functional fragment thereof or a sequence having at least 70% identity to SEQ ID No. 50. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 51, a functional fragment thereof or a sequence having at least 70% identity to SEQ ID No. 51. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 52, a functional fragment thereof or a sequence having at least 70% identity to SEQ ID No. 52. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 53, a functional fragment thereof or a sequence having at least 70% identity to SEQ ID No. 53. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 54, a functional fragment thereof or a sequence having at least 70% identity to SEQ ID No. 54. Suitably the enzyme may have at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase comprising SEQ ID No. 55, a functional fragment thereof or a sequence having at least 70% identity to SEQ ID No. 55. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 56 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 57 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 58 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 59 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 60 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 61 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 62 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 63 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 64 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 65 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 66 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 67 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 68 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 69 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 70 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 71 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 72 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 73 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 74 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 75 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 76 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 77 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 78 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 79 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 80 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 81 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 82 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 83 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 84 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 85 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 86 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 87 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 88 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 89 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 90 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 91 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 92 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 93 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 94 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a proline tolerant tripeptidyl peptidase encoded by a nucleotide sequence comprising the sequence shown as SEQ ID No. 95 or a sequence having at least 70% identity thereto. Suitably by a sequence having at least 80% or 85% identity thereto. Preferably at least 90% or 95% identity thereto. More preferably at least 97% or 99% identity thereto.

### Nucleotide Sequence

The scope of the present invention encompasses nucleotide sequences encoding proteins having the specific properties as defined herein.

The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or anti-sense strand.

The term “nucleotide sequence” in relation to the present invention includes genomic DNA, cDNA, synthetic DNA, and RNA. Preferably it means DNA, more preferably cDNA sequence coding for the present invention.

In a preferred embodiment, the nucleotide sequence when relating to and when encompassed by the per se scope of the present invention does not include the native nucleotide sequence according to the present invention when in its natural environment and when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. However, the amino acid sequence encompassed by scope the present invention can be isolated and/or purified post expression of a nucleotide sequence in its native organism. Preferably, however, the amino acid sequence encompassed by scope of the present invention may be expressed by a nucleotide sequence in its native organism but wherein the nucleotide sequence is not under the control of the promoter with which it is naturally associated within that organism.

Typically, the nucleotide sequence encompassed by the scope of the present invention is prepared using recombinant DNA techniques (i.e. recombinant DNA). However, in an alternative embodiment of the invention, the nucleotide sequence could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232).

### Preparation of the Nucleotide Sequence

A nucleotide sequence encoding either a protein which has the specific properties as defined herein or a protein which is suitable for modification may be identified and/or isolated and/or purified from any cell or organism producing said protein. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used. Alternatively, enzyme-encoding clones could be identified by inserting fragments of genomic DNA into an expression vector, such as a plasmid, transforming enzyme-negative bacteria with the resulting genomic DNA library, and then plating the transformed bacteria onto agar plates containing a substrate for enzyme (i.e. maltose), thereby allowing clones expressing the enzyme to be identified.

In a yet further alternative, the nucleotide sequence encoding the enzyme may be prepared synthetically by established standard methods, e.g. the phosphoroamidite method described by Beucage S. L. et al., (1981) Tetrahedron Letters 22, p 1859-1869, or the method described by Matthes et al., (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487-491) the teaching of these documents being incorporated herein by reference.

### Amino Acid Sequences

The scope of the present invention also encompasses amino acid sequences of enzymes having the specific properties as defined herein.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The amino acid sequence may be prepared/isolated from a suitable source, or it may be made synthetically or it may be prepared by use of recombinant DNA techniques.

The protein encompassed in the present invention may be used in conjunction with other proteins, particularly enzymes. Thus the present invention also covers a combination of proteins wherein the combination comprises the protein/enzyme of the present invention and another protein/enzyme, which may be another protein/enzyme according to the present invention. This aspect is discussed in a later section.

Preferably the amino acid sequence when relating to and when encompassed by the per se scope of the present invention is not a native enzyme. In this regard, the term “native enzyme” means an entire enzyme that is in its native environment and when it has been expressed by its native nucleotide sequence.

### Isolated

In one aspect, preferably the amino acid sequence, or nucleic acid, or enzyme according to the present invention is in an isolated form. The term “isolated” means that the sequence or enzyme or nucleic acid is at least substantially free from at least one other component with which the sequence, enzyme or nucleic acid is naturally associated in nature and as found in nature. The sequence, enzyme or nucleic acid of the present invention may be provided in a form that is substantially free of one or more contaminants with which the substance might otherwise be associated. Thus, for example it may be substantially free of one or more potentially contaminating polypeptides and/or nucleic acid molecules.

### Purified

In one aspect, preferably the sequence, enzyme or nucleic acid according to the present invention is in a purified form. The term “purified” means that the given component is present at a high level. The component is desirably the predominant component present in a composition. Preferably, it is present at a level of at least about 80% said level being determined on a dry weight/dry weight basis with respect to the total composition under consideration. Suitably it may be present at a level of at least about 90%, or at least about 95, or at least about 98% said level being determined on a dry weight/dry weight basis with respect to the total composition under consideration.

### Sequence Identity or Sequence Homology

The present invention also encompasses the use of sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

The homologous amino acid sequence and/or nucleotide sequence should provide and/or encode a polypeptide which retains the functional activity and/or enhances the activity of the enzyme.

In the present context, a homologous sequence is taken to include an amino acid or a nucleotide sequence which may be at least 75, 85 or 90% identical, preferably at least 95 or 98% identical to the subject sequence. Typically, the homologues will comprise the same active sites etc. as the subject amino acid sequence for instance. Although homology can also be considered in terms of similarity (i.e. amino acid residues having similar chemical properties/functions), in the context of the present invention it is preferred to express homology in terms of sequence identity.

In one embodiment, a homologous sequence is taken to include an amino acid sequence or nucleotide sequence which has one or several additions, deletions and/or substitutions compared with the subject sequence.

In one embodiment the present invention relates to a protein whose amino acid sequence is represented herein or a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

Suitably, the degree of identity with regard to an amino acid sequence is determined over at least 20 contiguous amino acids, preferably over at least 30 contiguous amino acids, preferably over at least 40 contiguous amino acids, preferably over at least 50 contiguous amino acids, preferably over at least 60 contiguous amino acids, preferably over at least 100 contiguous amino acids, preferably over at least 200 contiguous amino acids.

In one embodiment the present invention relates to a nucleic acid sequence (or gene) encoding a protein whose amino acid sequence is represented herein or encoding a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In the present context, a homologous sequence is taken to include a nucleotide sequence which may be at least 75, 85 or 90% identical, preferably at least 95 or 98% identical to a nucleotide sequence encoding a polypeptide of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology or % identity therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.gov), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60), such as for example in the GenomeQuest search tool (www.genomequest.com).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244).

Once the software has produced an optimal alignment, it is possible to calculate homology, preferably % sequence identity. The software typically does this as part of the sequence comparison and generates a numerical result.

Should Gap Penalties be used when determining sequence identity, then preferably the following parameters are used for pairwise alignment:

| FOR BLAST | |
|---|---|
| GAP OPEN | 9 |
| GAP EXTENSION | 2 |

| FOR CLUSTAL | DNA | PROTEIN |
|---|---|---|
| Weight Matrix | IUB | Gonnet 250 |
| GAP OPENING | 15 | 10 |
| GAP EXTEND | 6.66 | 0.1 |

In one embodiment, CLUSTAL may be used with the gap penalty and gap extension set as defined above.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides.

Suitably, the degree of identity with regard to a nucleotide sequence is determined over at least 100 contiguous nucleotides, preferably over at least 200 contiguous nucleotides, preferably over at least 300 contiguous nucleotides, preferably over at least 400 contiguous nucleotides, preferably over at least 500 contiguous nucleotides, preferably over at least 600 contiguous nucleotides, preferably over at least 700 contiguous nucleotides, preferably over at least 800 contiguous nucleotides.

Suitably, the degree of identity with regard to a nucleotide sequence may be determined over the whole sequence.

Suitably, the degree of identity with regard to a protein (amino acid) sequence is determined over at least 100 contiguous amino acids, preferably over at least 200 contiguous amino acids, preferably over at least 300 contiguous amino acids.

Suitably, the degree of identity with regard to an amino acid or protein sequence may be determined over the whole sequence taught herein.

In the present context, the term “query sequence” means a homologous sequence or a foreign sequence, which is aligned with a subject sequence in order to see if it falls within the scope of the present invention. Accordingly, such query sequence can for example be a prior art sequence or a third party sequence.

In one preferred embodiment, the sequences are aligned by a global alignment program and the sequence identity is calculated by identifying the number of exact matches identified by the program divided by the length of the subject sequence.

In one embodiment, the degree of sequence identity between a query sequence and a subject sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the subject sequence.

In yet a further preferred embodiment, the global alignment program is selected from the group consisting of CLUSTAL and BLAST (preferably BLAST) and the sequence identity is calculated by identifying the number of exact matches identified by the program divided by the length of the subject sequence.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by synthetic amino acids (e.g. unnatural amino acids) include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction enzyme recognition sites so that the amplified DNA can be cloned into a suitable cloning vector.

### Hybridisation

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under medium stringency conditions (e.g. 50° C. and 0.2×SSC {1×SSC=0.15 M NaCl, 0.015 M Na₃citrate pH 7.0}) to the nucleotide sequences presented herein.

More preferably, the term “variant” encompasses sequences that are complementary to sequences that are capable of hybridising under high stringency conditions (e.g. 65° C. and 0.1×SSC {1×SSC=0.15 M NaCl, 0.015 M Na₃citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers nucleotide sequences that can hybridise to the nucleotide sequence of the present invention, or the complement thereof, under medium stringency conditions (e.g. 50° C. and 0.2×SSC {1×SSC=0.15 M NaCl, 0.015 M Na₃citrate pH 7.0}).

In a more preferred aspect, the present invention covers nucleotide sequences that can hybridise to the nucleotide sequence of the present invention, or the complement thereof, under high stringent conditions (e.g. 65° C. and 0.1×SSC {1×SSC=0.15 M NaCl, 0.015 M Na₃citrate pH 7.0}).

Preferably hybridisation is analysed over the whole of the sequences taught herein.

### Molecular Evolution

As a non-limiting example, it is possible to produce numerous site directed or random mutations into a nucleotide sequence, either in vivo or in vitro, and to subsequently screen for improved functionality of the encoded polypeptide by various means.

In addition, mutations or natural variants of a polynucleotide sequence can be recombined with either the wildtype or other mutations or natural variants to produce new variants. Such new variants can also be screened for improved functionality of the encoded polypeptide. The production of new preferred variants can be achieved by various methods well established in the art, for example the Error Threshold Mutagenesis (WO 92/18645), oligonucleotide mediated random mutagenesis (U.S. Pat. No. 5,723,323), DNA shuffling (U.S. Pat. No. 5,605,793), exo-mediated gene assembly WO00/58517. The application of these and similar random directed molecular evolution methods allows the identification and selection of variants of the enzymes of the present invention which have preferred characteristics without any prior knowledge of protein structure or function, and allows the production of non-predictable but beneficial mutations or variants. There are numerous examples of the application of molecular evolution in the art for the optimisation or alteration of enzyme activity, such examples include, but are not limited to one or more of the following: optimised expression and/or activity in a host cell or in vitro, increased enzymatic activity, altered substrate and/or product specificity, increased or decreased enzymatic or structural stability, altered enzymatic activity/specificity in preferred environmental conditions, e.g. temperature, pH, substrate.

### Site-Directed Mutagenesis

Once a protein-encoding nucleotide sequence has been isolated, or a putative protein-encoding nucleotide sequence has been identified, it may be desirable to mutate the sequence in order to prepare a protein of the present invention.

Mutations may be introduced using synthetic oligonucleotides. These oligonucleotides contain nucleotide sequences flanking the desired mutation sites.

A suitable method is disclosed in Morinaga et al., (Biotechnology (1984) 2, p 646-649). Another method of introducing mutations into enzyme-encoding nucleotide sequences is described in Nelson and Long (Analytical Biochemistry (1989), 180, p 147-151).

### Recombinant

In one aspect the sequence for use in the present invention is a recombinant sequence—i.e. a sequence that has been prepared using recombinant DNA techniques.

These recombinant DNA techniques are within the capabilities of a person of ordinary skill in the art. Such techniques are explained in the literature, for example, J. Sambrook, E. F. Fritsch, and T. Maniatis, 1989, Molecular Cloning: A Laboratory Manual, Second Edition, Books 1-3, Cold Spring Harbor Laboratory Press.

### Synthetic

In one aspect the sequence for use in the present invention is a synthetic sequence—i.e. a sequence that has been prepared by in vitro chemical or enzymatic synthesis. It includes, but is not limited to, sequences made with optimal codon usage for host organisms—such as the methylotrophic yeasts Pichia and Hansenula.

Proteins and/or peptides for use in the present invention may also be of a synthetic origin.

### Expression of Enzymes

The nucleotide sequence for use in the present invention may be incorporated into a recombinant replicable vector. The vector may be used to replicate and express the nucleotide sequence, in protein/enzyme form, in and/or from a compatible host cell.

Expression may be controlled using control sequences e.g. regulatory sequences.

The protein produced by a host recombinant cell by expression of the nucleotide sequence may be secreted or may be contained intracellularly depending on the sequence and/or the vector used. The coding sequences may be designed with signal sequences which direct secretion of the substance coding sequences through a particular prokaryotic or eukaryotic cell membrane.

The term “expression vector” means a construct capable of in vivo or in vitro expression. In one embodiment the tripeptidyl peptidase for use in the present invention may be encoded by a vector. In other words the vector may comprise a nucleotide sequence encoding the tripeptidyl peptidase.

Preferably, the expression vector is incorporated into the genome of a suitable host organism. The term “incorporated” preferably covers stable incorporation into the genome.

The nucleotide sequence of the present invention may be present in a vector in which the nucleotide sequence is operably linked to regulatory sequences capable of providing for the expression of the nucleotide sequence by a suitable host organism.

The vectors for use in the present invention may be transformed into a suitable host cell as described below to provide for expression of a polypeptide of the present invention.

The choice of vector e.g. a plasmid, cosmid, or phage vector will often depend on the host cell into which it is to be introduced.

The vectors for use in the present invention may contain one or more selectable marker genes—such as a gene, which confers antibiotic resistance e.g. ampicillin, kanamycin, chloramphenicol or tetracyclin resistance. Alternatively, the selection may be accomplished by co-transformation (as described in WO91/17243).

Vectors may be used in vitro, for example for the production of RNA or used to transfect, transform, transduce or infect a host cell.

Thus, in a further embodiment, the invention provides a method of making nucleotide sequences of the present invention by introducing a nucleotide sequence of the present invention into a replicable vector, introducing the vector into a compatible host cell, and growing the host cell under conditions which bring about replication of the vector.

The vector may further comprise a nucleotide sequence enabling the vector to replicate in the host cell in question. Examples of such sequences are the origins of replication of plasmids pUC19, pACYC177, pUB110, pE194, pAMB1 and pIJ702.

The nucleotide sequence and/or vector encoding the tripeptidyl peptidase and/or the endoprotease may be codon optimised for expression in a particular host organism.

The nucleotide sequence and/or vector encoding the tripeptidyl peptidase and/or the endoprotease may be codon optimised for expression in a prokaryotic or eukaryotic cell. Suitably, the nucleotide sequence and/or vector encoding the tripeptidyl peptidase and/or the endoprotease may be codon optimised for expression in a fungal host organism (e.g. Trichoderma, preferably Trichoderma reesei).

Codon optimisation refers to a process of modifying a nucleic acid sequence for enhanced expression in a host cell of interest by replacing at least one codon (e.g. at least about more than 1, 2, 3, 4, 5, 10, 15, 20, 25, 50, 60, 70, 80 or 100 codons) of the native sequence with codons that are more frequently used in the genes of the host cell, whilst maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, amongst other things, the properties of the codons being translated and the availability of particular transfer RNA (tRNA) molecules. The predominance of selected tRNAs in a cell is generally a reflection of the codons used most frequently in peptide synthesis.

Accordingly, genes can be tailored for optimal gene expression in a given organism based on codon optimisation. A nucleotide sequence and/vector that has undergone this tailoring can be referred to therefore as a “codon optimised” nucleotide sequence and/or vector. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimising a particular sequence for expression in a particular host cell are also available, such as Gene Forge (Aptagen; Jacobus, Pa.). In some embodiments, one or more codons (e.g. 1, 2, 3, 4, 5, 10, 15, 20, 25, 50, or more, or all codons) in a sequence encoding a tripeptidyl peptidase and/or endoprotease for use in the present invention correspond to the most frequently used codon for a particular amino acid.

In one embodiment the nucleotide sequence encoding the tripeptidyl peptidase may be a nucleotide sequence which has been codon optimised for expression in Trichoderma reesei. In one embodiment the codon optimised sequence may comprise a nucleotide sequence shown as SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95 or a nucleotide sequence having at least 70% identity thereto. Suitably a sequence having at least 80% thereto or at least 90% thereto.

Preferably the codon optimised sequence may comprise a nucleotide sequence having at least 95% sequence identity to SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, more preferably at least 99% identity to SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95.

In one embodiment the proline tolerant tripeptidyl peptidase may be encoded by a nucleotide sequence which hybridises to SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95 under medium stringency conditions. Suitably, a nucleotide sequence which hybridises to SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95 under high stringency conditions.

In a further embodiment, the proline tolerant tripeptidyl peptidase may be encoded by a nucleotide sequence which differs from SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95 due to degeneracy of the genetic code.

There is also provided a vector (e.g. plasmid) comprising one or more of the sequences selected from the group consisting of: SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95.

In one embodiment the tripeptidyl peptidase for use in the present invention is part of a fermentate.

As used herein the term “fermentate” refers to the mixture of constituents present following (e.g. at the end of) the culturing of a host cell which fermentate includes the tripeptidyl peptidase, e.g. expressed by the host cell. The fermentate may comprises as well as the tripeptidyl peptidase in accordance with the present invention other components such as particulate matter, solids, substrates not utilised during culturing, debris, media, cell waste, etc. In one aspect, host cells (and particularly any spores) are removed from the fermentate and/or inactivated to provide a cell-free fermentate.

In other embodiments the tripeptidyl peptidase for use in the present invention is isolated or purified.

### Regulatory Sequences

In some applications, the nucleotide sequence for use in the present invention is operably linked to a regulatory sequence which is capable of providing for the expression of the nucleotide sequence, such as by the chosen host cell. By way of example, the present invention covers a vector comprising the nucleotide sequence of the present invention operably linked to such a regulatory sequence, i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the nucleotide sequence encoding the enzyme of the present invention may also be achieved by the selection of heterologous regulatory regions, e.g. promoter, secretion leader and terminator regions.

Preferably, the nucleotide sequence according to the present invention is operably linked to at least a promoter.

Other promoters may even be used to direct expression of the polypeptide of the present invention.

Examples of suitable promoters for directing the transcription of the nucleotide sequence in a bacterial, fungal or yeast host are well known in the art.

The promoter can additionally include features to ensure or to increase expression in a suitable host. For example, the features can be conserved regions such as a Pribnow Box or a TATA box.

### Constructs

The term “construct”—which is synonymous with terms such as “conjugate”, “cassette” and “hybrid”—includes a nucleotide sequence for use according to the present invention directly or indirectly attached to a promoter.

An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment.

The construct may even contain or express a marker, which allows for the selection of the genetic construct.

For some applications, preferably the construct of the present invention comprises at least the nucleotide sequence of the present invention operably linked to a promoter.

### Host Cells

The term “host cell”—in relation to the present invention includes any cell that comprises either the nucleotide sequence or an expression vector as described above and which is used in the recombinant production of a protein having the specific properties as defined herein.

Thus, a further embodiment of the present invention provides host cells transformed or transfected with a nucleotide sequence that expresses the protein of the present invention. The cells will be chosen to be compatible with the said vector and may for example be prokaryotic (for example bacterial), fungal, yeast or plant cells.

Examples of suitable bacterial host organisms are gram positive or gram negative bacterial species.

Depending on the nature of the nucleotide sequence encoding the polypeptide of the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

The use of suitable host cells—such as yeast, fungal and plant host cells—may provide for post-translational modifications (e.g. myristoylation, glycosylation, truncation, lipidation and tyrosine, serine or threonine phosphorylation) as may be needed to confer optimal biological activity on recombinant expression products of the present invention.

The host cell may be a protease deficient or protease minus strain. This may for example be the protease deficient strain Aspergillus oryzae JaL 125 having the alkaline protease gene named “alp” deleted. This strain is described in WO97/35956.

### Organism

The term “organism” in relation to the present invention includes any organism that could comprise the nucleotide sequence coding for the polypeptide according to the present invention and/or products obtained therefrom, and/or wherein a promoter can allow expression of the nucleotide sequence according to the present invention when present in the organism.

Suitable organisms may include a prokaryote, fungus, yeast or a plant.

The term “transgenic organism” in relation to the present invention includes any organism that comprises the nucleotide sequence coding for the polypeptide according to the present invention and/or the products obtained therefrom, and/or wherein a promoter can allow expression of the nucleotide sequence according to the present invention within the organism. Preferably the nucleotide sequence is incorporated in the genome of the organism.

The term “transgenic organism” does not cover native nucleotide coding sequences in their natural environment when they are under the control of their native promoter which is also in its natural environment.

Therefore, the transgenic organism of the present invention includes an organism comprising any one of, or combinations of, the nucleotide sequence coding for the polypeptide according to the present invention, constructs according to the present invention, vectors according to the present invention, plasmids according to the present invention, cells according to the present invention, tissues according to the present invention, or the products thereof.

For example the transgenic organism may also comprise the nucleotide sequence coding for the polypeptide of the present invention under the control of a heterologous promoter.

### Transformation of Host Cells/Organism

As indicated earlier, the host organism can be a prokaryotic or a eukaryotic organism. Examples of suitable prokaryotic hosts include E. coli and Bacillus subtilis, Bacillus licheniformis, Streptomyces, Clostridium, and the like.

Teachings on the transformation of prokaryotic hosts is well documented in the art, for example see Sambrook et al (Molecular Cloning: A Laboratory Manual, 2nd edition, 1989, Cold Spring Harbor Laboratory Press). If a prokaryotic host is used then the nucleotide sequence may need to be suitably modified before transformation—such as by removal of introns.

Filamentous fungi cells may be transformed using various methods known in the art—such as a process involving protoplast formation and transformation of the protoplasts followed by regeneration of the cell wall in a manner known. The use of Aspergillus as a host microorganism is described in EP 0 238 023.

Another host organism can be a plant. A review of the general techniques used for transforming plants may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

General teachings on the transformation of fungi, yeasts and plants are presented in following sections.

### Transformed Fungus

A host organism may be a fungus—such as a mould. Examples of suitable such hosts include any member belonging to the genera Thermomyces, Acremonium, Aspergillus, Penicillium, Mucor, Neurospora, Trichoderma, Rhizopus, Talaromyces, Humicola, and the like.

In one embodiment, the host organism may be a filamentous fungus.

Transforming filamentous fungi is discussed in U.S. Pat. No. 5,741,665 which states that standard techniques for transformation of filamentous fungi and culturing the fungi are well known in the art. An extensive review of techniques as applied to N. crassa is found, for example in Davis and de Serres, Methods Enzymol (1971) 17A: 79-143.

Further teachings which may also be utilised in transforming filamentous fungi are reviewed in U.S. Pat. No. 5,674,707.

In addition, gene expression in filamentous fungi is taught in in Punt et al. (2002) Trends Biotechnol 2002 May; 20(5):200-6, Archer & Peberdy Crit Rev Biotechnol (1997) 17(4):273-306.

The present invention encompasses the production of transgenic filamentous fungi according to the present invention prepared by use of these standard techniques.

Suitably the host organism is a Trichoderma host organism, e.g. a Trichoderma reesei host organism.

In another embodiment, the host organism can be of the genus Aspergillus, such as Aspergillus niger.

A transgenic Aspergillus according to the present invention can also be prepared by following, for example, the teachings of Turner G. 1994 (Vectors for genetic manipulation. In: Martinelli S. D., Kinghorn J. R. (Editors) Aspergillus: 50 years on. Progress in industrial microbiology vol 29. Elsevier Amsterdam 1994. pp. 641-666).

### Transformed Yeast

In another embodiment, the transgenic organism can be a yeast.

A review of the principles of heterologous gene expression in yeast are provided in, for example, Methods Mol Biol (1995), 49:341-54, and Curr Opin Biotechnol (1997) October; 8(5):554-60

In this regard, yeast—such as the species Saccharomyces cerevisiae or Pichia pastoris (see FEMS Microbiol Rev (2000 24(1):45-66), may be used as a vehicle for heterologous gene expression.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

For the transformation of yeast, several transformation protocols have been developed. For example, a transgenic Saccharomyces according to the present invention can be prepared by following the teachings of Hinnen et al., (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells may be selected using various selective markers—such as auxotrophic markers dominant antibiotic resistance markers.

### Culturing and Production

Host cells transformed with the nucleotide sequence of the present invention may be cultured under conditions conducive to the production of the encoded polypeptide and which facilitate recovery of the polypeptide from the cells and/or culture medium.

The medium used to cultivate the cells may be any conventional medium suitable for growing the host cell in questions and obtaining expression of the polypeptide.

The protein produced by a recombinant cell may be displayed on the surface of the cell.

The protein may be secreted from the host cells and may conveniently be recovered from the culture medium using well-known procedures.

### Secretion

Often, it is desirable for the protein to be secreted from the expression host into the culture medium from where the protein may be more easily recovered. According to the present invention, the secretion leader sequence may be selected on the basis of the desired expression host. Hybrid signal sequences may also be used with the context of the present invention.

Typical examples of heterologous secretion leader sequences are those originating from the fungal amyloglucosidase (AG) gene (glaA—both 18 and 24 amino acid versions e.g. from Aspergillus), the a-factor gene (yeasts e.g. Saccharomyces, Kluyveromyces and Hansenula) or the α-amylase gene (Bacillus).

By way of example, the secretion of heterologous proteins in E. coli is reviewed in Methods Enzymol (1990) 182:132-43.

### Post-Transcriptional and Post-Translational Modifications

Suitably the tripeptidyl peptidase and/or the endoprotease for use in the present invention may be encoded by any one of the nucleotide sequences taught herein.

Depending upon the host cell used post-transcriptional and/or post-translational modifications may be made. It is envisaged that the enzymes (e.g. the tripeptidyl peptidase and/or the endoprotease) for use in the present methods and/or uses encompasses enzymes (e.g. the tripeptidyl peptidase and/or the endoprotease) which have undergone post-transcriptional and/or post-translational modification.

One non-limiting example of a post-transcriptional and/or post-translational modifications is “clipping” or “cleavage” of a polypeptide (e.g. of the tripeptidyl peptidase and/or the endoprotease).

In some embodiments the polypeptide (e.g. the tripeptidyl peptidase and/or the endoprotease) may be clipped or cleaved. This may result in the conversion of the tripeptidyl peptidase and/or the endoprotease from an inactive or substantially inactive state to an active state (i.e. capable of performing the activity described herein).

The tripeptidyl peptidase may be a pro-peptide which undergoes further post-translational modification to a mature peptide, i.e. a polypeptide which has the tripeptidyl peptidase activity.

By way of example only SEQ ID No. 1 is the same as SEQ ID No. 29 except that SEQ ID No. 1 has undergone post-translational and/or post-transcriptional modification to remove some amino acids, more specifically 197 amino acids from the N-terminus. Therefore the polypeptide shown herein as SEQ ID No. 1 could be considered in some circumstances (i.e. in some host cells) as a pro-peptide—which is further processed to a mature peptide (SEQ ID No. 29) by post-translational and/or post-transcriptional modification. The precise modifications, e.g. cleavage site(s), in respect of the post-translational and/or post-transcriptional modification may vary slightly depending on host species. In some host species there may be no post translational and/or post-transcriptional modification, hence the pro-peptide would then be equivalent to the mature peptide (i.e. a polypeptide which has the tripeptidyl peptidase activity of the present invention). Without wishing to be bound by theory, the cleavage site(s) may be shifted by a few residues (e.g. 1, 2 or 3 residues) in either direction compared with the cleavage site shown by reference to SEQ ID No. 29 compared with SEQ ID No. 1. In other words, rather than cleavage at position 197 (R) for example, the cleavage may be at position 196-A, 195-A, 194-A, 198Q, 199E, 200P for example. In addition or alternatively, the cleavage may result in the removal of about 197 amino acids, in some embodiments the cleavage may result in the removal of between 194 and 200 residues.

Other examples of post-transcriptional and/or post-translational modifications include but are not limited to myristoylation, glycosylation, truncation, lipidation and tyrosine, serine or threonine phosphorylation. The skilled person will appreciate that the type of post-transcriptional and/or post-translational modifications that may occur to a protein (e.g. the tripeptidyl peptidase and/or the endoprotease) may depend on the host organism in which the protein (e.g. the tripeptidyl peptidase and/or the endoprotease) is expressed.

### Detection

A variety of protocols for detecting and measuring the expression of the amino acid sequence are known in the art. Examples include enzyme-linked immunosorbent assay (ELISA), radioimmunoassay (RIA) and fluorescent activated cell sorting (FACS).

A wide variety of labels and conjugation techniques are known by those skilled in the art and can be used in various nucleic and amino acid assays.

A number of companies such as Pharmacia Biotech (Piscataway, N.J.), Promega (Madison, Wis.), and US Biochemical Corp (Cleveland, Ohio) supply commercial kits and protocols for these procedures.

Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

Also, recombinant immunoglobulins may be produced as shown in U.S. Pat. No. 4,816,567.

### Fusion Proteins

The amino acid sequence for use according to the present invention may be produced as a fusion protein, for example to aid in extraction and purification. Examples of fusion protein partners include glutathione-S-transferase (GST), 6×His, GAL4 (DNA binding and/or transcriptional activation domains) and (β-galactosidase). It may also be convenient to include a proteolytic cleavage site between the fusion protein partner and the protein sequence of interest to allow removal of fusion protein sequences.

Preferably, the fusion protein will not hinder the activity of the protein sequence.

Gene fusion expression systems in E. coli have been reviewed in Curr Opin Biotechnol (1995) 6(5):501-6.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognised by a commercially available antibody.

### General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### Dosages

The tripeptidyl peptidase and/or the endoprotease for use in the methods and/or uses of the present invention may be dosed in any suitable amount.

In one embodiment the tripeptidyl peptidase may be dosed in an amount of about 5 mg to 3 g of enzyme per kg of feedstock.

In one embodiment suitably the tripeptidyl peptidase may be dosed in an amount of 25 mg to 1000 mg of enzyme per kg of feedstock.

In one embodiment the tripeptidyl peptidase may be dosed in an amount of about 0.01 mg-100 mg; 0.5 mg-100 mg; 1 mg-50 mg; 5 mg-100 mg; 5 mg-20 mg, 10 mg-100 mg; 0.05 mg-50 mg; or 0.10 mg-10 mg of enzyme per kg of feedstock.

In certain embodiments the tripeptidyl peptidase may be dosed in an amount of about 0.01 g to 1000 g of enzyme per kg of feedstock, such as 0.1 g to 500 g, such as 0.5 g to 700 g, such as in an amount of about 0.01 g-200 g, 0.01 g-100 g; 0.5 g-100 g; 1 g-50 g; 5 g-100 g; 5 g-20 g, 5 g 15 g, 10 g-100 g; 0.05 g-50 g; or 0.10 g-10 g of enzyme per kg of feedstock.

In one preferred embodiment, the tripeptidyl peptidase may be dosed in an amount of about 5 mg-20 mg of enzyme per kg of feedstock.

The exact amount will depend on the particular type of composition employed and on the specific protease activity per mg of protein.

In another embodiment the tripeptidyl peptidase may be dosed in an amount of about 1 mg to about 1 kg of enzyme per kg of feedstock. Suitably the tripeptidyl peptidase may be dosed at about 1 mg to about 250 g per kg of feedstock. Preferably at about 1 mg to about 100 g (more preferably at about 1 mg to about 1 g) per kg of feedstock.

In some embodiments the tripeptidyl peptidase may be dosed based on the number of tripeptidyl peptidase units (TPPU) per gram of dry solids present in a feedstock calculated according to the following assay:

the number of TPPU units of tripeptidyl peptidase may be calculated using 150 ul of 1 mM H-Ala-Ala-Phe-pNA (a para-nitroaniline derivatized substrate) in 0.1M NaOAc, pH4.5 in a well of 96 well microtiter plate mixed with various amounts of enzyme dilution to fit within a linear range of the assay. Absorbance at 410 nM is followed kinetically at room temp (25° C.). 1 U is defined as the amount of enzyme releasing 1 micromole of pNA per min. pNA molar adsorption at 410 is assumed to be 8800.

Therefore, in one embodiment, the tripeptidyl peptidase may be dosed in an amount of at least about 0.01 TPPU/g of dry solids present in a feedstock, suitably at least 0.02 TPPU/g of dry solids present in a feedstock.

In another embodiment the tripeptidyl peptidase may be dosed in an amount of between about 0.01 TPPU/g of dry solids present in a feedstock to about 0.5 TPPU/g of dry solids present in a feedstock. Suitably, the tripeptidyl peptidase may be dosed in an amount of between about 0.1 TPPU/g of dry solids present in a feedstock to about 0.25 TPPU/g of dry solids present in a feedstock. More suitably the tripeptidyl peptidase may be dosed in an amount of between about 0.01 TPPU/g of dry solids present in a feedstock to about 0.1 TPPU/g of dry solids present in a feedstock.

Preferably, the tripeptidyl peptidase may be dosed in an amount of between about 0.01 TPPU/g of dry solids present in a feedstock to about 0.05 TPPU/g of dry solids present in a feedstock.

Preferably the tripeptidyl peptidase may be dosed in an amount of about 0.02 TPPU/g of dry solids present in a feedstock.

The endoprotease may be dosed in an amount of about 50 to about 3000 mg of enzyme per kg of protein substrate, e.g. 0.05 to 3 g of enzyme per metric ton (MT) of feedstock.

Suitably, the endoprotease may be dosed in an amount of less than about 4.0 g of enzyme per MT of feedstock.

In another embodiment the endoprotease may be dosed at between about 0.5 g and about 5.0 g of enzyme per MT of feedstock. Suitably the endoprotease may be dosed at between about 0.5 g and about 3.0 g of enzyme per MT of feedstock. More suitably, the endoproteases may be dosed at about 1.0 g to about 2.0 g of enzyme per MT of feedstock.

In one embodiment the endoprotease may be dosed in an amount of at least about 0.01 SAPU/g of dry solids present in a feedstock, suitably at least 0.02 SAPU/g of dry solids present in a feedstock.

In another embodiment the endoprotease may be dosed in an amount of between about 0.01 SAPU/g of dry solids present in a feedstock to about 0.5 SAPU/g of dry solids present in a feedstock. Suitably, the endoprotease may be dosed in an amount of between about 0.1 SAPU/g of dry solids present in a feedstock to about 0.25 SAPU/g of dry solids present in a feedstock. More suitably the endoprotease may be dosed in an amount of between about 0.01 SAPU/g of dry solids present in a feedstock to about 0.1 SAPU/g of dry solids present in a feedstock.

Preferably, the endoprotease may be dosed in an amount of between about 0.01 SAPU/g of dry solids present in a feedstock to about 0.05 SAPU/g of dry solids present in a feedstock. Preferably the endoprotease may be dosed in an amount of about 0.02 SAPU/g of dry solids present in a feedstock.

In one embodiment the endoprotease may be dosed at at least about 0.1 SAPU/g of dry solids present in a feedstock. Suitably the endoprotease may be dosed at at least about 0.2 SAPU/g of dry solids present in a feedstock.

In further embodiments the endoprotease may be dosed at between about 0.1 to about 0.5 SAPU/g of dry solids present in a feedstock. Suitably the endoprotease may be dosed at between about 0.1 to about 0.3 SAPU/g of dry solids present in a feedstock. Preferably the endoprotease may be dosed at between about 0.15 to about 0.25 SAPU/g of dry solids present in a feedstock.

SAPU refers to a spectrophotometric acid protease unit, wherein 1 SAPU is the amount of protease enzyme activity that liberates one micromole of tyrosine per minute from a casein substrate under conditions of the assay.

### Grain-Based Material

The feedstock for use in accordance with the present invention may be a grain/cereal (e.g. wheat, barley, rye, rice, triticale, millet, milo, sorghum or corn), a root, a tuber (e.g. potato or cassava) a sugar (e.g. cane sugar, beet sugar, molasses or a sugar syrup), stillage, wet cake, DDGS or mixtures or portions thereof.

For the avoidance of doubt the grains can be mechanically broken. The grain-based material may be broken down or degraded to glucose. The glucose may subsequently be used as a feedstock for any fermentation process, e.g. for biofuel (e.g. bioethanol) production.

The grain-based material may be feedstock for a biofuel (e.g. bioethanol) production process.

Today most fuel ethanol is produced from corn (maize) grain, which is milled or grinded, treated with amylase enzymes to hydrolyse starch to sugars, saccharified and fermented, or subjected to SSF, and distilled. Other enzymes are often used in the process. While substantial progress has been made in reducing costs of ethanol production, substantial challenges remain. Improved techniques are still needed to reduce the cost of biofuel feedstocks for ethanol production. For example, in grain-based ethanol production degradation of arabinoxylans may increase accessibility of starch.

In some embodiments a xylanase may be used for use in the breakdown of hemicelluloses, e.g. arabinoxylan—particularly AXinsol and AXsol.

By way of example only, in the European fuel alcohol industry, small grains like wheat, barley and rye are common raw materials, in the US corn is mainly used. Wheat, barley and rye contain, next to starch, high levels of non-starch polysaccharide polymers (NSP), like cellulose, beta-glucan and hemicellulose.

The ratio in which the different NSPs are represented differ for each feedstock and vary depending on the methods for measurement used, but by way of example only the table below shows the different amounts of NSPs in wheat, barley and rye compared to some other feedstocks.

TABLE 1
Non-starch Polysaccharides present in different
feedstocks (g kg⁻¹ dry matter)

| | | | | Barley | | Oats | |
|---|---|---|---|---|---|---|---|
| | Corn | Wheat | Rye | Hulled | Hulless | Hulled | Hulless |
| Beta-Glucan | 1 | 8 | 16 | 42 | 42 | 28 | 41 |
| Cellulose | 22 | 17-20 | 15-16 | 43 | 10 | 82 | 14 |
| Soluble and Non-soluble NCP¹ | 75 | 89-99 | 116-136 | 144 | 114 | 150 | 113 |
| Total NSP | 97 | 107-119 | 132-152 | 186 | 124 | 232 | 116 |

¹Non Cellulosic Polysaccharides: pentosans, (arabino)xylans and other hemicelluloses

One advantage of the present invention is that use of the tripeptidyl peptidase of the present invention in alcohol (e.g. biofuel) production can also result in improved by-products from that process such as wet-cake, Distillers Dried Grains (DDG) or Distillers Dried Grains with Solubles (DDGS). Therefore one advantage of the present invention is since the wet-cake, DDG and DDGS are by-products of biofuel (e.g. bioethanol) production the use of the present invention can result in improved quality of these by-products.

### By-Product of Alcohol Production

The present invention provides a by-product of alcohol production obtainable (e.g. obtained) by the method of the present invention.

Suitably a by-product of alcohol production may be substantially enriched in one or more tripeptides.

The term “substantially enriched in tripeptides” as used herein means that of the total peptide concentration measured by any method known in the art (e.g. liquid chromatography-mass spectrometry (LC-MS)) at least about 20% (suitably at least about 30%) of those peptides are tripeptides. Suitably, at least about 40% of those peptides are tripeptides, more suitably at least about 50%.

In one embodiment the term “substantially enriched in tripeptides” as used herein means that of the total peptide concentration measured by any method known in the art (e.g. liquid chromatography-mass spectrometry (LC-MS)) at least about 70% of those peptides are tripeptides.

In some embodiments the by-product of alcohol production may be substantially enriched in one or more tripeptides having proline at the N-terminal, at the C-terminal or a combination thereof. Suitably the by-product may be enriched in one or more tripeptides having proline at the N-terminal and at the C-terminal.

The by-product can be any material obtainable following an alcohol fermentation process. Suitably a by-product of alcohol production may be whole stillage, thin stillage, wet-cake, Distillers Dried Grain (DDG) or Distillers Dried Grain Solubles (DDGS) or enriched protein DDG or DDGs, or a protein fraction.

Preferably the by-product may be a by-product of a biofuel production process.

Combination with Other Components/Forms

The tripeptidyl peptidase and/or endoprotease may be formulated in any manner known in the art.

In one embodiment the tripeptidyl peptidase and/or endoprotease for use in the present invention may be formulated as a liquid, a dry powder or a granule.

Preferably, the tripeptidyl peptidase and/or endoprotease may be formulated as a liquid formulation.

In other embodiments the tripeptidyl peptidase and/or endoprotease may be formulated as a dry powder.

In some embodiments further ingredients may be admixed with the tripeptidyl peptidase (e.g. proline tolerant tripeptidyl peptidase) such as salts such as Na₂SO₄, maltodextrin, limestone (calcium carbonate), cyclodextrin, wheat or a wheat component, starch, Talc, PVA, polyols such as sorbitol and glycerol, benzoate, sorbiate, sugars such as sucrose and glucose, propylene glycol, 1,3-propane diol, parabens, sodium chloride, citrate, acetate, sodium acetate, phosphate, calcium, metabisulfite, formate or mixtures thereof.

In a preferred embodiment the food additive composition or feed additive composition according to the present invention comprises the tripeptidyl peptidase (e.g. proline tolerant tripeptidyl peptidase) according to the present invention or fermentate according to the present invention and further comprises one or more ingredients selected from the group consisting of: a salt, polyol including sorbitol and glycerol, wheat or a wheat component, sodium acetate, sodium acetate trihydrate, potassium sorbate Talc, PVA, benzoate, sorbiate, 1,3-propane diol, glucose, parabens, sodium chloride, citrate, metabisulfite, formate or a combination thereof.

In one embodiment the salt may be selected from the group consisting of: Na₂SO₄, NaH₂PO₄, Na₂HPO₄, Na₃PO₄, (NH₄)H₂PO₄, K₂HPO₄, KH₂PO₄, K₂SO₄, KHSO₄, ZnSO₄, MgSO₄, CuSO₄, Mg(NO₃)₂, (NH₄)₂SO₄, sodium borate, magnesium acetate, sodium citrate or combinations thereof.

The dry powder or granules may be prepared by means known to those skilled in the art, such as, in top-spray fluid bed coater, in a buttom spray Wurster or by drum granulation (e.g. High sheer granulation), extrusion, pan coating or in a microingredients mixer.

Suitably, the tripeptidyl peptidase (optionally in combination with an endoprotease) may be dried with an alcohol production host as disclosed herein.

For some embodiments the tripeptidyl peptidase and/or endoprotease may be coated, for example encapsulated.

In one embodiment the coating protects the modified enzyme from heat and may be considered a thermoprotectant.

In some embodiments the tripeptidyl peptidase and/or endoprotease may be diluted using a diluent, such as starch powder, lime stone or the like.

In one embodiment, the tripeptidyl peptidase and/or endoprotease contains one or more of the following: a buffer, salt, sorbitol and/or glycerol.

In another embodiment the tripeptidyl peptidase and/or endoprotease may be formulated by applying, e.g. spraying, the enzyme(s) onto a carrier substrate, such as ground wheat for example.

Typical liquid formulations of food grade enzymes may include the following components (% is in w/w): enzyme of interest 0.2%-30%, preferably 2%-20%.

In some embodiment polyols may be admixed with the tripeptidyl peptidase and/or the endoprotease.

Polyols such as glycerol and/or sorbitol may be admixed in amounts from 5% (w/w)-50% (w/w), preferably 10-50% (w/w) and more preferably 10-30% (w/w) with % (w/w) meaning (weight polyol/weight solution), without wishing to be bound by theory a lower concentration of 10% polyol might help increasing the solubility and storage stability of the enzyme. However many commercial enzymes require 30% glycerol to keep the enzyme stable over time in the concentration of interest. Higher polyol at 50% might still improve stability further, but at this polyol level also the benefit of lower water activity is an advantage for microbial preservation. In particular for food enzymes this can be very important at neutral pH, where the choice of good preservatives are limited.

Sugars (in particular glucose) may be admixed with the tripeptidyl peptidase and/or endoprotease. Sugars like glucose, fructose, sucrose, maltose, lactose, trehalose are all examples of substances that for many enzymes can be an alternative to using polyols. Suitably they (particularly glucose) may be used in the range 5% (w/w)-50% (w/w) either alone or in combination with polyols.

The stability of the enzyme formulation might also be increased by using salts like NaCl, KCl, CaCl2, Na2SO4 or other food grade salts in concentrations from about 0.1% to about 20% (suitably from about 0.1% to about 5%). Without wishing to be bound by theory, it is believed that the high salt concentrations might again be a way of achieving microbial stability either alone or in combination with polyols or sugars. The mechanism of action may be due to lower water activity or a specific action between a certain enzyme and a salt. Therefore in some embodiments the tripeptidyl peptidase may be admixed with at least one salt.

Sodium acetate may be admixed in amounts from 5% (w/w)-50% (w/w), preferably 8-40% preferably 8-12% (w/w), preferably 10-50% and more preferably 10-30% (w/w) with (w/w) meaning % (weight sodium acetate/weight solution).

In one embodiment the tripeptidyl peptidase and/or endoprotease may be admixed with a preservative.

Suitably the preservative may be benzoate, such as sodium benzoate, and/or potassium sorbate. These preservatives can be typically used in a combined concentration of about 0.1-1%, suitably about 0.2-0.5%. Sodium benzoate is most efficient at pH<5.5 and sodium sorbate at pH<6.

In one embodiment the one or more ingredients (e.g. used for the formulation of the enzyme (e.g. the tripeptidyl peptidase and/or endoprotease)) may be selected from the group consisting of: a wheat carrier, a polyol, a sugar, a salt and a preservative.

Suitably the sugar is sorbitol.

Suitably the salt is sodium sulphate.

Suitably the polyol may be polyethylene glycol.

In one embodiment the one or more ingredients (e.g. used for the formulation of the enzyme (e.g. the tripeptidyl peptidase and/or endoprotease)) may be selected from the group consisting of: a wheat carrier, a polyol, a sorbitol, sodium sulphate and a preservative.

Suitably the one or more ingredients (e.g. used for the formulation of the tripeptidyl peptidase and/or endoprotease) may be selected from the group consisting of: a wheat carrier, sorbitol and sodium sulphate.

Suitably, the tripeptidyl peptidase and/or the endoprotease may be admixed with a wheat carrier.

Suitably, the tripeptidyl peptidase and/or the endoprotease may be admixed with sorbitol.

Suitably the tripeptidyl peptidase and/or the endoprotease may be admixed with sodium sulphate.

In one embodiment the enzyme for use in the present invention (e.g. a tripeptidyl peptidase and/or endoprotease) may be formulated with one or more ingredient selected from the group consisting of: polyols, such as glycerol and/or sorbitol; sugars, such as glucose, fructose, sucrose, maltose, lactose and trehalose; salts, such as NaCl, KCl, CaCl₂, Na₂SO₄ or other salts; a preservative, e.g. sodium benzoate and/or potassium sorbate; or combinations thereof.

In another embodiment the tripeptidyl peptidase for use in the methods and/or uses of the present invention may be formulated with a carrier comprising (or consisting essentially of; or consisting of) Na₂SO₄, NaH₂PO₄, Na₂HPO₄, Na₃PO₄, (NH₄)H₂PO₄, K₂HPO₄, KH₂PO₄, K₂SO₄, KHSO₄, ZnSO₄, MgSO₄, CuSO₄, Mg(NO₃)₂, (NH₄)₂SO₄, sodium borate, magnesium acetate, sodium citrate or combinations thereof.

In another embodiment, the tripeptidyl peptidase for use in the methods and/or uses of the present invention may be formulated with Na₂SO₄.

The tripeptidyl peptidase and/or endoprotease may be used in combination with other components.

In one embodiment the “another component” may be one or more enzymes.

Therefore, in one embodiment, the method and/or use of the present invention may further comprise the use of one or more cellulase activity, hemicellulase activity, further enzyme activity or a combination thereof.

Suitably the one or more cellulase activity, hemicellulase activity, further enzyme activity or combination thereof is selected from the group consisting of: one or more of the enzymes selected from the group consisting of: endoglucanases (E.C. 3.2.1.4); cellobiohydrolases (E.C. 3.2.1.91), β-glucosidases (E.C. 3.2.1.21), cellulases (E.C. 3.2.1.74), lichenases (E.C. 3.1.1.73), lipases (E.C. 3.1.1.3), lipid acyltransferases (generally classified as E.C. 2.3.1.x), phospholipases (E.C. 3.1.1.4, E.C. 3.1.1.32 or E.C. 3.1.1.5), phytases (e.g. 6-phytase (E.C. 3.1.3.26) or a 3-phytase (E.C. 3.1.3.8), acid phosphatase, amylases, alpha-amylases (E.C. 3.2.1.1), xylanases (e.g. endo-1,4-β-d-xylanase (E.C. 3.2.1.8) or 1,4 β-xylosidase (E.C. 3.2.1.37) or E.C. 3.2.1.32, E.C. 3.1.1.72, E.C. 3.1.1.73), glucoamylases (E.C. 3.2.1.3), pullulanases, hemicellulases, proteases (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)), debranching enzymes, cutinases, esterases and/or mannanases (e.g. a β-mannanase (E.C. 3.2.1.78)) transferases, glucosidases, arabinofuranosidase Suitably the other component may be a phytase (e.g. a 6-phytase (E.C. 3.1.3.26) or a 3-phytase (E.C. 3.1.3.8)).

In one embodiment the other component may be one or more of the enzymes selected from the group consisting of xylanases (E.C. 3.2.1.8, E.C. 3.2.1.32, E.C. 3.2.1.37, E.C. 3.1.1.72, E.C. 3.1.1.73), an amylase (including α-amylases (E.C. 3.2.1.1), α-forming amylases (E.C. 3.2.1.60), β-amylases (E.C. 3.2.1.2) and γ-amylases (E.C. 3.2.1.3); and/or a protease (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)).

In one embodiment the other component may be a combination of an amylase (e.g. α-amylases (E.C. 3.2.1.1)) and a protease (e.g. subtilisin (E.C. 3.4.21.62)).

In a preferred embodiment the tripeptidyl peptidase may be formulated with one or more glucoamylase, α-amylase and/or further protease.

Suitably, the further protease may be an endoprotease.

In one embodiment the other component may be a β-glucanase, e.g. an endo-1,3(4)-β-glucanases (E.C. 3.2.1.6).

In one embodiment the other component may be a mannanases (e.g. a β-mannanase (E.C. 3.2.1.78)).

In one embodiment the other component may be a lipase (E.C. 3.1.1.3), a lipid acyltransferase (generally classified as E.C. 2.3.1.x), or a phospholipase (E.C. 3.1.1.4, E.C. 3.1.1.32 or E.C. 3.1.1.5), suitably a lipase (E.C. 3.1.1.3).

In one embodiment the other component may be a protease (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)).

In one embodiment the further enzyme may be an α-amylases (E.C. 3.2.1.1), a pullulanase (EC 3.2.1.41), a β-amylases (E.C. 3.2.1.2), a maltogenic amylase (e.g. a glucan 1,4-alpha-maltohydrolase (EC 3.2.1.133)), G4-forming amylases (E.C. 3.2.1.60), an isoamylase (EC 3.2.1.68), glucoamylases (E.C. 3.2.1.3) or combinations thereof.

In some embodiments the methods and/or uses according to the present invention may further comprise the use of one or more glycohydrolase (GH) enzymes.

GH enzymes may include xylanases, mannanases, amylases, β-glucanases, cellulases, and other carbohydrases, and may be classified based on such properties as the sequence of amino acids, their three dimensional structure and the geometry of their catalytic site (Gilkes, et al., 1991, Microbiol. Reviews 55: 303-315).

In one embodiment the GH enzyme (e.g. a xylanase) according to the foregoing embodiment may be a GH family enzyme selected from one or more of the group consisting of: GH10, GH11, GH5, GH7, GH8 and GH43.

Initially all known and characterized xylanases belonged to the families GH10 or GH11. Further work then identified numerous other types of xylanases belonging to the families GH5, GH7, GH8 and GH43 (Collins et al (2005) FEMS Microbiol Rev., 29 (1), 3-23).

The structure of the GH11 xylanases can be described as a β-Jelly roll structure or an all β-strand sandwich fold structure (Himmel et al 1997 Appl. Biochem. Biotechnol. 63-65, 315-325). GH11 enzymes have a catalytic domain of around 20 kDa.

GH10 xylanases have a catalytic domain with molecular weights in the range of 32-39 kDa. The structure of the catalytic domain of GH10 xylanases consists of an eightfold β/α barrel (Harris et al 1996—Acta. Crystallog. Sec. D 52, 393-401).

Three-dimensional structures are available for a large number of Family GH10 enzymes, the first solved being those of the Streptomyces lividans xylanase A (Derewenda et al J Biol Chem 1994 Aug. 19; 269(33) 20811-4), the C. fimi endo-glycanase Cex (White et al Biochemistry 1994 Oct. 25; 33(42) 12546-52), and the Cellvibrio japonicus Xyn10A (previously Pseudomonas fluorescens subsp. xylanase A) (Harris et al Structure 1994 Nov. 15; 2(11) 1107-16.). As members of Clan GHA they have a classical (α/β)₈ TIM barrel fold with the two key active site glutamic acids located at the C-terminal ends of beta-strands 4 (acid/base) and 7 (nucleophile) (Henrissat et al Proc Natl Acad Sci USA 1995 Jul. 18; 92(15) 7090-4). GH family enzyme may be identified by the skilled person by techniques known in the art.

Protein similarity searches (e.g. protein blast at http://blast.ncbi.nlm.nih.gov/Blast.cgi?CMD=Web&PAGE_TYPE=BlastHome) may determine whether an unknown sequence falls under the term of e.g. a GH10 xylanase family member, particularly the GH families may be categorised based on sequence homology in key regions. In addition or alternatively, to determine whether an unknown protein sequence is a xylanase protein within the GH10 family, the evaluation can be done, not only on sequence similarity/homology/identity, but also on 3D structure similarity. The classification of GH-families is often based on the 3D fold. Software that will predict the 3D fold of an unknown protein sequence is HHpred (http://toolkit.tuebingen.mpg.de/hhpred). The power of this software for protein structure prediction relies on identifying homologous sequences with known structure to be used as template. This works so well because structures diverge much more slowly than primary sequences. Proteins of the same family may have very similar structures even when their sequences have diverged beyond recognition.

For example, an unknown sequence can be pasted into the software (http://toolkit.tuebingen.mpg.de/hhpred) in FASTA format. Having done this, the search can be submitted. The output of the search will show a list of sequences with known 3D structures. To confirm that the unknown sequence indeed is e.g. a GH10 xylanase, GH10 xylanases may be found within the list of homologues having a probability of >90. Not all proteins identified as homologues will be characterised as GH10 xylanases, but some will. The latter proteins are proteins with a known structure and biochemically characterisation identifying them as xylanases. The former have not been biochemically characterised as GH10 xylanases. Several references describes this protocol such as Söding J. (2005) Protein homology detection by HMM-HMM comparison—Bioinformatics 21, 951-960 (doi:10.1093/bioinformatics/bti125) and Söding J, Biegert A, and Lupas A N. (2005) The HHpred interactive server for protein homology detection and structure prediction—Nucleic Acids Research 33, W244-W248 (Web Server issue) (doi:10.1093/nar/gki40).

According to the Cazy site (http://www.cazy.org/), Family 10 glycoside hydrolases can be characterised as follows:

Known Activities: endo-1,4-β-xylanase (EC 3.2.1.8); endo-1,3-β-xylanase (EC 3.2.1.32); tomatinase (EC 3.2.1.-)

### Mechanism: Retaining

### Clan: GH-A

Catalytic Nucleophile/Base: Glu (experimental)
Catalytic Proton Donor: Glu (experimental)

### 3D Structure Status: (β/α)₈

GH10 xylanases for use in accordance with the present invention may have a catalytic domain with molecular weights in the range of 32-39 kDa. The structure of the catalytic domain of a GH10 xylanase consists of an eightfold β/α barrel (Harris et al 1996—Acta. Crystallog. Sec. D 52, 393-401).

Three-dimensional structures are available for a large number of Family GH10 enzymes, the first solved being those of the Streptomyces lividans xylanase A (Derewenda et al J Biol Chem 1994 Aug. 19; 269(33) 20811-4), the C. fimi endo-glycanase Cex (White et al Biochemistry 1994 Oct. 25; 33(42) 12546-52), and the Cellvibrio japonicus Xyn10A (previously Pseudomonas fluorescens subsp. xylanase A) (Harris et al Structure 1994 Nov. 15; 2(11) 1107-16.). As members of Clan GHA they have a classical (α/β)₈ TIM barrel fold with the two key active site glutamic acids located at the C-terminal ends of beta-strands 4 (acid/base) and 7 (nucleophile) (Henrissat et al Proc Natl Acad Sci USA 1995 Jul. 18; 92(15) 7090-4).

Therefore the term “GH10 xylanase” as used herein means a polypeptide having xylanase activity and having a (α/β)₈ TIM barrel fold with the two key active site glutamic acids located at the C-terminal ends of beta-strands 4 (acid/base) and 7 (nucleophile).

In a particularly preferred embodiment the tripeptidyl peptidase may not be combined with an enzyme having the following polypeptide sequence:

MRTAAASLTLAATCLFELASALMPRAPLIPAMKAKVALPSGNATFEQYID
HNNPGLGTFPQRYWYNPEFWAGPGSPVLLFTPGESDAADYDGFLTNKTIV
GRFAEEIGGAVILLEHRYWGASSPYPELTTETLQYLTLEQSIADLVHFAK
TVNLPFDEIHSSNADNAPWVMTGGSYSGALAAWTASIAPGTFWAYHASSA
PVQAIYDFWQYFVPVVEGMPKNCSKDLNRVVEYIDHVYESGDIERQQEIK
EMFGLGALKHFDDFAAAITNGPWLWQDMNFVSGYSRFYKFCDAVENVTPG
AKSVPGPEGVGLEKALQGYASWFNSTYLPGSCAEYKYWTDKDAVDCYDSY
ETNSPIYTDKAVNNTSNKQWTANFLCNEPLFYWQDGAPKDESTIVSRIVS
AEYWQRQCHAYFPEVNGYTFGSANGKTAEDVNKWTKGWDLTNTTRLIWAN
GQFDPWRDASVSSKTRPGGPLQSTEQAPVHVIPGGFHCSDQWLVYGEANA
GVQKVIDEEVAQIKAWVAEYPKYRKP

In another embodiment the other component may be a further protease. Suitably, the further protease may be selected from the group consisting of: an aminopeptidase and a carboxypeptidase.

The term “aminopeptidase” as used in this context refers to an exopeptidase which is able to cleave single amino acids, di-amino acids or combinations thereof from the N-terminus of a protein and/or peptide substrate. Preferably, an aminopeptidase is able to cleave single amino acids only from the N-terminus of a protein and/or peptide substrate.

The aminopeptidase may be obtainable (e.g. obtained) from Lactobacillus, suitably obtainable from Lactobacillus helveticus.

In one embodiment the aminopeptidase may be an aminopeptidase N (e.g. PepN) (EC 3.4.11.2)

In another embodiment the aminopeptidase may comprise the sequence shown as:

MAVKRFYKTFHPEHYDLRINVNRKNKTINGTSTITGDVIENPVFINQKFM
TIDSVKVDGKNVDFDVIEKDEAIKIKTGVTGKAVIEIAYSAPLTDTMMGI
YPSYYELEGKKKQIIGTQFETTFARQAFPCVDEPEAKATFSLALKWDEQD
GEVALANMPEVEVDKDGYHHFEETVRMSSYLVAFAFGELQSKTTHTKDGV
LIGVYATKAHKPKELDFALDIAKRAIEFYEEFYQTKYPLPQSLQLALPDF
SAGAMENWGLVTYREAYLLLDPDNTSLEMKKLVATVITHELAHQWFGDLV
TMKWWDNLWLNESFANMMEYLSVDGLEPDWHIWEMFQTSEAASALNRDAT
DGVQPIQMEINDPADIDSVFDGAIVYAKGSRMLVMVRSLLGDDALRKGLK
YYFDHHKEGNATGDDLWDALSTATDLDIGKIMHSWLKQPGYPVVNAFVAE
DGHLKLTQKQFFIGEGEDKGRQWQIPLNANFDAPKIMSDKEIDLGNYKVL
REEAGHPLRLNVGNNSHFIVEYDKTLLDDILSDVNELDPIDKLQLLQDLR
LLAEGKQISYASIVPLLVKFADSKSSLVINALYTTAAKLRQFVEPESNEE
KNLKKLYDLLSKDQVARLGWEVKPGESDEDVQIRPYELSASLYAENADSI
KAAHQIFTENEDNLEALNADIRPYVLINEVKNEGNAELVDKLIKEYQRTA
DPSYKVDLRSAVTSTKDLAAIKAIVGDFENADVVKPQDLCDWYRGLLANH
YGQQAAWDWIREDWDWLDKTVGGDMEFAKFITVTAGVFHTPERLKEFKEF
FEPKINVPLLSREIKMDVKVIESKVNLIEAEKDAVNDAVAKAID

The term “carboxypeptidase” as used herein has its usual meaning in the art and refers to an exopeptidase that is capable of cleaving n amino acids from the C-terminus of a peptide and/or protein substrate. In one embodiment n may be at least 1, suitably n may be at least 2. In other embodiments n may be at least 3, suitably at least 4.

In other embodiments, the tripeptidyl peptidase (optionally in combination with an endoprotease) may be used with one or more further exopeptidase.

In one embodiment the proline tolerant tripeptidyl peptidase (optionally in combination with an endoprotease) is not combined with a proline-specific exopeptidase.

In one embodiment the additional component may be a stabiliser or an emulsifier or a binder or carrier or an excipient or a diluent or a disintegrant.

The term “stabiliser” as used here is defined as an ingredient or combination of ingredients that keeps a product from changing over time.

The term “emulsifier” as used herein refers to an ingredient that prevents the separation of emulsions. Emulsions are two immiscible substances, one present in droplet form, contained within the other. Emulsions can consist of oil-in-water, where the droplet or dispersed phase is oil and the continuous phase is water; or water-in-oil, where the water becomes the dispersed phase and the continuous phase is oil. Foams, which are gas-in-liquid, and suspensions, which are solid-in-liquid, can also be stabilised through the use of emulsifiers.

As used herein the term “binder” refers to an ingredient that binds the product together through a physical or chemical reaction. During “gelation” for instance, water is absorbed, providing a binding effect. However, binders can absorb other liquids, such as oils, holding them within the product. In the context of the present invention binders would typically be used in solid or low-moisture products for instance baking products: pastries, doughnuts, bread and others. Examples of granulation binders include one or more of: polyvinylpyrrolidone, hydroxypropylmethylcellulose (HPMC), hydroxypropylcellulose (HPC), sucrose, maltose, gelatin and acacia.

“Carriers” mean materials suitable for administration of the enzyme and include any such material known in the art such as, for example, any liquid, gel, solvent, liquid diluent, solubilizer, or the like, which is non-toxic and which does not interact with any components of the composition in a deleterious manner.

The present invention provides the use of a composition comprising a tripeptidyl peptidase (optionally in combination with an endoprotease) in combination with at least one physiologically acceptable carrier selected from at least one of maltodextrin, limestone (calcium carbonate), cyclodextrin, wheat or a wheat component, sucrose, starch, Na₂SO₄, Talc, PVA, sorbitol, benzoate, sorbiate, glycerol, sucrose, propylene glycol, 1,3-propane diol, glucose, parabens, sodium chloride, citrate, acetate, phosphate, calcium, metabisulfite, formate and mixtures thereof, as well as methods for making the same.

In another embodiment the present invention provides a the use of a and methods of making the same comprising an enzyme of the present invention formulated with a compound selected from one or more of the group consisting of: Na₂SO₄, NaH₂PO₄, Na₂HPO₄, Na₃PO₄, (NH₄)H₂PO₄, K₂HPO₄, KH₂PO₄, K₂SO₄, KHSO₄, ZnSO₄, MgSO₄, CuSO₄, Mg(NO₃)₂, (NH₄)₂SO₄, sodium borate, magnesium acetate, sodium citrate or a combination thereof.

Examples of “excipients” include one or more of: microcrystalline cellulose and other celluloses, lactose, sodium citrate, calcium carbonate, dibasic calcium phosphate, glycine, starch, milk sugar and high molecular weight polyethylene glycols.

Examples of “disintegrants” include one or more of: starch (preferably corn, potato or tapioca starch), sodium starch glycollate, croscarmellose sodium and certain complex silicates.

Examples of “diluents” include one or more of: water, ethanol, propylene glycol and glycerin, and combinations thereof.

The other components may be used simultaneously (e.g. when they are in admixture together or even when they are delivered by different routes) or sequentially (e.g. they may be delivered by different routes) to the composition.

In one embodiment preferably the composition does not comprise chromium or organic chromium.

In one embodiment preferably the composition does not contain sorbic acid.

The tripeptidyl peptidase and/or endoprotease for use in the present invention may be used in any suitable form.

The tripeptidyl peptidase and/or endoprotease may be used in the form of solid or liquid preparations or alternatives thereof. Examples of solid preparations include powders, pastes, boluses, capsules, pellets, tablets, pills, granules, capsules, ovules, solutions or suspensions, dusts, and granules which may be wettable, spray-dried or freeze-dried. Examples of liquid preparations include, but are not limited to, aqueous, organic or aqueous-organic solutions, suspensions and emulsions.

The tripeptidyl peptidase and/or endoprotease may contain flavouring or colouring agents, for immediate-, delayed-, modified-, sustained-, pulsed- or controlled-release applications.

By way of example, if the tripeptidyl peptidase and/or endoprotease is used in a solid, e.g. pelleted form, it may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Preferred excipients for the forms include lactose, starch, a cellulose, milk sugar or high molecular weight polyethylene glycols.

For aqueous suspensions and/or elixirs, the composition of the present invention may be combined with various sweetening or flavouring agents, colouring matter or dyes, with emulsifying and/or suspending agents and with diluents such as water, propylene glycol and glycerin, and combinations thereof.

### Packaging

In one embodiment the tripeptidyl peptidase and/or endoprotease according to the present invention is packaged.

In one preferred embodiment tripeptidyl peptidase and/or endoprotease is packaged in a bag, such as a paper bag.

In an alternative embodiment the tripeptidyl peptidase and/or endoprotease may be sealed in a container. Any suitable container may be used.

### Advantages

The inventors herein have found that a tripeptidyl peptidase can cleave protein and/or peptide substrates present in a feedstock to liberate tripeptides, surprisingly this has been found to increase alcohol production e.g. during bioethanol production.

In another embodiment the use of a tripeptidyl peptidase may advantageously improve an alcohol production host's ability to ferment during alcohol production.

Without wishing to be bound by theory it is believed that the tripeptidyl peptidase of the present invention may increase the concentration of tripeptides present in a feedstock or fraction thereof, which tripeptides may be a good amino acid source and/or energy and/or nutrient source for an alcohol production host.

Therefore, an advantage of the invention is that it may improve the overall health of an alcohol production host.

Advantageously, use of the tripeptidyl peptidase of the invention may reduce the amount of urea that needs to be added to the feedstock.

One advantage of the present invention is that use of the tripeptidyl peptidase of the present invention in alcohol (e.g. biofuel) production can result in improved by-products from that process such as wet-cake, Distillers Dried Grains (DDG) or Distillers Dried Grains with Solubles (DDGS). Therefore one advantage of the present invention is since the wet-cake, DDG and DDGS are by-products of biofuel (e.g. bioethanol) production the use of the present invention can result is improved quality of these by-products. In particular, the by-products may be protein enriched. In one particular embodiment the (by) products may be enriched in tripeptides, e.g. proline-rich tripeptides.

Advantageously, a tripeptidyl peptidase taught for use in the present invention is capable of acting on a wide range of peptide and/or protein substrates and due to having such a broad substrate-specificity is not readily inhibited from cleaving substrates enriched in certain amino acids (e.g. proline and/or lysine and/or arginine and/or glycine). The use of such a tripeptidyl peptidase (e.g. a proline tolerant tripeptidyl peptidase) therefore may efficiently and/or rapidly breakdown protein substrates and yield tripeptides into the feedstock.

Preferably the tripeptidyl peptidase may have a high activity on peptides and/or proteins having one or more of lysine, arginine or glycine in the P1 position. Without wishing to be bound by theory peptide and/or protein substrates comprising these amino acids at the P1 position may be difficult to digest for many tripeptidyl peptidases and/or proteases in general and upon encountering such residues cleavage of the peptide and/or protein substrate by a tripeptidyl peptidase and/or protease may halt or slow. Advantageously, by using a tripeptidyl peptidase of the invention it is possible to digest protein and/or peptide substrates comprising lysine, arginine and/or glycine at P1 efficiently and/or without significantly slowing the cleavage reaction resulting in the more efficient digestion of a substrate and/or more efficient generation of tripeptides in a feedstock.

The present invention also provides for the use of proline tolerant tripeptidyl peptidase that, in addition to having the activities described above, may be tolerant of proline at position P2, P2′, P3 and P3′. This is advantageous as it allows the efficient cleavage of peptide and/or protein substrates having stretches of proline and allows cleavage of a wide range of peptide and/or protein substrates resulting in more efficient digestion of a feedstock.

Advantageously the tripeptidyl peptidase may have a preferential activity on peptides and/or proteins having lysine at the P1 position, this allows the efficient cleavage of substrates having high lysine content, such as whey protein.

The present invention also provides for thermostable tripeptidyl peptidases which are less prone to being denatured and/or will therefore retain activity for a longer period of time when compared to a non-thermostable variant.

Advantageously the proline tolerant tripeptidyl peptidase may have activity in a pH range of about pH 7 and can therefore be used with an alkaline endoprotease. This means that changing the pH of the reaction medium comprising the protein and/or peptide substrate for hydrolysate production is not necessary between enzyme treatments. In other words it allows the tripeptidyl peptidase and the endoprotease to be added to a reaction (e.g. during a method and/or use of the invention) simultaneously, which may make the process for quicker and/or more efficient and/or more cost-effective. Moreover, this allows for a more efficient reaction as at lower pH values the substrate may precipitate out of solution and therefore not be cleaved.

A tripeptidyl peptidase having activity at an acidic pH can be used in combination with an acid endoprotease and advantageously does not require the pH of the reaction medium to be changed between enzyme treatments.

Advantageously, the use of an endoprotease in combination with a tripeptidyl peptidase can increase the efficiency of substrate cleavage. Without wishing to be bound by theory, it is believed that an endoprotease is able to cleave a peptide and/or protein substrate at multiple regions away from the C or N-terminus, thereby producing more N-terminal ends for the tripeptidyl peptidase to use as a substrate, thereby advantageously increasing reaction efficiency and/or reducing reaction times.

Use of an endoprotease, a tripeptidyl peptidase and a further component e.g. carboxypeptidase and/or aminopeptidase has many advantages:
it allows for the efficient production of single amino acids and/or dipeptides and/or tripeptides which can efficiently be absorbed by an alcohol production host (e.g. due to having a better osmotic potential for uptake);
a protein and/or peptide substrate may be more efficiently and/or more quickly digested;
reduced end-point inhibition (i.e. inhibition by its reaction products) of a the proline tolerant tripeptidyl peptidase, particularly when used in vitro, such as in the manufacture of a hydrolysate by digesting the tripeptides into single amino acids and/or dipeptides; and/or
synergistic and/or additive activity on substrates containing high levels of proline, lysine, arginine and/or glycine.

Surprisingly, DDGS obtainable (e.g. obtained) by carrying out the method and/or uses of the present invention may have an improved taste. Advantageously therefore a DDGS obtainable by the present invention may be more palatable to a subject (e.g. an animal) fed with the DDGS.

### Additional Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Amino acids are referred to herein using the name of the amino acid, the three letter abbreviation or the single letter abbreviation.

The term “protein”, as used herein, includes proteins, polypeptides, and peptides.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a tripeptidyl peptidase”, “an endoprotease” or “an enzyme” includes a plurality of such candidate agents and reference to “the feedstock” includes reference to one or more feedstocks and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

The invention will now be described, by way of example only, with reference to the following Figures and Examples.

### EXAMPLES

### Example 1

### Subject: Corn Ethanol Fermentation—Use of Tripeptidyl Peptidase (3PP) to Increase Rate of Fermentation and Total Ethanol Levels

### Objectives:

To identify this enzyme's benefits to fermentation with the protease FermGen® already present in glucoamylase products.

### Introduction:

A tripeptidyl peptidase, (3PP) was isolated from Trichoderma reesei. This peptidase has high potential as an additive in corn ethanol fermentations as a supplier of nitrogen to the yeast in the form of tripeptides. This enzyme showed increase rates of fermentation when dosed by itself and in conjunction with the protease FermGen® (an acid fungal endoprotease of the aspartic acid protease family).

### Materials:

### Liquefact: Big River Dyersville (May 4, 2012)

### Yeast: Red Star, Ethanol Red

Urea: JT Baker, USP grade, Lot no. A23338

TABLE 1
Enzymes Used in this Experiment

| | | | | | SG |
|---|---|---|---|---|---|
| | | | | | (Specific |
| Enzyme | Lot no. | Sticker no. | Activity | | Gravity) |
| Trichoderma | 1681721701 | 2011-0651 | 1134 | GAU/g | 1.13 |
| reesei | | | | | |
| glucoamylase | | | | | |
| Aspergillus | 1681359791 | 2012-0003 | 10562 | SSU/g | 1.17 |
| kawachi alpha | | | | | |
| amylase | | | | | |
| FermGen ® | 1681719474 | 2012-0093 | 1018 | SAPU/g | 1.17 |
| (available from | | | | | |
| DuPont | | | | | |
| Industrial | | | | | |
| Biosciences - | | | | | |
| formerly | | | | | |
| Genencor) | | | | | |
| Tripeptidyl | N/A | 2012-0024 | 2358 | U/g | 1.06 |
| peptidase | | | | | |

### Experimental:

Liquefact was thawed in a 60° C. waterbath then the amount needed for each was weighed into a beaker. Solid urea was added to yield a final concentration of 600 ppm. The pH was adjusted to 4.5 with 6N H₂SO₄ prior to % dry solids (DS) determination by moisture balance prior to the collection of a 0 timepoint in triplicate. Yeast was weighed to yield a final concentration of 0.1% of the total mass and dry-pitched into the liquefact, followed by thorough mixing. The liquefact was then dispensed in 100 g quantities into 250 ml wide-mouthed Erlenmeyer flasks in triplicate for each condition to be tested. Enzymes were dosed as in Table 2.

The number of activity units of tripeptidyl peptidase displayed in Table 1 were calculated using 150 ul of 1 mM H-Ala-Ala-Phe-pNA (a para-nitroaniline derivatized substrate) in 0.1M NaOAc, pH4.5 in a well of 96 well microtiter plate mixed with various amounts of enzyme dilution to fit within a linear range of the assay. Absorbance at 410 nM is followed kinetically at room temp (25° C.). 1 U is defined as the amount of enzyme releasing 1 micromole of pNA per min. pNA molar adsorption at 410 is assumed to be 8800.

TABLE 2
Flask Conditions and Enzyme Dosages for the 600 ppm Urea Fermentation
Table 2: Trichoderma reesei glucoamylase (GA) and Aspergillus
kawachi alpha amylase (AkAA) were dosed at 0.325 GAU/g DS and
1.95 SSU/g DS, respectively. Both proteases were dosed at 0.02 U/g DS.

| | | 1:5 dilution | | 1:100 dilution | |
|---|---|---|---|---|---|
| | | μl | μl | μl | μl |
| Flask | Description | GA | AkAA | FermGen | Peptidase |
| 1, 2, 3 | Control | 38.8 | 24.1 | | |
| 4, 5, 6 | FermGen | 38.8 | 24.1 | 51.3 | |
| 7, 8, 9 | 3PP | 38.8 | 24.1 | | 24.5 |
| 10, 11, 12 | FermGen + 3PP | 38.8 | 24.1 | 51.3 | 24.5 |
| 13, 14, 15 | 2X FermGen | 38.8 | 24.1 | 102.5 | |
| 16, 17, 18 | 2X Peptidase | 38.8 | 24.1 | | 48.9 |

Flasks were stoppered with single-holed, number 8-sized rubber stoppers and placed in a 1″ orbit shaker at 150 rpm and 32° C. Fermentation samples of approximately 2 ml were collected in triplicate at the indicated timepoints and prepped by spinning down solids for 3 minutes at 16,000×g. Sample collection consists of collecting 500 μl of supernatant, inactivating enzymes with 50 μl of 1N H₂SO₄ for 5 minutes, diluting 1:10 in diH₂O and passing through a 13 mm diameter, 0.45 μm pore size nylon filter. As with the comparison to competitor's products below, these sampling differences were performed at the same time as the 3PP experiments, but not directly related. All data presented was collected by the normal method described above. It's not wrong to leave both methods in, but it might be confusing (it may also fall under basic knowledge to one skilled in the art). Collected samples were analyzed by HPLC under the following conditions:
Column: Phenomenex Rezex Organic Acid Column
Mobile Phase: 0.05N H₂SO₄
Detector: Refractive index
Temperature: 65° C.
Injection Volume: 20 μl
Separation time: 23 minutes

Liquefact was prepared and dispensed as described above except that urea was added to only 200 ppm. Enzymes were dosed as described in Table 3.

TABLE 3
Flask Conditions and Enzyme Dosages
for the 200 ppm Urea Fermentations
Table 3: Enzymes were dosed at the same
levels as the 600 ppm urea fermentation

| | | | | 1:50 | 1:100 |
|---|---|---|---|---|---|
| | | 1:5 dilution | | dilution | dilution |
| | | μl | μl | μl | μl |
| Flask | Description | GA | AkAA | FermGen | Peptidase |
| 1, 2, 3 | Control | 39.3 | 24.5 | | |
| 4, 5, 6 | FermGen | 39.3 | 24.5 | 26.0 | |
| 7, 8, 9 | 3PP | 39.3 | 24.5 | | 24.8 |
| 10, 11, 12 | FermGen + 3PP | 39.3 | 24.5 | 26.0 | 24.8 |
| 13, 14, 15 | 2X FermGen | 39.3 | 24.5 | 52.0 | |
| 16, 17, 18 | 2X 3PP | 39.3 | 24.5 | | 49.6 |

Flasks were incubated and samples collected and analyzed as described above.

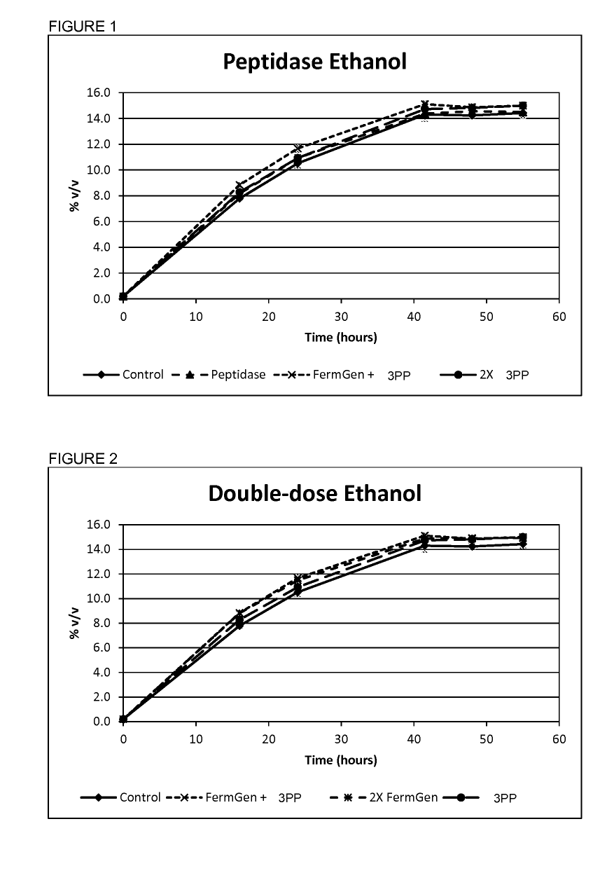

FIG. 1 shows that there was better breakout between the control and sample fermentations at 200 ppm urea. The peptidase did provide some benefit by itself, but there was no increase at twice the dose. However a large boost in rate and final levels was seen when combined with FermGen®.

Again, FIG. 2 shows that the 2× tripeptidyl peptidase only produced a minor increase while a mixture of the two enzymes produced an increased rate comparable to a double dose of FermGen®.

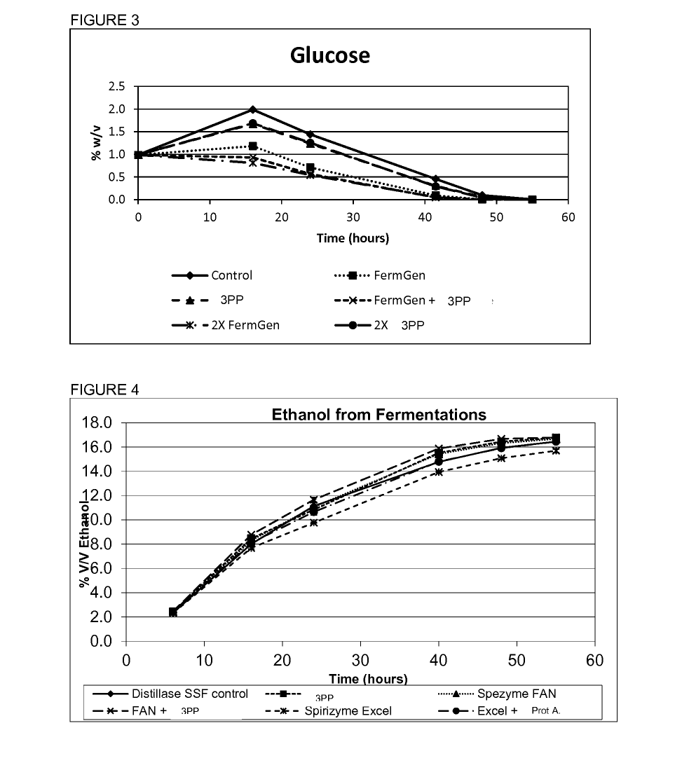

Another display of the health of yeast (i.e. the ability of the yeast to ferment) in fermentation is the rate of glucose consumption (FIG. 3). The peptidase shows an improvement over the control which doesn't change at a higher dose. FermGen® promotes more glucose uptake than the peptidase and shows a characteristic dose response. The mixture of the two is nearly identical to the double dose of FermGen®.

### Example 2

### Subject: Corn Ethanol Fermentations—Effectiveness of Tripeptidyl Peptidase and Protease A

### Objectives:

A new tripeptide generating peptidase needs to be analyzed for application for its ability to benefit S. cerevisiae in corn ethanol fermentations.

### Conclusions:

The new peptidase is effective at boosting ethanol levels in fermentation, both on its own and in concert with FermGen®.
Ethanol yield when the tripeptidyl peptidase of the invention is used is much higher than that seen when Protease A (Provia® protease (sourced from Nocardiopsis and which is a serine protein which has both endoprotease and exopeptidase activity) commercially available from Novozymes A/S) is used.
Unusually high levels of smaller sugars in the mid-range timepoints as well as significant wait times for HPLC analysis may indicate that the simple acid-kill step for inactivating enzymes in fermentation samples may be insufficient.

### Introduction:

A tripeptidogenic peptidase, Sedolisin 3PP from Trichoderma reesei was isolated and tested with the acid-fungal protease FermGen®. This peptidase has high potential as an additive in corn ethanol fermentations as a supplier of nitrogen to the yeast in the form of tripeptides. In this example fermentations will be performed with the tripeptidyl peptidase, FermGen®, and Spezyme® FAN (an alkaline protease produced in Bacillus) individually and together.

### Materials:

### Liquefact: Lincolnway Energy

### Yeast: Red Star, Ethanol Red

Urea: JT Baker, USP grade, Lot no. A23338

TABLE 4
Enzymes Used in this Experiment

| Enzyme | Lot no. | Sticker no. | Activity | | SG |
|---|---|---|---|---|---|
| Trichoderma | 1681721701 | 2011-0651 | 1134 | GAU/g | 1.13 |
| reesei | | | | | |
| glucoamylase | | | | | |
| Aspergillus | 1681359791 | 2012-0003 | 10562 | SSU/g | 1.17 |
| kawachi alpha | | | | | |
| amylase | | | | | |
| Spirizyme ® Excel | Unknown | 2011-0119 | Unknown | | 1.16 |
| (available from | | | | | |
| Novozymes) | | | | | |
| FermGen ® | 1681719474 | 2012-0093 | 1018 | SAPU/g | 1.17 |
| (available from | | | | | |
| DuPont Industrial | | | | | |
| Biosciences - | | | | | |
| formerly | | | | | |
| Genencor) | | | | | |
| Tripeptidyl | N/A | 2012-0024 | 2358 | U/g | 1.06 |
| Peptidase | | | | | |

| | | | | |
|---|---|---|---|---|
| Spezyme ® FAN | 1661306808 | 2011-0020 | N/A | 1.09 |
| (available from | | | | |
| DuPont Industrial | | | | |
| Biosciences - | | | | |
| formerly | | | | |
| Genencor) | | | | |
| Protease A | Unknown | 2011-0644 | Unknown | 1.23 |
| (Provia ® protease | | | | |
| available from | | | | |
| Novozymes A/S) | | | | |

### Experimental:

Liquefact from Lincolnway Energy was thawed in a 60° C. waterbath then the amount needed for each experiment was weighed into a beaker. Solid urea was added to yield a final concentration of either 400 ppm. The pH was adjusted to 4.5 with 6N H₂SO₄ prior to % DS determination by moisture balance. Yeast was weighed to yield a final concentration of 0.1% of the total mass and dry-pitched into the liquefact, followed by thorough mixing. The liquefact was then dispensed in 100 g quantities into 250 ml wide-mouthed Erlenmeyer flasks in triplicate for each condition to be tested. Enzymes were dosed as in Table 5.

TABLE 5
Flask Conditions and Enzyme Dosages for 400 ppm Urea Fermentations
Table 5: Distillase ® SSF dosed at 0.325 GAU/g DS; Spirizyme ®
Excel dosed at 0.058% wt/wt as is corn. Peptidase dosed at 0.02
U/g DS; Spezyme ® FAN and Protease A dosed at 0.1 kg/MT DS.

| | | | Spezyme ® | | Prot |
|---|---|---|---|---|---|
| Flask | Description | μl GA | FAN | Peptidase | A |
| 1, 2, 3 | Distillase ® | 109.5 | | | |
| | SSF control | | | | |
| 4, 5, 6 | 3PP | 109.5 | | 11.90 | |
| 7, 8, 9 | Spezyme ® | 109.5 | 27.2 | | |
| | FAN | | | | |
| 10, 11, 12 | FAN + 3PP | 109.5 | 27.2 | 11.90 | |
| 13, 14, 15 | Spirizyme ® | 95.2 | | | |
| | Excel | | | | |
| 16, 17, 18 | Excel + | 95.2 | | | 27.0 |
| | Prot A | | | | |

Flasks were stoppered with single-holed, number 8-sized rubber stoppers and placed in a 1″ orbit shaker at 150 rpm and 32° C. Fermentation samples of approximately 2 ml were collected at the indicated timepoints and prepped by spinning down solids for 3 minutes at 16,000×g, collecting 500 μl of supernatant, inactivating enzymes with 50 μl of 1N H₂SO₄ for 5 minutes, diluting 1:10 in diH₂O and passing through a 13 mm diameter, 0.45 μm pore size nylon filter. Collected samples were analyzed by HPLC under the following conditions:
Column: Phenomenex Rezex Organic Acid Column
Mobile Phase: 0.05N H₂SO₄
Detector: Refractive index
Temperature: 65° C.
Injection Volume: 20 μl
Separation time: 23 minutes

FIG. 4: While the tripeptidyl peptidase produced higher levels of ethanol than the control; the only sample without any protease, Spirizyme® Excel (a product containing glucoamylase or a glucoamylase/amylase combination), failed to reach 16% ethanol by volume and had elevated glucose levels at the end of fermentation. This likely indicates that, at only 400 ppm urea, the yeast cells are nitrogen starved. 400 ppm was chosen such that the fermentations would finish, but benefits from added proteases would still be observable; however, the fermentations didn't finish when no protease was added so this number will need to be increased to the standard 600 ppm dose.

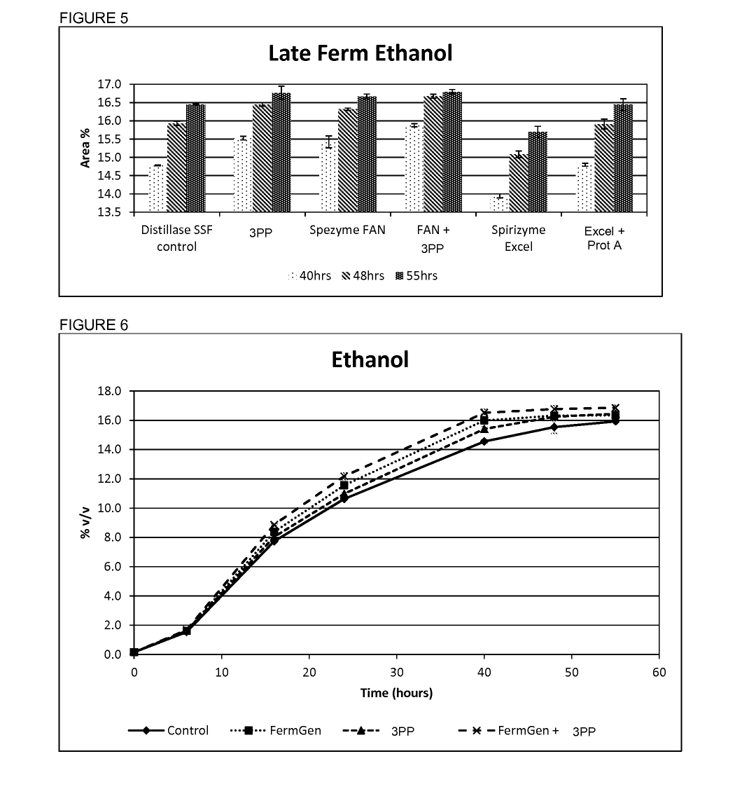

FIG. 5: The benefit of the tripeptidyl peptidase addition is easily observed. Interestingly, Spezyme® FAN showed an increase in ethanol production. A rate increase was observed when the two were added to the FermGen® already in Distillase® SSF (available from DuPont Industrial Biosciences—formerly Genencor).

Liquefact for another experiment was prepared as the first one except that urea was added to a final concentration of 600 ppm. Additionally, a 2 ml sample of the remaining liquefact was collected in triplicate as a 0 timepoint. Enzymes were dosed as shown in Table 6.

TABLE 6
Conditions and Enzyme Dosages for 600 ppm Urea Fermentations

| | | 1:5 dilution | | 1:100 dilution | | | |
|---|---|---|---|---|---|---|---|
| | | | | μl | μl | μl | μl |
| | | μl | μl | Ferm- | Pepti- | Spez. | Prot. |
| Flask | Description | GA | AkAA | Gen | dase | FAN | A |
| 1, 2, 3 | Control | 38.8 | 24.2 | | | | |
| 4, 5, 6 | FermGen ® | 38.8 | 24.2 | 51.4 | | | |
| 7, 8, 9 | 3PP | 38.8 | 24.2 | | 24.5 | | |
| 10, 11, | FermGen ® + | 38.8 | 24.2 | 51.4 | 24.5 | | |
| 12 | 3PP | | | | | | |
| 13, 14, | Spezyme ® | 38.8 | 24.2 | | | 70.2 | |
| 15 | FAN | | | | | | |
| 16, 17, | Spirizyme ® | 85.6 | | | | | |
| 18 | Excel | | | | | | |
| 19, 20, | Excel ® + | 85.6 | | | | | 62.3 |
| 21 | Protease A | | | | | | |

Table 6: Glucoamylases were dosed as described in Table 2 with AkAA dosed at 1.95 ^{SSU}/_{g DS}. FermGen ® and tripeptidyl peptidase were dosed at 0.02 ^{SAPU}/_{g DS} and Spezyme ® FAN and Protease A were dosed at 0.25 kg/MT DS.

FIG. 6: Both FermGen® and the new tripeptidyl peptidase generated higher ethanol levels in fermentations and an additive effect was observed when the two were added together.

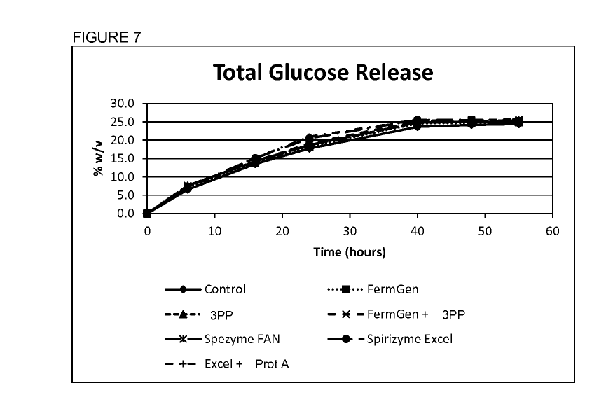

FIG. 7: The total glucose release was about 1% high for the % DS of this liquefact.

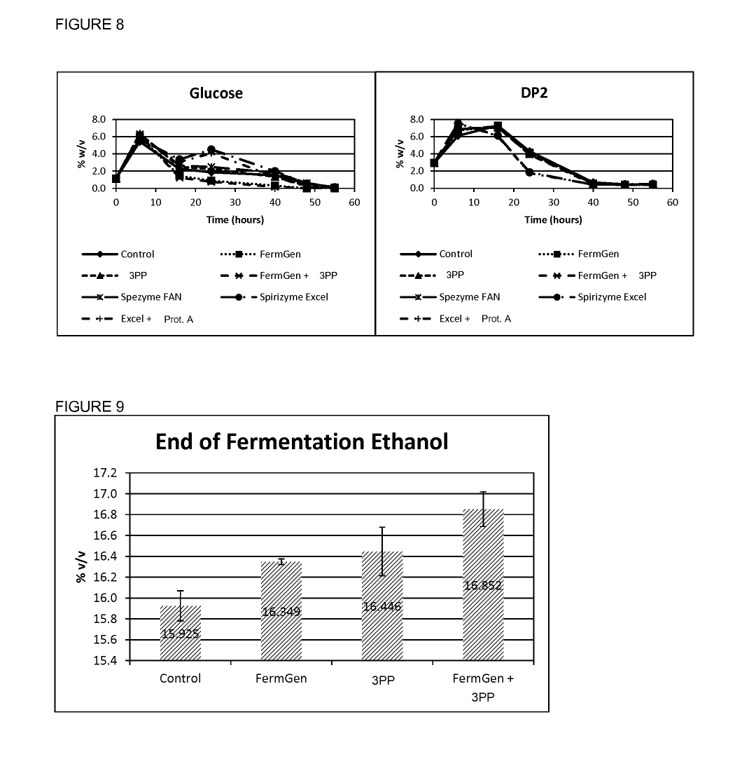

FIG. 8: The glucose and DP2 levels show some unusual trending in these particular fermentations. The glucose is higher than usual throughout most of the fermentation and the when the Protease A trials were run increased levels between the 16 and 24 hour timepoints were observed. This glucose increase is also seen with a concomitant decrease in DP2 at 24 hours. Given the amount of time between collection of the samples and analysis due to the load on HPLC System 5, it is believed that this may suggest that the acid-kill step is insufficient in completely inactivating the glucoamylase enzymes; especially those in Spirizyme® Excel.

### Example 3

### Materials:

### Liquefact: Big River Dyersville (May 4, 2012)

### Yeast: Red Star, Ethanol Red

Urea: JT Baker, USP grade, Lot no. A23338

TABLE 7
Enzymes Used in this Experiment

| Enzyme | Lot no. | Sticker no. | Activity | | SG |
|---|---|---|---|---|---|
| Trichoderma | 1681721701 | 2011-0651 | 1134 | GAU/g | 1.13 |
| reesei | | | | | |
| glucoamylase | | | | | |
| Aspergillus | 1681359791 | 2012-0003 | 10562 | SSU/g | 1.17 |
| kawachi alpha | | | | | |
| amylase | | | | | |
| FERMGEN ® | 1681719474 | 2012-0093 | 1018 | SAPU/g | 1.17 |
| 3PP Peptidase | N/A | 2012-0024 | 2358 | U/g | 1.06 |

### Experimental:

Whole ground corn liquefact was thawed in a 60° C. waterbath then the amount needed for each was weighed into a beaker. Solid urea was added to yield a final concentration of 600 ppm. The pH was adjusted to 4.5 with 6N H₂SO₄ and % DS was determined by moisture balance prior to the collection of a 0 timepoint in triplicate. Yeast was weighed to yield a final concentration of 0.1% of the total mass and dry-pitched into the liquefact, followed by thorough mixing. The liquefact was then dispensed in 100 g quantities into 250 ml wide-mouthed Erlenmeyer flasks in triplicate for each condition to be tested. Enzymes were dosed as in Table 8.

TABLE 8
Flask Conditions and Enzyme Dosages for the 600 ppm Urea Fermentation
Table 8: Trichoderma reesei glucoamylase and AkAA were dosed at
0.325 GAU/g DS and 1.95 SSU/g DS, respectively. Both proteases
were dosed at 0.02 U/g DS.

| | | 1:5 dilution | | 1:100 dilution | |
|---|---|---|---|---|---|
| | | μl | μl | μl | μl |
| Flask | Description | GA | AkAA | FermGen | Peptidase |
| 1, 2, 3 | Control | 38.8 | 24.1 | | |
| 4, 5, 6 | Fermgen ® | 38.8 | 24.1 | 51.3 | |
| 7, 8, 9 | 3PP | 38.8 | 24.1 | | 24.5 |
| 10, 11, 12 | Fermgen ® + | 38.8 | 24.1 | 51.3 | 24.5 |
| | 3PP | | | | |
| 13, 14, 15 | 2X Fermgen ® | 38.8 | 24.1 | 102.5 | |
| 16, 17, 18 | 2X 3PP | 38.8 | 24.1 | | 48.9 |

Flasks were stoppered with single-holed, number 8-sized rubber stoppers and placed in a 1″ orbit shaker at 150 rpm and 32° C. Fermentation samples of approximately 2 ml were collected in triplicate at the indicated timepoints and prepped by spinning down solids for 3 minutes at 16,000×g. 600 μl of supernatant was collected into a 1.5 ml, screw-top microfuge tube, 60 μl of 1N H₂SO₄ was added and tubes were boiled for five minutes in a 99° C. heat block without shaking. Samples were then cooled and diluted 1:10 in diH₂O prior to passing through a 13 mm diameter, 0.2 μm pore size, nylon filter. Collected samples were analyzed by HPLC under the following conditions:

### Column: Phenomenex Rezex Organic Acid Column

Mobile Phase: 0.01N H₂SO₄
Detector: Refractive index
Temperature: 65° C.
Injection Volume: 20 μl
Separation time: 23 minutes

FIG. 9: The benefit to ethanol yield of the 3PP peptidase appears to be additive; since the ethanol concentration increase for the Fermgen®+tripeptidyl peptidase sample is similar to the individual benefits of Fermgen® and tripeptidyl peptidase (1.959% vs. 2.288%, respectively).

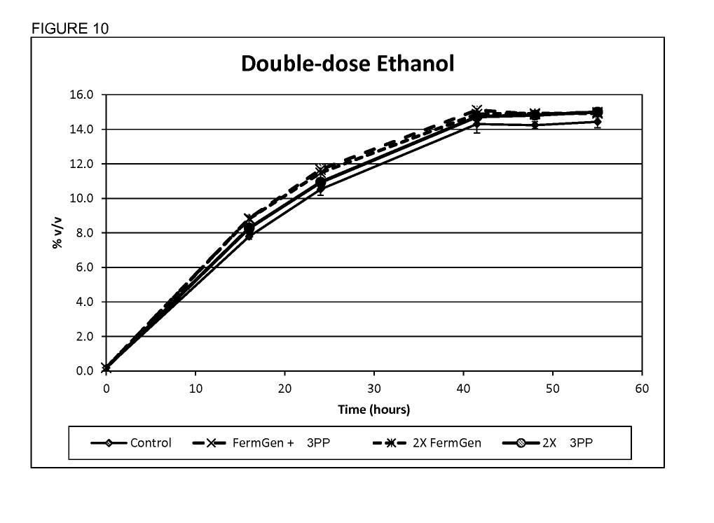

FIG. 10: When the peptidase is combined with Fermgen® a rate similar to that of a double dose of Fermgen® is observed. A double-dose of the peptidase improves the rate of fermentation, but not as much as the samples containing Fermgen®. However, a double dose of the peptidase does result in final ethanol levels similar to the samples containing Fermgen®.

### Conclusions:

This peptidase does provide a benefit to fermentation ethanol levels similar to the benefit seen with the current acid-fungal protease FERMGEN®.

### Example 4

Cloning and Expression of Tripeptidyl Peptidases in Trichoderma reesei.

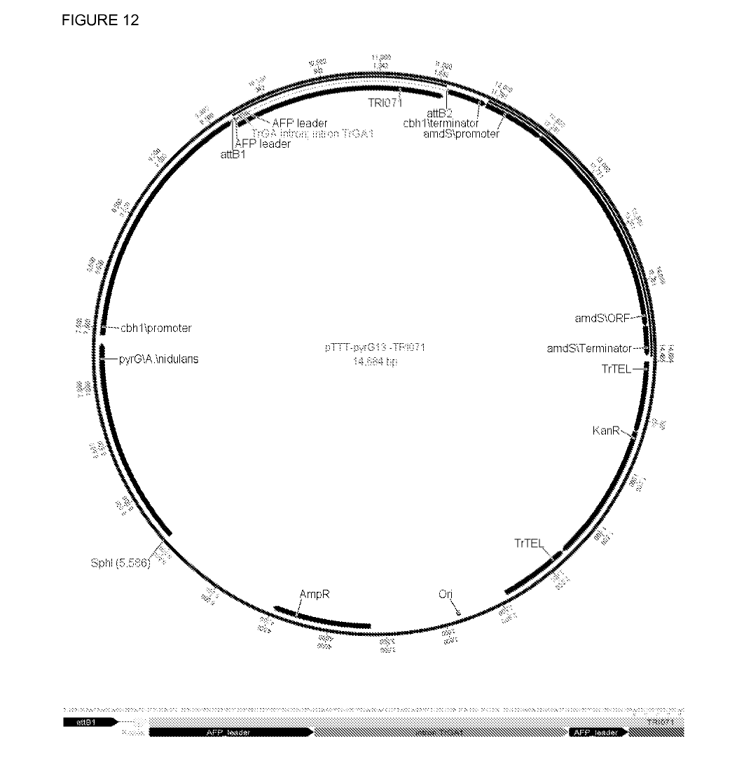

Synthetic genes encoding proline tolerant tripeptidyl peptidases were generated using preferred codons for expression in Trichoderma reesei except for TR1079 (SEQ ID No. 57) and TR1083 (SEQ ID No. 56) that were generated as genomic sequences. The predicted secretion signal sequences (SignalP 4.0: Discriminating signal peptides from transmembrane regions. Thomas Nordahl Petersen, Soren Brunak, Gunnar von Heijne & Henrik Nielsen. Nature Methods, 8:785-786, 2011) were replaced (except for TR1079 and TR1083) by the secretion signal sequence from the Trichoderma reesei acidic fungal protease (AFP) and an intron from a Trichoderma reesei glucoamylase gene (TrGA1) (see FIG. 12 lower panel).

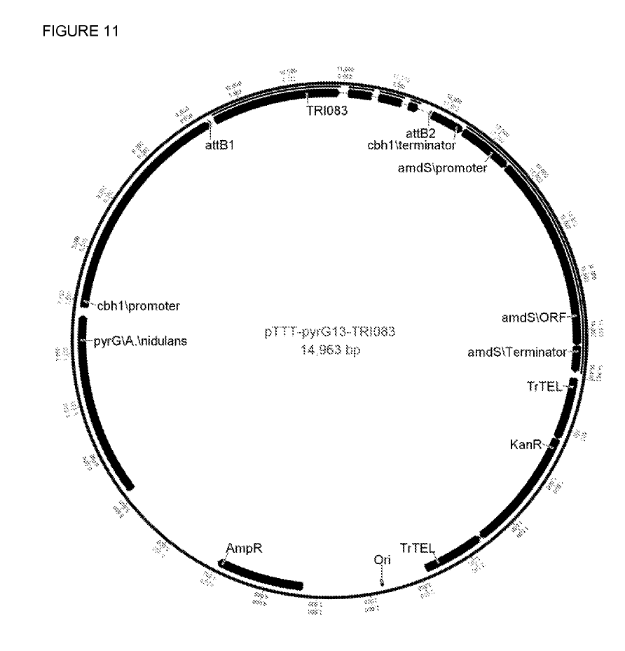

Synthetic genes were introduced into the destination vector pTTT-pyrG13 (as described in U.S. Pat. No. 8,592,194 B2 the teaching of which is incorporated herein by reference) using LR Clonase™ enzyme mix (Life Technologies) resulting in the construction of expression vectors pTTT-pyrG13 for the proline tolerant tripeptidyl peptidases herein. Expression vectors encoding SEQ ID No's 1, 2 and 29 are shown in FIG. 11 and encoding SEQ ID No's 12 and 39 are shown in FIG. 12.

5-10 μg of the expression vectors were transformed individually into a suitable Trichoderma reesei strain using PEG mediated protoplast transformation essentially as described in (U.S. Pat. No. 8,592,194 B2). Germinating spores were harvested by centrifugation, washed and treated with 45 mg/ml of lysing enzyme solution (Trichoderma harzianum, Sigma L1412) to lyse the fungal cell walls. Further preparation of protoplasts was performed by a standard method, as described by Penttilä et al. [Gene 61 (1987) 155-164] the contents of which are incorporated herein by reference.

Spores were harvested using a solution of 0.85% NaCl, 0.015% Tween 80. Spore suspensions were used to inoculate liquid cultures

Cultures were grown for 7 days at 28° C. and 80% humidity with shaking at 180 rpm. Culture supernatants were harvested by vacuum filtration and used to measure expression and enzyme performance.

### Purification of Tripeptidyl Peptidase

Desalting of samples was performed on PD10 column (GE Life Sciences, USA) equilibrated with 20 mM Na-acetate, pH 4.5 (buffer A). For ion exchange chromatography on Source S15 HR25/5 (GE Life Sciences, USA) the column was equilibrated with buffer A. The desalted sample (7 ml) was applied to the column at a flow rate of 6 ml/min and the column was washed with buffer A. The bound proteins were eluted with a linier gradient of 0-0.35 M NaCl in 20 mM Na-acetate, pH 4.5 (35 min). During the entire run 10 ml fractions were collected. The collected samples were assayed for tripeptidyl peptidase activity in accordance with the assays taught herein (e.g. EBSA assay). Protein concentration was calculated based on the absorbance measure at 280 nm and the theoretical absorbance of the protein calculated using the ExPASy ProtParam tool (http://web.expasy.org/cgi-bin/protparam/protparam).

### Example 5

### 3PP Peptidase Effect on Lactic Acid Fermentation

Bacillus coagulans is a strong lactic acid producer that can grow at temperatures up to 55° C. In this study, additional 3PP peptidase together with Spezyme Alpha was used during liquefaction for lactic acid (LA) simultaneous saccharification and fermentation (SSF) process to see if additional peptidase could hydrolyze more corn protein to amino acid as nitrogen source and result in more LA.

### Materials and Methods

Bacillus coagulans CICC 20138 was obtained from China Center of Industrial Culture Collection. Spezyme Alpha: 7201870341, 14264AAU/g, 2014.09.05. TrGA Conc.: 805TGAU/g, Lot 7202033738. 3PP peptidase cedar 2014-11-03

Liquefaction conditions are shown in Table 9. 15% DS corn was adjusted the pH to 5.7. One sample contained 0.3 Kg/MT Spezyme Alpha only as control; the other two contained 0.3 Kg/MT Alpha and 3PP peptidase at 0.1 or 0.5 Kg/MT. All samples were incubated at 65° C. for 30 min at 350 rpm, and then at 87° C., 350 rpm holding for 90 min. After liquefaction, the liquefact was centrifuged and filtered.

Seed medium (per litre) contained peptone 10.0 g, yeast extract 5.0 g, NaCl 5.0 g and glucose 5.0 g at pH 6.0. The medium was sterilised at 121° C. for 20 min. The seed was cultivated at 50° C., 130 rpm for approximately 18 hrs.

Fermentation medium for a 1 L fermentor contained 500 g corn liquefact, 50 ml strain seed. Simultaneous saccharification and fermentation (SSF) was carried out at 50° C., rotation speed 300 rpm, pH6.5 automatically adjusted by 20% (m/v) NH4OH. 0.75 TGAU/gds TrGA was added and then 50 mL seed to start fermentation. Agitation was maintained at 300 rpm and pH was kept constant at 6.5 using 20% (m/v) NH4OH. Samples were taken for HPLC analysis.

TABLE 9
Liquefaction conditions

| | Pretreatment | Liquefaction |
|---|---|---|
| 1 | 65° C., 30 min, Alpha@0.3 Kg/MT | 87° C., 90 min |
| 2 | 65° C., 30 min, Alpha@0.3 Kg/MT + | 87° C., 90 min |
| | Phytase@0.1 Kg/MT | |
| 3 | 65° C., 30 min, Alpha@0.3 Kg/MT + | 87° C., 90 min |
| | Phytase@0.1 Kg/MT | |

TABLE 10
Fermentation conditions

| | | | | | | Final |
|---|---|---|---|---|---|---|
| | | TrGA | | Temp. | | volume |
| Fermentor | | (TGAU/g) | Rotate | ° C. | pH | (ml) |
| 1, | Control | 0.75 | 300 rpm | 50 | 6.5 | 580 |
| 2 | 3PP | 0.75 | 300 rpm | 50 | 6.5 | 580 |
| | peptidase@0.1 | | | | | |
| | Kg/MT | | | | | |
| 3 | 3PP | 0.75 | 300 rpm | 50 | 6.5 | 590 |
| | peptidase@0.5 | | | | | |
| | Kg/MT | | | | | |

### Results

Compared with control, pre-treatment with 3PP peptidase before corn liquefaction resulted in:

1) Higher corn liquefact filtration speed, and lower residual starch in corn cake;
2) Higher lactic acid production and lower residual glucose during LA SSF process.

It appears that 0.1 Kg/tds dose was sufficient for pretreatment.

Since there are no external protein nutrients for LA SSF process, and the glucose was not entirely consumed at 70 hrs, it seems that the peptidase addition hydrolyzed some corn protein to amino acids, as nitrogen increased fermentation rate and produced higher LA yield and lower residual glucose.

TABLE 11
liquefaction performance

| | | Residual | |
|---|---|---|---|
| | | sugar | |
| | | (%) of | filtration |
| | brix | corn cake | speed |
| Sample | (%) | by NIR | (mL/min) |
| Control | 11.5 | 16.37 | 10 |
| 3PP peptidase@0.1 Kg/MT | 11.4 | 14.81 | 13.25 |
| 3PP peptidase@0.5 Kg/MT | 11.4 | 15.59 | 13 |

TABLE 12
fermentation performance

| | | | (m/v) % | Lactic | Acetic |
|---|---|---|---|---|---|
| | Time(hrs) | DP2 | Glucose | acid | acid |
| Control | 5 | 2.883 | 4.503 | 1.276 | 0.000 |
| 3PP peptidase@0.1 Kg/MT | 5 | 2.766 | 5.004 | 1.149 | 0.000 |
| 3PP peptidase@0.5 Kg/MT | 5 | 2.629 | 4.951 | 1.267 | 0.000 |
| Control | 70 | 0.484 | 2.532 | 6.325 | 0.131 |
| 3PP peptidase@0.1 Kg/MT | 70 | 0.354 | 1.642 | 7.892 | 0.160 |
| 3PP peptidase@0.5 Kg/MT | 70 | 0.325 | 1.584 | 7.713 | 0.229 |
| Control | 5 | 2.883 | 4.503 | 1.276 | 0.000 |
| 3PP peptidase@0.1 Kg/MT | 5 | 2.766 | 5.004 | 1.149 | 0.000 |
| 3PP peptidase@0.5 Kg/MT | 5 | 2.629 | 4.951 | 1.267 | 0.000 |
| Control | 70 | 0.484 | 2.532 | 6.325 | 0.131 |
| 3PP peptidase@0.1 Kg/MT | 70 | 0.354 | 1.642 | 7.892 | 0.160 |
| 3PP peptidase@0.5 Kg/MT | 70 | 0.325 | 1.584 | 7.713 | 0.229 |

TABLE 13
lactic acid yield

| | Fermen- | | | | | LA |
|---|---|---|---|---|---|---|
| | tation | | Residual | | LA | yield % |
| | time | | glucose | Lactic | yield | remove |
| Sample | (hrs) | Brix(g) | (g) | acid(g) | (%) | RS |
| Control | 70 | 57.5 | 14.68 | 36.69 | 63.80 | 85.68 |
| 3PP | 70 | 57 | 9.69 | 46.56 | 81.69 | 98.41 |
| peptidase@0.1 | | | | | | |
| Kg/MT | | | | | | |
| 3PP | 70 | 57 | 9.19 | 44.73 | 78.48 | 93.56 |
| peptidase@0.5 | | | | | | |
| Kg/MT | | | | | | |

### Example 6

### The Effect of 3pp Peptidase on Citric Acid Fermentation

### Materials and Methods:

DS=20% of corn flour slurry was prepared. 3PP peptidase was added at 0.2 kg/tds based on the dry substance and incubated at 60° C. for 40 min. The control test was performed under the same condition but without 3PP peptidase. Spezyme Alpha was added at 0.3 kg/tds, then liquefaction was carried out at 90 C for 1.5 hr. The slurry was centrifuged and the supernatant used as fermentation medium. The medium was sterilized at 115 C for 15 min and then cooled.

A strain of Aspergillus niger was grown on potato dextrose agar (PDA) slant at 35° C. for 5-7 d, then the spores were washed with sterilized water, and spores suspension was inoculated into fermentation medium and incubated at 300 rpm/min, 35° C. for 96 hr. Samples were taken at the end of fermentation. The fermentation broth was filtrated through filter paper, and the culture filtrate was used for analysis. The citric acid concentration and DP¹, DP², DP³, DP⁴⁺ concentration was analyzed by HPLC.

### Results:

TABLE 14

| | DP1 | DP2 | DP3 | DP4+ | CA |
|---|---|---|---|---|---|
| Conditions | (g/L) | (g/L) | (g/L) | (g/L) | (g/l) |
| 3pp peptidase @ 0.2 kg/tds, | 0.514 | 7.3 | 1.275 | 2.913 | 112.11 ± 0.67 |
| 60 C., 40 min | | | | | |
| Alpha @ 0.3 kg/tds, | | | | | |
| 90 C., 1.5 hr | | | | | |
| 60 C. 40 min | 0.584 | 8.646 | 1.52 | 2.932 | 105.69 ± 2.17 |
| Alpha @ 0.3 kg/tds, | | | | | |
| 90 C. 1.5 hr | | | | | |

The data showed that with the 3PP peptidase addition in pretreatment process, the final citric acid yield was significantly increased, while the residual sugar was significantly decreased. After liquefaction and centrifugation, we found that the supernatant was clearer compared to the control, which could result in better filtration performance in industrial production. At the end of fermentation, we also found that the viscosity was decreased remarkably, which could improve the downstream process.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for producing an alcohol comprising:
(a) admixing a tripeptidyl peptidase, predominantly having exopeptidase activity, with a feedstock or a fraction thereof before, during or after fermentation of said feedstock or a fraction thereof; and
(b) recovering an alcohol.

2. A method according to claim 1, wherein said tripeptidyl peptidase is expressed and secreted by an alcohol production host.

3. A method according to claim 2, wherein the alcohol production host co-expresses a tripeptidyl peptidase, and one or more of the enzymes selected from the group consisting of a glucoamylase, an amylase, a further starch modifying enzyme, a protease, a phytase, a cellulase, a hemicellulase (e.g. a xylanase) and combinations thereof.

4. A method according to claim 2 or claim 3, wherein the tripeptidyl peptidase is heterologous to the alcohol production host.

5. Use of a tripeptidyl peptidase, predominantly having exopeptidase activity, in the manufacture of an alcohol for improving yield of the alcohol.

6. Use of one or more tripeptidyl peptidases(s), predominantly having exopeptidase activity, in the manufacture of an alcohol for improving an alcohol production host's ability to ferment.

7. A use according to claim 6, wherein the ability of the alcohol production host to ferment is assessed by an increase in the amount of sugar (e.g. glucose) consumed during fermentation by said alcohol production host when compared to the level of sugar (e.g. glucose) consumed during fermentation by said alcohol production host not admixed with a tripeptidyl peptidase.

8. A use according to any one of claims 5-7, wherein the one or more tripeptidyl peptidase(s) is used in combination with an endoprotease.

9. A method or use according to any one of the preceding claims wherein the tripeptidyl peptidase is an exopeptidase.

10. A method or use according to any one of the preceding claims, wherein the tripeptidyl peptidase is a Trichoderma tripeptidyl peptidase.

11. A method or use according to any one of the preceding claims wherein the tripeptidyl peptidase is capable of cleaving tri-peptides from the N-terminus of peptides having proline in position P1 and/or P1′.

12. A method or use according to any one of the preceding claims wherein the tripeptidyl peptidase is capable of cleaving tri-peptides from the N-terminus of peptides having:
Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; and/or
peptides having:
Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′.

13. A method or use according any one of the preceding claims, wherein the at least one proline tolerant tripeptidyl peptidase:
(a) comprises the amino acid sequence SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof;
(b) comprises an amino acid having at least 70% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or a functional fragment thereof;
(c) is encoded by a nucleotide sequence comprising the sequence SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95;
(d) is encoded by a nucleotide sequence comprising at least about 70% sequence identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95;
(e) is encoded by a nucleotide sequence which hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95 under medium stringency conditions; or
(f) is encoded by a nucleotide sequence which differs from SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95 due to degeneracy of the genetic code.

14. A method or use according to any one of the preceding claims, wherein said at least one proline tolerant tripeptidyl peptidase is encoded by a nucleotide sequence comprising SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95 or a nucleotide sequence having at least 90% identity thereto or a sequence which hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94 or SEQ ID No. 95 under high stringency conditions.

15. A method according to any one of the preceding claims wherein the feedstock has been subjected to one or more processing steps selected from the group consisting of: milling, cooking, saccharification, fermentation and simultaneous saccharification and fermentation.

16. A method according to claim 15 wherein the tripeptidyl peptidase is admixed during any one or more of the processing steps, preferably wherein the tripeptidyl peptidase is admixed during simultaneous saccharification and fermentation.

17. A method according to any one of the preceding claims wherein one or more endoprotease(s) is further admixed.

18. A method according to any one of the preceding claims wherein the alcohol is a biofuel (e.g. an ethanol, a butanol or a combination thereof).

19. A method according to any one of the preceding claims, wherein the feedstock or the fraction thereof is a starch, a grain or cereal-based material (e.g. a cereal, wheat, barley, rye, rice, triticale, millet, milo, sorghum or corn), a tuber (e.g. potato or cassava), a root, a sugar (e.g. cane sugar, beet sugar, molasses or a sugar syrup), stillage, wet cake, DDGS, cellulosic biomass, a hemicellulosic biomass, a whey protein, soy based material, lignocellulosic biomass or combinations thereof.

20. A method according to claim 19 wherein the lignocellulosic biomass is any cellulosic or lignocellulosic material, for example agricultural residues, bioenergy crops, industrial solid waste, municipal solid waste, sludge from paper manufacture, yard waste, wood waste, forestry waste and combinations thereof.

21. A method according to claim 19 or 20 wherein the lignocellulosic biomass is selected from the group consisting of corn cobs, crop residues such as corn husks, corn gluten meal, corn stover, corn fiber, grasses, beet pulp, wheat straw, wheat chaff, oat straw, wheat middlings, wheat shorts, rice bran, rice hulls, wheat bran, oat hulls, wet cake, Distillers Dried Grain (DDG), Distillers Dried Grain Solubles (DDGS), palm kernel, citrus pulp, cotton, lignin, barley straw, hay, rice straw, rice hulls, switchgrass, miscanthus, cord grass, reed canary grass, waste paper, sugar cane bagasse, sorghum bagasse, forage sorghum, sorghum stover, soybean stover, soy, components obtained from milling of trees, branches, roots, leaves, wood chips, sawdust, shrubs and bushes, vegetables, fruits and flowers.

22. A method according to claim 19, wherein the grain-based material is one or more selected from the group consisting of: corn, wheat, barley, oats, rye, maize, millet, rice, cassava and sorghum.

23. A method according to any one of the preceding claims wherein the tripeptidyl peptidase(s) increases the concentration of tripeptides in the fermentation mixture when compared to a fermentation mixture not comprising one or more tripeptidyl peptidase(s).

24. A method according to any one of the preceding claims, wherein the tripeptidyl peptidase is admixed with the feedstock after fermentation and the admixture is further milled.

25. A method according to any one of the preceding claims wherein one or more additional fermentation is carried out.

26. A method or use according to any one of the preceding claims, wherein said method or use further comprises the use of one or more cellulase activity, hemicellulase activity (e.g. xylanase activity), further enzyme activity or a combination thereof.

27. A method or use according to claim 26, wherein the one or more cellulase activity, hemicellulase activity, further enzyme activity or combination thereof is selected from the group consisting of: one or more of the enzymes selected from the group consisting of: endoglucanases (E.C. 3.2.1.4); cellobiohydrolases (E.C. 3.2.1.91), β-glucosidases (E.C. 3.2.1.21), cellulases (E.C. 3.2.1.74), lichenases (E.C. 3.1.1.73), lipases (E.C. 3.1.1.3), lipid acyltransferases (generally classified as E.C. 2.3.1.x), phospholipases (E.C. 3.1.1.4, E.C. 3.1.1.32 or E.C. 3.1.1.5), phytases (e.g. 6-phytase (E.C. 3.1.3.26) or a 3-phytase (E.C. 3.1.3.8), acid phosphatase, amylases, alpha-amylases (E.C. 3.2.1.1), xylanases (e.g. endo-1,4-β-d-xylanase (E.C. 3.2.1.8) or 1,4 β-xylosidase (E.C. 3.2.1.37) or E.C. 3.2.1.32, E.C. 3.1.1.72, E.C. 3.1.1.73), glucoamylases (E.C. 3.2.1.3), pullulanases, hemicellulases, proteases (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)), debranching enzymes, cutinases, esterases and/or mannanases (e.g. a β-mannanase (E.C. 3.2.1.78)) transferases, glucosidases, arabinofuranosidase.

28. A by-product of alcohol production obtainable (e.g. obtained) by the method of any one of claim 1-4 or 9-27.

29. A by-product of alcohol production according to claim 28 wherein said by-product of alcohol production is substantially enriched in one or more tripeptides.

30. A by-product of alcohol production according to claim 29 wherein the by-product of alcohol production is substantially enriched in one or more tripeptides having proline at the N-terminal, at the C-terminal or a combination thereof.

31. A by-product of alcohol production according to any one of claims 28-30, wherein said by-product is whole stillage, thin stillage, wet-cake, Distillers Dried Grain (DDG) or Distillers Dried Grain Solubles (DDGS) or enriched protein DDG or DDGs, or protein fraction.

32. A by-product of alcohol production according to any one of claims 28-31, wherein the alcohol production process is a biofuel production process.

33. A method, use or by-product of alcohol production substantially as described herein with reference to the description, examples and figures.
